SEQUENCE LISTING (1188 sequences)

SEQ ID NO: 1 - DNA, 66 bp, Streptococcus mutans
ttcaaaagttggagcctttgtacgcctggttgtgcaaggacaggtagtttcaatagttactgttgc

SEQ ID NO: 2 - DNA, 66 bp, Artificial Sequence
  source: /note=“Description of Artificial Sequence Synthetic oligonucleotide”
tggaaaagttggagcctttgtacgcctggttgtgcaaggacaggtagtttcaatagttactgttgc

SEQ ID NO: 3 - DNA, 66 bp, Artificial Sequence
  source: /note=“Description of Artificial Sequence Synthetic oligonucleotide”
gctaaaagttggagcctttgtacgcctggttgtgcaaggacaggtagtttcaatagttactgttgc

SEQ ID NO: 4 - DNA, 66 bp, Artificial Sequence
  source: /note=“Description of Artificial Sequence Synthetic oligonucleotide”
cttaaaagttggagcctttgtacgcctggttgtgcaaggacaggtagtttcaatagttactgttgc

SEQ ID NO: 5 - DNA, 66 bp, Artificial Sequence
  source: /note=“Description of Artificial Sequence Synthetic oligonucleotide”
atgaaaagttggagcctttgtacgcctggttgtgcaaggacaggtagtttcaatagttactgttgc

SEQ ID NO: 6 - DNA, 66 bp, Artificial Sequence
  source: /note=“Description of Artificial Sequence Synthetic oligonucleotide”
tgtaaaagttggagcctttgtacgcctggttgtgcaaggacaggtagtttcaatagttactgttgc

SEQ ID NO: 7 - DNA, 66 bp, Artificial Sequence
  source: /note=“Description of Artificial Sequence Synthetic oligonucleotide”
aataaaagttggagcctttgtacgcctggttgtgcaaggacaggtagtttcaatagttactgttgc

SEQ ID NO: 8 - DNA, 66 bp, Artificial Sequence
  source: /note=“Description of Artificial Sequence Synthetic oligonucleotide”
tataaaagttggagcctttgtacgcctggttgtgcaaggacaggtagtttcaatagttactgttgc

SEQ ID NO: 9 - DNA, 66 bp, Artificial Sequence
  source: /note=“Description of Artificial Sequence Synthetic oligonucleotide”
gataaaagttggagcctttgtacgcctggttgtgcaaggacaggtagtttcaatagttactgttgc

SEQ ID NO: 10 - DNA, 66 bp, Artificial Sequence
  source: /note=“Description of Artificial Sequence Synthetic oligonucleotide”
gaaaaaagttggagcctttgtacgcctggttgtgcaaggacaggtagtttcaatagttactgttgc

SEQ ID NO: 11 - DNA, 66 bp, Artificial Sequence
  source: /note=“Description of Artificial Sequence Synthetic oligonucleotide”
cctaaaagttggagcctttgtacgcctggttgtgcaaggacaggtagtttcaatagttactgttgc

SEQ ID NO: 12 - DNA, 66 bp, Artificial Sequence
  source: /note=“Description of Artificial Sequence Synthetic oligonucleotide”
caaaaaagttggagcctttgtacgcctggttgtgcaaggacaggtagtttcaatagttactgttgc

SEQ ID NO: 13 - DNA, 66 bp, Artificial Sequence
  source: /note=“Description of Artificial Sequence Synthetic oligonucleotide”
tttaaaagttggagcctttgtacgcctggttgtgcaaggacaggtagtttcaatagttactgttgc

SEQ ID NO: 14 - DNA, 66 bp, Artificial Sequence
  source: /note=“Description of Artificial Sequence Synthetic oligonucleotide”
ggtaaaagttggagcctttgtacgcctggttgtgcaaggacaggtagtttcaatagttactgttgc

SEQ ID NO: 15 - DNA, 66 bp, Artificial Sequence
  source: /note=“Description of Artificial Sequence Synthetic oligonucleotide”
cgtaaaagttggagcctttgtacgcctggttgtgcaaggacaggtagtttcaatagttactgttgc

SEQ ID NO: 16 - DNA, 66 bp, Artificial Sequence
  source: /note=“Description of Artificial Sequence Synthetic oligonucleotide”
cataaaagttggagcctttgtacgcctggttgtgcaaggacaggtagtttcaatagttactgttgc

SEQ ID NO: 17 - DNA, 66 bp, Artificial Sequence
  source: /note=“Description of Artificial Sequence Synthetic oligonucleotide”
tctaaaagttggagcctttgtacgcctggttgtgcaaggacaggtagtttcaatagttactgttgc

SEQ ID NO: 18 - DNA, 66 bp, Artificial Sequence
  source: /note=“Description of Artificial Sequence Synthetic oligonucleotide”
acaaaaagttggagcctttgtacgcctggttgtgcaaggacaggtagtttcaatagttactgttgc

SEQ ID NO: 19 - DNA, 66 bp, Artificial Sequence
  source: /note=“Description of Artificial Sequence Synthetic oligonucleotide”
attaaaagttggagcctttgtacgcctggttgtgcaaggacaggtagtttcaatagttactgttgc

SEQ ID NO: 20 - DNA, 66 bp, Artificial Sequence
  source: /note=“Description of Artificial Sequence Synthetic oligonucleotide”
ttcgttagttggagcctttgtacgcctggttgtgcaaggacaggtagtttcaatagttactgttgc

SEQ ID NO: 21 - DNA, 66 bp, Artificial Sequence
  source: /note=“Description of Artificial Sequence Synthetic oligonucleotide”
ttctggagttggagcctttgtacgcctggttgtgcaaggacaggtagtttcaatagttactgttgc

SEQ ID NO: 22 - DNA, 66 bp, Artificial Sequence
  source: /note=“Description of Artificial Sequence Synthetic oligonucleotide”
ttcgctagttggagcctttgtacgcctggttgtgcaaggacaggtagtttcaatagttactgttgc

SEQ ID NO: 23 - DNA, 66 bp, Artificial Sequence
  source: /note=“Description of Artificial Sequence Synthetic oligonucleotide”
ttcttaagttggagcctttgtacgcctggttgtgcaaggacaggtagtttcaatagttactgttgc

SEQ ID NO: 24 - DNA, 66 bp, Artificial Sequence
  source: /note=“Description of Artificial Sequence Synthetic oligonucleotide”
ttcatgagttggagcctttgtacgcctggttgtgcaaggacaggtagtttcaatagttactgttgc

SEQ ID NO: 25 - DNA, 66 bp, Artificial Sequence
  source: /note=“Description of Artificial Sequence Synthetic oligonucleotide”
ttctgtagttggagcctttgtacgcctggttgtgcaaggacaggtagtttcaatagttactgttgc

SEQ ID NO: 26 - DNA, 66 bp, Artificial Sequence
  source: /note=“Description of Artificial Sequence Synthetic oligonucleotide”
ttcaatagttggagcctttgtacgcctggttgtgcaaggacaggtagtttcaatagttactgttgc

SEQ ID NO: 27 - DNA, 66 bp, Artificial Sequence
  source: /note=“Description of Artificial Sequence Synthetic oligonucleotide”
ttctatagttggagcctttgtacgcctggttgtgcaaggacaggtagtttcaatagttactgttgc

SEQ ID NO: 28 - DNA, 66 bp, Artificial Sequence
  source: /note=“Description of Artificial Sequence Synthetic oligonucleotide”
ttcgatagttggagcctttgtacgcctggttgtgcaaggacaggtagtttcaatagttactgttgc

SEQ ID NO: 29 - DNA, 66 bp, Artificial Sequence
  source: /note=“Description of Artificial Sequence Synthetic oligonucleotide”
ttcgaaagttggagcctttgtacgcctggttgtgcaaggacaggtagtttcaatagttactgttgc

SEQ ID NO: 30 - DNA, 66 bp, Artificial Sequence
  source: /note=“Description of Artificial Sequence Synthetic oligonucleotide”
ttccctagttggagcctttgtacgcctggttgtgcaaggacaggtagtttcaatagttactgttgc

SEQ ID NO: 31 - DNA, 66 bp, Artificial Sequence
  source: /note=“Description of Artificial Sequence Synthetic oligonucleotide”
ttccaaagttggagcctttgtacgcctggttgtgcaaggacaggtagtttcaatagttactgttgc

SEQ ID NO: 32 - DNA, 66 bp, Artificial Sequence
  source: /note=“Description of Artificial Sequence Synthetic oligonucleotide”
ttctttagttggagcctttgtacgcctggttgtgcaaggacaggtagtttcaatagttactgttgc

SEQ ID NO: 33 - DNA, 66 bp, Artificial Sequence
  source: /note=“Description of Artificial Sequence Synthetic oligonucleotide”
ttcggtagttggagcctttgtacgcctggttgtgcaaggacaggtagtttcaatagttactgttgc

SEQ ID NO: 34 - DNA, 66 bp, Artificial Sequence
  source: /note=“Description of Artificial Sequence Synthetic oligonucleotide”
ttccgtagttggagcctttgtacgcctggttgtgcaaggacaggtagtttcaatagttactgttgc

SEQ ID NO: 35 - DNA, 66 bp, Artificial Sequence
  source: /note=“Description of Artificial Sequence Synthetic oligonucleotide”
ttccatagttggagcctttgtacgcctggttgtgcaaggacaggtagtttcaatagttactgttgc

SEQ ID NO: 36 - DNA, 66 bp, Artificial Sequence
  source: /note=“Description of Artificial Sequence Synthetic oligonucleotide”
ttctctagttggagcctttgtacgcctggttgtgcaaggacaggtagtttcaatagttactgttgc

SEQ ID NO: 37 - DNA, 66 bp, Artificial Sequence
  source: /note=“Description of Artificial Sequence Synthetic oligonucleotide”
ttcacaagttggagcctttgtacgcctggttgtgcaaggacaggtagtttcaatagttactgttgc

SEQ ID NO: 38 - DNA, 66 bp, Artificial Sequence
  source: /note=“Description of Artificial Sequence Synthetic oligonucleotide”
ttcattagttggagcctttgtacgcctggttgtgcaaggacaggtagtttcaatagttactgttgc

SEQ ID NO: 39 - DNA, 66 bp, Artificial Sequence
  source: /note=“Description of Artificial Sequence Synthetic oligonucleotide”
ttcaaaaaatggagcctttgtacgcctggttgtgcaaggacaggtagtttcaatagttactgttgc

SEQ ID NO: 40 - DNA, 66 bp, Artificial Sequence
  source: /note=“Description of Artificial Sequence Synthetic oligonucleotide”
ttcaaagtttggagcctttgtacgcctggttgtgcaaggacaggtagtttcaatagttactgttgc

SEQ ID NO: 41 - DNA, 66 bp, Artificial Sequence
  source: /note=“Description of Artificial Sequence Synthetic oligonucleotide”
ttcaaatggtggagcctttgtacgcctggttgtgcaaggacaggtagtttcaatagttactgttgc

SEQ ID NO: 42 - DNA, 66 bp, Artificial Sequence
  source: /note=“Description of Artificial Sequence Synthetic oligonucleotide”
ttcaaagcttggagcctttgtacgcctggttgtgcaaggacaggtagtttcaatagttactgttgc

SEQ ID NO: 43 - DNA, 66 bp, Artificial Sequence
  source: /note=“Description of Artificial Sequence Synthetic oligonucleotide”
ttcaaattatggagcctttgtacgcctggttgtgcaaggacaggtagtttcaatagttactgttgc

SEQ ID NO: 44 - DNA, 66 bp, Artificial Sequence
  source: /note=“Description of Artificial Sequence Synthetic oligonucleotide”
ttcaaaatgtggagcctttgtacgcctggttgtgcaaggacaggtagtttcaatagttactgttgc

SEQ ID NO: 45 - DNA, 66 bp, Artificial Sequence
  source: /note=“Description of Artificial Sequence Synthetic oligonucleotide”
ttcaaatgttggagcctttgtacgcctggttgtgcaaggacaggtagtttcaatagttactgttgc

SEQ ID NO: 46 - DNA, 66 bp, Artificial Sequence
  source: /note=“Description of Artificial Sequence Synthetic oligonucleotide”
ttcaaaaattggagcctttgtacgcctggttgtgcaaggacaggtagtttcaatagttactgttgc

SEQ ID NO: 47 - DNA, 66 bp, Artificial Sequence
  source: /note=“Description of Artificial Sequence Synthetic oligonucleotide”
ttcaaatattggagcctttgtacgcctggttgtgcaaggacaggtagtttcaatagttactgttgc

SEQ ID NO: 48 - DNA, 66 bp, Artificial Sequence
  source: /note=“Description of Artificial Sequence Synthetic oligonucleotide”
ttcaaagattggagcctttgtacgcctggttgtgcaaggacaggtagtttcaatagttactgttgc

SEQ ID NO: 49 - DNA, 66 bp, Artificial Sequence
  source: /note=“Description of Artificial Sequence Synthetic oligonucleotide”
ttcaaagaatggagcctttgtacgcctggttgtgcaaggacaggtagtttcaatagttactgttgc

SEQ ID NO: 50 - DNA, 66 bp, Artificial Sequence
  source: /note=“Description of Artificial Sequence Synthetic oligonucleotide”
ttcaaaccttggagcctttgtacgcctggttgtgcaaggacaggtagtttcaatagttactgttgc

SEQ ID NO: 51 - DNA, 66 bp, Artificial Sequence
  source: /note=“Description of Artificial Sequence Synthetic oligonucleotide”
ttcaaacaatggagcctttgtacgcctggttgtgcaaggacaggtagtttcaatagttactgttgc

SEQ ID NO: 52 - DNA, 66 bp, Artificial Sequence
  source: /note=“Description of Artificial Sequence Synthetic oligonucleotide”
ttcaaattttggagcctttgtacgcctggttgtgcaaggacaggtagtttcaatagttactgttgc

SEQ ID NO: 53 - DNA, 66 bp, Artificial Sequence
  source: /note=“Description of Artificial Sequence Synthetic oligonucleotide”
ttcaaaggttggagcctttgtacgcctggttgtgcaaggacaggtagtttcaatagttactgttgc

SEQ ID NO: 54 - DNA, 66 bp, Artificial Sequence
  source: /note=“Description of Artificial Sequence Synthetic oligonucleotide”
ttcaaacgttggagcctttgtacgcctggttgtgcaaggacaggtagtttcaatagttactgttgc

SEQ ID NO: 55 - DNA, 66 bp, Artificial Sequence
  source: /note=“Description of Artificial Sequence Synthetic oligonucleotide”
ttcaaacattggagcctttgtacgcctggttgtgcaaggacaggtagtttcaatagttactgttgc

SEQ ID NO: 56 - DNA, 66 bp, Artificial Sequence
  source: /note=“Description of Artificial Sequence Synthetic oligonucleotide”
ttcaaatcttggagcctttgtacgcctggttgtgcaaggacaggtagtttcaatagttactgttgc

SEQ ID NO: 57 - DNA, 66 bp, Artificial Sequence
  source: /note=“Description of Artificial Sequence Synthetic oligonucleotide”
ttcaaaacatggagcctttgtacgcctggttgtgcaaggacaggtagtttcaatagttactgttgc

SEQ ID NO: 58 - DNA, 66 bp, Artificial Sequence
  source: /note=“Description of Artificial Sequence Synthetic oligonucleotide”
ttcaaaatttggagcctttgtacgcctggttgtgcaaggacaggtagtttcaatagttactgttgc

SEQ ID NO: 59 - DNA, 66 bp, Artificial Sequence
  source: /note=“Description of Artificial Sequence Synthetic oligonucleotide”
ttcaaaagtaaaagcctttgtacgcctggttgtgcaaggacaggtagtttcaatagttactgttgc

SEQ ID NO: 60 - DNA, 66 bp, Artificial Sequence
  source: /note=“Description of Artificial Sequence Synthetic oligonucleotide”
ttcaaaagtgttagcctttgtacgcctggttgtgcaaggacaggtagtttcaatagttactgttgc

SEQ ID NO: 61 - DNA, 66 bp, Artificial Sequence
  source: /note=“Description of Artificial Sequence Synthetic oligonucleotide”
ttcaaaagtgctagcctttgtacgcctggttgtgcaaggacaggtagtttcaatagttactgttgc

SEQ ID NO: 62 - DNA, 66 bp, Artificial Sequence
  source: /note=“Description of Artificial Sequence Synthetic oligonucleotide”
ttcaaaagtttaagcctttgtacgcctggttgtgcaaggacaggtagtttcaatagttactgttgc

SEQ ID NO: 63 - DNA, 66 bp, Artificial Sequence
  source: /note=“Description of Artificial Sequence Synthetic oligonucleotide”
ttcaaaagtatgagcctttgtacgcctggttgtgcaaggacaggtagtttcaatagttactgttgc

SEQ ID NO: 64 - DNA, 66 bp, Artificial Sequence
  source: /note=“Description of Artificial Sequence Synthetic oligonucleotide”
ttcaaaagttgtagcctttgtacgcctggttgtgcaaggacaggtagtttcaatagttactgttgc

SEQ ID NO: 65 - DNA, 66 bp, Artificial Sequence
  source: /note=“Description of Artificial Sequence Synthetic oligonucleotide”
ttcaaaagtaatagcctttgtacgcctggttgtgcaaggacaggtagtttcaatagttactgttgc

SEQ ID NO: 66 - DNA, 66 bp, Artificial Sequence
  source: /note=“Description of Artificial Sequence Synthetic oligonucleotide”
ttcaaaagttatagcctttgtacgcctggttgtgcaaggacaggtagtttcaatagttactgttgc

SEQ ID NO: 67 - DNA, 66 bp, Artificial Sequence
  source: /note=“Description of Artificial Sequence Synthetic oligonucleotide”
ttcaaaagtgatagcctttgtacgcctggttgtgcaaggacaggtagtttcaatagttactgttgc

SEQ ID NO: 68 - DNA, 66 bp, Artificial Sequence
  source: /note=“Description of Artificial Sequence Synthetic oligonucleotide”
ttcaaaagtgaaagcctttgtacgcctggttgtgcaaggacaggtagtttcaatagttactgttgc

SEQ ID NO: 69 - DNA, 66 bp, Artificial Sequence
  source: /note=“Description of Artificial Sequence Synthetic oligonucleotide”
ttcaaaagtcctagcctttgtacgcctggttgtgcaaggacaggtagtttcaatagttactgttgc

SEQ ID NO: 70 - DNA, 66 bp, Artificial Sequence
  source: /note=“Description of Artificial Sequence Synthetic oligonucleotide”
ttcaaaagtcaaagcctttgtacgcctggttgtgcaaggacaggtagtttcaatagttactgttgc

SEQ ID NO: 71 - DNA, 66 bp, Artificial Sequence
  source: /note=“Description of Artificial Sequence Synthetic oligonucleotide”
ttcaaaagttttagcctttgtacgcctggttgtgcaaggacaggtagtttcaatagttactgttgc

SEQ ID NO: 72 - DNA, 66 bp, Artificial Sequence
  source: /note=“Description of Artificial Sequence Synthetic oligonucleotide”
ttcaaaagtggtagcctttgtacgcctggttgtgcaaggacaggtagtttcaatagttactgttgc

SEQ ID NO: 73 - DNA, 66 bp, Artificial Sequence
  source: /note=“Description of Artificial Sequence Synthetic oligonucleotide”
ttcaaaagtcgtagcctttgtacgcctggttgtgcaaggacaggtagtttcaatagttactgttgc

SEQ ID NO: 74 - DNA, 66 bp, Artificial Sequence
  source: /note=“Description of Artificial Sequence Synthetic oligonucleotide”
ttcaaaagtcatagcctttgtacgcctggttgtgcaaggacaggtagtttcaatagttactgttgc

SEQ ID NO: 75 - DNA, 66 bp, Artificial Sequence
  source: /note=“Description of Artificial Sequence Synthetic oligonucleotide”
ttcaaaagttctagcctttgtacgcctggttgtgcaaggacaggtagtttcaatagttactgttgc

SEQ ID NO: 76 - DNA, 66 bp, Artificial Sequence
  source: /note=“Description of Artificial Sequence Synthetic oligonucleotide”
ttcaaaagtacaagcctttgtacgcctggttgtgcaaggacaggtagtttcaatagttactgttgc

SEQ ID NO: 77 - DNA, 66 bp, Artificial Sequence
  source: /note=“Description of Artificial Sequence Synthetic oligonucleotide”
ttcaaaagtattagcctttgtacgcctggttgtgcaaggacaggtagtttcaatagttactgttgc

SEQ ID NO: 78 - DNA, 66 bp, Artificial Sequence
  source: /note=“Description of Artificial Sequence Synthetic oligonucleotide”
ttcaaaagttggaaactttgtacgcctggttgtgcaaggacaggtagtttcaatagttactgttgc

SEQ ID NO: 79 - DNA, 66 bp, Artificial Sequence
  source: /note=“Description of Artificial Sequence Synthetic oligonucleotide”
ttcaaaagttgggttctttgtacgcctggttgtgcaaggacaggtagtttcaatagttactgttgc

SEQ ID NO: 80 - DNA, 66 bp, Artificial Sequence
  source: /note=“Description of Artificial Sequence Synthetic oligonucleotide”
ttcaaaagttggtggctttgtacgcctggttgtgcaaggacaggtagtttcaatagttactgttgc

SEQ ID NO: 81 - DNA, 66 bp, Artificial Sequence
  source: /note=“Description of Artificial Sequence Synthetic oligonucleotide”
ttcaaaagttgggctctttgtacgcctggttgtgcaaggacaggtagtttcaatagttactgttgc

SEQ ID NO: 82 - DNA, 66 bp, Artificial Sequence
  source: /note=“Description of Artificial Sequence Synthetic oligonucleotide”
ttcaaaagttggttactttgtacgcctggttgtgcaaggacaggtagtttcaatagttactgttgc

SEQ ID NO: 83 - DNA, 66 bp, Artificial Sequence
  source: /note=“Description of Artificial Sequence Synthetic oligonucleotide”
ttcaaaagttggatgctttgtacgcctggttgtgcaaggacaggtagtttcaatagttactgttgc

SEQ ID NO: 84 - DNA, 66 bp, Artificial Sequence
  source: /note=“Description of Artificial Sequence Synthetic oligonucleotide”
ttcaaaagttggtgtctttgtacgcctggttgtgcaaggacaggtagtttcaatagttactgttgc

SEQ ID NO: 85 - DNA, 66 bp, Artificial Sequence
  source: /note=“Description of Artificial Sequence Synthetic oligonucleotide”
ttcaaaagttggaatctttgtacgcctggttgtgcaaggacaggtagtttcaatagttactgttgc

SEQ ID NO: 86 - DNA, 66 bp, Artificial Sequence
  source: /note=“Description of Artificial Sequence Synthetic oligonucleotide”
ttcaaaagttggtatctttgtacgcctggttgtgcaaggacaggtagtttcaatagttactgttgc

SEQ ID NO: 87 - DNA, 66 bp, Artificial Sequence
  source: /note=“Description of Artificial Sequence Synthetic oligonucleotide”
ttcaaaagttgggatctttgtacgcctggttgtgcaaggacaggtagtttcaatagttactgttgc

SEQ ID NO: 88 - DNA, 66 bp, Artificial Sequence
  source: /note=“Description of Artificial Sequence Synthetic oligonucleotide”
ttcaaaagttgggaactttgtacgcctggttgtgcaaggacaggtagtttcaatagttactgttgc

SEQ ID NO: 89 - DNA, 66 bp, Artificial Sequence
  source: /note=“Description of Artificial Sequence Synthetic oligonucleotide”
ttcaaaagttggcctctttgtacgcctggttgtgcaaggacaggtagtttcaatagttactgttgc

SEQ ID NO: 90 - DNA, 66 bp, Artificial Sequence
  source: /note=“Description of Artificial Sequence Synthetic oligonucleotide”
ttcaaaagttggcaactttgtacgcctggttgtgcaaggacaggtagtttcaatagttactgttgc

SEQ ID NO: 91 - DNA, 66 bp, Artificial Sequence
  source: /note=“Description of Artificial Sequence Synthetic oligonucleotide”
ttcaaaagttggtttctttgtacgcctggttgtgcaaggacaggtagtttcaatagttactgttgc

SEQ ID NO: 92 - DNA, 66 bp, Artificial Sequence
  source: /note=“Description of Artificial Sequence Synthetic oligonucleotide”
ttcaaaagttggggtctttgtacgcctggttgtgcaaggacaggtagtttcaatagttactgttgc

SEQ ID NO: 93 - DNA, 66 bp, Artificial Sequence
  source: /note=“Description of Artificial Sequence Synthetic oligonucleotide”
ttcaaaagttggcgtctttgtacgcctggttgtgcaaggacaggtagtttcaatagttactgttgc

SEQ ID NO: 94 - DNA, 66 bp, Artificial Sequence
  source: /note=“Description of Artificial Sequence Synthetic oligonucleotide”
ttcaaaagttggcatctttgtacgcctggttgtgcaaggacaggtagtttcaatagttactgttgc

SEQ ID NO: 95 - DNA, 66 bp, Artificial Sequence
  source: /note=“Description of Artificial Sequence Synthetic oligonucleotide”
ttcaaaagttggtctctttgtacgcctggttgtgcaaggacaggtagtttcaatagttactgttgc

SEQ ID NO: 96 - DNA, 66 bp, Artificial Sequence
  source: /note=“Description of Artificial Sequence Synthetic oligonucleotide”
ttcaaaagttggacactttgtacgcctggttgtgcaaggacaggtagtttcaatagttactgttgc

SEQ ID NO: 97 - DNA, 66 bp, Artificial Sequence
  source: /note=“Description of Artificial Sequence Synthetic oligonucleotide”
ttcaaaagttggattctttgtacgcctggttgtgcaaggacaggtagtttcaatagttactgttgc

SEQ ID NO: 98 - DNA, 66 bp, Artificial Sequence
  source: /note=“Description of Artificial Sequence Synthetic oligonucleotide”
ttcaaaagttggagcaaatgtacgcctggttgtgcaaggacaggtagtttcaatagttactgttgc

SEQ ID NO: 99 - DNA, 66 bp, Artificial Sequence
  source: /note=“Description of Artificial Sequence Synthetic oligonucleotide”
ttcaaaagttggagcgtttgtacgcctggttgtgcaaggacaggtagtttcaatagttactgttgc

SEQ ID NO: 100 - DNA, 66 bp, Artificial Sequence
  source: /note=“Description of Artificial Sequence Synthetic oligonucleotide”
ttcaaaagttggagctggtgtacgcctggttgtgcaaggacaggtagtttcaatagttactgttgc

SEQ ID NO: 101 - DNA, 66 bp, Artificial Sequence
  source: /note=“Description of Artificial Sequence Synthetic oligonucleotide”
ttcaaaagttggagcgcttgtacgcctggttgtgcaaggacaggtagtttcaatagttactgttgc

SEQ ID NO: 102 - DNA, 66 bp, Artificial Sequence
  source: /note=“Description of Artificial Sequence Synthetic oligonucleotide”
ttcaaaagttggagcttatgtacgcctggttgtgcaaggacaggtagtttcaatagttactgttgc

SEQ ID NO: 103 - DNA, 66 bp, Artificial Sequence
  source: /note=“Description of Artificial Sequence Synthetic oligonucleotide”
ttcaaaagttggagcatgtgtacgcctggttgtgcaaggacaggtagtttcaatagttactgttgc

SEQ ID NO: 104 - DNA, 66 bp, Artificial Sequence
  source: /note=“Description of Artificial Sequence Synthetic oligonucleotide”
ttcaaaagttggagctgttgtacgcctggttgtgcaaggacaggtagtttcaatagttactgttgc

SEQ ID NO: 105 - DNA, 66 bp, Artificial Sequence
  source: /note=“Description of Artificial Sequence Synthetic oligonucleotide”
ttcaaaagttggagcaattgtacgcctggttgtgcaaggacaggtagtttcaatagttactgttgc

SEQ ID NO: 106 - DNA, 66 bp, Artificial Sequence
  source: /note=“Description of Artificial Sequence Synthetic oligonucleotide”
ttcaaaagttggagctattgtacgcctggttgtgcaaggacaggtagtttcaatagttactgttgc

SEQ ID NO: 107 - DNA, 66 bp, Artificial Sequence
  source: /note=“Description of Artificial Sequence Synthetic oligonucleotide”
ttcaaaagttggagcgattgtacgcctggttgtgcaaggacaggtagtttcaatagttactgttgc

SEQ ID NO: 108 - DNA, 66 bp, Artificial Sequence
  source: /note=“Description of Artificial Sequence Synthetic oligonucleotide”
ttcaaaagttggagcgaatgtacgcctggttgtgcaaggacaggtagtttcaatagttactgttgc

SEQ ID NO: 109 - DNA, 66 bp, Artificial Sequence
  source: /note=“Description of Artificial Sequence Synthetic oligonucleotide”
ttcaaaagttggagcccttgtacgcctggttgtgcaaggacaggtagtttcaatagttactgttgc

SEQ ID NO: 110 - DNA, 66 bp, Artificial Sequence
  source: /note=“Description of Artificial Sequence Synthetic oligonucleotide”
ttcaaaagttggagccaatgtacgcctggttgtgcaaggacaggtagtttcaatagttactgttgc

SEQ ID NO: 111 - DNA, 66 bp, Artificial Sequence
  source: /note=“Description of Artificial Sequence Synthetic oligonucleotide”
ttcaaaagttggagcttttgtacgcctggttgtgcaaggacaggtagtttcaatagttactgttgc

SEQ ID NO: 112 - DNA, 66 bp, Artificial Sequence
  source: /note=“Description of Artificial Sequence Synthetic oligonucleotide”
ttcaaaagttggagcggttgtacgcctggttgtgcaaggacaggtagtttcaatagttactgttgc

SEQ ID NO: 113 - DNA, 66 bp, Artificial Sequence
  source: /note=“Description of Artificial Sequence Synthetic oligonucleotide”
ttcaaaagttggagccgttgtacgcctggttgtgcaaggacaggtagtttcaatagttactgttgc

SEQ ID NO: 114 - DNA, 66 bp, Artificial Sequence
  source: /note=“Description of Artificial Sequence Synthetic oligonucleotide”
ttcaaaagttggagccattgtacgcctggttgtgcaaggacaggtagtttcaatagttactgttgc

SEQ ID NO: 115 - DNA, 66 bp, Artificial Sequence
  source: /note=“Description of Artificial Sequence Synthetic oligonucleotide”
ttcaaaagttggagctcttgtacgcctggttgtgcaaggacaggtagtttcaatagttactgttgc

SEQ ID NO: 116 - DNA, 66 bp, Artificial Sequence
  source: /note=“Description of Artificial Sequence Synthetic oligonucleotide”
ttcaaaagttggagcacatgtacgcctggttgtgcaaggacaggtagtttcaatagttactgttgc

SEQ ID NO: 117 - DNA, 66 bp, Artificial Sequence
  source: /note=“Description of Artificial Sequence Synthetic oligonucleotide”
ttcaaaagttggagcatttgtacgcctggttgtgcaaggacaggtagtttcaatagttactgttgc

SEQ ID NO: 118 - DNA, 66 bp, Artificial Sequence
  source: /note=“Description of Artificial Sequence Synthetic oligonucleotide”
ttcaaaagttggagccttaaaacgcctggttgtgcaaggacaggtagtttcaatagttactgttgc

SEQ ID NO: 119 - DNA, 66 bp, Artificial Sequence
  source: /note=“Description of Artificial Sequence Synthetic oligonucleotide”
ttcaaaagttggagccttgttacgcctggttgtgcaaggacaggtagtttcaatagttactgttgc

SEQ ID NO: 120 - DNA, 66 bp, Artificial Sequence
  source: /note=“Description of Artificial Sequence Synthetic oligonucleotide”
ttcaaaagttggagcctttggacgcctggttgtgcaaggacaggtagtttcaatagttactgttgc

SEQ ID NO: 121 - DNA, 66 bp, Artificial Sequence
  source: /note=“Description of Artificial Sequence Synthetic oligonucleotide”
ttcaaaagttggagccttgctacgcctggttgtgcaaggacaggtagtttcaatagttactgttgc

SEQ ID NO: 122 - DNA, 66 bp, Artificial Sequence
  source: /note=“Description of Artificial Sequence Synthetic oligonucleotide”
ttcaaaagttggagccttttaacgcctggttgtgcaaggacaggtagtttcaatagttactgttgc

SEQ ID NO: 123 - DNA, 66 bp, Artificial Sequence
  source: /note=“Description of Artificial Sequence Synthetic oligonucleotide”
ttcaaaagttggagccttatgacgcctggttgtgcaaggacaggtagtttcaatagttactgttgc

SEQ ID NO: 124 - DNA, 66 bp, Artificial Sequence
  source: /note=“Description of Artificial Sequence Synthetic oligonucleotide”
ttcaaaagttggagccttaatacgcctggttgtgcaaggacaggtagtttcaatagttactgttgc

SEQ ID NO: 125 - DNA, 66 bp, Artificial Sequence
  source: /note=“Description of Artificial Sequence Synthetic oligonucleotide”
ttcaaaagttggagcctttatacgcctggttgtgcaaggacaggtagtttcaatagttactgttgc

SEQ ID NO: 126 - DNA, 66 bp, Artificial Sequence
  source: /note=“Description of Artificial Sequence Synthetic oligonucleotide”
ttcaaaagttggagccttgatacgcctggttgtgcaaggacaggtagtttcaatagttactgttgc

SEQ ID NO: 127 - DNA, 66 bp, Artificial Sequence
  source: /note=“Description of Artificial Sequence Synthetic oligonucleotide”
ttcaaaagttggagccttgaaacgcctggttgtgcaaggacaggtagtttcaatagttactgttgc

SEQ ID NO: 128 - DNA, 66 bp, Artificial Sequence
  source: /note=“Description of Artificial Sequence Synthetic oligonucleotide”
ttcaaaagttggagccttcctacgcctggttgtgcaaggacaggtagtttcaatagttactgttgc

SEQ ID NO: 129 - DNA, 66 bp, Artificial Sequence
  source: /note=“Description of Artificial Sequence Synthetic oligonucleotide”
ttcaaaagttggagccttcaaacgcctggttgtgcaaggacaggtagtttcaatagttactgttgc

SEQ ID NO: 130 - DNA, 66 bp, Artificial Sequence
  source: /note=“Description of Artificial Sequence Synthetic oligonucleotide”
ttcaaaagttggagcctttttacgcctggttgtgcaaggacaggtagtttcaatagttactgttgc

SEQ ID NO: 131 - DNA, 66 bp, Artificial Sequence
  source: /note=“Description of Artificial Sequence Synthetic oligonucleotide”
ttcaaaagttggagccttggtacgcctggttgtgcaaggacaggtagtttcaatagttactgttgc

SEQ ID NO: 132 - DNA, 66 bp, Artificial Sequence
  source: /note=“Description of Artificial Sequence Synthetic oligonucleotide”
ttcaaaagttggagccttcgtacgcctggttgtgcaaggacaggtagtttcaatagttactgttgc

SEQ ID NO: 133 - DNA, 66 bp, Artificial Sequence
  source: /note=“Description of Artificial Sequence Synthetic oligonucleotide”
ttcaaaagttggagccttcatacgcctggttgtgcaaggacaggtagtttcaatagttactgttgc

SEQ ID NO: 134 - DNA, 66 bp, Artificial Sequence
  source: /note=“Description of Artificial Sequence Synthetic oligonucleotide”
ttcaaaagttggagcctttctacgcctggttgtgcaaggacaggtagtttcaatagttactgttgc

SEQ ID NO: 135 - DNA, 66 bp, Artificial Sequence
  source: /note=“Description of Artificial Sequence Synthetic oligonucleotide”
ttcaaaagttggagccttacaacgcctggttgtgcaaggacaggtagtttcaatagttactgttgc

SEQ ID NO: 136 - DNA, 66 bp, Artificial Sequence
  source: /note=“Description of Artificial Sequence Synthetic oligonucleotide”
ttcaaaagttggagccttattacgcctggttgtgcaaggacaggtagtttcaatagttactgttgc

SEQ ID NO: 137 - DNA, 66 bp, Artificial Sequence
  source: /note=“Description of Artificial Sequence Synthetic oligonucleotide”
ttcaaaagttggagcctttgtaaacctggttgtgcaaggacaggtagtttcaatagttactgttgc

SEQ ID NO: 138 - DNA, 66 bp, Artificial Sequence
  source: /note=“Description of Artificial Sequence Synthetic oligonucleotide”
ttcaaaagttggagcctttgtgttcctggttgtgcaaggacaggtagtttcaatagttactgttgc

SEQ ID NO: 139 - DNA, 66 bp, Artificial Sequence
  source: /note=“Description of Artificial Sequence Synthetic oligonucleotide”
ttcaaaagttggagcctttgttggcctggttgtgcaaggacaggtagtttcaatagttactgttgc

SEQ ID NO: 140 - DNA, 66 bp, Artificial Sequence
  source: /note=“Description of Artificial Sequence Synthetic oligonucleotide”
ttcaaaagttggagcctttgtgctcctggttgtgcaaggacaggtagtttcaatagttactgttgc

SEQ ID NO: 141 - DNA, 66 bp, Artificial Sequence
  source: /note=“Description of Artificial Sequence Synthetic oligonucleotide”
ttcaaaagttggagcctttgtttacctggttgtgcaaggacaggtagtttcaatagttactgttgc

SEQ ID NO: 142 - DNA, 66 bp, Artificial Sequence
  source: /note=“Description of Artificial Sequence Synthetic oligonucleotide”
ttcaaaagttggagcctttgtatgcctggttgtgcaaggacaggtagtttcaatagttactgttgc

SEQ ID NO: 143 - DNA, 66 bp, Artificial Sequence
  source: /note=“Description of Artificial Sequence Synthetic oligonucleotide”
ttcaaaagttggagcctttgttgtcctggttgtgcaaggacaggtagtttcaatagttactgttgc

SEQ ID NO: 144 - DNA, 66 bp, Artificial Sequence
  source: /note=“Description of Artificial Sequence Synthetic oligonucleotide”
ttcaaaagttggagcctttgtaatcctggttgtgcaaggacaggtagtttcaatagttactgttgc

SEQ ID NO: 145 - DNA, 66 bp, Artificial Sequence
  source: /note=“Description of Artificial Sequence Synthetic oligonucleotide”
ttcaaaagttggagcctttgttatcctggttgtgcaaggacaggtagtttcaatagttactgttgc

SEQ ID NO: 146 - DNA, 66 bp, Artificial Sequence
  source: /note=“Description of Artificial Sequence Synthetic oligonucleotide”
ttcaaaagttggagcctttgtgatcctggttgtgcaaggacaggtagtttcaatagttactgttgc

SEQ ID NO: 147 - DNA, 66 bp, Artificial Sequence
  source: /note=“Description of Artificial Sequence Synthetic oligonucleotide”
ttcaaaagttggagcctttgtgaacctggttgtgcaaggacaggtagtttcaatagttactgttgc

SEQ ID NO: 148 - DNA, 66 bp, Artificial Sequence
  source: /note=“Description of Artificial Sequence Synthetic oligonucleotide”
ttcaaaagttggagcctttgtcctcctggttgtgcaaggacaggtagtttcaatagttactgttgc

SEQ ID NO: 149 - DNA, 66 bp, Artificial Sequence
  source: /note=“Description of Artificial Sequence Synthetic oligonucleotide”
ttcaaaagttggagcctttgtcaacctggttgtgcaaggacaggtagtttcaatagttactgttgc

SEQ ID NO: 150 - DNA, 66 bp, Artificial Sequence
  source: /note=“Description of Artificial Sequence Synthetic oligonucleotide”
ttcaaaagttggagcctttgttttcctggttgtgcaaggacaggtagtttcaatagttactgttgc

SEQ ID NO: 151 - DNA, 66 bp, Artificial Sequence
  source: /note=“Description of Artificial Sequence Synthetic oligonucleotide”
ttcaaaagttggagcctttgtggtcctggttgtgcaaggacaggtagtttcaatagttactgttgc

SEQ ID NO: 152 - DNA, 66 bp, Artificial Sequence
  source: /note=“Description of Artificial Sequence Synthetic oligonucleotide”
ttcaaaagttggagcctttgtcgtcctggttgtgcaaggacaggtagtttcaatagttactgttgc

SEQ ID NO: 153 - DNA, 66 bp, Artificial Sequence
  source: /note=“Description of Artificial Sequence Synthetic oligonucleotide”
ttcaaaagttggagcctttgtcatcctggttgtgcaaggacaggtagtttcaatagttactgttgc

SEQ ID NO: 154 - DNA, 66 bp, Artificial Sequence
  source: /note=“Description of Artificial Sequence Synthetic oligonucleotide”
ttcaaaagttggagcctttgttctcctggttgtgcaaggacaggtagtttcaatagttactgttgc

SEQ ID NO: 155 - DNA, 66 bp, Artificial Sequence
  source: /note=“Description of Artificial Sequence Synthetic oligonucleotide”
ttcaaaagttggagcctttgtacacctggttgtgcaaggacaggtagtttcaatagttactgttgc

SEQ ID NO: 156 - DNA, 66 bp, Artificial Sequence
  source: /note=“Description of Artificial Sequence Synthetic oligonucleotide”
ttcaaaagttggagcctttgtattcctggttgtgcaaggacaggtagtttcaatagttactgttgc

SEQ ID NO: 157 - DNA, 66 bp, Artificial Sequence
  source: /note=“Description of Artificial Sequence Synthetic oligonucleotide”
ttcaaaagttggagcctttgtacgaaaggttgtgcaaggacaggtagtttcaatagttactgttgc

SEQ ID NO: 158 - DNA, 66 bp, Artificial Sequence
  source: /note=“Description of Artificial Sequence Synthetic oligonucleotide”
ttcaaaagttggagcctttgtacggttggttgtgcaaggacaggtagtttcaatagttactgttgc

SEQ ID NO: 159 - DNA, 66 bp, Artificial Sequence
  source: /note=“Description of Artificial Sequence Synthetic oligonucleotide”
ttcaaaagttggagcctttgtacgtggggttgtgcaaggacaggtagtttcaatagttactgttgc

SEQ ID NO: 160 - DNA, 66 bp, Artificial Sequence
  source: /note=“Description of Artificial Sequence Synthetic oligonucleotide”
ttcaaaagttggagcctttgtacggctggttgtgcaaggacaggtagtttcaatagttactgttgc

SEQ ID NO: 161 - DNA, 66 bp, Artificial Sequence
  source: /note=“Description of Artificial Sequence Synthetic oligonucleotide”
ttcaaaagttggagcctttgtacgttaggttgtgcaaggacaggtagtttcaatagttactgttgc

SEQ ID NO: 162 - DNA, 66 bp, Artificial Sequence
  source: /note=“Description of Artificial Sequence Synthetic oligonucleotide”
ttcaaaagttggagcctttgtacgatgggttgtgcaaggacaggtagtttcaatagttactgttgc

SEQ ID NO: 163 - DNA, 66 bp, Artificial Sequence
  source: /note=“Description of Artificial Sequence Synthetic oligonucleotide”
ttcaaaagttggagcctttgtacgtgtggttgtgcaaggacaggtagtttcaatagttactgttgc

SEQ ID NO: 164 - DNA, 66 bp, Artificial Sequence
  source: /note=“Description of Artificial Sequence Synthetic oligonucleotide”
ttcaaaagttggagcctttgtacgaatggttgtgcaaggacaggtagtttcaatagttactgttgc

SEQ ID NO: 165 - DNA, 66 bp, Artificial Sequence
  source: /note=“Description of Artificial Sequence Synthetic oligonucleotide”
ttcaaaagttggagcctttgtacgtatggttgtgcaaggacaggtagtttcaatagttactgttgc

SEQ ID NO: 166 - DNA, 66 bp, Artificial Sequence
  source: /note=“Description of Artificial Sequence Synthetic oligonucleotide”
ttcaaaagttggagcctttgtacggatggttgtgcaaggacaggtagtttcaatagttactgttgc

SEQ ID NO: 167 - DNA, 66 bp, Artificial Sequence
  source: /note=“Description of Artificial Sequence Synthetic oligonucleotide”
ttcaaaagttggagcctttgtacggaaggttgtgcaaggacaggtagtttcaatagttactgttgc

SEQ ID NO: 168 - DNA, 66 bp, Artificial Sequence
  source: /note=“Description of Artificial Sequence Synthetic oligonucleotide”
ttcaaaagttggagcctttgtacgcaaggttgtgcaaggacaggtagtttcaatagttactgttgc

SEQ ID NO: 169 - DNA, 66 bp, Artificial Sequence
  source: /note=“Description of Artificial Sequence Synthetic oligonucleotide”
ttcaaaagttggagcctttgtacgtttggttgtgcaaggacaggtagtttcaatagttactgttgc

SEQ ID NO: 170 - DNA, 66 bp, Artificial Sequence
  source: /note=“Description of Artificial Sequence Synthetic oligonucleotide”
ttcaaaagttggagcctttgtacgggtggttgtgcaaggacaggtagtttcaatagttactgttgc

SEQ ID NO: 171 - DNA, 66 bp, Artificial Sequence
  source: /note=“Description of Artificial Sequence Synthetic oligonucleotide”
ttcaaaagttggagcctttgtacgcgtggttgtgcaaggacaggtagtttcaatagttactgttgc

SEQ ID NO: 172 - DNA, 66 bp, Artificial Sequence
  source: /note=“Description of Artificial Sequence Synthetic oligonucleotide”
ttcaaaagttggagcctttgtacgcatggttgtgcaaggacaggtagtttcaatagttactgttgc

SEQ ID NO: 173 - DNA, 66 bp, Artificial Sequence
  source: /note=“Description of Artificial Sequence Synthetic oligonucleotide”
ttcaaaagttggagcctttgtacgtctggttgtgcaaggacaggtagtttcaatagttactgttgc

SEQ ID NO: 174 - DNA, 66 bp, Artificial Sequence
  source: /note=“Description of Artificial Sequence Synthetic oligonucleotide”
ttcaaaagttggagcctttgtacgacaggttgtgcaaggacaggtagtttcaatagttactgttgc

SEQ ID NO: 175 - DNA, 66 bp, Artificial Sequence
  source: /note=“Description of Artificial Sequence Synthetic oligonucleotide”
ttcaaaagttggagcctttgtacgattggttgtgcaaggacaggtagtttcaatagttactgttgc

SEQ ID NO: 176 - DNA, 66 bp, Artificial Sequence
  source: /note=“Description of Artificial Sequence Synthetic oligonucleotide”
ttcaaaagttggagcctttgtacgcctaaatgtgcaaggacaggtagtttcaatagttactgttgc

SEQ ID NO: 177 - DNA, 66 bp, Artificial Sequence
  source: /note=“Description of Artificial Sequence Synthetic oligonucleotide”
ttcaaaagttggagcctttgtacgcctgtttgtgcaaggacaggtagtttcaatagttactgttgc

SEQ ID NO: 178 - DNA, 66 bp, Artificial Sequence
  source: /note=“Description of Artificial Sequence Synthetic oligonucleotide”
ttcaaaagttggagcctttgtacgccttggtgtgcaaggacaggtagtttcaatagttactgttgc

SEQ ID NO: 179 - DNA, 66 bp, Artificial Sequence
  source: /note=“Description of Artificial Sequence Synthetic oligonucleotide”
ttcaaaagttggagcctttgtacgcctgcttgtgcaaggacaggtagtttcaatagttactgttgc

SEQ ID NO: 180 - DNA, 66 bp, Artificial Sequence
  source: /note=“Description of Artificial Sequence Synthetic oligonucleotide”
ttcaaaagttggagcctttgtacgcctttatgtgcaaggacaggtagtttcaatagttactgttgc

SEQ ID NO: 181 - DNA, 66 bp, Artificial Sequence
  source: /note=“Description of Artificial Sequence Synthetic oligonucleotide”
ttcaaaagttggagcctttgtacgcctatgtgtgcaaggacaggtagtttcaatagttactgttgc

SEQ ID NO: 182 - DNA, 66 bp, Artificial Sequence
  source: /note=“Description of Artificial Sequence Synthetic oligonucleotide”
ttcaaaagttggagcctttgtacgccttgttgtgcaaggacaggtagtttcaatagttactgttgc

SEQ ID NO: 183 - DNA, 66 bp, Artificial Sequence
  source: /note=“Description of Artificial Sequence Synthetic oligonucleotide”
ttcaaaagttggagcctttgtacgcctaattgtgcaaggacaggtagtttcaatagttactgttgc

SEQ ID NO: 184 - DNA, 66 bp, Artificial Sequence
  source: /note=“Description of Artificial Sequence Synthetic oligonucleotide”
ttcaaaagttggagcctttgtacgccttattgtgcaaggacaggtagtttcaatagttactgttgc

SEQ ID NO: 185 - DNA, 66 bp, Artificial Sequence
  source: /note=“Description of Artificial Sequence Synthetic oligonucleotide”
ttcaaaagttggagcctttgtacgcctgattgtgcaaggacaggtagtttcaatagttactgttgc

SEQ ID NO: 186 - DNA, 66 bp, Artificial Sequence
  source: /note=“Description of Artificial Sequence Synthetic oligonucleotide”
ttcaaaagttggagcctttgtacgcctgaatgtgcaaggacaggtagtttcaatagttactgttgc

SEQ ID NO: 187 - DNA, 66 bp, Artificial Sequence
  source: /note=“Description of Artificial Sequence Synthetic oligonucleotide”
ttcaaaagttggagcctttgtacgcctccttgtgcaaggacaggtagtttcaatagttactgttgc

SEQ ID NO: 188 - DNA, 66 bp, Artificial Sequence
  source: /note=“Description of Artificial Sequence Synthetic oligonucleotide”
ttcaaaagttggagcctttgtacgcctcaatgtgcaaggacaggtagtttcaatagttactgttgc

SEQ ID NO: 189 - DNA, 66 bp, Artificial Sequence
  source: /note=“Description of Artificial Sequence Synthetic oligonucleotide”
ttcaaaagttggagcctttgtacgcctttttgtgcaaggacaggtagtttcaatagttactgttgc

SEQ ID NO: 190 - DNA, 66 bp, Artificial Sequence
  source: /note=“Description of Artificial Sequence Synthetic oligonucleotide”
ttcaaaagttggagcctttgtacgcctcgttgtgcaaggacaggtagtttcaatagttactgttgc

SEQ ID NO: 191 - DNA, 66 bp, Artificial Sequence
  source: /note=“Description of Artificial Sequence Synthetic oligonucleotide”
ttcaaaagttggagcctttgtacgcctcattgtgcaaggacaggtagtttcaatagttactgttgc

SEQ ID NO: 192 - DNA, 66 bp, Artificial Sequence
  source: /note=“Description of Artificial Sequence Synthetic oligonucleotide”
ttcaaaagttggagcctttgtacgccttcttgtgcaaggacaggtagtttcaatagttactgttgc

SEQ ID NO: 193 - DNA, 66 bp, Artificial Sequence
  source: /note=“Description of Artificial Sequence Synthetic oligonucleotide”
ttcaaaagttggagcctttgtacgcctacatgtgcaaggacaggtagtttcaatagttactgttgc

SEQ ID NO: 194 - DNA, 66 bp, Artificial Sequence
  source: /note=“Description of Artificial Sequence Synthetic oligonucleotide”
ttcaaaagttggagcctttgtacgcctatttgtgcaaggacaggtagtttcaatagttactgttgc

SEQ ID NO: 195 - DNA, 66 bp, Artificial Sequence
  source: /note=“Description of Artificial Sequence Synthetic oligonucleotide”
ttcaaaagttggagcctttgtacgcctggtaaagcaaggacaggtagtttcaatagttactgttgc

SEQ ID NO: 196 - DNA, 66 bp, Artificial Sequence
  source: /note=“Description of Artificial Sequence Synthetic oligonucleotide”
ttcaaaagttggagcctttgtacgcctggtgttgcaaggacaggtagtttcaatagttactgttgc

SEQ ID NO: 197 - DNA, 66 bp, Artificial Sequence
  source: /note=“Description of Artificial Sequence Synthetic oligonucleotide”
ttcaaaagttggagcctttgtacgcctggttgggcaaggacaggtagtttcaatagttactgttgc

SEQ ID NO: 198 - DNA, 66 bp, Artificial Sequence
  source: /note=“Description of Artificial Sequence Synthetic oligonucleotide”
ttcaaaagttggagcctttgtacgcctggtgctgcaaggacaggtagtttcaatagttactgttgc

SEQ ID NO: 199 - DNA, 66 bp, Artificial Sequence
  source: /note=“Description of Artificial Sequence Synthetic oligonucleotide”
ttcaaaagttggagcctttgtacgcctggtttagcaaggacaggtagtttcaatagttactgttgc

SEQ ID NO: 200 - DNA, 66 bp, Artificial Sequence
  source: /note=“Description of Artificial Sequence Synthetic oligonucleotide”
ttcaaaagttggagcctttgtacgcctggtatggcaaggacaggtagtttcaatagttactgttgc

SEQ ID NO: 201 - DNA, 66 bp, Artificial Sequence
  source: /note=“Description of Artificial Sequence Synthetic oligonucleotide”
ttcaaaagttggagcctttgtacgcctggtaatgcaaggacaggtagtttcaatagttactgttgc

SEQ ID NO: 202 - DNA, 66 bp, Artificial Sequence
  source: /note=“Description of Artificial Sequence Synthetic oligonucleotide”
ttcaaaagttggagcctttgtacgcctggttatgcaaggacaggtagtttcaatagttactgttgc

SEQ ID NO: 203 - DNA, 66 bp, Artificial Sequence
  source: /note=“Description of Artificial Sequence Synthetic oligonucleotide”
ttcaaaagttggagcctttgtacgcctggtgatgcaaggacaggtagtttcaatagttactgttgc

SEQ ID NO: 204 - DNA, 66 bp, Artificial Sequence
  source: /note=“Description of Artificial Sequence Synthetic oligonucleotide”
ttcaaaagttggagcctttgtacgcctggtgaagcaaggacaggtagtttcaatagttactgttgc

SEQ ID NO: 205 - DNA, 66 bp, Artificial Sequence
  source: /note=“Description of Artificial Sequence Synthetic oligonucleotide”
ttcaaaagttggagcctttgtacgcctggtcctgcaaggacaggtagtttcaatagttactgttgc

SEQ ID NO: 206 - DNA, 66 bp, Artificial Sequence
  source: /note=“Description of Artificial Sequence Synthetic oligonucleotide”
ttcaaaagttggagcctttgtacgcctggtcaagcaaggacaggtagtttcaatagttactgttgc

SEQ ID NO: 207 - DNA, 66 bp, Artificial Sequence
  source: /note=“Description of Artificial Sequence Synthetic oligonucleotide”
ttcaaaagttggagcctttgtacgcctggttttgcaaggacaggtagtttcaatagttactgttgc

SEQ ID NO: 208 - DNA, 66 bp, Artificial Sequence
  source: /note=“Description of Artificial Sequence Synthetic oligonucleotide”
ttcaaaagttggagcctttgtacgcctggtggtgcaaggacaggtagtttcaatagttactgttgc

SEQ ID NO: 209 - DNA, 66 bp, Artificial Sequence
  source: /note=“Description of Artificial Sequence Synthetic oligonucleotide”
ttcaaaagttggagcctttgtacgcctggtcgtgcaaggacaggtagtttcaatagttactgttgc

SEQ ID NO: 210 - DNA, 66 bp, Artificial Sequence
  source: /note=“Description of Artificial Sequence Synthetic oligonucleotide”
ttcaaaagttggagcctttgtacgcctggtcatgcaaggacaggtagtttcaatagttactgttgc

SEQ ID NO: 211 - DNA, 66 bp, Artificial Sequence
  source: /note=“Description of Artificial Sequence Synthetic oligonucleotide”
ttcaaaagttggagcctttgtacgcctggttctgcaaggacaggtagtttcaatagttactgttgc

SEQ ID NO: 212 - DNA, 66 bp, Artificial Sequence
  source: /note=“Description of Artificial Sequence Synthetic oligonucleotide”
ttcaaaagttggagcctttgtacgcctggtacagcaaggacaggtagtttcaatagttactgttgc

SEQ ID NO: 213 - DNA, 66 bp, Artificial Sequence
  source: /note=“Description of Artificial Sequence Synthetic oligonucleotide”
ttcaaaagttggagcctttgtacgcctggtattgcaaggacaggtagtttcaatagttactgttgc

SEQ ID NO: 214 - DNA, 66 bp, Artificial Sequence
  source: /note=“Description of Artificial Sequence Synthetic oligonucleotide”
ttcaaaagttggagcctttgtacgcctggttgtaaaaggacaggtagtttcaatagttactgttgc

SEQ ID NO: 215 - DNA, 66 bp, Artificial Sequence
  source: /note=“Description of Artificial Sequence Synthetic oligonucleotide”
ttcaaaagttggagcctttgtacgcctggttgtgttaggacaggtagtttcaatagttactgttgc

SEQ ID NO: 216 - DNA, 66 bp, Artificial Sequence
  source: /note=“Description of Artificial Sequence Synthetic oligonucleotide”
ttcaaaagttggagcctttgtacgcctggttgttggaggacaggtagtttcaatagttactgttgc

SEQ ID NO: 217 - DNA, 66 bp, Artificial Sequence
  source: /note=“Description of Artificial Sequence Synthetic oligonucleotide”
ttcaaaagttggagcctttgtacgcctggttgtgctaggacaggtagtttcaatagttactgttgc

SEQ ID NO: 218 - DNA, 66 bp, Artificial Sequence
  source: /note=“Description of Artificial Sequence Synthetic oligonucleotide”
ttcaaaagttggagcctttgtacgcctggttgtttaaggacaggtagtttcaatagttactgttgc

SEQ ID NO: 219 - DNA, 66 bp, Artificial Sequence
  source: /note=“Description of Artificial Sequence Synthetic oligonucleotide”
ttcaaaagttggagcctttgtacgcctggttgtatgaggacaggtagtttcaatagttactgttgc

SEQ ID NO: 220 - DNA, 66 bp, Artificial Sequence
  source: /note=“Description of Artificial Sequence Synthetic oligonucleotide”
ttcaaaagttggagcctttgtacgcctggttgttgtaggacaggtagtttcaatagttactgttgc

SEQ ID NO: 221 - DNA, 66 bp, Artificial Sequence
  source: /note=“Description of Artificial Sequence Synthetic oligonucleotide”
ttcaaaagttggagcctttgtacgcctggttgtaataggacaggtagtttcaatagttactgttgc

SEQ ID NO: 222 - DNA, 66 bp, Artificial Sequence
  source: /note=“Description of Artificial Sequence Synthetic oligonucleotide”
ttcaaaagttggagcctttgtacgcctggttgttataggacaggtagtttcaatagttactgttgc

SEQ ID NO: 223 - DNA, 66 bp, Artificial Sequence
  source: /note=“Description of Artificial Sequence Synthetic oligonucleotide”
ttcaaaagttggagcctttgtacgcctggttgtgataggacaggtagtttcaatagttactgttgc

SEQ ID NO: 224 - DNA, 66 bp, Artificial Sequence
  source: /note=“Description of Artificial Sequence Synthetic oligonucleotide”
ttcaaaagttggagcctttgtacgcctggttgtgaaaggacaggtagtttcaatagttactgttgc

SEQ ID NO: 225 - DNA, 66 bp, Artificial Sequence
  source: /note=“Description of Artificial Sequence Synthetic oligonucleotide”
ttcaaaagttggagcctttgtacgcctggttgtcctaggacaggtagtttcaatagttactgttgc

SEQ ID NO: 226 - DNA, 66 bp, Artificial Sequence
  source: /note=“Description of Artificial Sequence Synthetic oligonucleotide”
ttcaaaagttggagcctttgtacgcctggttgtcaaaggacaggtagtttcaatagttactgttgc

SEQ ID NO: 227 - DNA, 66 bp, Artificial Sequence
  source: /note=“Description of Artificial Sequence Synthetic oligonucleotide”
ttcaaaagttggagcctttgtacgcctggttgttttaggacaggtagtttcaatagttactgttgc

SEQ ID NO: 228 - DNA, 66 bp, Artificial Sequence
  source: /note=“Description of Artificial Sequence Synthetic oligonucleotide”
ttcaaaagttggagcctttgtacgcctggttgtggtaggacaggtagtttcaatagttactgttgc

SEQ ID NO: 229 - DNA, 66 bp, Artificial Sequence
  source: /note=“Description of Artificial Sequence Synthetic oligonucleotide”
ttcaaaagttggagcctttgtacgcctggttgtcgtaggacaggtagtttcaatagttactgttgc

SEQ ID NO: 230 - DNA, 66 bp, Artificial Sequence
  source: /note=“Description of Artificial Sequence Synthetic oligonucleotide”
ttcaaaagttggagcctttgtacgcctggttgtcataggacaggtagtttcaatagttactgttgc

SEQ ID NO: 231 - DNA, 66 bp, Artificial Sequence
  source: /note=“Description of Artificial Sequence Synthetic oligonucleotide”
ttcaaaagttggagcctttgtacgcctggttgttctaggacaggtagtttcaatagttactgttgc

SEQ ID NO: 232 - DNA, 66 bp, Artificial Sequence
  source: /note=“Description of Artificial Sequence Synthetic oligonucleotide”
ttcaaaagttggagcctttgtacgcctggttgtacaaggacaggtagtttcaatagttactgttgc

SEQ ID NO: 233 - DNA, 66 bp, Artificial Sequence
  source: /note=“Description of Artificial Sequence Synthetic oligonucleotide”
ttcaaaagttggagcctttgtacgcctggttgtattaggacaggtagtttcaatagttactgttgc

SEQ ID NO: 234 - DNA, 66 bp, Artificial Sequence
  source: /note=“Description of Artificial Sequence Synthetic oligonucleotide”
ttcaaaagttggagcctttgtacgcctggttgtgcaaaaacaggtagtttcaatagttactgttgc

SEQ ID NO: 235 - DNA, 66 bp, Artificial Sequence
  source: /note=“Description of Artificial Sequence Synthetic oligonucleotide”
ttcaaaagttggagcctttgtacgcctggttgtgcagttacaggtagtttcaatagttactgttgc

SEQ ID NO: 236 - DNA, 66 bp, Artificial Sequence
  source: /note=“Description of Artificial Sequence Synthetic oligonucleotide”
ttcaaaagttggagcctttgtacgcctggttgtgcatggacaggtagtttcaatagttactgttgc

SEQ ID NO: 237 - DNA, 66 bp, Artificial Sequence
  source: /note=“Description of Artificial Sequence Synthetic oligonucleotide”
ttcaaaagttggagcctttgtacgcctggttgtgcagctacaggtagtttcaatagttactgttgc

SEQ ID NO: 238 - DNA, 66 bp, Artificial Sequence
  source: /note=“Description of Artificial Sequence Synthetic oligonucleotide”
ttcaaaagttggagcctttgtacgcctggttgtgcattaacaggtagtttcaatagttactgttgc

SEQ ID NO: 239 - DNA, 66 bp, Artificial Sequence
  source: /note=“Description of Artificial Sequence Synthetic oligonucleotide”
ttcaaaagttggagcctttgtacgcctggttgtgcaatgacaggtagtttcaatagttactgttgc

SEQ ID NO: 240 - DNA, 66 bp, Artificial Sequence
  source: /note=“Description of Artificial Sequence Synthetic oligonucleotide”
ttcaaaagttggagcctttgtacgcctggttgtgcatgtacaggtagtttcaatagttactgttgc

SEQ ID NO: 241 - DNA, 66 bp, Artificial Sequence
  source: /note=“Description of Artificial Sequence Synthetic oligonucleotide”
ttcaaaagttggagcctttgtacgcctggttgtgcaaatacaggtagtttcaatagttactgttgc

SEQ ID NO: 242 - DNA, 66 bp, Artificial Sequence
  source: /note=“Description of Artificial Sequence Synthetic oligonucleotide”
ttcaaaagttggagcctttgtacgcctggttgtgcatatacaggtagtttcaatagttactgttgc

SEQ ID NO: 243 - DNA, 66 bp, Artificial Sequence
  source: /note=“Description of Artificial Sequence Synthetic oligonucleotide”
ttcaaaagttggagcctttgtacgcctggttgtgcagatacaggtagtttcaatagttactgttgc

SEQ ID NO: 244 - DNA, 66 bp, Artificial Sequence
  source: /note=“Description of Artificial Sequence Synthetic oligonucleotide”
ttcaaaagttggagcctttgtacgcctggttgtgcagaaacaggtagtttcaatagttactgttgc

SEQ ID NO: 245 - DNA, 66 bp, Artificial Sequence
  source: /note=“Description of Artificial Sequence Synthetic oligonucleotide”
ttcaaaagttggagcctttgtacgcctggttgtgcacctacaggtagtttcaatagttactgttgc

SEQ ID NO: 246 - DNA, 66 bp, Artificial Sequence
  source: /note=“Description of Artificial Sequence Synthetic oligonucleotide”
ttcaaaagttggagcctttgtacgcctggttgtgcacaaacaggtagtttcaatagttactgttgc

SEQ ID NO: 247 - DNA, 66 bp, Artificial Sequence
  source: /note=“Description of Artificial Sequence Synthetic oligonucleotide”
ttcaaaagttggagcctttgtacgcctggttgtgcatttacaggtagtttcaatagttactgttgc

SEQ ID NO: 248 - DNA, 66 bp, Artificial Sequence
  source: /note=“Description of Artificial Sequence Synthetic oligonucleotide”
ttcaaaagttggagcctttgtacgcctggttgtgcaggtacaggtagtttcaatagttactgttgc

SEQ ID NO: 249 - DNA, 66 bp, Artificial Sequence
  source: /note=“Description of Artificial Sequence Synthetic oligonucleotide”
ttcaaaagttggagcctttgtacgcctggttgtgcacgtacaggtagtttcaatagttactgttgc

SEQ ID NO: 250 - DNA, 66 bp, Artificial Sequence
  source: /note=“Description of Artificial Sequence Synthetic oligonucleotide”
ttcaaaagttggagcctttgtacgcctggttgtgcacatacaggtagtttcaatagttactgttgc

SEQ ID NO: 251 - DNA, 66 bp, Artificial Sequence
  source: /note=“Description of Artificial Sequence Synthetic oligonucleotide”
ttcaaaagttggagcctttgtacgcctggttgtgcatctacaggtagtttcaatagttactgttgc

SEQ ID NO: 252 - DNA, 66 bp, Artificial Sequence
  source: /note=“Description of Artificial Sequence Synthetic oligonucleotide”
ttcaaaagttggagcctttgtacgcctggttgtgcaacaacaggtagtttcaatagttactgttgc

SEQ ID NO: 253 - DNA, 66 bp, Artificial Sequence
  source: /note=“Description of Artificial Sequence Synthetic oligonucleotide”
ttcaaaagttggagcctttgtacgcctggttgtgcaattacaggtagtttcaatagttactgttgc

SEQ ID NO: 254 - DNA, 66 bp, Artificial Sequence
  source: /note=“Description of Artificial Sequence Synthetic oligonucleotide”
ttcaaaagttggagcctttgtacgcctggttgtgcaaggaaaggtagtttcaatagttactgttgc

SEQ ID NO: 255 - DNA, 66 bp, Artificial Sequence
  source: /note=“Description of Artificial Sequence Synthetic oligonucleotide”
ttcaaaagttggagcctttgtacgcctggttgtgcaagggttggtagtttcaatagttactgttgc

SEQ ID NO: 256 - DNA, 66 bp, Artificial Sequence
  source: /note=“Description of Artificial Sequence Synthetic oligonucleotide”
ttcaaaagttggagcctttgtacgcctggttgtgcaaggtggggtagtttcaatagttactgttgc

SEQ ID NO: 257 - DNA, 66 bp, Artificial Sequence
  source: /note=“Description of Artificial Sequence Synthetic oligonucleotide”
ttcaaaagttggagcctttgtacgcctggttgtgcaagggctggtagtttcaatagttactgttgc

SEQ ID NO: 258 - DNA, 66 bp, Artificial Sequence
  source: /note=“Description of Artificial Sequence Synthetic oligonucleotide”
ttcaaaagttggagcctttgtacgcctggttgtgcaaggttaggtagtttcaatagttactgttgc

SEQ ID NO: 259 - DNA, 66 bp, Artificial Sequence
  source: /note=“Description of Artificial Sequence Synthetic oligonucleotide”
ttcaaaagttggagcctttgtacgcctggttgtgcaaggatgggtagtttcaatagttactgttgc

SEQ ID NO: 260 - DNA, 66 bp, Artificial Sequence
  source: /note=“Description of Artificial Sequence Synthetic oligonucleotide”
ttcaaaagttggagcctttgtacgcctggttgtgcaaggtgtggtagtttcaatagttactgttgc

SEQ ID NO: 261 - DNA, 66 bp, Artificial Sequence
  source: /note=“Description of Artificial Sequence Synthetic oligonucleotide”
ttcaaaagttggagcctttgtacgcctggttgtgcaaggaatggtagtttcaatagttactgttgc

SEQ ID NO: 262 - DNA, 66 bp, Artificial Sequence
  source: /note=“Description of Artificial Sequence Synthetic oligonucleotide”
ttcaaaagttggagcctttgtacgcctggttgtgcaaggtatggtagtttcaatagttactgttgc

SEQ ID NO: 263 - DNA, 66 bp, Artificial Sequence
  source: /note=“Description of Artificial Sequence Synthetic oligonucleotide”
ttcaaaagttggagcctttgtacgcctggttgtgcaagggatggtagtttcaatagttactgttgc

SEQ ID NO: 264 - DNA, 66 bp, Artificial Sequence
  source: /note=“Description of Artificial Sequence Synthetic oligonucleotide”
ttcaaaagttggagcctttgtacgcctggttgtgcaagggaaggtagtttcaatagttactgttgc

SEQ ID NO: 265 - DNA, 66 bp, Artificial Sequence
  source: /note=“Description of Artificial Sequence Synthetic oligonucleotide”
ttcaaaagttggagcctttgtacgcctggttgtgcaaggcctggtagtttcaatagttactgttgc

SEQ ID NO: 266 - DNA, 66 bp, Artificial Sequence
  source: /note=“Description of Artificial Sequence Synthetic oligonucleotide”
ttcaaaagttggagcctttgtacgcctggttgtgcaaggcaaggtagtttcaatagttactgttgc

SEQ ID NO: 267 - DNA, 66 bp, Artificial Sequence
  source: /note=“Description of Artificial Sequence Synthetic oligonucleotide”
ttcaaaagttggagcctttgtacgcctggttgtgcaaggtttggtagtttcaatagttactgttgc

SEQ ID NO: 268 - DNA, 66 bp, Artificial Sequence
  source: /note=“Description of Artificial Sequence Synthetic oligonucleotide”
ttcaaaagttggagcctttgtacgcctggttgtgcaaggggtggtagtttcaatagttactgttgc

SEQ ID NO: 269 - DNA, 66 bp, Artificial Sequence
  source: /note=“Description of Artificial Sequence Synthetic oligonucleotide”
ttcaaaagttggagcctttgtacgcctggttgtgcaaggcgtggtagtttcaatagttactgttgc

SEQ ID NO: 270 - DNA, 66 bp, Artificial Sequence
  source: /note=“Description of Artificial Sequence Synthetic oligonucleotide”
ttcaaaagttggagcctttgtacgcctggttgtgcaaggcatggtagtttcaatagttactgttgc

SEQ ID NO: 271 - DNA, 66 bp, Artificial Sequence
  source: /note=“Description of Artificial Sequence Synthetic oligonucleotide”
ttcaaaagttggagcctttgtacgcctggttgtgcaaggtctggtagtttcaatagttactgttgc

SEQ ID NO: 272 - DNA, 66 bp, Artificial Sequence
  source: /note=“Description of Artificial Sequence Synthetic oligonucleotide”
ttcaaaagttggagcctttgtacgcctggttgtgcaaggattggtagtttcaatagttactgttgc

SEQ ID NO: 273 - DNA, 66 bp, Artificial Sequence
  source: /note=“Description of Artificial Sequence Synthetic oligonucleotide”
ttcaaaagttggagcctttgtacgcctggttgtgcaaggacaaaaagtttcaatagttactgttgc

SEQ ID NO: 274 - DNA, 66 bp, Artificial Sequence
  source: /note=“Description of Artificial Sequence Synthetic oligonucleotide”
ttcaaaagttggagcctttgtacgcctggttgtgcaaggacagttagtttcaatagttactgttgc

SEQ ID NO: 275 - DNA, 66 bp, Artificial Sequence
  source: /note=“Description of Artificial Sequence Synthetic oligonucleotide”
ttcaaaagttggagcctttgtacgcctggttgtgcaaggacatggagtttcaatagttactgttgc

SEQ ID NO: 276 - DNA, 66 bp, Artificial Sequence
  source: /note=“Description of Artificial Sequence Synthetic oligonucleotide”
ttcaaaagttggagcctttgtacgcctggttgtgcaaggacagctagtttcaatagttactgttgc

SEQ ID NO: 277 - DNA, 66 bp, Artificial Sequence
  source: /note=“Description of Artificial Sequence Synthetic oligonucleotide”
ttcaaaagttggagcctttgtacgcctggttgtgcaaggacattaagtttcaatagttactgttgc

SEQ ID NO: 278 - DNA, 66 bp, Artificial Sequence
  source: /note=“Description of Artificial Sequence Synthetic oligonucleotide”
ttcaaaagttggagcctttgtacgcctggttgtgcaaggacaatgagtttcaatagttactgttgc

SEQ ID NO: 279 - DNA, 66 bp, Artificial Sequence
  source: /note=“Description of Artificial Sequence Synthetic oligonucleotide”
ttcaaaagttggagcctttgtacgcctggttgtgcaaggacatgtagtttcaatagttactgttgc

SEQ ID NO: 280 - DNA, 66 bp, Artificial Sequence
  source: /note=“Description of Artificial Sequence Synthetic oligonucleotide”
ttcaaaagttggagcctttgtacgcctggttgtgcaaggacaaatagtttcaatagttactgttgc

SEQ ID NO: 281 - DNA, 66 bp, Artificial Sequence
  source: /note=“Description of Artificial Sequence Synthetic oligonucleotide”
ttcaaaagttggagcctttgtacgcctggttgtgcaaggacatatagtttcaatagttactgttgc

SEQ ID NO: 282 - DNA, 66 bp, Artificial Sequence
  source: /note=“Description of Artificial Sequence Synthetic oligonucleotide”
ttcaaaagttggagcctttgtacgcctggttgtgcaaggacagatagtttcaatagttactgttgc

SEQ ID NO: 283 - DNA, 66 bp, Artificial Sequence
  source: /note=“Description of Artificial Sequence Synthetic oligonucleotide”
ttcaaaagttggagcctttgtacgcctggttgtgcaaggacagaaagtttcaatagttactgttgc

SEQ ID NO: 284 - DNA, 66 bp, Artificial Sequence
  source: /note=“Description of Artificial Sequence Synthetic oligonucleotide”
ttcaaaagttggagcctttgtacgcctggttgtgcaaggacacctagtttcaatagttactgttgc

SEQ ID NO: 285 - DNA, 66 bp, Artificial Sequence
  source: /note=“Description of Artificial Sequence Synthetic oligonucleotide”
ttcaaaagttggagcctttgtacgcctggttgtgcaaggacacaaagtttcaatagttactgttgc

SEQ ID NO: 286 - DNA, 66 bp, Artificial Sequence
  source: /note=“Description of Artificial Sequence Synthetic oligonucleotide”
ttcaaaagttggagcctttgtacgcctggttgtgcaaggacatttagtttcaatagttactgttgc

SEQ ID NO: 287 - DNA, 66 bp, Artificial Sequence
  source: /note=“Description of Artificial Sequence Synthetic oligonucleotide”
ttcaaaagttggagcctttgtacgcctggttgtgcaaggacacgtagtttcaatagttactgttgc

SEQ ID NO: 288 - DNA, 66 bp, Artificial Sequence
  source: /note=“Description of Artificial Sequence Synthetic oligonucleotide”
ttcaaaagttggagcctttgtacgcctggttgtgcaaggacacatagtttcaatagttactgttgc

SEQ ID NO: 289 - DNA, 66 bp, Artificial Sequence
  source: /note=“Description of Artificial Sequence Synthetic oligonucleotide”
ttcaaaagttggagcctttgtacgcctggttgtgcaaggacatctagtttcaatagttactgttgc

SEQ ID NO: 290 - DNA, 66 bp, Artificial Sequence
  source: /note=“Description of Artificial Sequence Synthetic oligonucleotide”
ttcaaaagttggagcctttgtacgcctggttgtgcaaggacaacaagtttcaatagttactgttgc

SEQ ID NO: 291 - DNA, 66 bp, Artificial Sequence
  source: /note=“Description of Artificial Sequence Synthetic oligonucleotide”
ttcaaaagttggagcctttgtacgcctggttgtgcaaggacaattagtttcaatagttactgttgc

SEQ ID NO: 292 - DNA, 66 bp, Artificial Sequence
  source: /note=“Description of Artificial Sequence Synthetic oligonucleotide”
ttcaaaagttggagcctttgtacgcctggttgtgcaaggacaggtaaattcaatagttactgttgc

SEQ ID NO: 293 - DNA, 66 bp, Artificial Sequence
  source: /note=“Description of Artificial Sequence Synthetic oligonucleotide”
ttcaaaagttggagcctttgtacgcctggttgtgcaaggacaggtgttttcaatagttactgttgc

SEQ ID NO: 294 - DNA, 66 bp, Artificial Sequence
  source: /note=“Description of Artificial Sequence Synthetic oligonucleotide”
ttcaaaagttggagcctttgtacgcctggttgtgcaaggacaggttggttcaatagttactgttgc

SEQ ID NO: 295 - DNA, 66 bp, Artificial Sequence
  source: /note=“Description of Artificial Sequence Synthetic oligonucleotide”
ttcaaaagttggagcctttgtacgcctggttgtgcaaggacaggtgctttcaatagttactgttgc

SEQ ID NO: 296 - DNA, 66 bp, Artificial Sequence
  source: /note=“Description of Artificial Sequence Synthetic oligonucleotide”
ttcaaaagttggagcctttgtacgcctggttgtgcaaggacaggtttattcaatagttactgttgc

SEQ ID NO: 297 - DNA, 66 bp, Artificial Sequence
  source: /note=“Description of Artificial Sequence Synthetic oligonucleotide”
ttcaaaagttggagcctttgtacgcctggttgtgcaaggacaggtatgttcaatagttactgttgc

SEQ ID NO: 298 - DNA, 66 bp, Artificial Sequence
  source: /note=“Description of Artificial Sequence Synthetic oligonucleotide”
ttcaaaagttggagcctttgtacgcctggttgtgcaaggacaggttgtttcaatagttactgttgc

SEQ ID NO: 299 - DNA, 66 bp, Artificial Sequence
  source: /note=“Description of Artificial Sequence Synthetic oligonucleotide”
ttcaaaagttggagcctttgtacgcctggttgtgcaaggacaggtaatttcaatagttactgttgc

SEQ ID NO: 300 - DNA, 66 bp, Artificial Sequence
  source: /note=“Description of Artificial Sequence Synthetic oligonucleotide”
ttcaaaagttggagcctttgtacgcctggttgtgcaaggacaggttatttcaatagttactgttgc

SEQ ID NO: 301 - DNA, 66 bp, Artificial Sequence
  source: /note=“Description of Artificial Sequence Synthetic oligonucleotide”
ttcaaaagttggagcctttgtacgcctggttgtgcaaggacaggtgatttcaatagttactgttgc

SEQ ID NO: 302 - DNA, 66 bp, Artificial Sequence
  source: /note=“Description of Artificial Sequence Synthetic oligonucleotide”
ttcaaaagttggagcctttgtacgcctggttgtgcaaggacaggtgaattcaatagttactgttgc

SEQ ID NO: 303 - DNA, 66 bp, Artificial Sequence
  source: /note=“Description of Artificial Sequence Synthetic oligonucleotide”
ttcaaaagttggagcctttgtacgcctggttgtgcaaggacaggtcctttcaatagttactgttgc

SEQ ID NO: 304 - DNA, 66 bp, Artificial Sequence
  source: /note=“Description of Artificial Sequence Synthetic oligonucleotide”
ttcaaaagttggagcctttgtacgcctggttgtgcaaggacaggtcaattcaatagttactgttgc

SEQ ID NO: 305 - DNA, 66 bp, Artificial Sequence
  source: /note=“Description of Artificial Sequence Synthetic oligonucleotide”
ttcaaaagttggagcctttgtacgcctggttgtgcaaggacaggttttttcaatagttactgttgc

SEQ ID NO: 306 - DNA, 66 bp, Artificial Sequence
  source: /note=“Description of Artificial Sequence Synthetic oligonucleotide”
ttcaaaagttggagcctttgtacgcctggttgtgcaaggacaggtggtttcaatagttactgttgc

SEQ ID NO: 307 - DNA, 66 bp, Artificial Sequence
  source: /note=“Description of Artificial Sequence Synthetic oligonucleotide”
ttcaaaagttggagcctttgtacgcctggttgtgcaaggacaggtcgtttcaatagttactgttgc

SEQ ID NO: 308 - DNA, 66 bp, Artificial Sequence
  source: /note=“Description of Artificial Sequence Synthetic oligonucleotide”
ttcaaaagttggagcctttgtacgcctggttgtgcaaggacaggtcatttcaatagttactgttgc

SEQ ID NO: 309 - DNA, 66 bp, Artificial Sequence
  source: /note=“Description of Artificial Sequence Synthetic oligonucleotide”
ttcaaaagttggagcctttgtacgcctggttgtgcaaggacaggttctttcaatagttactgttgc

SEQ ID NO: 310 - DNA, 66 bp, Artificial Sequence
  source: /note=“Description of Artificial Sequence Synthetic oligonucleotide”
ttcaaaagttggagcctttgtacgcctggttgtgcaaggacaggtacattcaatagttactgttgc

SEQ ID NO: 311 - DNA, 66 bp, Artificial Sequence
  source: /note=“Description of Artificial Sequence Synthetic oligonucleotide”
ttcaaaagttggagcctttgtacgcctggttgtgcaaggacaggtattttcaatagttactgttgc

SEQ ID NO: 312 - DNA, 66 bp, Artificial Sequence
  source: /note=“Description of Artificial Sequence Synthetic oligonucleotide”
ttcaaaagttggagcctttgtacgcctggttgtgcaaggacaggtagtaaaaatagttactgttgc

SEQ ID NO: 313 - DNA, 66 bp, Artificial Sequence
  source: /note=“Description of Artificial Sequence Synthetic oligonucleotide”
ttcaaaagttggagcctttgtacgcctggttgtgcaaggacaggtagtgttaatagttactgttgc

SEQ ID NO: 314 - DNA, 66 bp, Artificial Sequence
  source: /note=“Description of Artificial Sequence Synthetic oligonucleotide”
ttcaaaagttggagcctttgtacgcctggttgtgcaaggacaggtagttggaatagttactgttgc

SEQ ID NO: 315 - DNA, 66 bp, Artificial Sequence
  source: /note=“Description of Artificial Sequence Synthetic oligonucleotide”
ttcaaaagttggagcctttgtacgcctggttgtgcaaggacaggtagtgctaatagttactgttgc

SEQ ID NO: 316 - DNA, 66 bp, Artificial Sequence
  source: /note=“Description of Artificial Sequence Synthetic oligonucleotide”
ttcaaaagttggagcctttgtacgcctggttgtgcaaggacaggtagtttaaatagttactgttgc

SEQ ID NO: 317 - DNA, 66 bp, Artificial Sequence
  source: /note=“Description of Artificial Sequence Synthetic oligonucleotide”
ttcaaaagttggagcctttgtacgcctggttgtgcaaggacaggtagtatgaatagttactgttgc

SEQ ID NO: 318 - DNA, 66 bp, Artificial Sequence
  source: /note=“Description of Artificial Sequence Synthetic oligonucleotide”
ttcaaaagttggagcctttgtacgcctggttgtgcaaggacaggtagttgtaatagttactgttgc

SEQ ID NO: 319 - DNA, 66 bp, Artificial Sequence
  source: /note=“Description of Artificial Sequence Synthetic oligonucleotide”
ttcaaaagttggagcctttgtacgcctggttgtgcaaggacaggtagtaataatagttactgttgc

SEQ ID NO: 320 - DNA, 66 bp, Artificial Sequence
  source: /note=“Description of Artificial Sequence Synthetic oligonucleotide”
ttcaaaagttggagcctttgtacgcctggttgtgcaaggacaggtagttataatagttactgttgc

SEQ ID NO: 321 - DNA, 66 bp, Artificial Sequence
  source: /note=“Description of Artificial Sequence Synthetic oligonucleotide”
ttcaaaagttggagcctttgtacgcctggttgtgcaaggacaggtagtgataatagttactgttgc

SEQ ID NO: 322 - DNA, 66 bp, Artificial Sequence
  source: /note=“Description of Artificial Sequence Synthetic oligonucleotide”
ttcaaaagttggagcctttgtacgcctggttgtgcaaggacaggtagtgaaaatagttactgttgc

SEQ ID NO: 323 - DNA, 66 bp, Artificial Sequence
  source: /note=“Description of Artificial Sequence Synthetic oligonucleotide”
ttcaaaagttggagcctttgtacgcctggttgtgcaaggacaggtagtcctaatagttactgttgc

SEQ ID NO: 324 - DNA, 66 bp, Artificial Sequence
  source: /note=“Description of Artificial Sequence Synthetic oligonucleotide”
ttcaaaagttggagcctttgtacgcctggttgtgcaaggacaggtagtcaaaatagttactgttgc

SEQ ID NO: 325 - DNA, 66 bp, Artificial Sequence
  source: /note=“Description of Artificial Sequence Synthetic oligonucleotide”
ttcaaaagttggagcctttgtacgcctggttgtgcaaggacaggtagttttaatagttactgttgc

SEQ ID NO: 326 - DNA, 66 bp, Artificial Sequence
  source: /note=“Description of Artificial Sequence Synthetic oligonucleotide”
ttcaaaagttggagcctttgtacgcctggttgtgcaaggacaggtagtggtaatagttactgttgc

SEQ ID NO: 327 - DNA, 66 bp, Artificial Sequence
  source: /note=“Description of Artificial Sequence Synthetic oligonucleotide”
ttcaaaagttggagcctttgtacgcctggttgtgcaaggacaggtagtcgtaatagttactgttgc

SEQ ID NO: 328 - DNA, 66 bp, Artificial Sequence
  source: /note=“Description of Artificial Sequence Synthetic oligonucleotide”
ttcaaaagttggagcctttgtacgcctggttgtgcaaggacaggtagtcataatagttactgttgc

SEQ ID NO: 329 - DNA, 66 bp, Artificial Sequence
  source: /note=“Description of Artificial Sequence Synthetic oligonucleotide”
ttcaaaagttggagcctttgtacgcctggttgtgcaaggacaggtagttctaatagttactgttgc

SEQ ID NO: 330 - DNA, 66 bp, Artificial Sequence
  source: /note=“Description of Artificial Sequence Synthetic oligonucleotide”
ttcaaaagttggagcctttgtacgcctggttgtgcaaggacaggtagtacaaatagttactgttgc

SEQ ID NO: 331 - DNA, 66 bp, Artificial Sequence
  source: /note=“Description of Artificial Sequence Synthetic oligonucleotide”
ttcaaaagttggagcctttgtacgcctggttgtgcaaggacaggtagtattaatagttactgttgc

SEQ ID NO: 332 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaaaagttactgttgc

SEQ ID NO: 333 - DNA, 66 bp, Artificial Sequence
  source: /note=“Description of Artificial Sequence Synthetic oligonucleotide”
ttcaaaagttggagcctttgtacgcctggttgtgcaaggacaggtagtttcgttagttactgttgc

SEQ ID NO: 334 - DNA, 66 bp, Artificial Sequence
  source: /note=“Description of Artificial Sequence Synthetic oligonucleotide”
ttcaaaagttggagcctttgtacgcctggttgtgcaaggacaggtagtttctggagttactgttgc

SEQ ID NO: 335 - DNA, 66 bp, Artificial Sequence
  source: /note=“Description of Artificial Sequence Synthetic oligonucleotide”
ttcaaaagttggagcctttgtacgcctggttgtgcaaggacaggtagtttcgctagttactgttgc

SEQ ID NO: 336 - DNA, 66 bp, Artificial Sequence
  source: /note=“Description of Artificial Sequence Synthetic oligonucleotide”
ttcaaaagttggagcctttgtacgcctggttgtgcaaggacaggtagtttcttaagttactgttgc

SEQ ID NO: 337 - DNA, 66 bp, Artificial Sequence
  source: /note=“Description of Artificial Sequence Synthetic oligonucleotide”
ttcaaaagttggagcctttgtacgcctggttgtgcaaggacaggtagtttcatgagttactgttgc

SEQ ID NO: 338 - DNA, 66 bp, Artificial Sequence
  source: /note=“Description of Artificial Sequence Synthetic oligonucleotide”
ttcaaaagttggagcctttgtacgcctggttgtgcaaggacaggtagtttctgtagttactgttgc

SEQ ID NO: 339 - DNA, 66 bp, Artificial Sequence
  source: /note=“Description of Artificial Sequence Synthetic oligonucleotide”
ttcaaaagttggagcctttgtacgcctggttgtgcaaggacaggtagtttctatagttactgttgc

SEQ ID NO: 340 - DNA, 66 bp, Artificial Sequence
  source: /note=“Description of Artificial Sequence Synthetic oligonucleotide”
ttcaaaagttggagcctttgtacgcctggttgtgcaaggacaggtagtttcgatagttactgttgc

SEQ ID NO: 341 - DNA, 66 bp, Artificial Sequence
  source: /note=“Description of Artificial Sequence Synthetic oligonucleotide”
ttcaaaagttggagcctttgtacgcctggttgtgcaaggacaggtagtttcgaaagttactgttgc

SEQ ID NO: 342 - DNA, 66 bp, Artificial Sequence
  source: /note=“Description of Artificial Sequence Synthetic oligonucleotide”
ttcaaaagttggagcctttgtacgcctggttgtgcaaggacaggtagtttccctagttactgttgc

SEQ ID NO: 343 - DNA, 66 bp, Artificial Sequence
  source: /note=“Description of Artificial Sequence Synthetic oligonucleotide”
ttcaaaagttggagcctttgtacgcctggttgtgcaaggacaggtagtttccaaagttactgttgc

SEQ ID NO: 344 - DNA, 66 bp, Artificial Sequence
  source: /note=“Description of Artificial Sequence Synthetic oligonucleotide”
ttcaaaagttggagcctttgtacgcctggttgtgcaaggacaggtagtttctttagttactgttgc

SEQ ID NO: 345 - DNA, 66 bp, Artificial Sequence
  source: /note=“Description of Artificial Sequence Synthetic oligonucleotide”
ttcaaaagttggagcctttgtacgcctggttgtgcaaggacaggtagtttcggtagttactgttgc

SEQ ID NO: 346 - DNA, 66 bp, Artificial Sequence
  source: /note=“Description of Artificial Sequence Synthetic oligonucleotide”
ttcaaaagttggagcctttgtacgcctggttgtgcaaggacaggtagtttccgtagttactgttgc

SEQ ID NO: 347 - DNA, 66 bp, Artificial Sequence
  source: /note=“Description of Artificial Sequence Synthetic oligonucleotide”
ttcaaaagttggagcctttgtacgcctggttgtgcaaggacaggtagtttccatagttactgttgc

SEQ ID NO: 348 - DNA, 66 bp, Artificial Sequence
  source: /note=“Description of Artificial Sequence Synthetic oligonucleotide”
ttcaaaagttggagcctttgtacgcctggttgtgcaaggacaggtagtttctctagttactgttgc

SEQ ID NO: 349 - DNA, 66 bp, Artificial Sequence
  source: /note=“Description of Artificial Sequence Synthetic oligonucleotide”
ttcaaaagttggagcctttgtacgcctggttgtgcaaggacaggtagtttcacaagttactgttgc

SEQ ID NO: 350 - DNA, 66 bp, Artificial Sequence
  source: /note=“Description of Artificial Sequence Synthetic oligonucleotide”
ttcaaaagttggagcctttgtacgcctggttgtgcaaggacaggtagtttcattagttactgttgc

SEQ ID NO: 351 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaataaatactgttgc

SEQ ID NO: 352 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatgtttactgttgc

SEQ ID NO: 353 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaattggtactgttgc

SEQ ID NO: 354 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatgcttactgttgc

SEQ ID NO: 355 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatttatactgttgc

SEQ ID NO: 356 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatatgtactgttgc

SEQ ID NO: 357 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaattgttactgttgc

SEQ ID NO: 358 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaataattactgttgc

SEQ ID NO: 359 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaattattactgttgc

SEQ ID NO: 360 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatgattactgttgc

SEQ ID NO: 361 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatgaatactgttgc

SEQ ID NO: 362 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatccttactgttgc

SEQ ID NO: 363 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatcaatactgttgc

SEQ ID NO: 364 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatttttactgttgc

SEQ ID NO: 365 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatggttactgttgc

SEQ ID NO: 366 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatcgttactgttgc

SEQ ID NO: 367 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatcattactgttgc

SEQ ID NO: 368 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaattcttactgttgc

SEQ ID NO: 369 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatacatactgttgc

SEQ ID NO: 370 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatatttactgttgc

SEQ ID NO: 371 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagtaaatgttgc

SEQ ID NO: 372 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagtgtttgttgc

SEQ ID NO: 373 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttggtgttgc

SEQ ID NO: 374 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagtgcttgttgc

SEQ ID NO: 375 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagtttatgttgc

SEQ ID NO: 376 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagtatgtgttgc

SEQ ID NO: 377 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttgttgttgc

SEQ ID NO: 378 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagtaattgttgc

SEQ ID NO: 379 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttattgttgc

SEQ ID NO: 380 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagtgattgttgc

SEQ ID NO: 381 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagtgaatgttgc

SEQ ID NO: 382 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagtccttgttgc

SEQ ID NO: 383 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagtcaatgttgc

SEQ ID NO: 384 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagtttttgttgc

SEQ ID NO: 385 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagtggttgttgc

SEQ ID NO: 386 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagtcgttgttgc

SEQ ID NO: 387 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagtcattgttgc

SEQ ID NO: 388 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttcttgttgc

SEQ ID NO: 389 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagtacatgttgc

SEQ ID NO: 390 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagtatttgttgc

SEQ ID NO: 391 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttacaaatgc

SEQ ID NO: 392 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttacgtttgc

SEQ ID NO: 393 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactggtgc

SEQ ID NO: 394 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttacgcttgc

SEQ ID NO: 395 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttacttatgc

SEQ ID NO: 396 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttacatgtgc

SEQ ID NO: 397 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttacaattgc

SEQ ID NO: 398 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactattgc

SEQ ID NO: 399 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttacgattgc

SEQ ID NO: 400 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttacgaatgc

SEQ ID NO: 401 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttacccttgc

SEQ ID NO: 402 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttaccaatgc

SEQ ID NO: 403 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttacttttgc

SEQ ID NO: 404 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttacggttgc

SEQ ID NO: 405 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttaccgttgc

SEQ ID NO: 406 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttaccattgc

SEQ ID NO: 407 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactcttgc

SEQ ID NO: 408 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttacacatgc

SEQ ID NO: 409 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttacatttgc

SEQ ID NO: 410 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgtaaa

SEQ ID NO: 411 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgtgtt

SEQ ID NO: 412 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgttgg

SEQ ID NO: 413 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgtgct

SEQ ID NO: 414 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgttta

SEQ ID NO: 415 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgtatg

SEQ ID NO: 416 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgttgt

SEQ ID NO: 417 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgtaat

SEQ ID NO: 418 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgttat

SEQ ID NO: 419 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgtgat

SEQ ID NO: 420 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgtgaa

SEQ ID NO: 421 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgtcct

SEQ ID NO: 422 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgtcaa

SEQ ID NO: 423 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgtttt

SEQ ID NO: 424 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgtggt

SEQ ID NO: 425 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgtcgt

SEQ ID NO: 426 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgtcat

SEQ ID NO: 427 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgttct

SEQ ID NO: 428 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgtaca

SEQ ID NO: 429 - DNA, 66 bp, Artificial Sequence
  source: /note=“Description of Artificial Sequence Synthetic oligonucleotide”
ttcaaaagttggagcctttgtacgcctggttgtgcaaggacaggtagtttcaatagttactgtatt

SEQ ID NO: 430 - DNA, 66 bp, Artificial Sequence; identical to SEQ ID NO: 400
  source: /note=“Description of Artificial Sequence Synthetic oligonucleotide”

SEQ ID NO: 431 - DNA, 66 bp, Artificial Sequence; identical to SEQ ID NO: 420
  source: /note=“Description of Artificial Sequence Synthetic oligonucleotide”

SEQ ID NO: 432 - protein, 22 aa, Streptococcus mutans
FKSWSLCTPGCARTGSFNSYCC

SEQ ID NO: 433 - protein, 22 aa, Artificial Sequence
  source: /note=“Description of Artificial Sequence Synthetic peptide”
IKSWSLCTPGCANTGSFNSYCC

SEQ ID NO: 434 - protein, 22 aa, Artificial Sequence
  source: /note=“Description of Artificial Sequence Synthetic peptide”
IKSWSLCTPGCADTGSFNSYCC

SEQ ID NO: 435 - protein, 22 aa, Artificial Sequence
  source: /note=“Description of Artificial Sequence Synthetic peptide”
IKSWSLCTPGCAATGSFNSYCC

SEQ ID NO: 436 - protein, 22 aa, Artificial Sequence
  source: /note=“Description of Artificial Sequence Synthetic peptide”
IKSWSLCTPGCATTGSFNSYCC

SEQ ID NO: 437 - protein, 22 aa, Artificial Sequence
  source: /note=“Description of Artificial Sequence Synthetic peptide”
IKSWSLCTPGCAKTGSFNSYCC

SEQ ID NO: 438 - protein, 22 aa, Artificial Sequence
  source: /note=“Description of Artificial Sequence Synthetic peptide”
IKSWSLCTPGCASTGSFNSYCC

SEQ ID NO: 439 - protein, 22 aa, Artificial Sequence
  source: /note=“Description of Artificial Sequence Synthetic peptide”
IKSWSLCTPGCAGTGSFNSYCC

SEQ ID NO: 440 - protein, 22 aa, Artificial Sequence
  source: /note=“Description of Artificial Sequence Synthetic peptide”
IKSWSLCTPGCAVTGSFNSYCC

SEQ ID NO: 441 - protein, 22 aa, Artificial Sequence
  source: /note=“Description of Artificial Sequence Synthetic peptide”
IKSWSLCTPGCAITGSFNSYCC

SEQ ID NO: 442 - protein, 22 aa, Artificial Sequence
  source: /note=“Description of Artificial Sequence Synthetic peptide”
IKSWSLCTPGCAPTGSFNSYCC

SEQ ID NO: 443 - protein, 22 aa, Artificial Sequence
  source: /note=“Description of Artificial Sequence Synthetic peptide”
IKSWSLCTPGCAQTGSFNSYCC

SEQ ID NO: 444 - protein, 22 aa, Artificial Sequence
  source: /note=“Description of Artificial Sequence Synthetic peptide”
IKSWSLCTPGCAETGSFNSYCC

SEQ ID NO: 445 - protein, 22 aa, Artificial Sequence
  source: /note=“Description of Artificial Sequence Synthetic peptide”
IKSWSLCTPGCAHTGSFNSYCC

SEQ ID NO: 446 - protein, 22 aa, Artificial Sequence
  source: /note=“Description of Artificial Sequence Synthetic peptide”
LKSWSLCTPGCANTGSFNSYCC

SEQ ID NO: 447 - protein, 22 aa, Artificial Sequence
  source: /note=“Description of Artificial Sequence Synthetic peptide”
LKSWSLCTPGCADTGSFNSYCC

SEQ ID NO: 448 - protein, 22 aa, Artificial Sequence
  source: /note=“Description of Artificial Sequence Synthetic peptide”
LKSWSLCTPGCAATGSFNSYCC

SEQ ID NO: 449 - protein, 22 aa, Artificial Sequence
  source: /note=“Description of Artificial Sequence Synthetic peptide”
LKSWSLCTPGCATTGSFNSYCC

SEQ ID NO: 450 - protein, 22 aa, Artificial Sequence
  source: /note=“Description of Artificial Sequence Synthetic peptide”
LKSWSLCTPGCAKTGSFNSYCC

SEQ ID NO: 451 - protein, 22 aa, Artificial Sequence
  source: /note=“Description of Artificial Sequence Synthetic peptide”
LKSWSLCTPGCASTGSFNSYCC

SEQ ID NO: 452 - protein, 22 aa, Artificial Sequence
  source: /note=“Description of Artificial Sequence Synthetic peptide”
LKSWSLCTPGCAGTGSFNSYCC

SEQ ID NO: 453 - protein, 22 aa, Artificial Sequence
  source: /note=“Description of Artificial Sequence Synthetic peptide”
LKSWSLCTPGCAVTGSFNSYCC

SEQ ID NO: 454 - protein, 22 aa, Artificial Sequence
  source: /note=“Description of Artificial Sequence Synthetic peptide”
LKSWSLCTPGCAITGSFNSYCC

SEQ ID NO: 455 - protein, 22 aa, Artificial Sequence
  source: /note=“Description of Artificial Sequence Synthetic peptide”
LKSWSLCTPGCAPTGSFNSYCC

SEQ ID NO: 456 - protein, 22 aa, Artificial Sequence
  source: /note=“Description of Artificial Sequence Synthetic peptide”
LKSWSLCTPGCAQTGSFNSYCC

SEQ ID NO: 457 - protein, 22 aa, Artificial Sequence
  source: /note=“Description of Artificial Sequence Synthetic peptide”
LKSWSLCTPGCAETGSFNSYCC

SEQ ID NO: 458 - protein, 22 aa, Artificial Sequence
  source: /note=“Description of Artificial Sequence Synthetic peptide”
LKSWSLCTPGCAHTGSFNSYCC

SEQ ID NO: 459 - protein, 22 aa, Artificial Sequence
  source: /note=“Description of Artificial Sequence Synthetic peptide”
AKSWSLCTPGCANTGSFNSYCC

SEQ ID NO: 460 - protein, 22 aa, Artificial Sequence
  source: /note=“Description of Artificial Sequence Synthetic peptide”
AKSWSLCTPGCADTGSFNSYCC

SEQ ID NO: 461 - protein, 22 aa, Artificial Sequence
  source: /note=“Description of Artificial Sequence Synthetic peptide”
AKSWSLCTPGCAATGSFNSYCC

SEQ ID NO: 462 - protein, 22 aa, Artificial Sequence
  source: /note=“Description of Artificial Sequence Synthetic peptide”
AKSWSLCTPGCATTGSFNSYCC

SEQ ID NO: 463 - protein, 22 aa, Artificial Sequence
  source: /note=“Description of Artificial Sequence Synthetic peptide”
AKSWSLCTPGCAKTGSFNSYCC

SEQ ID NO: 464 - protein, 22 aa, Artificial Sequence
  source: /note=“Description of Artificial Sequence Synthetic peptide”
AKSWSLCTPGCASTGSFNSYCC

SEQ ID NO: 465 - protein, 22 aa, Artificial Sequence
  source: /note=“Description of Artificial Sequence Synthetic peptide”
AKSWSLCTPGCAGTGSFNSYCC

SEQ ID NO: 466 - protein, 22 aa, Artificial Sequence
  source: /note=“Description of Artificial Sequence Synthetic peptide”
AKSWSLCTPGCAVTGSFNSYCC

SEQ ID NO: 467 - protein, 22 aa, Artificial Sequence
  source: /note=“Description of Artificial Sequence Synthetic peptide”
AKSWSLCTPGCAITGSFNSYCC

SEQ ID NO: 468 - protein, 22 aa, Artificial Sequence
  source: /note=“Description of Artificial Sequence Synthetic peptide”
AKSWSLCTPGCAPTGSFNSYCC

SEQ ID NO: 469 - protein, 22 aa, Artificial Sequence
  source: /note=“Description of Artificial Sequence Synthetic peptide”
AKSWSLCTPGCAQTGSFNSYCC

SEQ ID NO: 470 - protein, 22 aa, Artificial Sequence
  source: /note=“Description of Artificial Sequence Synthetic peptide”
AKSWSLCTPGCAETGSFNSYCC

SEQ ID NO: 471 - protein, 22 aa, Artificial Sequence
  source: /note=“Description of Artificial Sequence Synthetic peptide”
AKSWSLCTPGCAHTGSFNSYCC

SEQ ID NO: 472 - protein, 22 aa, Artificial Sequence
  source: /note=“Description of Artificial Sequence Synthetic peptide”
NKSWSLCTPGCANTGSFNSYCC

SEQ ID NO: 473 - protein, 22 aa, Artificial Sequence
  source: /note=“Description of Artificial Sequence Synthetic peptide”
NKSWSLCTPGCADTGSFNSYCC

SEQ ID NO: 474 - protein, 22 aa, Artificial Sequence
  source: /note=“Description of Artificial Sequence Synthetic peptide”
NKSWSLCTPGCAATGSFNSYCC

SEQ ID NO: 475 - protein, 22 aa, Artificial Sequence
  source: /note=“Description of Artificial Sequence Synthetic peptide”
NKSWSLCTPGCATTGSFNSYCC

SEQ ID NO: 476 - protein, 22 aa, Artificial Sequence
  source: /note=“Description of Artificial Sequence Synthetic peptide”
NKSWSLCTPGCAKTGSFNSYCC

SEQ ID NO: 477 - protein, 22 aa, Artificial Sequence
  source: /note=“Description of Artificial Sequence Synthetic peptide”
NKSWSLCTPGCASTGSFNSYCC

SEQ ID NO: 478 - protein, 22 aa, Artificial Sequence
  source: /note=“Description of Artificial Sequence Synthetic peptide”
NKSWSLCTPGCAGTGSFNSYCC

SEQ ID NO: 479 - protein, 22 aa, Artificial Sequence
  source: /note=“Description of Artificial Sequence Synthetic peptide”
NKSWSLCTPGCAVTGSFNSYCC

SEQ ID NO: 480 - protein, 22 aa, Artificial Sequence
  source: /note=“Description of Artificial Sequence Synthetic peptide”
NKSWSLCTPGCAITGSFNSYCC

SEQ ID NO: 481 - protein, 22 aa, Artificial Sequence
  source: /note=“Description of Artificial Sequence Synthetic peptide”
NKSWSLCTPGCAPTGSFNSYCC

SEQ ID NO: 482 - protein, 22 aa, Artificial Sequence
  source: /note=“Description of Artificial Sequence Synthetic peptide”
NKSWSLCTPGCAQTGSFNSYCC

SEQ ID NO: 483 - protein, 22 aa, Artificial Sequence
  source: /note=“Description of Artificial Sequence Synthetic peptide”
NKSWSLCTPGCAETGSFNSYCC

SEQ ID NO: 484 - protein, 22 aa, Artificial Sequence
  source: /note=“Description of Artificial Sequence Synthetic peptide”
NKSWSLCTPGCAHTGSFNSYCC

SEQ ID NO: 485 - protein, 22 aa, Artificial Sequence
  source: /note=“Description of Artificial Sequence Synthetic peptide”
SKSWSLCTPGCANTGSFNSYCC

SEQ ID NO: 486 - protein, 22 aa, Artificial Sequence
  source: /note=“Description of Artificial Sequence Synthetic peptide”
SKSWSLCTPGCADTGSFNSYCC

SEQ ID NO: 487 - protein, 22 aa, Artificial Sequence
  source: /note=“Description of Artificial Sequence Synthetic peptide”
SKSWSLCTPGCAATGSFNSYCC

SEQ ID NO: 488 - protein, 22 aa, Artificial Sequence
  source: /note=“Description of Artificial Sequence Synthetic peptide”
SKSWSLCTPGCATTGSFNSYCC

SEQ ID NO: 489 - protein, 22 aa, Artificial Sequence
  source: /note=“Description of Artificial Sequence Synthetic peptide”
SKSWSLCTPGCAKTGSFNSYCC

SEQ ID NO: 490 - protein, 22 aa, Artificial Sequence
  source: /note=“Description of Artificial Sequence Synthetic peptide”
SKSWSLCTPGCASTGSFNSYCC

SEQ ID NO: 491 - protein, 22 aa, Artificial Sequence
  source: /note=“Description of Artificial Sequence Synthetic peptide”
SKSWSLCTPGCAGTGSFNSYCC

SEQ ID NO: 492 - protein, 22 aa, Artificial Sequence
  source: /note=“Description of Artificial Sequence Synthetic peptide”
SKSWSLCTPGCAVTGSFNSYCC

SEQ ID NO: 493 - protein, 22 aa, Artificial Sequence
  source: /note=“Description of Artificial Sequence Synthetic peptide”
SKSWSLCTPGCAITGSFNSYCC

SEQ ID NO: 494 - protein, 22 aa, Artificial Sequence
  source: /note=“Description of Artificial Sequence Synthetic peptide”
SKSWSLCTPGCAPTGSFNSYCC

SEQ ID NO: 495 - protein, 22 aa, Artificial Sequence
  source: /note=“Description of Artificial Sequence Synthetic peptide”
SKSWSLCTPGCAQTGSFNSYCC

SEQ ID NO: 496 - protein, 22 aa, Artificial Sequence
  source: /note=“Description of Artificial Sequence Synthetic peptide”
SKSWSLCTPGCAETGSFNSYCC

SEQ ID NO: 497 - protein, 22 aa, Artificial Sequence
  source: /note=“Description of Artificial Sequence Synthetic peptide”
SKSWSLCTPGCAHTGSFNSYCC

SEQ ID NO: 498 - protein, 22 aa, Artificial Sequence
  source: /note=“Description of Artificial Sequence Synthetic peptide”
TKSWSLCTPGCANTGSFNSYCC

SEQ ID NO: 499 - protein, 22 aa, Artificial Sequence
  source: /note=“Description of Artificial Sequence Synthetic peptide”
TKSWSLCTPGCADTGSFNSYCC

SEQ ID NO: 500 - protein, 22 aa, Artificial Sequence
  source: /note=“Description of Artificial Sequence Synthetic peptide”
TKSWSLCTPGCAATGSFNSYCC

SEQ ID NO: 501 - protein, 22 aa, Artificial Sequence
  source: /note=“Description of Artificial Sequence Synthetic peptide”
TKSWSLCTPGCATTGSFNSYCC

SEQ ID NO: 502 - protein, 22 aa, Artificial Sequence
  source: /note=“Description of Artificial Sequence Synthetic peptide”
TKSWSLCTPGCAKTGSFNSYCC

SEQ ID NO: 503 - protein, 22 aa, Artificial Sequence
  source: /note=“Description of Artificial Sequence Synthetic peptide”
TKSWSLCTPGCASTGSFNSYCC

SEQ ID NO: 504 - protein, 22 aa, Artificial Sequence
  source: /note=“Description of Artificial Sequence Synthetic peptide”
TKSWSLCTPGCAGTGSFNSYCC

SEQ ID NO: 505 - protein, 22 aa, Artificial Sequence
  source: /note=“Description of Artificial Sequence Synthetic peptide”
TKSWSLCTPGCAVTGSFNSYCC

SEQ ID NO: 506 - protein, 22 aa, Artificial Sequence
  source: /note=“Description of Artificial Sequence Synthetic peptide”
TKSWSLCTPGCAITGSFNSYCC

SEQ ID NO: 507 - protein, 22 aa, Artificial Sequence
  source: /note=“Description of Artificial Sequence Synthetic peptide”
TKSWSLCTPGCAPTGSFNSYCC

SEQ ID NO: 508 - protein, 22 aa, Artificial Sequence
  source: /note=“Description of Artificial Sequence Synthetic peptide”
TKSWSLCTPGCAQTGSFNSYCC

SEQ ID NO: 509 - protein, 22 aa, Artificial Sequence
  source: /note=“Description of Artificial Sequence Synthetic peptide”
TKSWSLCTPGCAETGSFNSYCC

SEQ ID NO: 510 - protein, 22 aa, Artificial Sequence
  source: /note=“Description of Artificial Sequence Synthetic peptide”
TKSWSLCTPGCAHTGSFNSYCC

SEQ ID NO: 511 - protein, 22 aa, Artificial Sequence
  source: /note=“Description of Artificial Sequence Synthetic peptide”
YKSWSLCTPGCANTGSFNSYCC

SEQ ID NO: 512 - protein, 22 aa, Artificial Sequence
  source: /note=“Description of Artificial Sequence Synthetic peptide”
YKSWSLCTPGCADTGSFNSYCC

SEQ ID NO: 513 - protein, 22 aa, Artificial Sequence
  source: /note=“Description of Artificial Sequence Synthetic peptide”
YKSWSLCTPGCAATGSFNSYCC

SEQ ID NO: 514 - protein, 22 aa, Artificial Sequence
  source: /note=“Description of Artificial Sequence Synthetic peptide”
YKSWSLCTPGCATTGSFNSYCC

SEQ ID NO: 515 - protein, 22 aa, Artificial Sequence
  source: /note=“Description of Artificial Sequence Synthetic peptide”
YKSWSLCTPGCAKTGSFNSYCC

SEQ ID NO: 516 - protein, 22 aa, Artificial Sequence
  source: /note=“Description of Artificial Sequence Synthetic peptide”
YKSWSLCTPGCASTGSFNSYCC

SEQ ID NO: 517 - protein, 22 aa, Artificial Sequence
  source: /note=“Description of Artificial Sequence Synthetic peptide”
YKSWSLCTPGCAGTGSFNSYCC

SEQ ID NO: 518 - protein, 22 aa, Artificial Sequence
  source: /note=“Description of Artificial Sequence Synthetic peptide”
YKSWSLCTPGCAVTGSFNSYCC

SEQ ID NO: 519 - protein, 22 aa, Artificial Sequence
  source: /note=“Description of Artificial Sequence Synthetic peptide”
YKSWSLCTPGCAITGSFNSYCC

SEQ ID NO: 520 - protein, 22 aa, Artificial Sequence
  source: /note=“Description of Artificial Sequence Synthetic peptide”
YKSWSLCTPGCAPTGSFNSYCC

SEQ ID NO: 521 - protein, 22 aa, Artificial Sequence
  source: /note=“Description of Artificial Sequence Synthetic peptide”
YKSWSLCTPGCAQTGSFNSYCC

SEQ ID NO: 522 - protein, 22 aa, Artificial Sequence
  source: /note=“Description of Artificial Sequence Synthetic peptide”
YKSWSLCTPGCAETGSFNSYCC

SEQ ID NO: 523 - protein, 22 aa, Artificial Sequence
  source: /note=“Description of Artificial Sequence Synthetic peptide”
YKSWSLCTPGCAHTGSFNSYCC

SEQ ID NO: 524 - protein, 22 aa, Artificial Sequence
  source: /note=“Description of Artificial Sequence Synthetic peptide”
HKSWSLCTPGCANTGSFNSYCC

SEQ ID NO: 525 - protein, 22 aa, Artificial Sequence
  source: /note=“Description of Artificial Sequence Synthetic peptide”
HKSWSLCTPGCADTGSFNSYCC

SEQ ID NO: 526 - protein, 22 aa, Artificial Sequence
  source: /note=“Description of Artificial Sequence Synthetic peptide”
HKSWSLCTPGCAATGSFNSYCC

SEQ ID NO: 527 - protein, 22 aa, Artificial Sequence
  source: /note=“Description of Artificial Sequence Synthetic peptide”
HKSWSLCTPGCATTGSFNSYCC

SEQ ID NO: 528 - protein, 22 aa, Artificial Sequence
  source: /note=“Description of Artificial Sequence Synthetic peptide”
HKSWSLCTPGCAKTGSFNSYCC

SEQ ID NO: 529 - protein, 22 aa, Artificial Sequence
  source: /note=“Description of Artificial Sequence Synthetic peptide”
HKSWSLCTPGCASTGSFNSYCC

SEQ ID NO: 530 - protein, 22 aa, Artificial Sequence
  source: /note=“Description of Artificial Sequence Synthetic peptide”
HKSWSLCTPGCAGTGSFNSYCC

SEQ ID NO: 531 - protein, 22 aa, Artificial Sequence
  source: /note=“Description of Artificial Sequence Synthetic peptide”
HKSWSLCTPGCAVTGSFNSYCC

SEQ ID NO: 532 - protein, 22 aa, Artificial Sequence
  source: /note=“Description of Artificial Sequence Synthetic peptide”
HKSWSLCTPGCAITGSFNSYCC

SEQ ID NO: 533 - protein, 22 aa, Artificial Sequence
  source: /note=“Description of Artificial Sequence Synthetic peptide”
HKSWSLCTPGCAPTGSFNSYCC

SEQ ID NO: 534 - protein, 22 aa, Artificial Sequence
  source: /note=“Description of Artificial Sequence Synthetic peptide”
HKSWSLCTPGCAQTGSFNSYCC

SEQ ID NO: 535 - protein, 22 aa, Artificial Sequence
  source: /note=“Description of Artificial Sequence Synthetic peptide”
HKSWSLCTPGCAETGSFNSYCC

SEQ ID NO: 536 - protein, 22 aa, Artificial Sequence
  source: /note=“Description of Artificial Sequence Synthetic peptide”
HKSWSLCTPGCAHTGSFNSYCC

SEQ ID NO: 537 - protein, 22 aa, Artificial Sequence
  source: /note=“Description of Artificial Sequence Synthetic peptide”
PKSWSLCTPGCANTGSFNSYCC

SEQ ID NO: 538 - protein, 22 aa, Artificial Sequence
  source: /note=“Description of Artificial Sequence Synthetic peptide”
PKSWSLCTPGCADTGSFNSYCC

SEQ ID NO: 539 - protein, 22 aa, Artificial Sequence
  source: /note=“Description of Artificial Sequence Synthetic peptide”
PKSWSLCTPGCAATGSFNSYCC

SEQ ID NO: 540 - protein, 22 aa, Artificial Sequence
  source: /note=“Description of Artificial Sequence Synthetic peptide”
PKSWSLCTPGCATTGSFNSYCC

SEQ ID NO: 541 - protein, 22 aa, Artificial Sequence
  source: /note=“Description of Artificial Sequence Synthetic peptide”
PKSWSLCTPGCAKTGSFNSYCC

SEQ ID NO: 542 - protein, 22 aa, Artificial Sequence
  source: /note=“Description of Artificial Sequence Synthetic peptide”
PKSWSLCTPGCASTGSFNSYCC

SEQ ID NO: 543 - protein, 22 aa, Artificial Sequence
  source: /note=“Description of Artificial Sequence Synthetic peptide”
PKSWSLCTPGCAGTGSFNSYCC

SEQ ID NO: 544 - protein, 22 aa, Artificial Sequence
  source: /note=“Description of Artificial Sequence Synthetic peptide”
PKSWSLCTPGCAVTGSFNSYCC

SEQ ID NO: 545 - protein, 22 aa, Artificial Sequence
  source: /note=“Description of Artificial Sequence Synthetic peptide”
PKSWSLCTPGCAITGSFNSYCC

SEQ ID NO: 546 - protein, 22 aa, Artificial Sequence
  source: /note=“Description of Artificial Sequence Synthetic peptide”
PKSWSLCTPGCAPTGSFNSYCC

SEQ ID NO: 547 - protein, 22 aa, Artificial Sequence
  source: /note=“Description of Artificial Sequence Synthetic peptide”
PKSWSLCTPGCAQTGSFNSYCC

SEQ ID NO: 548 - protein, 22 aa, Artificial Sequence
  source: /note=“Description of Artificial Sequence Synthetic peptide”
PKSWSLCTPGCAETGSFNSYCC

SEQ ID NO: 549 - protein, 22 aa, Artificial Sequence
  source: /note=“Description of Artificial Sequence Synthetic peptide”
PKSWSLCTPGCAHTGSFNSYCC

SEQ ID NO: 550 - protein, 22 aa, Artificial Sequence
  source: /note=“Description of Artificial Sequence Synthetic peptide”
VKSWSLCTPGCANTGSFNSYCC

SEQ ID NO: 551 - protein, 22 aa, Artificial Sequence
  source: /note=“Description of Artificial Sequence Synthetic peptide”
VKSWSLCTPGCADTGSFNSYCC

SEQ ID NO: 552 - protein, 22 aa, Artificial Sequence
  source: /note=“Description of Artificial Sequence Synthetic peptide”
VKSWSLCTPGCAATGSFNSYCC

SEQ ID NO: 553 - protein, 22 aa, Artificial Sequence
  source: /note=“Description of Artificial Sequence Synthetic peptide”
VKSWSLCTPGCATTGSFNSYCC

SEQ ID NO: 554 - protein, 22 aa, Artificial Sequence
  source: /note=“Description of Artificial Sequence Synthetic peptide”
VKSWSLCTPGCAKTGSFNSYCC

SEQ ID NO: 555 - protein, 22 aa, Artificial Sequence
  source: /note=“Description of Artificial Sequence Synthetic peptide”
VKSWSLCTPGCASTGSFNSYCC

SEQ ID NO: 556 - protein, 22 aa, Artificial Sequence
  source: /note=“Description of Artificial Sequence Synthetic peptide”
VKSWSLCTPGCAGTGSFNSYCC

SEQ ID NO: 557 - protein, 22 aa, Artificial Sequence
  source: /note=“Description of Artificial Sequence Synthetic peptide”
VKSWSLCTPGCAVTGSFNSYCC

SEQ ID NO: 558 - protein, 22 aa, Artificial Sequence
  source: /note=“Description of Artificial Sequence Synthetic peptide”
VKSWSLCTPGCAITGSFNSYCC

SEQ ID NO: 559 - protein, 22 aa, Artificial Sequence
  source: /note=“Description of Artificial Sequence Synthetic peptide”
VKSWSLCTPGCAPTGSFNSYCC

SEQ ID NO: 560 - protein, 22 aa, Artificial Sequence
  source: /note=“Description of Artificial Sequence Synthetic peptide”
VKSWSLCTPGCAQTGSFNSYCC

SEQ ID NO: 561 - protein, 22 aa, Artificial Sequence
  source: /note=“Description of Artificial Sequence Synthetic peptide”
VKSWSLCTPGCAETGSFNSYCC

SEQ ID NO: 562 - protein, 22 aa, Artificial Sequence
  source: /note=“Description of Artificial Sequence Synthetic peptide”
VKSWSLCTPGCAHTGSFNSYCC

SEQ ID NO: 563 - protein, 22 aa, Artificial Sequence
  source: /note=“Description of Artificial Sequence Synthetic peptide”
GKSWSLCTPGCANTGSFNSYCC

SEQ ID NO: 564 - protein, 22 aa, Artificial Sequence
  source: /note=“Description of Artificial Sequence Synthetic peptide”
GKSWSLCTPGCADTGSFNSYCC

SEQ ID NO: 565 - protein, 22 aa, Artificial Sequence
  source: /note=“Description of Artificial Sequence Synthetic peptide”
GKSWSLCTPGCAATGSFNSYCC

SEQ ID NO: 566 - protein, 22 aa, Artificial Sequence
  source: /note=“Description of Artificial Sequence Synthetic peptide”
GKSWSLCTPGCATTGSFNSYCC

SEQ ID NO: 567 - protein, 22 aa, Artificial Sequence
  source: /note=“Description of Artificial Sequence Synthetic peptide”
GKSWSLCTPGCAKTGSFNSYCC

SEQ ID NO: 568 - protein, 22 aa, Artificial Sequence
  source: /note=“Description of Artificial Sequence Synthetic peptide”
GKSWSLCTPGCASTGSFNSYCC

SEQ ID NO: 569 - protein, 22 aa, Artificial Sequence
  source: /note=“Description of Artificial Sequence Synthetic peptide”
GKSWSLCTPGCAGTGSFNSYCC

SEQ ID NO: 570 - protein, 22 aa, Artificial Sequence
  source: /note=“Description of Artificial Sequence Synthetic peptide”
GKSWSLCTPGCAVTGSFNSYCC

SEQ ID NO: 571 - protein, 22 aa, Artificial Sequence
  source: /note=“Description of Artificial Sequence Synthetic peptide”
GKSWSLCTPGCAITGSFNSYCC

SEQ ID NO: 572 - protein, 22 aa, Artificial Sequence
  source: /note=“Description of Artificial Sequence Synthetic peptide”
GKSWSLCTPGCAPTGSFNSYCC

SEQ ID NO: 573 - protein, 22 aa, Artificial Sequence
  source: /note=“Description of Artificial Sequence Synthetic peptide”
GKSWSLCTPGCAQTGSFNSYCC

SEQ ID NO: 574 - protein, 22 aa, Artificial Sequence
  source: /note=“Description of Artificial Sequence Synthetic peptide”
GKSWSLCTPGCAETGSFNSYCC

SEQ ID NO: 575 - protein, 22 aa, Artificial Sequence
  source: /note=“Description of Artificial Sequence Synthetic peptide”
GKSWSLCTPGCAHTGSFNSYCC

SEQ ID NO: 576 - protein, 22 aa, Artificial Sequence
  source: /note=“Description of Artificial Sequence Synthetic peptide”
EKSWSLCTPGCANTGSFNSYCC

SEQ ID NO: 577 - protein, 22 aa, Artificial Sequence
  source: /note=“Description of Artificial Sequence Synthetic peptide”
EKSWSLCTPGCADTGSFNSYCC

SEQ ID NO: 578 - protein, 22 aa, Artificial Sequence
  source: /note=“Description of Artificial Sequence Synthetic peptide”
EKSWSLCTPGCAATGSFNSYCC

SEQ ID NO: 579 - protein, 22 aa, Artificial Sequence
  source: /note=“Description of Artificial Sequence Synthetic peptide”
EKSWSLCTPGCATTGSFNSYCC

SEQ ID NO: 580 - protein, 22 aa, Artificial Sequence
  source: /note=“Description of Artificial Sequence Synthetic peptide”
EKSWSLCTPGCAKTGSFNSYCC

SEQ ID NO: 581 - protein, 22 aa, Artificial Sequence
  source: /note=“Description of Artificial Sequence Synthetic peptide”
EKSWSLCTPGCASTGSFNSYCC

SEQ ID NO: 582 - protein, 22 aa, Artificial Sequence
  source: /note=“Description of Artificial Sequence Synthetic peptide”
EKSWSLCTPGCAGTGSFNSYCC

SEQ ID NO: 583 - protein, 22 aa, Artificial Sequence
  source: /note=“Description of Artificial Sequence Synthetic peptide”
EKSWSLCTPGCAVTGSFNSYCC

SEQ ID NO: 584 - protein, 22 aa, Artificial Sequence
  source: /note=“Description of Artificial Sequence Synthetic peptide”
EKSWSLCTPGCAITGSFNSYCC

SEQ ID NO: 585 - protein, 22 aa, Artificial Sequence
  source: /note=“Description of Artificial Sequence Synthetic peptide”
EKSWSLCTPGCAPTGSFNSYCC

SEQ ID NO: 586 - protein, 22 aa, Artificial Sequence
  source: /note=“Description of Artificial Sequence Synthetic peptide”
EKSWSLCTPGCAQTGSFNSYCC

SEQ ID NO: 587 - protein, 22 aa, Artificial Sequence
  source: /note=“Description of Artificial Sequence Synthetic peptide”
EKSWSLCTPGCAETGSFNSYCC

SEQ ID NO: 588 - protein, 22 aa, Artificial Sequence
  source: /note=“Description of Artificial Sequence Synthetic peptide”
EKSWSLCTPGCAHTGSFNSYCC

SEQ ID NO: 589 - protein, 22 aa, Artificial Sequence
  source: /note=“Description of Artificial Sequence Synthetic peptide”
LKSWSLCTPGCANTGSFNSFCC

SEQ ID NO: 590 - protein, 22 aa, Artificial Sequence
  source: /note=“Description of Artificial Sequence Synthetic peptide”
LKSWSLCTPGCADTGSFNSFCC

SEQ ID NO: 591 - protein, 22 aa, Artificial Sequence
  source: /note=“Description of Artificial Sequence Synthetic peptide”
LKSWSLCTPGCAATGSFNSFCC

SEQ ID NO: 592 - protein, 22 aa, Artificial Sequence
  source: /note=“Description of Artificial Sequence Synthetic peptide”
IKSWSLCTPGCANTGSFNSFCC

SEQ ID NO: 593 - protein, 22 aa, Artificial Sequence
  source: /note=“Description of Artificial Sequence Synthetic peptide”
IKSWSLCTPGCADTGSFNSFCC

SEQ ID NO: 594 - protein, 22 aa, Artificial Sequence
  source: /note=“Description of Artificial Sequence Synthetic peptide”
IKSWSLCTPGCAATGSFNSFCC

SEQ ID NO: 595 - protein, 22 aa, Artificial Sequence
  source: /note=“Description of Artificial Sequence Synthetic peptide”
IASWSLCTPGCANTGSFNSYCC

SEQ ID NO: 596 - protein, 22 aa, Artificial Sequence
  source: /note=“Description of Artificial Sequence Synthetic peptide”
IASWSLCTPGCADTGSFNSYCC

SEQ ID NO: 597 - protein, 22 aa, Artificial Sequence
  source: /note=“Description of Artificial Sequence Synthetic peptide”
IASWSLCTPGCAATGSFNSYCC

SEQ ID NO: 598 - protein, 22 aa, Artificial Sequence
  source: /note=“Description of Artificial Sequence Synthetic peptide”
IASWSLCTPGCATTGSFNSYCC

SEQ ID NO: 599 - protein, 22 aa, Artificial Sequence
  source: /note=“Description of Artificial Sequence Synthetic peptide”
IASWSLCTPGCAKTGSFNSYCC

SEQ ID NO: 600 - protein, 22 aa, Artificial Sequence
  source: /note=“Description of Artificial Sequence Synthetic peptide”
IASWSLCTPGCASTGSFNSYCC

SEQ ID NO: 601 - protein, 22 aa, Artificial Sequence
  source: /note=“Description of Artificial Sequence Synthetic peptide”
IASWSLCTPGCAGTGSFNSYCC

SEQ ID NO: 602 - protein, 22 aa, Artificial Sequence
  source: /note=“Description of Artificial Sequence Synthetic peptide”
IASWSLCTPGCAVTGSFNSYCC

SEQ ID NO: 603 - protein, 22 aa, Artificial Sequence
  source: /note=“Description of Artificial Sequence Synthetic peptide”
IASWSLCTPGCAITGSFNSYCC

SEQ ID NO: 604 - protein, 22 aa, Artificial Sequence
  source: /note=“Description of Artificial Sequence Synthetic peptide”
IASWSLCTPGCAPTGSFNSYCC

SEQ ID NO: 605 - protein, 22 aa, Artificial Sequence
  source: /note=“Description of Artificial Sequence Synthetic peptide”
IASWSLCTPGCAQTGSFNSYCC

SEQ ID NO: 606 - protein, 22 aa, Artificial Sequence
  source: /note=“Description of Artificial Sequence Synthetic peptide”
IASWSLCTPGCAETGSFNSYCC

SEQ ID NO: 607 - protein, 22 aa, Artificial Sequence
  source: /note=“Description of Artificial Sequence Synthetic peptide”
IASWSLCTPGCAHTGSFNSYCC

SEQ ID NO: 608 - protein, 22 aa, Artificial Sequence
  source: /note=“Description of Artificial Sequence Synthetic peptide”
IASWSLCTPGCANTGSFNSFCC

SEQ ID NO: 609 - protein, 22 aa, Artificial Sequence
  source: /note=“Description of Artificial Sequence Synthetic peptide”
IASWSLCTPGCADTGSFNSFCC

SEQ ID NO: 610 - protein, 22 aa, Artificial Sequence
  source: /note=“Description of Artificial Sequence Synthetic peptide”
IASWSLCTPGCAATGSFNSFCC

SEQ ID NO: 611 - protein, 22 aa, Artificial Sequence
  source: /note=“Description of Artificial Sequence Synthetic peptide”
IASWSLCTPGCATTGSFNSFCC

SEQ ID NO: 612 - protein, 22 aa, Artificial Sequence
  source: /note=“Description of Artificial Sequence Synthetic peptide”
IASWSLCTPGCAKTGSFNSFCC

SEQ ID NO: 613 - protein, 22 aa, Artificial Sequence
  source: /note=“Description of Artificial Sequence Synthetic peptide”
IASWSLCTPGCASTGSFNSFCC

SEQ ID NO: 614 - protein, 22 aa, Artificial Sequence
  source: /note=“Description of Artificial Sequence Synthetic peptide”
IASWSLCTPGCAGTGSFNSFCC

SEQ ID NO: 615 - protein, 22 aa, Artificial Sequence
  source: /note=“Description of Artificial Sequence Synthetic peptide”
IASWSLCTPGCAVTGSFNSFCC

SEQ ID NO: 616 - protein, 22 aa, Artificial Sequence
  source: /note=“Description of Artificial Sequence Synthetic peptide”
IASWSLCTPGCAITGSFNSFCC

SEQ ID NO: 617 - protein, 22 aa, Artificial Sequence
  source: /note=“Description of Artificial Sequence Synthetic peptide”
IASWSLCTPGCAPTGSFNSFCC

SEQ ID NO: 618 - protein, 22 aa, Artificial Sequence
  source: /note=“Description of Artificial Sequence Synthetic peptide”
IASWSLCTPGCAQTGSFNSFCC

SEQ ID NO: 619 - protein, 22 aa, Artificial Sequence
  source: /note=“Description of Artificial Sequence Synthetic peptide”
IASWSLCTPGCAETGSFNSFCC

SEQ ID NO: 620 - protein, 22 aa, Artificial Sequence
  source: /note=“Description of Artificial Sequence Synthetic peptide”
IASWSLCTPGCAHTGSFNSFCC

SEQ ID NO: 621 - protein, 22 aa, Artificial Sequence
  source: /note=“Description of Artificial Sequence Synthetic peptide”
IASKSLCTPGCANTGSFNSYCC

SEQ ID NO: 622 - protein, 22 aa, Artificial Sequence
  source: /note=“Description of Artificial Sequence Synthetic peptide”
IASKSLCTPGCADTGSFNSYCC

SEQ ID NO: 623 - protein, 22 aa, Artificial Sequence
  source: /note=“Description of Artificial Sequence Synthetic peptide”
IASKSLCTPGCAATGSFNSYCC

SEQ ID NO: 624 - protein, 22 aa, Artificial Sequence
  source: /note=“Description of Artificial Sequence Synthetic peptide”
IASKSLCTPGCANTGSFNSFCC

SEQ ID NO: 625 - protein, 22 aa, Artificial Sequence
  source: /note=“Description of Artificial Sequence Synthetic peptide”
IASKSLCTPGCADTGSFNSFCC

SEQ ID NO: 626 - protein, 22 aa, Artificial Sequence
  source: /note=“Description of Artificial Sequence Synthetic peptide”
IASKSLCTPGCAATGSFNSFCC

SEQ ID NO: 627 - protein, 22 aa, Artificial Sequence
  source: /note=“Description of Artificial Sequence Synthetic peptide”
IASKFLCTPGCANTGSFNSYCC

SEQ ID NO: 628 - protein, 22 aa, Artificial Sequence
  source: /note=“Description of Artificial Sequence Synthetic peptide”
IASKFLCTPGCADTGSFNSYCC

SEQ ID NO: 629 - protein, 22 aa, Artificial Sequence
  source: /note=“Description of Artificial Sequence Synthetic peptide”
IASKFLCTPGCAATGSFNSYCC

SEQ ID NO: 630 - protein, 22 aa, Artificial Sequence
  source: /note=“Description of Artificial Sequence Synthetic peptide”
IASKFLCTPGCANTGSFNSFCC

SEQ ID NO: 631 - protein, 22 aa, Artificial Sequence
  source: /note=“Description of Artificial Sequence Synthetic peptide”
IASKFLCTPGCADTGSFNSFCC

SEQ ID NO: 632 - protein, 22 aa, Artificial Sequence
  source: /note=“Description of Artificial Sequence Synthetic peptide”
IASKFLCTPGCAATGSFNSFCC

SEQ ID NO: 633 - protein, 22 aa, Artificial Sequence
  source: /note=“Description of Artificial Sequence Synthetic peptide”
ITSWSLCTPGCANTGSFNSYCC

SEQ ID NO: 634 - protein, 22 aa, Artificial Sequence
  source: /note=“Description of Artificial Sequence Synthetic peptide”
ITSWSLCTPGCADTGSFNSYCC

SEQ ID NO: 635 - protein, 22 aa, Artificial Sequence
  source: /note=“Description of Artificial Sequence Synthetic peptide”
ITSWSLCTPGCAATGSFNSYCC

SEQ ID NO: 636 - protein, 22 aa, Artificial Sequence
  source: /note=“Description of Artificial Sequence Synthetic peptide”
ITSWSLCTPGCATTGSFNSYCC

SEQ ID NO: 637 - protein, 22 aa, Artificial Sequence
  source: /note=“Description of Artificial Sequence Synthetic peptide”
ITSWSLCTPGCAKTGSFNSYCC

SEQ ID NO: 638 - protein, 22 aa, Artificial Sequence
  source: /note=“Description of Artificial Sequence Synthetic peptide”
ITSWSLCTPGCASTGSFNSYCC

SEQ ID NO: 639 - protein, 22 aa, Artificial Sequence
  source: /note=“Description of Artificial Sequence Synthetic peptide”
ITSWSLCTPGCAGTGSFNSYCC

SEQ ID NO: 640 - protein, 22 aa, Artificial Sequence
  source: /note=“Description of Artificial Sequence Synthetic peptide”
ITSWSLCTPGCAVTGSFNSYCC

SEQ ID NO: 641 - protein, 22 aa, Artificial Sequence
  source: /note=“Description of Artificial Sequence Synthetic peptide”
ITSWSLCTPGCAITGSFNSYCC

SEQ ID NO: 642 - protein, 22 aa, Artificial Sequence
  source: /note=“Description of Artificial Sequence Synthetic peptide”
ITSWSLCTPGCAPTGSFNSYCC

SEQ ID NO: 643 - protein, 22 aa, Artificial Sequence
  source: /note=“Description of Artificial Sequence Synthetic peptide”
ITSWSLCTPGCAQTGSFNSYCC

SEQ ID NO: 644 - protein, 22 aa, Artificial Sequence
  source: /note=“Description of Artificial Sequence Synthetic peptide”
ITSWSLCTPGCAETGSFNSYCC

SEQ ID NO: 645 - protein, 22 aa, Artificial Sequence
  source: /note=“Description of Artificial Sequence Synthetic peptide”
ITSWSLCTPGCAHTGSFNSYCC

SEQ ID NO: 646 - protein, 22 aa, Artificial Sequence
  source: /note=“Description of Artificial Sequence Synthetic peptide”
ITSWSLCTPGCANTGSFNSFCC

SEQ ID NO: 647 - protein, 22 aa, Artificial Sequence
  source: /note=“Description of Artificial Sequence Synthetic peptide”
ITSWSLCTPGCADTGSFNSFCC

SEQ ID NO: 648 - protein, 22 aa, Artificial Sequence
  source: /note=“Description of Artificial Sequence Synthetic peptide”
ITSWSLCTPGCAATGSFNSFCC

SEQ ID NO: 649 - protein, 22 aa, Artificial Sequence
  source: /note=“Description of Artificial Sequence Synthetic peptide”
ITSWSLCTPGCATTGSFNSFCC

SEQ ID NO: 650 - protein, 22 aa, Artificial Sequence
  source: /note=“Description of Artificial Sequence Synthetic peptide”
ITSWSLCTPGCAKTGSFNSFCC

SEQ ID NO: 651 - protein, 22 aa, Artificial Sequence
  source: /note=“Description of Artificial Sequence Synthetic peptide”
ITSWSLCTPGCASTGSFNSFCC

SEQ ID NO: 652 - protein, 22 aa, Artificial Sequence
  source: /note=“Description of Artificial Sequence Synthetic peptide”
ITSWSLCTPGCAGTGSFNSFCC

SEQ ID NO: 653 - protein, 22 aa, Artificial Sequence
  source: /note=“Description of Artificial Sequence Synthetic peptide”
ITSWSLCTPGCAVTGSFNSFCC

SEQ ID NO: 654 - protein, 22 aa, Artificial Sequence
  source: /note=“Description of Artificial Sequence Synthetic peptide”
ITSWSLCTPGCAITGSFNSFCC

SEQ ID NO: 655 - protein, 22 aa, Artificial Sequence
  source: /note=“Description of Artificial Sequence Synthetic peptide”
ITSWSLCTPGCAPTGSFNSFCC

SEQ ID NO: 656 - protein, 22 aa, Artificial Sequence
  source: /note=“Description of Artificial Sequence Synthetic peptide”
ITSWSLCTPGCAQTGSFNSFCC

SEQ ID NO: 657 - protein, 22 aa, Artificial Sequence
  source: /note=“Description of Artificial Sequence Synthetic peptide”
ITSWSLCTPGCAETGSFNSFCC

SEQ ID NO: 658 - protein, 22 aa, Artificial Sequence
  source: /note=“Description of Artificial Sequence Synthetic peptide”
ITSWSLCTPGCAHTGSFNSFCC

SEQ ID NO: 659 - protein, 22 aa, Artificial Sequence
  source: /note=“Description of Artificial Sequence Synthetic peptide”
ITSWFIGTPGCANTGSFNSFCC

SEQ ID NO: 660 - protein, 22 aa, Artificial Sequence
  source: /note=“Description of Artificial Sequence Synthetic peptide”
ITSWFIGTPGCADTGSFNSFCC

SEQ ID NO: 661 - protein, 22 aa, Artificial Sequence
  source: /note=“Description of Artificial Sequence Synthetic peptide”
ITSWFIGTPGCAATGSFNSFCC

SEQ ID NO: 662 - protein, 22 aa, Artificial Sequence
  source: /note=“Description of Artificial Sequence Synthetic peptide”
ITSWFIGTPGCGNTGSFNSFCC

SEQ ID NO: 663 - protein, 22 aa, Artificial Sequence
  source: /note=“Description of Artificial Sequence Synthetic peptide”
ITSWFIGTPGCGDTGSFNSFCC

SEQ ID NO: 664 - protein, 22 aa, Artificial Sequence
  source: /note=“Description of Artificial Sequence Synthetic peptide”
ITSWFIGTPGCGATGSFNSFCC

SEQ ID NO: 665 - protein, 22 aa, Artificial Sequence
  source: /note=“Description of Artificial Sequence Synthetic peptide”
ITSKSLCTPGCANTGSFNSYCC

SEQ ID NO: 666 - protein, 22 aa, Artificial Sequence
  source: /note=“Description of Artificial Sequence Synthetic peptide”
ITSKSLCTPGCADTGSFNSYCC

SEQ ID NO: 667 - protein, 22 aa, Artificial Sequence
  source: /note=“Description of Artificial Sequence Synthetic peptide”
ITSKSLCTPGCAATGSFNSYCC

SEQ ID NO: 668 - protein, 22 aa, Artificial Sequence
  source: /note=“Description of Artificial Sequence Synthetic peptide”
ITSKSLCTPGCANTGSFNSFCC

SEQ ID NO: 669 - protein, 22 aa, Artificial Sequence
  source: /note=“Description of Artificial Sequence Synthetic peptide”
ITSKSLCTPGCADTGSFNSFCC

SEQ ID NO: 670 - protein, 22 aa, Artificial Sequence
  source: /note=“Description of Artificial Sequence Synthetic peptide”
ITSKSLCTPGCAATGSFNSFCC

SEQ ID NO: 671 - protein, 22 aa, Artificial Sequence
  source: /note=“Description of Artificial Sequence Synthetic peptide”
VTSWSLCTPGCANTGSFNSYCC

SEQ ID NO: 672 - protein, 22 aa, Artificial Sequence
  source: /note=“Description of Artificial Sequence Synthetic peptide”
VTSWSLCTPGCADTGSFNSYCC

SEQ ID NO: 673 - protein, 22 aa, Artificial Sequence
  source: /note=“Description of Artificial Sequence Synthetic peptide”
VTSWSLCTPGCAATGSFNSYCC

SEQ ID NO: 674 - protein, 22 aa, Artificial Sequence
  source: /note=“Description of Artificial Sequence Synthetic peptide”
VTSWSLCTPGCANTGSFNSFCC

SEQ ID NO: 675 - protein, 22 aa, Artificial Sequence
  source: /note=“Description of Artificial Sequence Synthetic peptide”
VTSWSLCTPGCADTGSFNSFCC

SEQ ID NO: 676 - protein, 22 aa, Artificial Sequence
  source: /note=“Description of Artificial Sequence Synthetic peptide”
VTSWSLCTPGCAATGSFNSFCC

SEQ ID NO: 677 - protein, 22 aa, Artificial Sequence
  source: /note=“Description of Artificial Sequence Synthetic peptide”
VTSKSLCTPGCANTGSFNSYCC

SEQ ID NO: 678 - protein, 22 aa, Artificial Sequence
  source: /note=“Description of Artificial Sequence Synthetic peptide”
VTSKSLCTPGCADTGSFNSYCC

SEQ ID NO: 679 - protein, 22 aa, Artificial Sequence
  source: /note=“Description of Artificial Sequence Synthetic peptide”
VTSKSLCTPGCAATGSFNSYCC

SEQ ID NO: 680 - protein, 22 aa, Artificial Sequence
  source: /note=“Description of Artificial Sequence Synthetic peptide”
VTSKSLCTPGCANTGSFNSFCC

SEQ ID NO: 681 - protein, 22 aa, Artificial Sequence
  source: /note=“Description of Artificial Sequence Synthetic peptide”
VTSKSLCTPGCADTGSFNSFCC

SEQ ID NO: 682 - protein, 22 aa, Artificial Sequence
  source: /note=“Description of Artificial Sequence Synthetic peptide”
VTSKSLCTPGCAATGSFNSFCC

SEQ ID NO: 683 - protein, 22 aa, Artificial Sequence
  source: /note=“Description of Artificial Sequence Synthetic peptide”
IKSMSLCTPGCANTGSFNSYCC

SEQ ID NO: 684 - protein, 22 aa, Artificial Sequence
  source: /note=“Description of Artificial Sequence Synthetic peptide”
IKSMSLCTPGCADTGSFNSYCC

SEQ ID NO: 685 - protein, 22 aa, Artificial Sequence
  source: /note=“Description of Artificial Sequence Synthetic peptide”
IKSMSLCTPGCAATGSFNSYCC

SEQ ID NO: 686 - protein, 22 aa, Artificial Sequence
  source: /note=“Description of Artificial Sequence Synthetic peptide”
IKSISLCTPGCANTGSFNSYCC

SEQ ID NO: 687 - protein, 22 aa, Artificial Sequence
  source: /note=“Description of Artificial Sequence Synthetic peptide”
IKSISLCTPGCADTGSFNSYCC

SEQ ID NO: 688 - protein, 22 aa, Artificial Sequence
  source: /note=“Description of Artificial Sequence Synthetic peptide”
IKSISLCTPGCAATGSFNSYCC

SEQ ID NO: 689 - protein, 22 aa, Artificial Sequence
  source: /note=“Description of Artificial Sequence Synthetic peptide”
IKSWSVCTPGCANTGSFNSYCC

SEQ ID NO: 690 - protein, 22 aa, Artificial Sequence
  source: /note=“Description of Artificial Sequence Synthetic peptide”
IKSWSVCTPGCADTGSFNSYCC

SEQ ID NO: 691 - protein, 22 aa, Artificial Sequence
  source: /note=“Description of Artificial Sequence Synthetic peptide”
IKSWSVCTPGCAATGSFNSYCC

SEQ ID NO: 692 - protein, 22 aa, Artificial Sequence
  source: /note=“Description of Artificial Sequence Synthetic peptide”
IKSWSLCTPGCTNTGSFNSYCC

SEQ ID NO: 693 - protein, 22 aa, Artificial Sequence
  source: /note=“Description of Artificial Sequence Synthetic peptide”
IKSWSLCTPGCTDTGSFNSYCC

SEQ ID NO: 694 - protein, 22 aa, Artificial Sequence
  source: /note=“Description of Artificial Sequence Synthetic peptide”
IKSWSLCTPGCTATGSFNSYCC

SEQ ID NO: 695 - protein, 22 aa, Artificial Sequence
  source: /note=“Description of Artificial Sequence Synthetic peptide”
IKSWSLCTPGCANTASFNSYCC

SEQ ID NO: 696 - protein, 22 aa, Artificial Sequence
  source: /note=“Description of Artificial Sequence Synthetic peptide”
IKSWSLCTPGCADTASFNSYCC

SEQ ID NO: 697 - protein, 22 aa, Artificial Sequence
  source: /note=“Description of Artificial Sequence Synthetic peptide”
IKSWSLCTPGCAATASFNSYCC

SEQ ID NO: 698 - protein, 22 aa, Artificial Sequence
  source: /note=“Description of Artificial Sequence Synthetic peptide”
ITSVSLCTPGCANTGSFNSYCC

SEQ ID NO: 699 - protein, 22 aa, Artificial Sequence
  source: /note=“Description of Artificial Sequence Synthetic peptide”
ITSVSLCTPGCADTGSFNSYCC

SEQ ID NO: 700 - protein, 22 aa, Artificial Sequence
  source: /note=“Description of Artificial Sequence Synthetic peptide”
ITSVSLCTPGCAATGSFNSYCC

SEQ ID NO: 701 - protein, 22 aa, Artificial Sequence
  source: /note=“Description of Artificial Sequence Synthetic peptide”
ITSMSLCTPGCANTGSFNSYCC

SEQ ID NO: 702 - protein, 22 aa, Artificial Sequence
  source: /note=“Description of Artificial Sequence Synthetic peptide”
ITSMSLCTPGCADTGSFNSYCC

SEQ ID NO: 703 - protein, 22 aa, Artificial Sequence
  source: /note=“Description of Artificial Sequence Synthetic peptide”
FKSWSLCTPGCANTGSFNSYCC

SEQ ID NO: 704 - protein, 22 aa, Artificial Sequence
  source: /note=“Description of Artificial Sequence Synthetic peptide”
FKSWSLCTPGCARTGSYNSYCC

SEQ ID NO: 705 - protein, 22 aa, Artificial Sequence
  source: /note=“Description of Artificial Sequence Synthetic peptide”
FKSWSLCTPGCARTGSLNSYCC

SEQ ID NO: 706 - protein, 22 aa, Artificial Sequence
  source: /note=“Description of Artificial Sequence Synthetic peptide”
FKSWSLCTPGCARTGSFASYCC

SEQ ID NO: 707 - protein, 22 aa, Artificial Sequence
  source: /note=“Description of Artificial Sequence Synthetic peptide”
FKSWSLCTPGCARTGSFNSFCC

SEQ ID NO: 708 - DNA, 66 bp, Artificial Sequence
  source: /note=“Description of Artificial Sequence Synthetic oligonucleotide”
gctaaaagttggagcctttgtacgcctggttgtgcaggtacaggtagtttcaatagttactgttgc

SEQ ID NO: 709 - DNA, 66 bp, Artificial Sequence
  source: /note=“Description of Artificial Sequence Synthetic oligonucleotide”
gctaaaagttggagcctttgtacgcctggttgtgcaaatacaggtagtttcaatagttactgttgc

SEQ ID NO: 710 - DNA, 66 bp, Artificial Sequence
  source: /note=“Description of Artificial Sequence Synthetic oligonucleotide”
gctaaaagttggagcctttgtacgcctggttgtgcaagtacaggtagtttcaatagttactgttgc

SEQ ID NO: 711 - DNA, 66 bp, Artificial Sequence
  source: /note=“Description of Artificial Sequence Synthetic oligonucleotide”
gctaaaagttggagcctttgtacgcctggttgtgcagttacaggtagtttcaatagttactgttgc

SEQ ID NO: 712 - DNA, 66 bp, Artificial Sequence
  source: /note=“Description of Artificial Sequence Synthetic oligonucleotide”
ggtaaaagttggagcctttgtacgcctggttgtgcagctacaggtagtttcaatagttactgttgc

SEQ ID NO: 713 - DNA, 66 bp, Artificial Sequence
  source: /note=“Description of Artificial Sequence Synthetic oligonucleotide”
ggtaaaagttggagcctttgtacgcctggttgtgcaggtacaggtagtttcaatagttactgttgc

SEQ ID NO: 714 - DNA, 66 bp, Artificial Sequence
  source: /note=“Description of Artificial Sequence Synthetic oligonucleotide”
ggtaaaagttggagcctttgtacgcctggttgtgcaaatacaggtagtttcaatagttactgttgc

SEQ ID NO: 715 - DNA, 66 bp, Artificial Sequence
  source: /note=“Description of Artificial Sequence Synthetic oligonucleotide”
ggtaaaagttggagcctttgtacgcctggttgtgcagttacaggtagtttcaatagttactgttgc

SEQ ID NO: 716 - DNA, 66 bp, Artificial Sequence
  source: /note=“Description of Artificial Sequence Synthetic oligonucleotide”
cataaaagttggagcctttgtacgcctggttgtgcaaatacaggtagtttcaatagttactgttgc

SEQ ID NO: 717 - DNA, 66 bp, Artificial Sequence
  source: /note=“Description of Artificial Sequence Synthetic oligonucleotide”
attaaaagttggagcctttgtacgcctggttgtgcagctacagctagtttcaatagttactgttgc

SEQ ID NO: 718 - DNA, 66 bp, Artificial Sequence
  source: /note=“Description of Artificial Sequence Synthetic oligonucleotide”
attaaaagttggagcctttgtacgcctggttgtgcagatacagctagtttcaatagttactgttgc

SEQ ID NO: 719 - DNA, 66 bp, Artificial Sequence
  source: /note=“Description of Artificial Sequence Synthetic oligonucleotide”
attaaaagttggagcctttgtacgcctggttgtgcaaatacagctagtttcaatagttactgttgc

SEQ ID NO: 720 - DNA, 66 bp, Artificial Sequence
  source: /note=“Description of Artificial Sequence Synthetic oligonucleotide”
attaaaagtattagcctttgtacgcctggttgtgcagctacaggtagtttcaatagttactgttgc

SEQ ID NO: 721 - DNA, 66 bp, Artificial Sequence
  source: /note=“Description of Artificial Sequence Synthetic oligonucleotide”
attaaaagtattagcctttgtacgcctggttgtgcagatacaggtagtttcaatagttactgttgc

SEQ ID NO: 722 - DNA, 66 bp, Artificial Sequence
  source: /note=“Description of Artificial Sequence Synthetic oligonucleotide”
attaaaagtatgagcctttgtacgcctggttgtgcagctacaggtagtttcaatagttactgttgc

SEQ ID NO: 723 - DNA, 66 bp, Artificial Sequence
  source: /note=“Description of Artificial Sequence Synthetic oligonucleotide”
attaaaagtatgagcctttgtacgcctggttgtgcaaatacaggtagtttcaatagttactgttgc

SEQ ID NO: 724 - DNA, 66 bp, Artificial Sequence
  source: /note=“Description of Artificial Sequence Synthetic oligonucleotide”
attgctagttggagcctttgtacgcctggttgtgcagctacaggtagtttcaatagttactgttgc

SEQ ID NO: 725 - DNA, 66 bp, Artificial Sequence
  source: /note=“Description of Artificial Sequence Synthetic oligonucleotide”
attgctagtaaaagcctttgtacgcctggttgtgcagctacaggtagtttcaatagttactgttgc

SEQ ID NO: 726 - DNA, 66 bp, Artificial Sequence
  source: /note=“Description of Artificial Sequence Synthetic oligonucleotide”
attgctagtaaaagcctttgtacgcctggttgtgcagatacaggtagtttcaatagttactgttgc

SEQ ID NO: 727 - DNA, 66 bp, Artificial Sequence
  source: /note=“Description of Artificial Sequence Synthetic oligonucleotide”
attgctagtaaaagcctttgtacgcctggttgtgcagctacaggtagtttcaatagtttttgttgc

SEQ ID NO: 728 - DNA, 66 bp, Artificial Sequence
  source: /note=“Description of Artificial Sequence Synthetic oligonucleotide”
attaaaagttggagcgtttgtacgcctggttgtgcagctacaggtagtttcaatagttactgttgc

SEQ ID NO: 729 - DNA, 66 bp, Artificial Sequence
  source: /note=“Description of Artificial Sequence Synthetic oligonucleotide”
attaaaagttggagcctttgtacgcctggttgtgcagctacaggtagtttcaatagttactgttgc

SEQ ID NO: 730 - DNA, 66 bp, Artificial Sequence
  source: /note=“Description of Artificial Sequence Synthetic oligonucleotide”
attaaaagttggagcctttgtacgcctggttgtgcagctacaggtagtttcaatagtttttgttgc

SEQ ID NO: 731 - DNA, 66 bp, Artificial Sequence
  source: /note=“Description of Artificial Sequence Synthetic oligonucleotide”
attaaaagttggagcctttgtacgcctggttgtgcagatacaggtagtttcaatagttactgttgc

SEQ ID NO: 732 - DNA, 66 bp, Artificial Sequence
  source: /note=“Description of Artificial Sequence Synthetic oligonucleotide”
attaaaagttggagcctttgtacgcctggttgtgcagatacaggtagtttcaatagtttttgttgc

SEQ ID NO: 733 - DNA, 66 bp, Artificial Sequence
  source: /note=“Description of Artificial Sequence Synthetic oligonucleotide”
attaaaagttggagcctttgtacgcctggttgtgcaggtacaggtagtttcaatagttactgttgc

SEQ ID NO: 734 - DNA, 66 bp, Artificial Sequence
  source: /note=“Description of Artificial Sequence Synthetic oligonucleotide”
attaaaagttggagcctttgtacgcctggttgtgcaattacaggtagtttcaatagttactgttgc

SEQ ID NO: 735 - DNA, 66 bp, Artificial Sequence
  source: /note=“Description of Artificial Sequence Synthetic oligonucleotide”
attaaaagttggagcctttgtacgcctggttgtgcaaatacaggtagtttcaatagttactgttgc

SEQ ID NO: 736 - DNA, 66 bp, Artificial Sequence
  source: /note=“Description of Artificial Sequence Synthetic oligonucleotide”
attaaaagttggagcctttgtacgcctggttgtgcaaatacaggtagtttcaatagtttttgttgc

SEQ ID NO: 737 - DNA, 66 bp, Artificial Sequence
  source: /note=“Description of Artificial Sequence Synthetic oligonucleotide”
attaaaagttggagcctttgtacgcctggttgtgcacctacaggtagtttcaatagttactgttgc

SEQ ID NO: 738 - DNA, 66 bp, Artificial Sequence
  source: /note=“Description of Artificial Sequence Synthetic oligonucleotide”
attaaaagttggagcctttgtacgcctggttgtgcacaaacaggtagtttcaatagttactgttgc

SEQ ID NO: 739 - DNA, 66 bp, Artificial Sequence
  source: /note=“Description of Artificial Sequence Synthetic oligonucleotide”
attaaaagttggagcctttgtacgcctggttgtgcaagtacaggtagtttcaatagttactgttgc

SEQ ID NO: 740 - DNA, 66 bp, Artificial Sequence
  source: /note=“Description of Artificial Sequence Synthetic oligonucleotide”
attaaaagttggagcctttgtacgcctggttgtgcagttacaggtagtttcaatagttactgttgc

SEQ ID NO: 741 - DNA, 66 bp, Artificial Sequence
  source: /note=“Description of Artificial Sequence Synthetic oligonucleotide”
attaaaagttggagcctttgtacgcctggttgtgcagaaacaggtagtttcaatagttactgttgc

SEQ ID NO: 742 - DNA, 66 bp, Artificial Sequence
  source: /note=“Description of Artificial Sequence Synthetic oligonucleotide”
cttaaaagttggagcctttgtacgcctggttgtgcagctacaggtagtttcaatagttactgttgc

SEQ ID NO: 743 - DNA, 66 bp, Artificial Sequence
  source: /note=“Description of Artificial Sequence Synthetic oligonucleotide”
cttaaaagttggagcctttgtacgcctggttgtgcagctacaggtagtttcaatagtttttgttgc

SEQ ID NO: 744 - DNA, 66 bp, Artificial Sequence
  source: /note=“Description of Artificial Sequence Synthetic oligonucleotide”
cttaaaagttggagcctttgtacgcctggttgtgcagatacaggtagtttcaatagttactgttgc

SEQ ID NO: 745 - DNA, 66 bp, Artificial Sequence
  source: /note=“Description of Artificial Sequence Synthetic oligonucleotide”
cttaaaagttggagcctttgtacgcctggttgtgcagatacaggtagtttcaatagtttttgttgc

SEQ ID NO: 746 - DNA, 66 bp, Artificial Sequence
  source: /note=“Description of Artificial Sequence Synthetic oligonucleotide”
cttaaaagttggagcctttgtacgcctggttgtgcaggtacaggtagtttcaatagttactgttgc

SEQ ID NO: 747 - DNA, 66 bp, Artificial Sequence
  source: /note=“Description of Artificial Sequence Synthetic oligonucleotide”
cttaaaagttggagcctttgtacgcctggttgtgcaaatacaggtagtttcaatagttactgttgc

SEQ ID NO: 748 - DNA, 66 bp, Artificial Sequence
  source: /note=“Description of Artificial Sequence Synthetic oligonucleotide”
cttaaaagttggagcctttgtacgcctggttgtgcacctacaggtagtttcaatagttactgttgc

SEQ ID NO: 749 - DNA, 66 bp, Artificial Sequence
  source: /note=“Description of Artificial Sequence Synthetic oligonucleotide”
cttaaaagttggagcctttgtacgcctggttgtgcacaaacaggtagtttcaatagttactgttgc

SEQ ID NO: 750 - DNA, 66 bp, Artificial Sequence
  source: /note=“Description of Artificial Sequence Synthetic oligonucleotide”
cttaaaagttggagcctttgtacgcctggttgtgcaaatacaggtagtttcaatagtttttgttgc

SEQ ID NO: 751 - DNA, 66 bp, Artificial Sequence
  source: /note=“Description of Artificial Sequence Synthetic oligonucleotide”
agtaaaagttggagcctttgtacgcctggttgtgcaaatacaggtagtttcaatagttactgttgc

SEQ ID NO: 752 - DNA, 66 bp, Artificial Sequence
  source: /note=“Description of Artificial Sequence Synthetic oligonucleotide”
acaaaaagttggagcctttgtacgcctggttgtgcagctacaggtagtttcaatagttactgttgc

SEQ ID NO: 753 - DNA, 66 bp, Artificial Sequence
  source: /note=“Description of Artificial Sequence Synthetic oligonucleotide”
acaaaaagttggagcctttgtacgcctggttgtgcaggtacaggtagtttcaatagttactgttgc

SEQ ID NO: 754 - DNA, 66 bp, Artificial Sequence
  source: /note=“Description of Artificial Sequence Synthetic oligonucleotide”
acaaaaagttggagcctttgtacgcctggttgtgcaaatacaggtagtttcaatagttactgttgc

SEQ ID NO: 755 - DNA, 66 bp, Artificial Sequence
  source: /note=“Description of Artificial Sequence Synthetic oligonucleotide”
acaaaaagttggagcctttgtacgcctggttgtgcagttacaggtagtttcaatagttactgttgc

SEQ ID NO: 756 - DNA, 66 bp, Artificial Sequence
  source: /note=“Description of Artificial Sequence Synthetic oligonucleotide”
gttaaaagttggagcctttgtacgcctggttgtgcagctacaggtagtttcaatagttactgttgc

SEQ ID NO: 757 - DNA, 66 bp, Artificial Sequence
  source: /note=“Description of Artificial Sequence Synthetic oligonucleotide”
gttaaaagttggagcctttgtacgcctggttgtgcagatacaggtagtttcaatagttactgttgc

SEQ ID NO: 758 - DNA, 66 bp, Artificial Sequence
  source: /note=“Description of Artificial Sequence Synthetic oligonucleotide”
gttaaaagttggagcctttgtacgcctggttgtgcaaatacaggtagtttcaatagttactgttgc

SEQ ID NO: 759 - DNA, 66 bp, Artificial Sequence
  source: /note=“Description of Artificial Sequence Synthetic oligonucleotide”
gttaaaagttggagcctttgtacgcctggttgtgcacaaacaggtagtttcaatagttactgttgc

SEQ ID NO: 760 - DNA, 66 bp, Artificial Sequence
  source: /note=“Description of Artificial Sequence Synthetic oligonucleotide”
gttaaaagttggagcctttgtacgcctggttgtgcagttacaggtagtttcaatagttactgttgc

SEQ ID NO: 761 - DNA, 66 bp, Artificial Sequence
  source: /note=“Description of Artificial Sequence Synthetic oligonucleotide”
gttaaaagttggagcctttgtacgcctggttgtgcacctacaggtagtttcaatagttactgttgc

SEQ ID NO: 762 - DNA, 66 bp, Artificial Sequence
  source: /note=“Description of Artificial Sequence Synthetic oligonucleotide”
tataaaagttggagcctttgtacgcctggttgtgcagatacaggtagtttcaatagttactgttgc

SEQ ID NO: 763 - DNA, 66 bp, Artificial Sequence
  source: /note=“Description of Artificial Sequence Synthetic oligonucleotide”
tataaaagttggagcctttgtacgcctggttgtgcaggtacaggtagtttcaatagttactgttgc

SEQ ID NO: 764 - protein, 22 aa, Artificial Sequence
  source: /note=“Description of Artificial Sequence Synthetic peptide”
WKSWSLCTPGCARTGSFNSYCC

SEQ ID NO: 765 - protein, 22 aa, Artificial Sequence
  source: /note=“Description of Artificial Sequence Synthetic peptide”
AKSWSLCTPGCARTGSFNSYCC

SEQ ID NO: 766 - protein, 22 aa, Artificial Sequence
  source: /note=“Description of Artificial Sequence Synthetic peptide”
LKSWSLCTPGCARTGSFNSYCC

SEQ ID NO: 767 - protein, 22 aa, Artificial Sequence
  source: /note=“Description of Artificial Sequence Synthetic peptide”
MKSWSLCTPGCARTGSFNSYCC

SEQ ID NO: 768 - protein, 22 aa, Artificial Sequence
  source: /note=“Description of Artificial Sequence Synthetic peptide”
CKSWSLCTPGCARTGSFNSYCC

SEQ ID NO: 769 - protein, 22 aa, Artificial Sequence
  source: /note=“Description of Artificial Sequence Synthetic peptide”
NKSWSLCTPGCARTGSFNSYCC

SEQ ID NO: 770 - protein, 22 aa, Artificial Sequence
  source: /note=“Description of Artificial Sequence Synthetic peptide”
YKSWSLCTPGCARTGSFNSYCC

SEQ ID NO: 771 - protein, 22 aa, Artificial Sequence
  source: /note=“Description of Artificial Sequence Synthetic peptide”
DKSWSLCTPGCARTGSFNSYCC

SEQ ID NO: 772 - protein, 22 aa, Artificial Sequence
  source: /note=“Description of Artificial Sequence Synthetic peptide”
EKSWSLCTPGCARTGSFNSYCC

SEQ ID NO: 773 - protein, 22 aa, Artificial Sequence
  source: /note=“Description of Artificial Sequence Synthetic peptide”
PKSWSLCTPGCARTGSFNSYCC

SEQ ID NO: 774 - protein, 22 aa, Artificial Sequence
  source: /note=“Description of Artificial Sequence Synthetic peptide”
QKSWSLCTPGCARTGSFNSYCC

SEQ ID NO: 775 - protein, 22 aa, Artificial Sequence
  source: /note=“Description of Artificial Sequence Synthetic peptide”
GKSWSLCTPGCARTGSFNSYCC

SEQ ID NO: 776 - protein, 22 aa, Artificial Sequence
  source: /note=“Description of Artificial Sequence Synthetic peptide”
RKSWSLCTPGCARTGSFNSYCC

SEQ ID NO: 777 - protein, 22 aa, Artificial Sequence
  source: /note=“Description of Artificial Sequence Synthetic peptide”
HKSWSLCTPGCARTGSFNSYCC

SEQ ID NO: 778 - protein, 22 aa, Artificial Sequence
  source: /note=“Description of Artificial Sequence Synthetic peptide”
SKSWSLCTPGCARTGSFNSYCC

SEQ ID NO: 779 - protein, 22 aa, Artificial Sequence
  source: /note=“Description of Artificial Sequence Synthetic peptide”
TKSWSLCTPGCARTGSFNSYCC

SEQ ID NO: 780 - protein, 22 aa, Artificial Sequence
  source: /note=“Description of Artificial Sequence Synthetic peptide”
IKSWSLCTPGCARTGSFNSYCC

SEQ ID NO: 781 - protein, 22 aa, Artificial Sequence
  source: /note=“Description of Artificial Sequence Synthetic peptide”
FVSWSLCTPGCARTGSFNSYCC

SEQ ID NO: 782 - protein, 22 aa, Artificial Sequence
  source: /note=“Description of Artificial Sequence Synthetic peptide”
FWSWSLCTPGCARTGSFNSYCC

SEQ ID NO: 783 - protein, 22 aa, Artificial Sequence
  source: /note=“Description of Artificial Sequence Synthetic peptide”
FASWSLCTPGCARTGSFNSYCC

SEQ ID NO: 784 - protein, 22 aa, Artificial Sequence
  source: /note=“Description of Artificial Sequence Synthetic peptide”
FLSWSLCTPGCARTGSFNSYCC

SEQ ID NO: 785 - protein, 22 aa, Artificial Sequence
  source: /note=“Description of Artificial Sequence Synthetic peptide”
FMSWSLCTPGCARTGSFNSYCC

SEQ ID NO: 786 - protein, 22 aa, Artificial Sequence
  source: /note=“Description of Artificial Sequence Synthetic peptide”
FCSWSLCTPGCARTGSFNSYCC

SEQ ID NO: 787 - protein, 22 aa, Artificial Sequence
  source: /note=“Description of Artificial Sequence Synthetic peptide”
FNSWSLCTPGCARTGSFNSYCC

SEQ ID NO: 788 - protein, 22 aa, Artificial Sequence
  source: /note=“Description of Artificial Sequence Synthetic peptide”
FYSWSLCTPGCARTGSFNSYCC

SEQ ID NO: 789 - protein, 22 aa, Artificial Sequence
  source: /note=“Description of Artificial Sequence Synthetic peptide”
FDSWSLCTPGCARTGSFNSYCC

SEQ ID NO: 790 - protein, 22 aa, Artificial Sequence
  source: /note=“Description of Artificial Sequence Synthetic peptide”
FESWSLCTPGCARTGSFNSYCC

SEQ ID NO: 791 - protein, 22 aa, Artificial Sequence
  source: /note=“Description of Artificial Sequence Synthetic peptide”
FPSWSLCTPGCARTGSFNSYCC

SEQ ID NO: 792 - protein, 22 aa, Artificial Sequence
  source: /note=“Description of Artificial Sequence Synthetic peptide”
FQSWSLCTPGCARTGSFNSYCC

SEQ ID NO: 793 - protein, 22 aa, Artificial Sequence
  source: /note=“Description of Artificial Sequence Synthetic peptide”
FFSWSLCTPGCARTGSFNSYCC

SEQ ID NO: 794 - protein, 22 aa, Artificial Sequence
  source: /note=“Description of Artificial Sequence Synthetic peptide”
FGSWSLCTPGCARTGSFNSYCC

SEQ ID NO: 795 - protein, 22 aa, Artificial Sequence
  source: /note=“Description of Artificial Sequence Synthetic peptide”
FRSWSLCTPGCARTGSFNSYCC

SEQ ID NO: 796 - protein, 22 aa, Artificial Sequence
  source: /note=“Description of Artificial Sequence Synthetic peptide”
FHSWSLCTPGCARTGSFNSYCC

SEQ ID NO: 797 - protein, 22 aa, Artificial Sequence
  source: /note=“Description of Artificial Sequence Synthetic peptide”
FSSWSLCTPGCARTGSFNSYCC

SEQ ID NO: 798 - protein, 22 aa, Artificial Sequence
  source: /note=“Description of Artificial Sequence Synthetic peptide”
FTSWSLCTPGCARTGSFNSYCC

SEQ ID NO: 799 - protein, 22 aa, Artificial Sequence
  source: /note=“Description of Artificial Sequence Synthetic peptide”
FISWSLCTPGCARTGSFNSYCC

SEQ ID NO: 800 - protein, 22 aa, Artificial Sequence
  source: /note=“Description of Artificial Sequence Synthetic peptide”
FKSVSLCTPGCARTGSFNSYCC

SEQ ID NO: 801 - protein, 22 aa, Artificial Sequence
  source: /note=“Description of Artificial Sequence Synthetic peptide”
FKSASLCTPGCARTGSFNSYCC

SEQ ID NO: 802 - protein, 22 aa, Artificial Sequence
  source: /note=“Description of Artificial Sequence Synthetic peptide”
FKSLSLCTPGCARTGSFNSYCC

SEQ ID NO: 803 - protein, 22 aa, Artificial Sequence
  source: /note=“Description of Artificial Sequence Synthetic peptide”
FKSMSLCTPGCARTGSFNSYCC

SEQ ID NO: 804 - protein, 22 aa, Artificial Sequence
  source: /note=“Description of Artificial Sequence Synthetic peptide”
FKSCSLCTPGCARTGSFNSYCC

SEQ ID NO: 805 - protein, 22 aa, Artificial Sequence
  source: /note=“Description of Artificial Sequence Synthetic peptide”
FKSNSLCTPGCARTGSFNSYCC

SEQ ID NO: 806 - protein, 22 aa, Artificial Sequence
  source: /note=“Description of Artificial Sequence Synthetic peptide”
FKSYSLCTPGCARTGSFNSYCC

SEQ ID NO: 807 - protein, 22 aa, Artificial Sequence
  source: /note=“Description of Artificial Sequence Synthetic peptide”
FKSDSLCTPGCARTGSFNSYCC

SEQ ID NO: 808 - protein, 22 aa, Artificial Sequence
  source: /note=“Description of Artificial Sequence Synthetic peptide”
FKSESLCTPGCARTGSFNSYCC

SEQ ID NO: 809 - protein, 22 aa, Artificial Sequence
  source: /note=“Description of Artificial Sequence Synthetic peptide”
FKSPSLCTPGCARTGSFNSYCC

SEQ ID NO: 810 - protein, 22 aa, Artificial Sequence
  source: /note=“Description of Artificial Sequence Synthetic peptide”
FKSQSLCTPGCARTGSFNSYCC

SEQ ID NO: 811 - protein, 22 aa, Artificial Sequence
  source: /note=“Description of Artificial Sequence Synthetic peptide”
FKSFSLCTPGCARTGSFNSYCC

SEQ ID NO: 812 - protein, 22 aa, Artificial Sequence
  source: /note=“Description of Artificial Sequence Synthetic peptide”
FKSGSLCTPGCARTGSFNSYCC

SEQ ID NO: 813 - protein, 22 aa, Artificial Sequence
  source: /note=“Description of Artificial Sequence Synthetic peptide”
FKSRSLCTPGCARTGSFNSYCC

SEQ ID NO: 814 - protein, 22 aa, Artificial Sequence
  source: /note=“Description of Artificial Sequence Synthetic peptide”
FKSHSLCTPGCARTGSFNSYCC

SEQ ID NO: 815 - protein, 22 aa, Artificial Sequence
  source: /note=“Description of Artificial Sequence Synthetic peptide”
FKSSSLCTPGCARTGSFNSYCC

SEQ ID NO: 816 - protein, 22 aa, Artificial Sequence
  source: /note=“Description of Artificial Sequence Synthetic peptide”
FKSTSLCTPGCARTGSFNSYCC

SEQ ID NO: 817 - protein, 22 aa, Artificial Sequence
  source: /note=“Description of Artificial Sequence Synthetic peptide”
FKSISLCTPGCARTGSFNSYCC

SEQ ID NO: 818 - protein, 22 aa, Artificial Sequence
  source: /note=“Description of Artificial Sequence Synthetic peptide”
FKSWKLCTPGCARTGSFNSYCC

SEQ ID NO: 819 - protein, 22 aa, Artificial Sequence
  source: /note=“Description of Artificial Sequence Synthetic peptide”
FKSWVLCTPGCARTGSFNSYCC

SEQ ID NO: 820 - protein, 22 aa, Artificial Sequence
  source: /note=“Description of Artificial Sequence Synthetic peptide”
FKSWWLCTPGCARTGSFNSYCC

SEQ ID NO: 821 - protein, 22 aa, Artificial Sequence
  source: /note=“Description of Artificial Sequence Synthetic peptide”
FKSWALCTPGCARTGSFNSYCC

SEQ ID NO: 822 - protein, 22 aa, Artificial Sequence
  source: /note=“Description of Artificial Sequence Synthetic peptide”
FKSWLLCTPGCARTGSFNSYCC

SEQ ID NO: 823 - protein, 22 aa, Artificial Sequence
  source: /note=“Description of Artificial Sequence Synthetic peptide”
FKSWMLCTPGCARTGSFNSYCC

SEQ ID NO: 824 - protein, 22 aa, Artificial Sequence
  source: /note=“Description of Artificial Sequence Synthetic peptide”
FKSWCLCTPGCARTGSFNSYCC

SEQ ID NO: 825 - protein, 22 aa, Artificial Sequence
  source: /note=“Description of Artificial Sequence Synthetic peptide”
FKSWNLCTPGCARTGSFNSYCC

SEQ ID NO: 826 - protein, 22 aa, Artificial Sequence
  source: /note=“Description of Artificial Sequence Synthetic peptide”
FKSWYLCTPGCARTGSFNSYCC

SEQ ID NO: 827 - protein, 22 aa, Artificial Sequence
  source: /note=“Description of Artificial Sequence Synthetic peptide”
FKSWDLCTPGCARTGSFNSYCC

SEQ ID NO: 828 - protein, 22 aa, Artificial Sequence
  source: /note=“Description of Artificial Sequence Synthetic peptide”
FKSWELCTPGCARTGSFNSYCC

SEQ ID NO: 829 - protein, 22 aa, Artificial Sequence
  source: /note=“Description of Artificial Sequence Synthetic peptide”
FKSWPLCTPGCARTGSFNSYCC

SEQ ID NO: 830 - protein, 22 aa, Artificial Sequence
  source: /note=“Description of Artificial Sequence Synthetic peptide”
FKSWQLCTPGCARTGSFNSYCC

SEQ ID NO: 831 - protein, 22 aa, Artificial Sequence
  source: /note=“Description of Artificial Sequence Synthetic peptide”
FKSWFLCTPGCARTGSFNSYCC

SEQ ID NO: 832 - protein, 22 aa, Artificial Sequence
  source: /note=“Description of Artificial Sequence Synthetic peptide”
FKSWGLCTPGCARTGSFNSYCC

SEQ ID NO: 833 - protein, 22 aa, Artificial Sequence
  source: /note=“Description of Artificial Sequence Synthetic peptide”
FKSWRLCTPGCARTGSFNSYCC

SEQ ID NO: 834 - protein, 22 aa, Artificial Sequence
  source: /note=“Description of Artificial Sequence Synthetic peptide”
FKSWHLCTPGCARTGSFNSYCC

SEQ ID NO: 835 - protein, 22 aa, Artificial Sequence
  source: /note=“Description of Artificial Sequence Synthetic peptide”
FKSWTLCTPGCARTGSFNSYCC

SEQ ID NO: 836 - protein, 22 aa, Artificial Sequence
  source: /note=“Description of Artificial Sequence Synthetic peptide”
FKSWILCTPGCARTGSFNSYCC

SEQ ID NO: 837 - protein, 22 aa, Artificial Sequence
  source: /note=“Description of Artificial Sequence Synthetic peptide”
FKSWSKCTPGCARTGSFNSYCC

SEQ ID NO: 838 - protein, 22 aa, Artificial Sequence
  source: /note=“Description of Artificial Sequence Synthetic peptide”
FKSWSVCTPGCARTGSFNSYCC

SEQ ID NO: 839 - protein, 22 aa, Artificial Sequence
  source: /note=“Description of Artificial Sequence Synthetic peptide”
FKSWSWCTPGCARTGSFNSYCC

SEQ ID NO: 840 - protein, 22 aa, Artificial Sequence
  source: /note=“Description of Artificial Sequence Synthetic peptide”
FKSWSACTPGCARTGSFNSYCC

SEQ ID NO: 841 - protein, 22 aa, Artificial Sequence
  source: /note=“Description of Artificial Sequence Synthetic peptide”
FKSWSMCTPGCARTGSFNSYCC

SEQ ID NO: 842 - protein, 22 aa, Artificial Sequence
  source: /note=“Description of Artificial Sequence Synthetic peptide”
FKSWSCCTPGCARTGSFNSYCC

SEQ ID NO: 843 - protein, 22 aa, Artificial Sequence
  source: /note=“Description of Artificial Sequence Synthetic peptide”
FKSWSNCTPGCARTGSFNSYCC

SEQ ID NO: 844 - protein, 22 aa, Artificial Sequence
  source: /note=“Description of Artificial Sequence Synthetic peptide”
FKSWSYCTPGCARTGSFNSYCC

SEQ ID NO: 845 - protein, 22 aa, Artificial Sequence
  source: /note=“Description of Artificial Sequence Synthetic peptide”
FKSWSDCTPGCARTGSFNSYCC

SEQ ID NO: 846 - protein, 22 aa, Artificial Sequence
  source: /note=“Description of Artificial Sequence Synthetic peptide”
FKSWSECTPGCARTGSFNSYCC

SEQ ID NO: 847 - protein, 22 aa, Artificial Sequence
  source: /note=“Description of Artificial Sequence Synthetic peptide”
FKSWSPCTPGCARTGSFNSYCC

SEQ ID NO: 848 - protein, 22 aa, Artificial Sequence
  source: /note=“Description of Artificial Sequence Synthetic peptide”
FKSWSQCTPGCARTGSFNSYCC

SEQ ID NO: 849 - protein, 22 aa, Artificial Sequence
  source: /note=“Description of Artificial Sequence Synthetic peptide”
FKSWSFCTPGCARTGSFNSYCC

SEQ ID NO: 850 - protein, 22 aa, Artificial Sequence
  source: /note=“Description of Artificial Sequence Synthetic peptide”
FKSWSGCTPGCARTGSFNSYCC

SEQ ID NO: 851 - protein, 22 aa, Artificial Sequence
  source: /note=“Description of Artificial Sequence Synthetic peptide”
FKSWSRCTPGCARTGSFNSYCC

SEQ ID NO: 852 - protein, 22 aa, Artificial Sequence
  source: /note=“Description of Artificial Sequence Synthetic peptide”
FKSWSHCTPGCARTGSFNSYCC

SEQ ID NO: 853 - protein, 22 aa, Artificial Sequence
  source: /note=“Description of Artificial Sequence Synthetic peptide”
FKSWSSCTPGCARTGSFNSYCC

SEQ ID NO: 854 - protein, 22 aa, Artificial Sequence
  source: /note=“Description of Artificial Sequence Synthetic peptide”
FKSWSTCTPGCARTGSFNSYCC

SEQ ID NO: 855 - protein, 22 aa, Artificial Sequence
  source: /note=“Description of Artificial Sequence Synthetic peptide”
FKSWSICTPGCARTGSFNSYCC

SEQ ID NO: 856 - protein, 22 aa, Artificial Sequence
  source: /note=“Description of Artificial Sequence Synthetic peptide”
FKSWSLKTPGCARTGSFNSYCC

SEQ ID NO: 857 - protein, 22 aa, Artificial Sequence
  source: /note=“Description of Artificial Sequence Synthetic peptide”
FKSWSLVTPGCARTGSFNSYCC

SEQ ID NO: 858 - protein, 22 aa, Artificial Sequence
  source: /note=“Description of Artificial Sequence Synthetic peptide”
FKSWSLWTPGCARTGSFNSYCC

SEQ ID NO: 859 - protein, 22 aa, Artificial Sequence
  source: /note=“Description of Artificial Sequence Synthetic peptide”
FKSWSLATPGCARTGSFNSYCC

SEQ ID NO: 860 - protein, 22 aa, Artificial Sequence
  source: /note=“Description of Artificial Sequence Synthetic peptide”
FKSWSLLTPGCARTGSFNSYCC

SEQ ID NO: 861 - protein, 22 aa, Artificial Sequence
  source: /note=“Description of Artificial Sequence Synthetic peptide”
FKSWSLMTPGCARTGSFNSYCC

SEQ ID NO: 862 - protein, 22 aa, Artificial Sequence
  source: /note=“Description of Artificial Sequence Synthetic peptide”
FKSWSLNTPGCARTGSFNSYCC

SEQ ID NO: 863 - protein, 22 aa, Artificial Sequence
  source: /note=“Description of Artificial Sequence Synthetic peptide”
FKSWSLYTPGCARTGSFNSYCC

SEQ ID NO: 864 - protein, 22 aa, Artificial Sequence
  source: /note=“Description of Artificial Sequence Synthetic peptide”
FKSWSLDTPGCARTGSFNSYCC

SEQ ID NO: 865 - protein, 22 aa, Artificial Sequence
  source: /note=“Description of Artificial Sequence Synthetic peptide”
FKSWSLETPGCARTGSFNSYCC

SEQ ID NO: 866 - protein, 22 aa, Artificial Sequence
  source: /note=“Description of Artificial Sequence Synthetic peptide”
FKSWSLPTPGCARTGSFNSYCC

SEQ ID NO: 867 - protein, 22 aa, Artificial Sequence
  source: /note=“Description of Artificial Sequence Synthetic peptide”
FKSWSLQTPGCARTGSFNSYCC

SEQ ID NO: 868 - protein, 22 aa, Artificial Sequence
  source: /note=“Description of Artificial Sequence Synthetic peptide”
FKSWSLFTPGCARTGSFNSYCC

SEQ ID NO: 869 - protein, 22 aa, Artificial Sequence
  source: /note=“Description of Artificial Sequence Synthetic peptide”
FKSWSLGTPGCARTGSFNSYCC

SEQ ID NO: 870 - protein, 22 aa, Artificial Sequence
  source: /note=“Description of Artificial Sequence Synthetic peptide”
FKSWSLRTPGCARTGSFNSYCC

SEQ ID NO: 871 - protein, 22 aa, Artificial Sequence
  source: /note=“Description of Artificial Sequence Synthetic peptide”
FKSWSLHTPGCARTGSFNSYCC

SEQ ID NO: 872 - protein, 22 aa, Artificial Sequence
  source: /note=“Description of Artificial Sequence Synthetic peptide”
FKSWSLSTPGCARTGSFNSYCC

SEQ ID NO: 873 - protein, 22 aa, Artificial Sequence
  source: /note=“Description of Artificial Sequence Synthetic peptide”
FKSWSLTTPGCARTGSFNSYCC

SEQ ID NO: 874 - protein, 22 aa, Artificial Sequence
  source: /note=“Description of Artificial Sequence Synthetic peptide”
FKSWSLITPGCARTGSFNSYCC

SEQ ID NO: 875 - protein, 22 aa, Artificial Sequence
  source: /note=“Description of Artificial Sequence Synthetic peptide”
FKSWSLCKPGCARTGSFNSYCC

SEQ ID NO: 876 - protein, 22 aa, Artificial Sequence
  source: /note=“Description of Artificial Sequence Synthetic peptide”
FKSWSLCVPGCARTGSFNSYCC

SEQ ID NO: 877 - protein, 22 aa, Artificial Sequence
  source: /note=“Description of Artificial Sequence Synthetic peptide”
FKSWSLCWPGCARTGSFNSYCC

SEQ ID NO: 878 - protein, 22 aa, Artificial Sequence
  source: /note=“Description of Artificial Sequence Synthetic peptide”
FKSWSLCAPGCARTGSFNSYCC

SEQ ID NO: 879 - protein, 22 aa, Artificial Sequence
  source: /note=“Description of Artificial Sequence Synthetic peptide”
FKSWSLCLPGCARTGSFNSYCC

SEQ ID NO: 880 - protein, 22 aa, Artificial Sequence
  source: /note=“Description of Artificial Sequence Synthetic peptide”
FKSWSLCMPGCARTGSFNSYCC

SEQ ID NO: 881 - protein, 22 aa, Artificial Sequence
  source: /note=“Description of Artificial Sequence Synthetic peptide”
FKSWSLCCPGCARTGSFNSYCC

SEQ ID NO: 882 - protein, 22 aa, Artificial Sequence
  source: /note=“Description of Artificial Sequence Synthetic peptide”
FKSWSLCNPGCARTGSFNSYCC

SEQ ID NO: 883 - protein, 22 aa, Artificial Sequence
  source: /note=“Description of Artificial Sequence Synthetic peptide”
FKSWSLCYPGCARTGSFNSYCC

SEQ ID NO: 884 - protein, 22 aa, Artificial Sequence
  source: /note=“Description of Artificial Sequence Synthetic peptide”
FKSWSLCDPGCARTGSFNSYCC

SEQ ID NO: 885 - protein, 22 aa, Artificial Sequence
  source: /note=“Description of Artificial Sequence Synthetic peptide”
FKSWSLCEPGCARTGSFNSYCC

SEQ ID NO: 886 - protein, 22 aa, Artificial Sequence
  source: /note=“Description of Artificial Sequence Synthetic peptide”
FKSWSLCPPGCARTGSFNSYCC

SEQ ID NO: 887 - protein, 22 aa, Artificial Sequence
  source: /note=“Description of Artificial Sequence Synthetic peptide”
FKSWSLCQPGCARTGSFNSYCC

SEQ ID NO: 888 - protein, 22 aa, Artificial Sequence
  source: /note=“Description of Artificial Sequence Synthetic peptide”
FKSWSLCFPGCARTGSFNSYCC

SEQ ID NO: 889 - protein, 22 aa, Artificial Sequence
  source: /note=“Description of Artificial Sequence Synthetic peptide”
FKSWSLCGPGCARTGSFNSYCC

SEQ ID NO: 890 - protein, 22 aa, Artificial Sequence
  source: /note=“Description of Artificial Sequence Synthetic peptide”
FKSWSLCRPGCARTGSFNSYCC

SEQ ID NO: 891 - protein, 22 aa, Artificial Sequence
  source: /note=“Description of Artificial Sequence Synthetic peptide”
FKSWSLCHPGCARTGSFNSYCC

SEQ ID NO: 892 - protein, 22 aa, Artificial Sequence
  source: /note=“Description of Artificial Sequence Synthetic peptide”
FKSWSLCSPGCARTGSFNSYCC

SEQ ID NO: 893 - protein, 22 aa, Artificial Sequence
  source: /note=“Description of Artificial Sequence Synthetic peptide”
FKSWSLCIPGCARTGSFNSYCC

SEQ ID NO: 894 - protein, 22 aa, Artificial Sequence
  source: /note=“Description of Artificial Sequence Synthetic peptide”
FKSWSLCTKGCARTGSFNSYCC

SEQ ID NO: 895 - protein, 22 aa, Artificial Sequence
  source: /note=“Description of Artificial Sequence Synthetic peptide”
FKSWSLCTVGCARTGSFNSYCC

SEQ ID NO: 896 - protein, 22 aa, Artificial Sequence
  source: /note=“Description of Artificial Sequence Synthetic peptide”
FKSWSLCTWGCARTGSFNSYCC

SEQ ID NO: 897 - protein, 22 aa, Artificial Sequence
  source: /note=“Description of Artificial Sequence Synthetic peptide”
FKSWSLCTAGCARTGSFNSYCC

SEQ ID NO: 898 - protein, 22 aa, Artificial Sequence
  source: /note=“Description of Artificial Sequence Synthetic peptide”
FKSWSLCTLGCARTGSFNSYCC

SEQ ID NO: 899 - protein, 22 aa, Artificial Sequence
  source: /note=“Description of Artificial Sequence Synthetic peptide”
FKSWSLCTMGCARTGSFNSYCC

SEQ ID NO: 900 - protein, 22 aa, Artificial Sequence
  source: /note=“Description of Artificial Sequence Synthetic peptide”
FKSWSLCTCGCARTGSFNSYCC

SEQ ID NO: 901 - protein, 22 aa, Artificial Sequence
  source: /note=“Description of Artificial Sequence Synthetic peptide”
FKSWSLCTNGCARTGSFNSYCC

SEQ ID NO: 902 - protein, 22 aa, Artificial Sequence
  source: /note=“Description of Artificial Sequence Synthetic peptide”
FKSWSLCTYGCARTGSFNSYCC

SEQ ID NO: 903 - protein, 22 aa, Artificial Sequence
  source: /note=“Description of Artificial Sequence Synthetic peptide”
FKSWSLCTDGCARTGSFNSYCC

SEQ ID NO: 904 - protein, 22 aa, Artificial Sequence
  source: /note=“Description of Artificial Sequence Synthetic peptide”
FKSWSLCTEGCARTGSFNSYCC

SEQ ID NO: 905 - protein, 22 aa, Artificial Sequence
  source: /note=“Description of Artificial Sequence Synthetic peptide”
FKSWSLCTQGCARTGSFNSYCC

SEQ ID NO: 906 - protein, 22 aa, Artificial Sequence
  source: /note=“Description of Artificial Sequence Synthetic peptide”
FKSWSLCTFGCARTGSFNSYCC

SEQ ID NO: 907 - protein, 22 aa, Artificial Sequence
  source: /note=“Description of Artificial Sequence Synthetic peptide”
FKSWSLCTGGCARTGSFNSYCC

SEQ ID NO: 908 - protein, 22 aa, Artificial Sequence
  source: /note=“Description of Artificial Sequence Synthetic peptide”
FKSWSLCTRGCARTGSFNSYCC

SEQ ID NO: 909 - protein, 22 aa, Artificial Sequence
  source: /note=“Description of Artificial Sequence Synthetic peptide”
FKSWSLCTHGCARTGSFNSYCC

SEQ ID NO: 910 - protein, 22 aa, Artificial Sequence
  source: /note=“Description of Artificial Sequence Synthetic peptide”
FKSWSLCTSGCARTGSFNSYCC

SEQ ID NO: 911 - protein, 22 aa, Artificial Sequence
  source: /note=“Description of Artificial Sequence Synthetic peptide”
FKSWSLCTTGCARTGSFNSYCC

SEQ ID NO: 912 - protein, 22 aa, Artificial Sequence
  source: /note=“Description of Artificial Sequence Synthetic peptide”
FKSWSLCTIGCARTGSFNSYCC

SEQ ID NO: 913 - protein, 22 aa, Artificial Sequence
  source: /note=“Description of Artificial Sequence Synthetic peptide”
FKSWSLCTPKCARTGSFNSYCC

SEQ ID NO: 914 - protein, 22 aa, Artificial Sequence
  source: /note=“Description of Artificial Sequence Synthetic peptide”
FKSWSLCTPVCARTGSFNSYCC

SEQ ID NO: 915 - protein, 22 aa, Artificial Sequence
  source: /note=“Description of Artificial Sequence Synthetic peptide”
FKSWSLCTPWCARTGSFNSYCC

SEQ ID NO: 916 - protein, 22 aa, Artificial Sequence
  source: /note=“Description of Artificial Sequence Synthetic peptide”
FKSWSLCTPACARTGSFNSYCC

SEQ ID NO: 917 - protein, 22 aa, Artificial Sequence
  source: /note=“Description of Artificial Sequence Synthetic peptide”
FKSWSLCTPLCARTGSFNSYCC

SEQ ID NO: 918 - protein, 22 aa, Artificial Sequence
  source: /note=“Description of Artificial Sequence Synthetic peptide”
FKSWSLCTPMCARTGSFNSYCC

SEQ ID NO: 919 - protein, 22 aa, Artificial Sequence
  source: /note=“Description of Artificial Sequence Synthetic peptide”
FKSWSLCTPCCARTGSFNSYCC

SEQ ID NO: 920 - protein, 22 aa, Artificial Sequence
  source: /note=“Description of Artificial Sequence Synthetic peptide”
FKSWSLCTPNCARTGSFNSYCC

SEQ ID NO: 921 - protein, 22 aa, Artificial Sequence
  source: /note=“Description of Artificial Sequence Synthetic peptide”
FKSWSLCTPYCARTGSFNSYCC

SEQ ID NO: 922 - protein, 22 aa, Artificial Sequence
  source: /note=“Description of Artificial Sequence Synthetic peptide”
FKSWSLCTPDCARTGSFNSYCC

SEQ ID NO: 923 - protein, 22 aa, Artificial Sequence
  source: /note=“Description of Artificial Sequence Synthetic peptide”
FKSWSLCTPECARTGSFNSYCC

SEQ ID NO: 924 - protein, 22 aa, Artificial Sequence
  source: /note=“Description of Artificial Sequence Synthetic peptide”
FKSWSLCTPPCARTGSFNSYCC

SEQ ID NO: 925 - protein, 22 aa, Artificial Sequence
  source: /note=“Description of Artificial Sequence Synthetic peptide”
FKSWSLCTPQCARTGSFNSYCC

SEQ ID NO: 926 - protein, 22 aa, Artificial Sequence
  source: /note=“Description of Artificial Sequence Synthetic peptide”
FKSWSLCTPFCARTGSFNSYCC

SEQ ID NO: 927 - protein, 22 aa, Artificial Sequence
  source: /note=“Description of Artificial Sequence Synthetic peptide”
FKSWSLCTPRCARTGSFNSYCC

SEQ ID NO: 928 - protein, 22 aa, Artificial Sequence
  source: /note=“Description of Artificial Sequence Synthetic peptide”
FKSWSLCTPHCARTGSFNSYCC

SEQ ID NO: 929 - protein, 22 aa, Artificial Sequence
  source: /note=“Description of Artificial Sequence Synthetic peptide”
FKSWSLCTPSCARTGSFNSYCC

SEQ ID NO: 930 - protein, 22 aa, Artificial Sequence
  source: /note=“Description of Artificial Sequence Synthetic peptide”
FKSWSLCTPTCARTGSFNSYCC

SEQ ID NO: 931 - protein, 22 aa, Artificial Sequence
  source: /note=“Description of Artificial Sequence Synthetic peptide”
FKSWSLCTPICARTGSFNSYCC

SEQ ID NO: 932 - protein, 22 aa, Artificial Sequence
  source: /note=“Description of Artificial Sequence Synthetic peptide”
FKSWSLCTPGKARTGSFNSYCC

SEQ ID NO: 933 - protein, 22 aa, Artificial Sequence
  source: /note=“Description of Artificial Sequence Synthetic peptide”
FKSWSLCTPGVARTGSFNSYCC

SEQ ID NO: 934 - protein, 22 aa, Artificial Sequence
  source: /note=“Description of Artificial Sequence Synthetic peptide”
FKSWSLCTPGWARTGSFNSYCC

SEQ ID NO: 935 - protein, 22 aa, Artificial Sequence
  source: /note=“Description of Artificial Sequence Synthetic peptide”
FKSWSLCTPGAARTGSFNSYCC

SEQ ID NO: 936 - protein, 22 aa, Artificial Sequence
  source: /note=“Description of Artificial Sequence Synthetic peptide”
FKSWSLCTPGLARTGSFNSYCC

SEQ ID NO: 937 - protein, 22 aa, Artificial Sequence
  source: /note=“Description of Artificial Sequence Synthetic peptide”
FKSWSLCTPGMARTGSFNSYCC

SEQ ID NO: 938 - protein, 22 aa, Artificial Sequence
  source: /note=“Description of Artificial Sequence Synthetic peptide”
FKSWSLCTPGNARTGSFNSYCC

SEQ ID NO: 939 - protein, 22 aa, Artificial Sequence
  source: /note=“Description of Artificial Sequence Synthetic peptide”
FKSWSLCTPGYARTGSFNSYCC

SEQ ID NO: 940 - protein, 22 aa, Artificial Sequence
  source: /note=“Description of Artificial Sequence Synthetic peptide”
FKSWSLCTPGDARTGSFNSYCC

SEQ ID NO: 941 - protein, 22 aa, Artificial Sequence
  source: /note=“Description of Artificial Sequence Synthetic peptide”
FKSWSLCTPGEARTGSFNSYCC

SEQ ID NO: 942 - protein, 22 aa, Artificial Sequence
  source: /note=“Description of Artificial Sequence Synthetic peptide”
FKSWSLCTPGPARTGSFNSYCC

SEQ ID NO: 943 - protein, 22 aa, Artificial Sequence
  source: /note=“Description of Artificial Sequence Synthetic peptide”
FKSWSLCTPGQARTGSFNSYCC

SEQ ID NO: 944 - protein, 22 aa, Artificial Sequence
  source: /note=“Description of Artificial Sequence Synthetic peptide”
FKSWSLCTPGFARTGSFNSYCC

SEQ ID NO: 945 - protein, 22 aa, Artificial Sequence
  source: /note=“Description of Artificial Sequence Synthetic peptide”
FKSWSLCTPGGARTGSFNSYCC

SEQ ID NO: 946 - protein, 22 aa, Artificial Sequence
  source: /note=“Description of Artificial Sequence Synthetic peptide”
FKSWSLCTPGRARTGSFNSYCC

SEQ ID NO: 947 - protein, 22 aa, Artificial Sequence
  source: /note=“Description of Artificial Sequence Synthetic peptide”
FKSWSLCTPGHARTGSFNSYCC

SEQ ID NO: 948 - protein, 22 aa, Artificial Sequence
  source: /note=“Description of Artificial Sequence Synthetic peptide”
FKSWSLCTPGSARTGSFNSYCC

SEQ ID NO: 949 - protein, 22 aa, Artificial Sequence
  source: /note=“Description of Artificial Sequence Synthetic peptide”
FKSWSLCTPGTARTGSFNSYCC

SEQ ID NO: 950 - protein, 22 aa, Artificial Sequence
  source: /note=“Description of Artificial Sequence Synthetic peptide”
FKSWSLCTPGIARTGSFNSYCC

SEQ ID NO: 951 - protein, 22 aa, Artificial Sequence
  source: /note=“Description of Artificial Sequence Synthetic peptide”
FKSWSLCTPGCKRTGSFNSYCC

SEQ ID NO: 952 - protein, 22 aa, Artificial Sequence
  source: /note=“Description of Artificial Sequence Synthetic peptide”
FKSWSLCTPGCVRTGSFNSYCC

SEQ ID NO: 953 - protein, 22 aa, Artificial Sequence
  source: /note=“Description of Artificial Sequence Synthetic peptide”
FKSWSLCTPGCWRTGSFNSYCC

SEQ ID NO: 954 - protein, 22 aa, Artificial Sequence
  source: /note=“Description of Artificial Sequence Synthetic peptide”
FKSWSLCTPGCLRTGSFNSYCC

SEQ ID NO: 955 - protein, 22 aa, Artificial Sequence
  source: /note=“Description of Artificial Sequence Synthetic peptide”
FKSWSLCTPGCMRTGSFNSYCC

SEQ ID NO: 956 - protein, 22 aa, Artificial Sequence
  source: /note=“Description of Artificial Sequence Synthetic peptide”
FKSWSLCTPGCCRTGSFNSYCC

SEQ ID NO: 957 - protein, 22 aa, Artificial Sequence
  source: /note=“Description of Artificial Sequence Synthetic peptide”
FKSWSLCTPGCNRTGSFNSYCC

SEQ ID NO: 958 - protein, 22 aa, Artificial Sequence
  source: /note=“Description of Artificial Sequence Synthetic peptide”
FKSWSLCTPGCYRTGSFNSYCC

SEQ ID NO: 959 - protein, 22 aa, Artificial Sequence
  source: /note=“Description of Artificial Sequence Synthetic peptide”
FKSWSLCTPGCDRTGSFNSYCC

SEQ ID NO: 960 - protein, 22 aa, Artificial Sequence
  source: /note=“Description of Artificial Sequence Synthetic peptide”
FKSWSLCTPGCERTGSFNSYCC

SEQ ID NO: 961 - protein, 22 aa, Artificial Sequence
  source: /note=“Description of Artificial Sequence Synthetic peptide”
FKSWSLCTPGCPRTGSFNSYCC

SEQ ID NO: 962 - protein, 22 aa, Artificial Sequence
  source: /note=“Description of Artificial Sequence Synthetic peptide”
FKSWSLCTPGCQRTGSFNSYCC

SEQ ID NO: 963 - protein, 22 aa, Artificial Sequence
  source: /note=“Description of Artificial Sequence Synthetic peptide”
FKSWSLCTPGCFRTGSFNSYCC

SEQ ID NO: 964 - protein, 22 aa, Artificial Sequence
  source: /note=“Description of Artificial Sequence Synthetic peptide”
FKSWSLCTPGCGRTGSFNSYCC

SEQ ID NO: 965 - protein, 22 aa, Artificial Sequence
  source: /note=“Description of Artificial Sequence Synthetic peptide”
FKSWSLCTPGCRRTGSFNSYCC

SEQ ID NO: 966 - protein, 22 aa, Artificial Sequence
  source: /note=“Description of Artificial Sequence Synthetic peptide”
FKSWSLCTPGCHRTGSFNSYCC

SEQ ID NO: 967 - protein, 22 aa, Artificial Sequence
  source: /note=“Description of Artificial Sequence Synthetic peptide”
FKSWSLCTPGCSRTGSFNSYCC

SEQ ID NO: 968 - protein, 22 aa, Artificial Sequence
  source: /note=“Description of Artificial Sequence Synthetic peptide”
FKSWSLCTPGCTRTGSFNSYCC

SEQ ID NO: 969 - protein, 22 aa, Artificial Sequence
  source: /note=“Description of Artificial Sequence Synthetic peptide”
FKSWSLCTPGCIRTGSFNSYCC

SEQ ID NO: 970 - protein, 22 aa, Artificial Sequence
  source: /note=“Description of Artificial Sequence Synthetic peptide”
FKSWSLCTPGCAKTGSFNSYCC

SEQ ID NO: 971 - protein, 22 aa, Artificial Sequence
  source: /note=“Description of Artificial Sequence Synthetic peptide”
FKSWSLCTPGCAVTGSFNSYCC

SEQ ID NO: 972 - protein, 22 aa, Artificial Sequence
  source: /note=“Description of Artificial Sequence Synthetic peptide”
FKSWSLCTPGCAWTGSFNSYCC

SEQ ID NO: 973 - protein, 22 aa, Artificial Sequence
  source: /note=“Description of Artificial Sequence Synthetic peptide”
FKSWSLCTPGCAATGSFNSYCC

SEQ ID NO: 974 - protein, 22 aa, Artificial Sequence
  source: /note=“Description of Artificial Sequence Synthetic peptide”
FKSWSLCTPGCALTGSFNSYCC

SEQ ID NO: 975 - protein, 22 aa, Artificial Sequence
  source: /note=“Description of Artificial Sequence Synthetic peptide”
FKSWSLCTPGCAMTGSFNSYCC

SEQ ID NO: 976 - protein, 22 aa, Artificial Sequence
  source: /note=“Description of Artificial Sequence Synthetic peptide”
FKSWSLCTPGCACTGSFNSYCC

SEQ ID NO: 977 - protein, 22 aa, Artificial Sequence
  source: /note=“Description of Artificial Sequence Synthetic peptide”
FKSWSLCTPGCAYTGSFNSYCC

SEQ ID NO: 978 - protein, 22 aa, Artificial Sequence
  source: /note=“Description of Artificial Sequence Synthetic peptide”
FKSWSLCTPGCADTGSFNSYCC

SEQ ID NO: 979 - protein, 22 aa, Artificial Sequence
  source: /note=“Description of Artificial Sequence Synthetic peptide”
FKSWSLCTPGCAETGSFNSYCC

SEQ ID NO: 980 - protein, 22 aa, Artificial Sequence
  source: /note=“Description of Artificial Sequence Synthetic peptide”
FKSWSLCTPGCAPTGSFNSYCC

SEQ ID NO: 981 - protein, 22 aa, Artificial Sequence
  source: /note=“Description of Artificial Sequence Synthetic peptide”
FKSWSLCTPGCAQTGSFNSYCC

SEQ ID NO: 982 - protein, 22 aa, Artificial Sequence
  source: /note=“Description of Artificial Sequence Synthetic peptide”
FKSWSLCTPGCAFTGSFNSYCC

SEQ ID NO: 983 - protein, 22 aa, Artificial Sequence
  source: /note=“Description of Artificial Sequence Synthetic peptide”
FKSWSLCTPGCAGTGSFNSYCC

SEQ ID NO: 984 - protein, 22 aa, Artificial Sequence
  source: /note=“Description of Artificial Sequence Synthetic peptide”
FKSWSLCTPGCAHTGSFNSYCC

SEQ ID NO: 985 - protein, 22 aa, Artificial Sequence
  source: /note=“Description of Artificial Sequence Synthetic peptide”
FKSWSLCTPGCASTGSFNSYCC

SEQ ID NO: 986 - protein, 22 aa, Artificial Sequence
  source: /note=“Description of Artificial Sequence Synthetic peptide”
FKSWSLCTPGCATTGSFNSYCC

SEQ ID NO: 987 - protein, 22 aa, Artificial Sequence
  source: /note=“Description of Artificial Sequence Synthetic peptide”
FKSWSLCTPGCAITGSFNSYCC

SEQ ID NO: 988 - protein, 22 aa, Artificial Sequence
  source: /note=“Description of Artificial Sequence Synthetic peptide”
FKSWSLCTPGCARKGSFNSYCC

SEQ ID NO: 989 - protein, 22 aa, Artificial Sequence
  source: /note=“Description of Artificial Sequence Synthetic peptide”
FKSWSLCTPGCARVGSFNSYCC

SEQ ID NO: 990 - protein, 22 aa, Artificial Sequence
  source: /note=“Description of Artificial Sequence Synthetic peptide”
FKSWSLCTPGCARWGSFNSYCC

SEQ ID NO: 991 - protein, 22 aa, Artificial Sequence
  source: /note=“Description of Artificial Sequence Synthetic peptide”
FKSWSLCTPGCARAGSFNSYCC

SEQ ID NO: 992 - protein, 22 aa, Artificial Sequence
  source: /note=“Description of Artificial Sequence Synthetic peptide”
FKSWSLCTPGCARLGSFNSYCC

SEQ ID NO: 993 - protein, 22 aa, Artificial Sequence
  source: /note=“Description of Artificial Sequence Synthetic peptide”
FKSWSLCTPGCARMGSFNSYCC

SEQ ID NO: 994 - protein, 22 aa, Artificial Sequence
  source: /note=“Description of Artificial Sequence Synthetic peptide”
FKSWSLCTPGCARCGSFNSYCC

SEQ ID NO: 995 - protein, 22 aa, Artificial Sequence
  source: /note=“Description of Artificial Sequence Synthetic peptide”
FKSWSLCTPGCARNGSFNSYCC

SEQ ID NO: 996 - protein, 22 aa, Artificial Sequence
  source: /note=“Description of Artificial Sequence Synthetic peptide”
FKSWSLCTPGCARYGSFNSYCC

SEQ ID NO: 997 - protein, 22 aa, Artificial Sequence
  source: /note=“Description of Artificial Sequence Synthetic peptide”
FKSWSLCTPGCARDGSFNSYCC

SEQ ID NO: 998 - protein, 22 aa, Artificial Sequence
  source: /note=“Description of Artificial Sequence Synthetic peptide”
FKSWSLCTPGCAREGSFNSYCC

SEQ ID NO: 999 - protein, 22 aa, Artificial Sequence
  source: /note=“Description of Artificial Sequence Synthetic peptide”
FKSWSLCTPGCARPGSFNSYCC

SEQ ID NO: 1000 - protein, 22 aa, Artificial Sequence
  source: /note=“Description of Artificial Sequence Synthetic peptide”
FKSWSLCTPGCARQGSFNSYCC

SEQ ID NO: 1001 - protein, 22 aa, Artificial Sequence
  source: /note=“Description of Artificial Sequence Synthetic peptide”
FKSWSLCTPGCARFGSFNSYCC

SEQ ID NO: 1002 - protein, 22 aa, Artificial Sequence
  source: /note=“Description of Artificial Sequence Synthetic peptide”
FKSWSLCTPGCARGGSFNSYCC

SEQ ID NO: 1003 - protein, 22 aa, Artificial Sequence
  source: /note=“Description of Artificial Sequence Synthetic peptide”
FKSWSLCTPGCARRGSFNSYCC

SEQ ID NO: 1004 - protein, 22 aa, Artificial Sequence
  source: /note=“Description of Artificial Sequence Synthetic peptide”
FKSWSLCTPGCARHGSFNSYCC

SEQ ID NO: 1005 - protein, 22 aa, Artificial Sequence
  source: /note=“Description of Artificial Sequence Synthetic peptide”
FKSWSLCTPGCARSGSFNSYCC

SEQ ID NO: 1006 - protein, 22 aa, Artificial Sequence
  source: /note=“Description of Artificial Sequence Synthetic peptide”
FKSWSLCTPGCARIGSFNSYCC

SEQ ID NO: 1007 - protein, 22 aa, Artificial Sequence
  source: /note=“Description of Artificial Sequence Synthetic peptide”
FKSWSLCTPGCARTKSFNSYCC

SEQ ID NO: 1008 - protein, 22 aa, Artificial Sequence
  source: /note=“Description of Artificial Sequence Synthetic peptide”
FKSWSLCTPGCARTVSFNSYCC

SEQ ID NO: 1009 - protein, 22 aa, Artificial Sequence
  source: /note=“Description of Artificial Sequence Synthetic peptide”
FKSWSLCTPGCARTWSFNSYCC

SEQ ID NO: 1010 - protein, 22 aa, Artificial Sequence
  source: /note=“Description of Artificial Sequence Synthetic peptide”
FKSWSLCTPGCARTASFNSYCC

SEQ ID NO: 1011 - protein, 22 aa, Artificial Sequence
  source: /note=“Description of Artificial Sequence Synthetic peptide”
FKSWSLCTPGCARTLSFNSYCC

SEQ ID NO: 1012 - protein, 22 aa, Artificial Sequence
  source: /note=“Description of Artificial Sequence Synthetic peptide”
FKSWSLCTPGCARTMSFNSYCC

SEQ ID NO: 1013 - protein, 22 aa, Artificial Sequence
  source: /note=“Description of Artificial Sequence Synthetic peptide”
FKSWSLCTPGCARTCSFNSYCC

SEQ ID NO: 1014 - protein, 22 aa, Artificial Sequence
  source: /note=“Description of Artificial Sequence Synthetic peptide”
FKSWSLCTPGCARTNSFNSYCC

SEQ ID NO: 1015 - protein, 22 aa, Artificial Sequence
  source: /note=“Description of Artificial Sequence Synthetic peptide”
FKSWSLCTPGCARTYSFNSYCC

SEQ ID NO: 1016 - protein, 22 aa, Artificial Sequence
  source: /note=“Description of Artificial Sequence Synthetic peptide”
FKSWSLCTPGCARTDSFNSYCC

SEQ ID NO: 1017 - protein, 22 aa, Artificial Sequence
  source: /note=“Description of Artificial Sequence Synthetic peptide”
FKSWSLCTPGCARTESFNSYCC

SEQ ID NO: 1018 - protein, 22 aa, Artificial Sequence
  source: /note=“Description of Artificial Sequence Synthetic peptide”
FKSWSLCTPGCARTPSFNSYCC

SEQ ID NO: 1019 - protein, 22 aa, Artificial Sequence
  source: /note=“Description of Artificial Sequence Synthetic peptide”
FKSWSLCTPGCARTQSFNSYCC

SEQ ID NO: 1020 - protein, 22 aa, Artificial Sequence
  source: /note=“Description of Artificial Sequence Synthetic peptide”
FKSWSLCTPGCARTFSFNSYCC

SEQ ID NO: 1021 - protein, 22 aa, Artificial Sequence
  source: /note=“Description of Artificial Sequence Synthetic peptide”
FKSWSLCTPGCARTRSFNSYCC

SEQ ID NO: 1022 - protein, 22 aa, Artificial Sequence
  source: /note=“Description of Artificial Sequence Synthetic peptide”
FKSWSLCTPGCARTHSFNSYCC

SEQ ID NO: 1023 - protein, 22 aa, Artificial Sequence
  source: /note=“Description of Artificial Sequence Synthetic peptide”
FKSWSLCTPGCARTSSFNSYCC

SEQ ID NO: 1024 - protein, 22 aa, Artificial Sequence
  source: /note=“Description of Artificial Sequence Synthetic peptide”
FKSWSLCTPGCARTTSFNSYCC

SEQ ID NO: 1025 - protein, 22 aa, Artificial Sequence
  source: /note=“Description of Artificial Sequence Synthetic peptide”
FKSWSLCTPGCARTISFNSYCC

SEQ ID NO: 1026 - protein, 22 aa, Artificial Sequence
  source: /note=“Description of Artificial Sequence Synthetic peptide”
FKSWSLCTPGCARTGKFNSYCC

SEQ ID NO: 1027 - protein, 22 aa, Artificial Sequence
  source: /note=“Description of Artificial Sequence Synthetic peptide”
FKSWSLCTPGCARTGVFNSYCC

SEQ ID NO: 1028 - protein, 22 aa, Artificial Sequence
  source: /note=“Description of Artificial Sequence Synthetic peptide”
FKSWSLCTPGCARTGWFNSYCC

SEQ ID NO: 1029 - protein, 22 aa, Artificial Sequence
  source: /note=“Description of Artificial Sequence Synthetic peptide”
FKSWSLCTPGCARTGAFNSYCC

SEQ ID NO: 1030 - protein, 22 aa, Artificial Sequence
  source: /note=“Description of Artificial Sequence Synthetic peptide”
FKSWSLCTPGCARTGLFNSYCC

SEQ ID NO: 1031 - protein, 22 aa, Artificial Sequence
  source: /note=“Description of Artificial Sequence Synthetic peptide”
FKSWSLCTPGCARTGMFNSYCC

SEQ ID NO: 1032 - protein, 22 aa, Artificial Sequence
  source: /note=“Description of Artificial Sequence Synthetic peptide”
FKSWSLCTPGCARTGCFNSYCC

SEQ ID NO: 1033 - protein, 22 aa, Artificial Sequence
  source: /note=“Description of Artificial Sequence Synthetic peptide”
FKSWSLCTPGCARTGNFNSYCC

SEQ ID NO: 1034 - protein, 22 aa, Artificial Sequence
  source: /note=“Description of Artificial Sequence Synthetic peptide”
FKSWSLCTPGCARTGYFNSYCC

SEQ ID NO: 1035 - protein, 22 aa, Artificial Sequence
  source: /note=“Description of Artificial Sequence Synthetic peptide”
FKSWSLCTPGCARTGDFNSYCC

SEQ ID NO: 1036 - protein, 22 aa, Artificial Sequence
  source: /note=“Description of Artificial Sequence Synthetic peptide”
FKSWSLCTPGCARTGEFNSYCC

SEQ ID NO: 1037 - protein, 22 aa, Artificial Sequence
  source: /note=“Description of Artificial Sequence Synthetic peptide”
FKSWSLCTPGCARTGPFNSYCC

SEQ ID NO: 1038 - protein, 22 aa, Artificial Sequence
  source: /note=“Description of Artificial Sequence Synthetic peptide”
FKSWSLCTPGCARTGQFNSYCC

SEQ ID NO: 1039 - protein, 22 aa, Artificial Sequence
  source: /note=“Description of Artificial Sequence Synthetic peptide”
FKSWSLCTPGCARTGFFNSYCC

SEQ ID NO: 1040 - protein, 22 aa, Artificial Sequence
  source: /note=“Description of Artificial Sequence Synthetic peptide”
FKSWSLCTPGCARTGGFNSYCC

SEQ ID NO: 1041 - protein, 22 aa, Artificial Sequence
  source: /note=“Description of Artificial Sequence Synthetic peptide”
FKSWSLCTPGCARTGRFNSYCC

SEQ ID NO: 1042 - protein, 22 aa, Artificial Sequence
  source: /note=“Description of Artificial Sequence Synthetic peptide”
FKSWSLCTPGCARTGHFNSYCC

SEQ ID NO: 1043 - protein, 22 aa, Artificial Sequence
  source: /note=“Description of Artificial Sequence Synthetic peptide”
FKSWSLCTPGCARTGTFNSYCC

SEQ ID NO: 1044 - protein, 22 aa, Artificial Sequence
  source: /note=“Description of Artificial Sequence Synthetic peptide”
FKSWSLCTPGCARTGIFNSYCC

SEQ ID NO: 1045 - protein, 22 aa, Artificial Sequence
  source: /note=“Description of Artificial Sequence Synthetic peptide”
FKSWSLCTPGCARTGSKNSYCC

SEQ ID NO: 1046 - protein, 22 aa, Artificial Sequence
  source: /note=“Description of Artificial Sequence Synthetic peptide”
FKSWSLCTPGCARTGSVNSYCC

SEQ ID NO: 1047 - protein, 22 aa, Artificial Sequence
  source: /note=“Description of Artificial Sequence Synthetic peptide”
FKSWSLCTPGCARTGSWNSYCC

SEQ ID NO: 1048 - protein, 22 aa, Artificial Sequence
  source: /note=“Description of Artificial Sequence Synthetic peptide”
FKSWSLCTPGCARTGSANSYCC

SEQ ID NO: 1049 - protein, 22 aa, Artificial Sequence
  source: /note=“Description of Artificial Sequence Synthetic peptide”
FKSWSLCTPGCARTGSMNSYCC

SEQ ID NO: 1050 - protein, 22 aa, Artificial Sequence
  source: /note=“Description of Artificial Sequence Synthetic peptide”
FKSWSLCTPGCARTGSCNSYCC

SEQ ID NO: 1051 - protein, 22 aa, Artificial Sequence
  source: /note=“Description of Artificial Sequence Synthetic peptide”
FKSWSLCTPGCARTGSNNSYCC

SEQ ID NO: 1052 - protein, 22 aa, Artificial Sequence
  source: /note=“Description of Artificial Sequence Synthetic peptide”
FKSWSLCTPGCARTGSDNSYCC

SEQ ID NO: 1053 - protein, 22 aa, Artificial Sequence
  source: /note=“Description of Artificial Sequence Synthetic peptide”
FKSWSLCTPGCARTGSENSYCC

SEQ ID NO: 1054 - protein, 22 aa, Artificial Sequence
  source: /note=“Description of Artificial Sequence Synthetic peptide”
FKSWSLCTPGCARTGSPNSYCC

SEQ ID NO: 1055 - protein, 22 aa, Artificial Sequence
  source: /note=“Description of Artificial Sequence Synthetic peptide”
FKSWSLCTPGCARTGSQNSYCC

SEQ ID NO: 1056 - protein, 22 aa, Artificial Sequence
  source: /note=“Description of Artificial Sequence Synthetic peptide”
FKSWSLCTPGCARTGSGNSYCC

SEQ ID NO: 1057 - protein, 22 aa, Artificial Sequence
  source: /note=“Description of Artificial Sequence Synthetic peptide”
FKSWSLCTPGCARTGSRNSYCC

SEQ ID NO: 1058 - protein, 22 aa, Artificial Sequence
  source: /note=“Description of Artificial Sequence Synthetic peptide”
FKSWSLCTPGCARTGSHNSYCC

SEQ ID NO: 1059 - protein, 22 aa, Artificial Sequence
  source: /note=“Description of Artificial Sequence Synthetic peptide”
FKSWSLCTPGCARTGSSNSYCC

SEQ ID NO: 1060 - protein, 22 aa, Artificial Sequence
  source: /note=“Description of Artificial Sequence Synthetic peptide”
FKSWSLCTPGCARTGSTNSYCC

SEQ ID NO: 1061 - protein, 22 aa, Artificial Sequence
  source: /note=“Description of Artificial Sequence Synthetic peptide”
FKSWSLCTPGCARTGSINSYCC

SEQ ID NO: 1062 - protein, 22 aa, Artificial Sequence
  source: /note=“Description of Artificial Sequence Synthetic peptide”
FKSWSLCTPGCARTGSFKSYCC

SEQ ID NO: 1063 - protein, 22 aa, Artificial Sequence
  source: /note=“Description of Artificial Sequence Synthetic peptide”
FKSWSLCTPGCARTGSFVSYCC

SEQ ID NO: 1064 - protein, 22 aa, Artificial Sequence
  source: /note=“Description of Artificial Sequence Synthetic peptide”
FKSWSLCTPGCARTGSFWSYCC

SEQ ID NO: 1065 - protein, 22 aa, Artificial Sequence
  source: /note=“Description of Artificial Sequence Synthetic peptide”
FKSWSLCTPGCARTGSFLSYCC

SEQ ID NO: 1066 - protein, 22 aa, Artificial Sequence
  source: /note=“Description of Artificial Sequence Synthetic peptide”
FKSWSLCTPGCARTGSFMSYCC

SEQ ID NO: 1067 - protein, 22 aa, Artificial Sequence
  source: /note=“Description of Artificial Sequence Synthetic peptide”
FKSWSLCTPGCARTGSFCSYCC

SEQ ID NO: 1068 - protein, 22 aa, Artificial Sequence
  source: /note=“Description of Artificial Sequence Synthetic peptide”
FKSWSLCTPGCARTGSFYSYCC

SEQ ID NO: 1069 - protein, 22 aa, Artificial Sequence
  source: /note=“Description of Artificial Sequence Synthetic peptide”
FKSWSLCTPGCARTGSFDSYCC

SEQ ID NO: 1070 - protein, 22 aa, Artificial Sequence
  source: /note=“Description of Artificial Sequence Synthetic peptide”
FKSWSLCTPGCARTGSFESYCC

SEQ ID NO: 1071 - protein, 22 aa, Artificial Sequence
  source: /note=“Description of Artificial Sequence Synthetic peptide”
FKSWSLCTPGCARTGSFPSYCC

SEQ ID NO: 1072 - protein, 22 aa, Artificial Sequence
  source: /note=“Description of Artificial Sequence Synthetic peptide”
FKSWSLCTPGCARTGSFQSYCC

SEQ ID NO: 1073 - protein, 22 aa, Artificial Sequence
  source: /note=“Description of Artificial Sequence Synthetic peptide”
FKSWSLCTPGCARTGSFFSYCC

SEQ ID NO: 1074 - protein, 22 aa, Artificial Sequence
  source: /note=“Description of Artificial Sequence Synthetic peptide”
FKSWSLCTPGCARTGSFGSYCC

SEQ ID NO: 1075 - protein, 22 aa, Artificial Sequence
  source: /note=“Description of Artificial Sequence Synthetic peptide”
FKSWSLCTPGCARTGSFRSYCC

SEQ ID NO: 1076 - protein, 22 aa, Artificial Sequence
  source: /note=“Description of Artificial Sequence Synthetic peptide”
FKSWSLCTPGCARTGSFHSYCC

SEQ ID NO: 1077 - protein, 22 aa, Artificial Sequence
  source: /note=“Description of Artificial Sequence Synthetic peptide”
FKSWSLCTPGCARTGSFSSYCC

SEQ ID NO: 1078 - protein, 22 aa, Artificial Sequence
  source: /note=“Description of Artificial Sequence Synthetic peptide”
FKSWSLCTPGCARTGSFTSYCC

SEQ ID NO: 1079 - protein, 22 aa, Artificial Sequence
  source: /note=“Description of Artificial Sequence Synthetic peptide”
FKSWSLCTPGCARTGSFISYCC

SEQ ID NO: 1080 - protein, 22 aa, Artificial Sequence
  source: /note=“Description of Artificial Sequence Synthetic peptide”
FKSWSLCTPGCARTGSFNKYCC

SEQ ID NO: 1081 - protein, 22 aa, Artificial Sequence
  source: /note=“Description of Artificial Sequence Synthetic peptide”
FKSWSLCTPGCARTGSFNVYCC

SEQ ID NO: 1082 - protein, 22 aa, Artificial Sequence
  source: /note=“Description of Artificial Sequence Synthetic peptide”
FKSWSLCTPGCARTGSFNWYCC

SEQ ID NO: 1083 - protein, 22 aa, Artificial Sequence
  source: /note=“Description of Artificial Sequence Synthetic peptide”
FKSWSLCTPGCARTGSFNAYCC

SEQ ID NO: 1084 - protein, 22 aa, Artificial Sequence
  source: /note=“Description of Artificial Sequence Synthetic peptide”
FKSWSLCTPGCARTGSFNLYCC

SEQ ID NO: 1085 - protein, 22 aa, Artificial Sequence
  source: /note=“Description of Artificial Sequence Synthetic peptide”
FKSWSLCTPGCARTGSFNMYCC

SEQ ID NO: 1086 - protein, 22 aa, Artificial Sequence
  source: /note=“Description of Artificial Sequence Synthetic peptide”
FKSWSLCTPGCARTGSFNCYCC

SEQ ID NO: 1087 - protein, 22 aa, Artificial Sequence
  source: /note=“Description of Artificial Sequence Synthetic peptide”
FKSWSLCTPGCARTGSFNNYCC

SEQ ID NO: 1088 - protein, 22 aa, Artificial Sequence
  source: /note=“Description of Artificial Sequence Synthetic peptide”
FKSWSLCTPGCARTGSFNYYCC

SEQ ID NO: 1089 - protein, 22 aa, Artificial Sequence
  source: /note=“Description of Artificial Sequence Synthetic peptide”
FKSWSLCTPGCARTGSFNDYCC

SEQ ID NO: 1090 - protein, 22 aa, Artificial Sequence
  source: /note=“Description of Artificial Sequence Synthetic peptide”
FKSWSLCTPGCARTGSFNEYCC

SEQ ID NO: 1091 - protein, 22 aa, Artificial Sequence
  source: /note=“Description of Artificial Sequence Synthetic peptide”
FKSWSLCTPGCARTGSFNPYCC

SEQ ID NO: 1092 - protein, 22 aa, Artificial Sequence
  source: /note=“Description of Artificial Sequence Synthetic peptide”
FKSWSLCTPGCARTGSFNQYCC

SEQ ID NO: 1093 - protein, 22 aa, Artificial Sequence
  source: /note=“Description of Artificial Sequence Synthetic peptide”
FKSWSLCTPGCARTGSFNFYCC

SEQ ID NO: 1094 - protein, 22 aa, Artificial Sequence
  source: /note=“Description of Artificial Sequence Synthetic peptide”
FKSWSLCTPGCARTGSFNGYCC

SEQ ID NO: 1095 - protein, 22 aa, Artificial Sequence
  source: /note=“Description of Artificial Sequence Synthetic peptide”
FKSWSLCTPGCARTGSFNRYCC

SEQ ID NO: 1096 - protein, 22 aa, Artificial Sequence
  source: /note=“Description of Artificial Sequence Synthetic peptide”
FKSWSLCTPGCARTGSFNHYCC

SEQ ID NO: 1097 - protein, 22 aa, Artificial Sequence
  source: /note=“Description of Artificial Sequence Synthetic peptide”
FKSWSLCTPGCARTGSFNTYCC

SEQ ID NO: 1098 - protein, 22 aa, Artificial Sequence
  source: /note=“Description of Artificial Sequence Synthetic peptide”
FKSWSLCTPGCARTGSFNIYCC

SEQ ID NO: 1099 - protein, 22 aa, Artificial Sequence
  source: /note=“Description of Artificial Sequence Synthetic peptide”
FKSWSLCTPGCARTGSFNSKCC

SEQ ID NO: 1100 - protein, 22 aa, Artificial Sequence
  source: /note=“Description of Artificial Sequence Synthetic peptide”
FKSWSLCTPGCARTGSFNSVCC

SEQ ID NO: 1101 - protein, 22 aa, Artificial Sequence
  source: /note=“Description of Artificial Sequence Synthetic peptide”
FKSWSLCTPGCARTGSFNSWCC

SEQ ID NO: 1102 - protein, 22 aa, Artificial Sequence
  source: /note=“Description of Artificial Sequence Synthetic peptide”
FKSWSLCTPGCARTGSFNSACC

SEQ ID NO: 1103 - protein, 22 aa, Artificial Sequence
  source: /note=“Description of Artificial Sequence Synthetic peptide”
FKSWSLCTPGCARTGSFNSLCC

SEQ ID NO: 1104 - protein, 22 aa, Artificial Sequence
  source: /note=“Description of Artificial Sequence Synthetic peptide”
FKSWSLCTPGCARTGSFNSMCC

SEQ ID NO: 1105 - protein, 22 aa, Artificial Sequence
  source: /note=“Description of Artificial Sequence Synthetic peptide”
FKSWSLCTPGCARTGSFNSCCC

SEQ ID NO: 1106 - protein, 22 aa, Artificial Sequence
  source: /note=“Description of Artificial Sequence Synthetic peptide”
FKSWSLCTPGCARTGSFNSNCC

SEQ ID NO: 1107 - protein, 22 aa, Artificial Sequence
  source: /note=“Description of Artificial Sequence Synthetic peptide”
FKSWSLCTPGCARTGSFNSDCC

SEQ ID NO: 1108 - protein, 22 aa, Artificial Sequence
  source: /note=“Description of Artificial Sequence Synthetic peptide”
FKSWSLCTPGCARTGSFNSECC

SEQ ID NO: 1109 - protein, 22 aa, Artificial Sequence
  source: /note=“Description of Artificial Sequence Synthetic peptide”
FKSWSLCTPGCARTGSFNSPCC

SEQ ID NO: 1110 - protein, 22 aa, Artificial Sequence
  source: /note=“Description of Artificial Sequence Synthetic peptide”
FKSWSLCTPGCARTGSFNSQCC

SEQ ID NO: 1111 - protein, 22 aa, Artificial Sequence
  source: /note=“Description of Artificial Sequence Synthetic peptide”
FKSWSLCTPGCARTGSFNSGCC

SEQ ID NO: 1112 - protein, 22 aa, Artificial Sequence
  source: /note=“Description of Artificial Sequence Synthetic peptide”
FKSWSLCTPGCARTGSFNSRCC

SEQ ID NO: 1113 - protein, 22 aa, Artificial Sequence
  source: /note=“Description of Artificial Sequence Synthetic peptide”
FKSWSLCTPGCARTGSFNSHCC

SEQ ID NO: 1114 - protein, 22 aa, Artificial Sequence
  source: /note=“Description of Artificial Sequence Synthetic peptide”
FKSWSLCTPGCARTGSFNSSCC

SEQ ID NO: 1115 - protein, 22 aa, Artificial Sequence
  source: /note=“Description of Artificial Sequence Synthetic peptide”
FKSWSLCTPGCARTGSFNSTCC

SEQ ID NO: 1116 - protein, 22 aa, Artificial Sequence
  source: /note=“Description of Artificial Sequence Synthetic peptide”
FKSWSLCTPGCARTGSFNSICC

SEQ ID NO: 1117 - protein, 22 aa, Artificial Sequence
  source: /note=“Description of Artificial Sequence Synthetic peptide”
FKSWSLCTPGCARTGSFNSYKC

SEQ ID NO: 1118 - protein, 22 aa, Artificial Sequence
  source: /note=“Description of Artificial Sequence Synthetic peptide”
FKSWSLCTPGCARTGSFNSYVC

SEQ ID NO: 1119 - protein, 22 aa, Artificial Sequence
  source: /note=“Description of Artificial Sequence Synthetic peptide”
FKSWSLCTPGCARTGSFNSYWC

SEQ ID NO: 1120 - protein, 22 aa, Artificial Sequence
  source: /note=“Description of Artificial Sequence Synthetic peptide”
FKSWSLCTPGCARTGSFNSYAC

SEQ ID NO: 1121 - protein, 22 aa, Artificial Sequence
  source: /note=“Description of Artificial Sequence Synthetic peptide”
FKSWSLCTPGCARTGSFNSYLC

SEQ ID NO: 1122 - protein, 22 aa, Artificial Sequence
  source: /note=“Description of Artificial Sequence Synthetic peptide”
FKSWSLCTPGCARTGSFNSYMC

SEQ ID NO: 1123 - protein, 22 aa, Artificial Sequence
  source: /note=“Description of Artificial Sequence Synthetic peptide”
FKSWSLCTPGCARTGSFNSYNC

SEQ ID NO: 1124 - protein, 22 aa, Artificial Sequence
  source: /note=“Description of Artificial Sequence Synthetic peptide”
FKSWSLCTPGCARTGSFNSYYC

SEQ ID NO: 1125 - protein, 22 aa, Artificial Sequence
  source: /note=“Description of Artificial Sequence Synthetic peptide”
FKSWSLCTPGCARTGSFNSYDC

SEQ ID NO: 1126 - protein, 22 aa, Artificial Sequence
  source: /note=“Description of Artificial Sequence Synthetic peptide”
FKSWSLCTPGCARTGSFNSYEC

SEQ ID NO: 1127 - protein, 22 aa, Artificial Sequence
  source: /note=“Description of Artificial Sequence Synthetic peptide”
FKSWSLCTPGCARTGSFNSYPC

SEQ ID NO: 1128 - protein, 22 aa, Artificial Sequence
  source: /note=“Description of Artificial Sequence Synthetic peptide”
FKSWSLCTPGCARTGSFNSYQC

SEQ ID NO: 1129 - protein, 22 aa, Artificial Sequence
  source: /note=“Description of Artificial Sequence Synthetic peptide”
FKSWSLCTPGCARTGSFNSYFC

SEQ ID NO: 1130 - protein, 22 aa, Artificial Sequence
  source: /note=“Description of Artificial Sequence Synthetic peptide”
FKSWSLCTPGCARTGSFNSYGC

SEQ ID NO: 1131 - protein, 22 aa, Artificial Sequence
  source: /note=“Description of Artificial Sequence Synthetic peptide”
FKSWSLCTPGCARTGSFNSYRC

SEQ ID NO: 1132 - protein, 22 aa, Artificial Sequence
  source: /note=“Description of Artificial Sequence Synthetic peptide”
FKSWSLCTPGCARTGSFNSYHC

SEQ ID NO: 1133 - protein, 22 aa, Artificial Sequence
  source: /note=“Description of Artificial Sequence Synthetic peptide”
FKSWSLCTPGCARTGSFNSYSC

SEQ ID NO: 1134 - protein, 22 aa, Artificial Sequence
  source: /note=“Description of Artificial Sequence Synthetic peptide”
FKSWSLCTPGCARTGSFNSYTC

SEQ ID NO: 1135 - protein, 22 aa, Artificial Sequence
  source: /note=“Description of Artificial Sequence Synthetic peptide”
FKSWSLCTPGCARTGSFNSYIC

SEQ ID NO: 1136 - protein, 22 aa, Artificial Sequence
  source: /note=“Description of Artificial Sequence Synthetic peptide”
FKSWSLCTPGCARTGSFNSYCK

SEQ ID NO: 1137 - protein, 22 aa, Artificial Sequence
  source: /note=“Description of Artificial Sequence Synthetic peptide”
FKSWSLCTPGCARTGSFNSYCV

SEQ ID NO: 1138 - protein, 22 aa, Artificial Sequence
  source: /note=“Description of Artificial Sequence Synthetic peptide”
FKSWSLCTPGCARTGSFNSYCW

SEQ ID NO: 1139 - protein, 22 aa, Artificial Sequence
  source: /note=“Description of Artificial Sequence Synthetic peptide”
FKSWSLCTPGCARTGSFNSYCA

SEQ ID NO: 1140 - protein, 22 aa, Artificial Sequence
  source: /note=“Description of Artificial Sequence Synthetic peptide”
FKSWSLCTPGCARTGSFNSYCL

SEQ ID NO: 1141 - protein, 22 aa, Artificial Sequence
  source: /note=“Description of Artificial Sequence Synthetic peptide”
FKSWSLCTPGCARTGSFNSYCM

SEQ ID NO: 1142 - protein, 22 aa, Artificial Sequence
  source: /note=“Description of Artificial Sequence Synthetic peptide”
FKSWSLCTPGCARTGSFNSYCN

SEQ ID NO: 1143 - protein, 22 aa, Artificial Sequence
  source: /note=“Description of Artificial Sequence Synthetic peptide”
FKSWSLCTPGCARTGSFNSYCY

SEQ ID NO: 1144 - protein, 22 aa, Artificial Sequence
  source: /note=“Description of Artificial Sequence Synthetic peptide”
FKSWSLCTPGCARTGSFNSYCD

SEQ ID NO: 1145 - protein, 22 aa, Artificial Sequence
  source: /note=“Description of Artificial Sequence Synthetic peptide”
FKSWSLCTPGCARTGSFNSYCE

SEQ ID NO: 1146 - protein, 22 aa, Artificial Sequence
  source: /note=“Description of Artificial Sequence Synthetic peptide”
FKSWSLCTPGCARTGSFNSYCP

SEQ ID NO: 1147 - protein, 22 aa, Artificial Sequence
  source: /note=“Description of Artificial Sequence Synthetic peptide”
FKSWSLCTPGCARTGSFNSYCQ

SEQ ID NO: 1148 - protein, 22 aa, Artificial Sequence
  source: /note=“Description of Artificial Sequence Synthetic peptide”
FKSWSLCTPGCARTGSFNSYCF

SEQ ID NO: 1149 - protein, 22 aa, Artificial Sequence
  source: /note=“Description of Artificial Sequence Synthetic peptide”
FKSWSLCTPGCARTGSFNSYCG

SEQ ID NO: 1150 - protein, 22 aa, Artificial Sequence
  source: /note=“Description of Artificial Sequence Synthetic peptide”
FKSWSLCTPGCARTGSFNSYCR

SEQ ID NO: 1151 - protein, 22 aa, Artificial Sequence
  source: /note=“Description of Artificial Sequence Synthetic peptide”
FKSWSLCTPGCARTGSFNSYCH

SEQ ID NO: 1152 - protein, 22 aa, Artificial Sequence
  source: /note=“Description of Artificial Sequence Synthetic peptide”
FKSWSLCTPGCARTGSFNSYCS

SEQ ID NO: 1153 - protein, 22 aa, Artificial Sequence
  source: /note=“Description of Artificial Sequence Synthetic peptide”
FKSWSLCTPGCARTGSFNSYCT

SEQ ID NO: 1154 - protein, 22 aa, Artificial Sequence
  source: /note=“Description of Artificial Sequence Synthetic peptide”
FKSWSLCTPGCARTGSFNSYCI

SEQ ID NO: 1155 - protein, 22 aa, Artificial Sequence
  source: /note=“Description of Artificial Sequence Synthetic peptide”
ITSMSLCTPGCAATGSFNSYCC

SEQ ID NO: 1156 - protein, 21 aa, Artificial Sequence
  source: /note=“Description of Artificial Sequence Synthetic peptide”
  MISC_FEATURE (3)..(7): /note=“Lanthionine ring between residues”
  MOD_RES (5)..(5): 2,3-didehydroalanine
  MOD_RES (8)..(8): Aminobutyrate
  MISC_FEATURE (8)..(11): /note=“3-methyl lanthionine ring between residues”
  MOD_RES (14)..(14): 2, 3-didehydrobutyrine
  MISC_FEATURE (16)..(21): /note=“Lanthionine ring between residues”
  MISC_FEATURE (19)..(21): /note=“Ala in position 19 linked to an aminovinyl group by a thioether linkage (Ala19-S-CH=CH-NH-) forming a ring with Ala at position 21”
FKAWXLAXPGAARXGAFNAYA

SEQ ID NO: 1157 - protein, 21 aa, Artificial Sequence
  source: /note=“Description of Artificial Sequence Synthetic peptide”
  MISC_FEATURE (3)..(7): /note=“Lanthionine ring between residues”
  MOD_RES (5)..(5): 2,3-didehydroalanine
  MOD_RES (8)..(8): Aminobutyrate
  MISC_FEATURE (8)..(11): /note=“3-methyl lanthionine ring between residues”
  MOD_RES (14)..(14): 2, 3-didehydrobutyrine
  MISC_FEATURE (16)..(21): /note=“Lanthionine ring between residues”
  MISC_FEATURE (19)..(21): /note=“Ala in position 19 linked to an aminovinyl group by a thioether linkage (Ala19-S-CH=CH-NH-) forming a ring with Ala at position 21”
VKAWXLAXPGAANXGAFNAYA

SEQ ID NO: 1158 - protein, 22 aa, Artificial Sequence
  source: /note=“Description of Artificial Sequence Synthetic peptide”
  MOD_RES (5)..(5): 2,3-didehydroalanine
  MOD_RES (8)..(8): Aminobutyrate
  MOD_RES (14)..(14): 2, 3-didehydrobutyrine
VKAWXLAXPGAANXGAFNAYAC

SEQ ID NO: 1159 - protein, 22 aa, Artificial Sequence
  source: /note=“Description of Artificial Sequence Synthetic peptide”
  MOD_RES (5)..(5): 2,3-didehydroalanine
  MOD_RES (8)..(8): Aminobutyrate
  MOD_RES (14)..(14): 2, 3-didehydrobutyrine
FKAWXLAXPGAARXGAFNAYAC

SEQ ID NO: 1160 - protein, 63 aa, Streptococcus mutans
MSNTQLLEVLGTETFDVQEDLFAFDTTDTTIVASNDDPDTRFKSWSLCTPGCARTGSFNSYCC

SEQ ID NO: 1161 - protein, 63 aa, Artificial Sequence
  source: /note=“Description of Artificial Sequence Synthetic polypeptide”
MSNTQLLEVLGTETFDVQEDLFAFDTTDTTIVASNDDPDTRVKSWSLCTPGCANTGSFNSYCC

SEQ ID NO: 1162 - DNA, 189 bp, Streptococcus mutans
atgtcaaacacacaattattagaagtccttggtactgaaacttttgatgttcaagaagatctctttgcttttgatacaacagatactactattgtggcaagcaacgacgatccagatactcgtttcaaaagttggagcctttgtacgcctggttgtgcaaggacaggtagtttcaatagttactgttgc

SEQ ID NO: 1163 - DNA, 189 bp, Artificial Sequence
  source: /note=“Description of Artificial Sequence Synthetic polynucleotide”
atgtcaaacacacaattattagaagtccttggtactgaaacttttgatgttcaagaagatctctttgcttttgatacaacagatactactattgtggcaagcaacgacgatccagatactcgtgttaaaagttggagcctttgtacgcctggttgtgcaaatacaggtagtttcaatagttactgttgc

SEQ ID NO: 1164 - protein, 41 aa, Artificial Sequence
  source: /note=“Description of Artificial Sequence Synthetic polypeptide”
MSNTQLLEVLGTETFDVQEDLFAFDTTDTTIVASNDDPDTR

SEQ ID NO: 1165 - DNA, 123 bp, Artificial Sequence
  source: /note=“Description of Artificial Sequence Synthetic polynucleotide”
atgtcaaacacacaattattagaagtccttggtactgaaacttttgatgttcaagaagatctctttgcttttgatacaacagatactactattgtggcaagcaacgacgatccagatactcgt

SEQ ID NO: 1166 - protein, 6 aa, Artificial Sequence
  source: /note=“Description of Artificial Sequence Synthetic 6xHis tag”
HHHHHH

SEQ ID NO: 1167 - DNA, 66 bp, Artificial Sequence
  source: /note=“Description of Artificial Sequence Synthetic oligonucleotide”
gctaaaagttggagcctttgtacgcctggttgtgcagctacaggtagtttcaatagttactgttgc

SEQ ID NO: 1168 - DNA, 66 bp, Artificial Sequence
  source: /note=“Description of Artificial Sequence Synthetic oligonucleotide”
ttaaaaagttggagcctttgtacgcctggttgtgcaaggacaggtagtttcaatagttactgttgc

SEQ ID NO: 1169 - protein, 22 aa, Artificial Sequence
  source: /note=“Description of Artificial Sequence Synthetic peptide”
FKKWSLCTPGCARTGSFNSYCC

SEQ ID NO: 1170 - protein, 22 aa, Artificial Sequence
  source: /note=“Description of Artificial Sequence Synthetic peptide”
FKVWSLCTPGCARTGSFNSYCC

SEQ ID NO: 1171 - protein, 22 aa, Artificial Sequence
  source: /note=“Description of Artificial Sequence Synthetic peptide”
FKWWSLCTPGCARTGSFNSYCC

SEQ ID NO: 1172 - protein, 22 aa, Artificial Sequence
  source: /note=“Description of Artificial Sequence Synthetic peptide”
FKAWSLCTPGCARTGSFNSYCC

SEQ ID NO: 1173 - protein, 22 aa, Artificial Sequence
  source: /note=“Description of Artificial Sequence Synthetic peptide”
FKLWSLCTPGCARTGSFNSYCC

SEQ ID NO: 1174 - protein, 22 aa, Artificial Sequence
  source: /note=“Description of Artificial Sequence Synthetic peptide”
FKMWSLCTPGCARTGSFNSYCC

SEQ ID NO: 1175 - protein, 22 aa, Artificial Sequence
  source: /note=“Description of Artificial Sequence Synthetic peptide”
FKCWSLCTPGCARTGSFNSYCC

SEQ ID NO: 1176 - protein, 22 aa, Artificial Sequence
  source: /note=“Description of Artificial Sequence Synthetic peptide”
FKNWSLCTPGCARTGSFNSYCC

SEQ ID NO: 1177 - protein, 22 aa, Artificial Sequence
  source: /note=“Description of Artificial Sequence Synthetic peptide”
FKYWSLCTPGCARTGSFNSYCC

SEQ ID NO: 1178 - protein, 22 aa, Artificial Sequence
  source: /note=“Description of Artificial Sequence Synthetic peptide”
FKDWSLCTPGCARTGSFNSYCC

SEQ ID NO: 1179 - protein, 22 aa, Artificial Sequence
  source: /note=“Description of Artificial Sequence Synthetic peptide”
FKEWSLCTPGCARTGSFNSYCC

SEQ ID NO: 1180 - protein, 22 aa, Artificial Sequence
  source: /note=“Description of Artificial Sequence Synthetic peptide”
FKPWSLCTPGCARTGSFNSYCC

SEQ ID NO: 1181 - protein, 22 aa, Artificial Sequence
  source: /note=“Description of Artificial Sequence Synthetic peptide”
FKQWSLCTPGCARTGSFNSYCC

SEQ ID NO: 1182 - protein, 22 aa, Artificial Sequence
  source: /note=“Description of Artificial Sequence Synthetic peptide”
FKFWSLCTPGCARTGSFNSYCC

SEQ ID NO: 1183 - protein, 22 aa, Artificial Sequence
  source: /note=“Description of Artificial Sequence Synthetic peptide”
FKGWSLCTPGCARTGSFNSYCC

SEQ ID NO: 1184 - protein, 22 aa, Artificial Sequence
  source: /note=“Description of Artificial Sequence Synthetic peptide”
FKRWSLCTPGCARTGSFNSYCC

SEQ ID NO: 1185 - protein, 22 aa, Artificial Sequence
  source: /note=“Description of Artificial Sequence Synthetic peptide”
FKHWSLCTPGCARTGSFNSYCC

SEQ ID NO: 1186 - protein, 22 aa, Artificial Sequence
  source: /note=“Description of Artificial Sequence Synthetic peptide”
FKTWSLCTPGCARTGSFNSYCC

SEQ ID NO: 1187 - protein, 22 aa, Artificial Sequence
  source: /note=“Description of Artificial Sequence Synthetic peptide”
FKIWSLCTPGCARTGSFNSYCC

SEQ ID NO: 1188 - protein, 22 aa, Artificial Sequence
  source: /note=“Description of Artificial Sequence Synthetic peptide”
FKSKSLCTPGCARTGSFNSYCC

(19) United States
(12) Patent Application Publication
(10) Pub. No.: US 2019/0315814 A1
(43) Pub. Date: Oct. 17, 2019

(54) LANTIBIOTIC VARIANTS AND USES THEREOF

(71) Applicant: INTREXON CORPORATION, Blacksburg, VA (US)

(72) Inventors: Martin HANDFIELD, Gainesville, FL (US); Johan Anthony KERS, Wellesley, MA (US); Sheela MULEY, Fremont, CA (US); Melissa MAYO, Foster City, CA (US); Jeffrey COLBECK, Belmont, CA (US); Robert Eryl SHARP, Belmont, CA (US); Albert G. FOSMOE, II, Gainsville, FL (US); Anthony W. DEFUSCO, Newberry, FL (US)

(21) Appl. No.: 16/316,328

(22) PCT Filed: Jul. 14, 2017
(86) PCT No.: PCT/US2017/042206

§ 371 Date: Jan. 8, 2019

Related U.S. Application Data
(60) Provisional application No. 62/362,788, filed on Jul. 15, 2016.
(60) Provisional application No. 62/362,809, filed on Jul. 15, 2016.
(60) Provisional application No. 62/420,328, filed on Nov. 10, 2016.

(51) Int. Cl. C07K 14/315; A61K 45/06; A61K 38/16; A61K 9/68; A61Q 11/00; A61K 9/00; A01N 63/02; A61P 31/04; A61K 8/64

(52) U.S. Cl. CPC C07K 14/315; A61K 45/06; A61K 38/164; A61K 9/0058; A61K 8/64; A61K 9/0056; A01N 63/02; A61P 31/04; A61Q 11/00

(57) ABSTRACT

The disclosure relates to lantibiotic structural variants that have improved antimicrobial properties when compared to wild-type lantibiotics, and uses thereof. Embodiments include non-naturally occurring lantibiotics.

### CROSS-REFERENCE TO RELATED APPLICATIONS

The present application claims the benefit of U.S. Provisional Patent Application Nos., 62/362,788 filed Jul. 15, 2016, 62/362,809 filed Jul. 15, 2016 and 62/420,328 filed Nov. 10, 2016; each of which is hereby incorporated by reference in its entirety.

### REFERENCE TO SEQUENCE LISTING

The present application contains a Sequence Listing which has been filed electronically in ASCII format and is hereby incorporated by reference in its entirety. Said ASCII copy, created on Jul. 12, 2017, is named 0100-0017WO1_SL.txt and is 421,465 bytes in size.

### FIELD OF THE INVENTION

The present invention is generally related to the fields of molecular biology, infectious disease and polypeptide-based antibiotics. More specifically, certain aspects of the invention are related to novel lantibiotic (polypeptide) variants and nucleic acid sequences encoding one or more lantibiotic (polypeptide) variants. Certain other aspects of the invention are related to recombinant vectors comprising one or more nucleic acid sequences encoding one or more lantibiotic variants, host cells transformed with said vectors, methods for producing lantibiotic variants and methods of use thereof.

### BACKGROUND OF THE INVENTION

Many strains of disease-causing bacteria have become increasingly resistant to currently available antibiotics. Healthcare-associated infections caused by multi-drug resistant pathogens are particularly vexing. Worldwide, millions suffer from antibiotic-resistant infections, which results in immense cost to the healthcare system. The need for new antibiotics has become a critical, unmet need in the medical community.

Lantibiotics, a class of antibiotics with potential clinical relevance (reviewed in Smith & Hillman, (2008) Curr. Opin. Microbial. 11:401-408), acquired their name because of the characteristic lanthionine rings that are present. Lantibiotics are also known to have various unusual amino acids such as 2,3-didehydroalanine (Dha), 2, 3-didehydrobutyrine (Dhb), S-amino vinyi-D-cysteine (AviCys), aminobutyrate (Abu), 2-oxopropionyl, 2-oxobutyryl, and hydroxypropionyl. Hasper et al. (2006) Science 313:1636-1637. Mutacin 1140 (“MU1140”) is one type of lantibiotic that can be produced by a particular strain of the oral microorganism Streptococcus mutans. Smith et al. (2000) Eur. J. Biochem. 267:6810-6816.

### SUMMARY OF THE INVENTION

The present disclosure provides non-naturally occurring lantibiotic comprising a single amino acid mutation of MU1140, the lantibiotic having an amino acid sequence encoded by of any one of SEQ ID NOS:2 to 431.

The present disclosure also provides a non-naturally occurring lantibiotic comprising multisite amino acid mutations of MU1140, the lantibiotic being a variant as described in FIG. 7.

The present disclosure also provides a non-naturally occurring lantibiotic comprising any variant as described in FIG. 8: Group 1, Group 2 or Group 3. In some embodiments, the non-naturally occurring lantibiotic comprises any variant as described in FIG. 8: Group 1.

The present disclosure also provides a non-naturally occurring lantibiotic comprising a single amino acid mutation of MU1140 as described in FIG. 6.

The present disclosure also provides a non-naturally occurring lantibiotic comprising an amino acid sequence of MU1140 (e.g., SEQ ID NO:1 156) and further comprising a mutation that is: (a) arginine at position 13 changed to asparagine (R13N); (b) phenylalanine at position 17 changed to leucine (F17L) or tyrosine (F17Y); (c) asparagine at position 18 changed to alanine (N18A); (d) tyrosine at position 20 changed to phenylalanine (Y20F); or (e) combinations thereof. In some embodiments, the non-naturally occurring lantibiotic comprises an amino acid sequence of MU1140 (e.g., SEQ ID NO: 1156) and further comprises two mutations, wherein one mutation is: (a) arginine at position 13 changed to asparagine (R13N); (b) phenylalanine at position 17 changed to leucine (F17L) or tyrosine (F17Y); (c) asparagine at position 18 changed to alanine (N18A); (d) tyrosine at position 20 changed to phenylalanine (Y20F); or (e) combinations thereof. In some embodiments, the non-naturally occurring lantibiotic comprises an amino acid sequence of MU1140 (e.g., SEQ ID NO:1156) and further comprises two mutations, wherein one mutation is arginine at position 13 changed to asparagine (R13N).

In some embodiments, the disclosure provides a non-naturally occurring lantibiotic comprising an amino acid sequence of MU1140 (e.g., SEQ ID NO: 1156) and further comprises two mutations, wherein one mutation is: (a) phenylalanine at position 1 changed to valine (F1V); (b) phenylalanine at position 1 changed to alanine (F1A); (c) phenylalanine at position 1 changed to isoleucine (F1I); (d) phenylalanine at position 1 changed to leucine (F1L); (e) phenylalanine at position 1 changed to threonine (F1T); or (f) phenylalanine at position 1 changed to tyrosine (F1Y). In some embodiments, the disclosure provides a non-naturally occurring lantibiotic comprising an amino acid sequence of MU1140 (e.g., SEQ ID NO: 1156) and further comprises two mutations, wherein one mutation is phenylalanine at position 1 changed to valine (F1V). The present disclosure also provides a non-naturally occurring lantibiotic comprising an amino acid sequence of MU1140 (e.g., SEQ ID NO:1156) and further comprising two mutations including: (a) phenylalanine at position 1 changed to alanine in combination with arginine at position 13 changed to: (i) alanine (F1A R13A), (ii) valine (F1A R13V), (iii) asparagine (F1A R13N), or (iv) serine (F1A R13S); (b) phenylalanine at position 1 changed to glycine in combination with arginine at position 13 changed to: (i) glycine (F1G R13G); (c) phenylalanine a position 1 changed to histidine in combination with arginine at position 13 changed to: (i) asparagine (F1H R13N); (d) phenylalanine at position 1 changed to isoleucine in combination with arginine at position 13 changed to: (i) alanine (F1I R13A), (ii) glycine (F1I R13G), (iii) isoleucine (F1I R13I), (iv) asparagine (F1I R13N), (v) proline (F1I R13P), (vi) glutamine (F1I R13Q), (vi) glutamic acid (F1I R13S), (vii) serine (F1I R13V), or (viii) valine (F1I R13E); (e) phenylalanine at position 1 changed to leucine in combination with arginine at position 13 changed to: (i) alanine (F1L R13A), (ii) aspartic acid (F1L R13D), (iii) glycine (F1L R13G), (iv) asparagine (F1L R13N), (v) proline (F1L R13P), or (vi) glutamine (F1L R13Q); (f) phenylalanine at position 1 changed to threonine in combination with arginine at position 13 changed to: (i) alanine (F1T R13A), (ii) asparagine (F1T R13N), or (iii) valine (F1T R13V); (g) phenylalanine at position 1 changed to valine in combination with arginine at position 13 changed to: (i) alanine (F1V R13A), (ii) asparagine (F1V R13N), (iii) glutamine (F1V R13Q), (iv) aspartic acid (F1V R13D), (v) valine (F1V R13V), or (vi) proline (F1V R13P); or (h) phenylalanine at position 1 changed to tyrosine in combination with arginine at position 13 changed to: (i) aspartic acid (F1Y R13D), or (ii) glycine (F1Y R13G). In some embodiments, the disclosure provides a non-naturally occurring lantibiotic comprising an amino acid sequence of MU1140 (e.g., SEQ ID NO: 1156) and further comprising two mutations including phenylalanine at position 1 changed to valine in combination with arginine at position 13 changed to: (i) alanine (F1V R13A), (ii) asparagine (F1V R13N), (iii) glutamine (F1V R13Q), (iv) aspartic acid (F1V R13D), (v) valine (F1V R13V), or (vi) proline (F1V R13P). In some embodiments, the disclosure provides a non-naturally occurring lantibiotic comprising an amino acid sequence of MU1140 (e.g., SEQ ID NO: 1156) having two mutations, wherein the two mutations consist of phenylalanine at position 1 changed to valine in combination with arginine at position 13 changed to asparagine (F1V R13N) (e.g., SEQ ID NO:1157).

The present disclosure also provides a non-naturally occurring lantibiotic comprising an amino acid sequence of MU1140 (e.g., SEQ ID NO: 1156) and further comprising three mutations including: (a) phenylalanine at position 1 changed to isoleucine, in combination with arginine at position 13 changed to alanine, in combination with glycine at position 15 changed to alanine (F1I R13A G15A); (b) phenylalanine at position 1 changed to isoleucine, in combination with arginine at position 13 changed to aspartic acid, in combination with glycine at position 15 changed to alanine (F1I R13D G15A); (c) phenylalanine at position 1 changed to isoleucine, in combination with tryptophan at position 4 changed to isoleucine, in combination with arginine at position 13 changed to alanine (F1I W4I R13A); (d) phenylalanine at position 1 changed to isoleucine, in combination with tryptophan at position 4 changed to methionine, in combination with arginine at position 13 changed to aspartic acid (F1I W4M R13D); (e) phenylalanine at position 1 changed to isoleucine, in combination with tryptophan at position 4 changed to methionine, in combination with arginine at position 13 changed to asparagine (F1I W4M R13N); (f) phenylalanine at position 1 changed to isoleucine, in combination with lysine at position 2 changed to alanine, in combination with arginine at position 13 changed to alanine (F1I K2A R13A); (g) phenylalanine at position 1 changed to isoleucine, in combination with leucine at position 6 changed to valine, in combination with arginine at position 13 changed to alanine (F1I L6V R13A); (h) phenylalanine at position 1 changed to isoleucine, in combination with arginine at position 13 changed to alanine, in combination with tyrosine at position 20 changed to phenylalanine (F1I R13A Y20F); (i) phenylalanine at position 1 changed to isoleucine, in combination with arginine at position 13 changed to aspartic acid, in combination with tyrosine at position 20 changed to phenylalanine (F1I R13D Y20F); (i) phenylalanine at position 1 changed to isoleucine, in combination with arginine at position 13 changed to asparagine, in combination with tyrosine at position 20 changed to phenylalanine (F1I R13N Y20F); (k) phenylalanine at position 1 changed to leucine, in combination with arginine at position 13 changed to asparagine, in combination with tyrosine at position 20 changed to phenylalanine (F1L R13N Y20F); (l) phenylalanine at position 1 changed to leucine, in combination with arginine at position 13 changed to alanine, in combination with tyrosine at position 20 changed to phenylalanine (F1L R13A Y20F); or (m) phenylalanine at position 1 changed to leucine, in combination with arginine at position 13 changed to aspartic acid, in combination with tyrosine at position 20 changed to phenylalanine (F1L R13D Y20F).

The present disclosure also provides a non-naturally occurring lantibiotic comprising an amino acid sequence of MU1140 (e.g., SEQ ID NO: 1156) and further comprising mutations including: (a) phenylalanine at position 1 changed to isoleucine, in combination with lysine at position 2 changed to alanine, in combination with tryptophan at position 4 changed to lysine, in combination with arginine at position 13 changed to alanine (F1I K2A W4K R13A); and (b) phenylalanine at position 1 changed to isoleucine, in combination with lysine at position 2 changed to alanine, in combination with tryptophan at position 4 changed to lysine, in combination with arginine at position 13 changed to aspartic acid (F1I K2A W4K R13D).

The present disclosure also provides a non-naturally occurring lantibiotic comprising an amino acid sequence of MU1140 (e.g., SEQ ID NO: 1156) and further comprising mutations including: phenylalanine at position 1 changed to isoleucine, in combination with lysine at position 2 changed to alanine, in combination with tryptophan at position 4 changed to methionine, in combination with arginine at position 13 changed to alanine, in combination with tyrosine at position 20 changed to phenylalanine (F1I K2A W4K R13A Y20F).

In some embodiments, the disclosure is directed to an antimicrobial composition comprising a non-naturally occurring lantibiotic as described herein and a pharmaceutically acceptable carrier, pharmaceutically acceptable diluent, other diluent or excipient. In some embodiments, the composition further comprises an antifungal agent, an additional antimicrobial agent, a membrane disrupting agent, or a combination thereof. In some embodiments, the additional antimicrobial agent has Gram negative bacteriostatic or bacteriocidal activity and the membrane disrupting agent renders Gram negative bacteria susceptible to the variant lantibiotic.

In some embodiments, the one or more isolated lantibiotics are present in the composition at about 0.001, 0.01, 0.1, 1, 5, 10, 20, 30, 40, 50, 75, 100, 150, 200, 300, 400, 500, 600, 700, 800, 900, or 1,000 mg/kg or mg/L.

In some embodiments, the disclosure is directed to a method of reducing reproduction of bacteria or reducing numbers of bacteria present in or on a subject, comprising administering to the subject a therapeutically effective amount of the antimicrobial composition as described herein. In some embodiments, the subject is a human.

In some embodiments, the composition is administered orally, topically, nasally, buccally, sublingually, transmucosally, rectally, transdermally, by inhalation, by injection or intrathecally. In some embodiments, the injection is intramuscular, intravenous, intrapulmonary, intramuscular, intradermal, intraperitoneal, intrathecal, or subcutaneous injection.

In some embodiments, the disclosure is directed to a preservative comprising an effective amount of the non-naturally occurring lantibiotic as described herein in a physiological solution at a pH of between 3 and 8.

In some embodiments, the disclosure is directed to a food, beverage, gum, or dentifrice composition comprising an amount of the non-naturally occurring lantibiotic as described herein sufficient to reduce the reproduction of bacteria or numbers of bacteria in the composition.

In some embodiments, the disclosure is directed to a method of reducing reproduction of bacteria or reducing numbers of bacteria present in or on a composition or object, comprising contacting the antimicrobial composition as described herein with the composition or object for a period effective to reduce reproduction of bacteria or reduce numbers of bacteria in or on the composition or object. In some embodiments, the composition is a food, beverage, gum, or dentifrice.

In some embodiments, the disclosure is directed to a purified polynucleotide comprising any one of SEQ ID NOs: 2-431 or encoding a variant as described in FIG. 7 or combinations thereof. In some embodiments, the disclosure provides a purified polynucleotide comprising any one of SEQ ID NOs: 756, 757, 758, 559, 760, or 761. In some embodiments, the disclosure provides a purified polynucleotide comprising any one of SEQ ID NOs: 709, 714, 716, 735, 747, 751, 754, or 758. In some embodiments, the disclosure ir directed to a purified polynucleotide comprising SEQ ID NO: 758.

In some embodiments, the disclosure is directed to a composition comprising a solid surface or a textile with the lantibiotic composition as described herein or coated onto, immobilized, linked, or bound to the solid surface or textile.

In some embodiments, the disclosure is directed to a method of reducing a biofilm or biofouling condition comprising contacting the antimicrobial composition as described herein with the biofilm or biofouling condition for a period effective to reduce reproduction of bacteria or reduce numbers of bacteria in or on the biofilm or biofouling condition.

In some embodiments, the disclosure is directed to a kit comprising the lantibiotic as described herein and one or more applicators.

In some embodiments, the disclosure is directed to a method of preventing or treating a subject diagnosed with a bacterial infection, comprising administering the non-naturally occurring lantibiotic as described herein. In some embodiments, the subject is a human. In some embodiments, the subject is infected with a Gram-positive bacteria. In some embodiments, the Gram-positive bacteria is one or more of Staphylococcus epidermidis, vancomycin resistant Enterococci, vancomycin resistant Enterococcus faecalis, Enterococcus faecalis, Enterococcus faecium, Propionibacterium acnes, Streptococcus salivarius, Streptococcus sanguis, Streptococcus mitis, Streptococcus pyogenes, Lactobacillus salivarius, Listeria monocytogenes, Actinomyces israelii, Actinomyces naeslundii, Actinomyces viscosus, Bacillus anthracis, Streptococcus agalactiae, Streptococcus intermedius, Streptococcus pneumoniae, Corynebacterium diphtheria, Clostridium sporogenes Clostridium botulinum, Clostridium perfringens, Clostridium tetani, and Clostridium difficile. In some embodiments, the Gram-positive bacteria is Clostridium difficile.

In some embodiments, the subject is infected with a Gram-negative bacteria. In some embodiments, the Gram-negative bacteria is one or more of Acinetobacter baumanii, Bordatella pertussis, Borrelia burgdotieri, Brucella abortus, Brucella canis, Brucella melitensis, Brucella suis, Campylobacter jejuni, Coxiella burnetii, Escherichia coli, Francisella tularensis, Haemophilus influenza, Helicobacter pylori, Klebsiella pneumoniae, Legionella pneumophila, Leptospira interrogans, Neisseria gonorrhoeae, Neisseria meningitides, Pseudomonas aeruginosa, Rickettsia rickettsii, Salmonella enteritidis, Salmonella typhi, Salmonella typhimurium, Serratia marcescens, Shigella sonnei, Treponema pallidum, Vibrio cholera, Yersinia enterocolitica, and Yersinia pestis. In some embodiments, the non-naturally occurring lantibiotic further comprises one or more additional antimicrobial agents, membrane disrupting agents, or combinations thereof.

In some embodiments, the disclosure is directed to an isolated recombinant Streptococcus mutans strain comprising: (a) a mutation in a polynucleotide involved in lactic acid synthesis such that expression of lactic acid is diminished by about 80% or more as compared to a wildtype S. mutans strain; (b) a recombinant alcohol dehydrogenase polynucleotide; and (c) a recombinant polynucleotide encoding a non-naturally occurring lantibiotic as described in FIG. 5 or FIG. 7.

### BRIEF DESCRIPTION OF THE FIGURES

FIG. 1 shows the primary amino acid sequence, secondary structure and macrocyclic rings of the wild-type (native) mature MU1140 lantibiotic polypeptide (SEQ ID NO:1156) (FIGS. 1A and 1B), and the primary amino acid sequence, secondary structure and macrocyclic rings of the multisite variant F1V R13N of mature MU1140 lantibiotic polypeptide, (SEQ ID NO: 1157) (FIGS. 1C and 1D). Abbreviations of amino acids include the following: Ala-S-Ala is lanthionine; Abu-S-Ala is 3-methyl lanthionine; Dha is α, β-didehydroalanine; Dhb is α,β-didehydrobutyrine (Smith et al., Biochemistry, 42(35): 10372-84 (2003)).

FIG. 2 shows a plasmid vector map of pLAN042.

FIG. 3 shows a plasmid map of pLAN126.

FIG. 4 is a listing of single amino acid variants of MU1140, including polynucleotides SEQ ID NO:1 (MU1140) and SEQ ID NOS:2-431 (variants of MU1140) which can encode the variants.

FIG. 5 is a listing of SEQ ID NO: 432 (MU1140) and single amino acid variants of MU1140.

FIG. 6 is a listing of single amino acid variants of MU1140 that were chromosomally integrated into S. mutans.

FIG. 7 is a listing of SEQ ID NO: 432 (MU1140) featuring single and multiple amino acid variants of MU1140.

FIG. 8: Summary of zone clearing results of various mutants. The zone clearing ability is divided into six groups: Group 1: >activity than SM253 control strain (>0.319 cm² zone of clearing); Group 2: >activity than SM253 in 2 replicate experiments (1 replicate experiment; Group 3: >activity than SM253 in 1 replicate and ≤activity in 1 replicate experiment; Group 4: >activity than SM253 control strain (>0.167 cm² zone of clearing but <0.319 cm² zone of clearing); Group 5: ≤activity than SM253 control (≤0.167 cm² zone of clearing); and Group 6: undetermined.

FIG. 9: Example of sequences of post-translationally modified lantibiotic wild-type (SEQ ID NO: 1159) and lantibiotic variants F1V R13N (SEQ ID NO: 1158) without the secondary structures, e.g., rings, indicated.

FIG. 10A: Sequence of full-length 63 amino acid pre-protein of MU1140 (SEQ ID NO: 1160, denoting the “core peptide” residues which can be part of the mature, biologically active lantibiotic, and the “cleaved peptide” residues which are cleaved during post-translational modification. FIG. 10B: Sequence of full-length 63 amino acid pre-protein of F1V R13N (SEQ ID NO:1161), also denoting the “core peptide” residues and the “cleaved peptide” residues.

FIG. 11A: Nucleotide sequence of full-length 63 amino acid pre-protein of MU1140 (SEQ ID NO: 1162). FIG. 11B: Nucleotide sequence of full-length 63 amino acid pre-protein of variant F1V R13N (SEQ ID NO: 1163). FIG. 11C: amino acid sequence of the cleaved protein, i.e., full-length 63 amino acid pre-protein minus the core peptide. FIG. 11D: nucleotide sequence of the cleaved protein.

### DETAILED DESCRIPTION OF THE INVENTION

### Lantibiotic MU1140 Variants

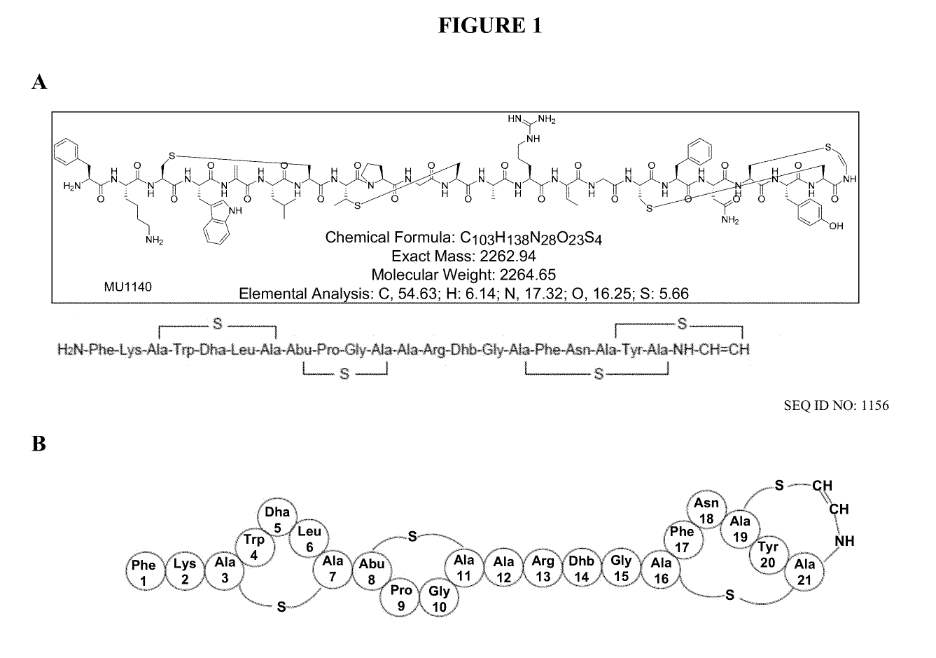

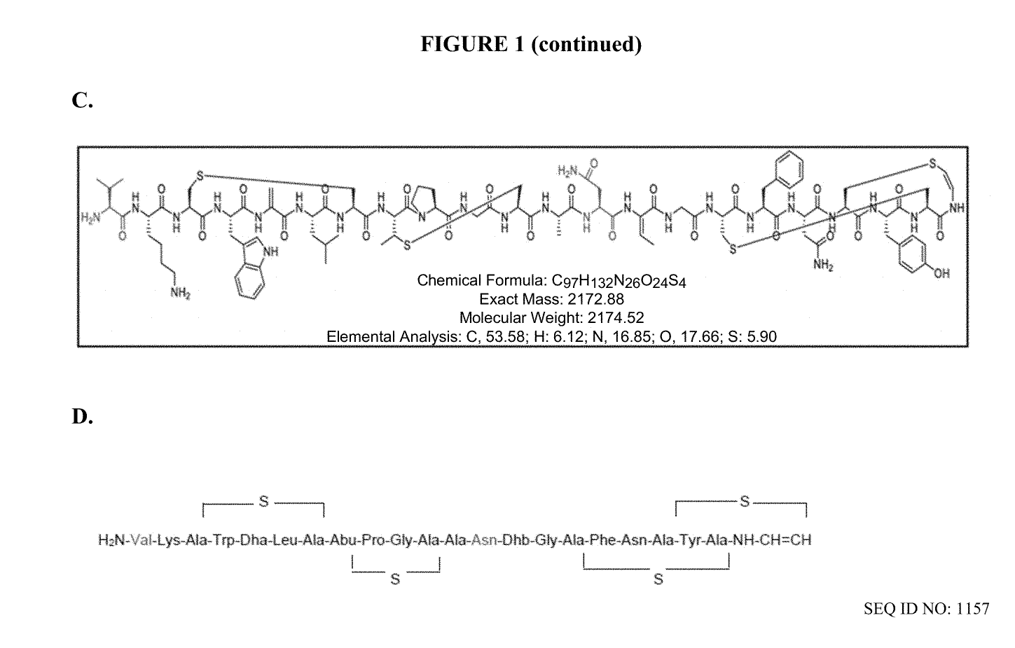

Wild-type MU1140 is shown in FIG. 1A and FIG. 1B. The term “MU1140” refers to (1) the translated, full-length 63 amino acid pre-protein of MU1140 (SEQ ID NO: 1160), (2) the translated, cleaved 22 amino acid polypeptide of MU1140 (SEQ ID NO: 432), as well as (3) the post-translationally modified biologically active MU1140 with secondary structure rings indicated (SEQ ID NO: 1156), or without secondary structure rings indicated (SEQ ID NO: 1159). SEQ ID NO: 1160 is a pre-protein form of a lantibiotic which, after proteolytic cleavage and processing by other factors present in a host cell, results in the synthesis of MU1140 (SEQ ID NO: 1156 or SEQ ID NO: 1159). SEQ ID NO: 432 is the cleaved 22 amino acid protein (“core peptide”) in which the post-translational modifications, e.g., positional mutations and ring formations, are not designated. For clarity, the phrase “lantibiotic comprising an amino acid sequence of MU1140” refers to various forms of the polypeptide that includes (1) the full-length 63 amino acid pre-protein of MU1140, (2) the post translated, cleaved 22 amino acid polypeptide of MU1140, as well as (3) the post-translationally modified biologically active MU1140. MU1140 can include forms of polypeptides as described above that may or may not have “lantibiotic” activity. Thus, a full-length 63 amino acid pre-protein of MU1140 which may lack lantibiotic activity still is considered a “lantibiotic comprising an amino acid sequence of MU1140,” because such a pre-protein can be post-translationally modified to obtain lantibiotic activity.

The term “post-translated cleaved polypeptide” refers to the polypeptide cleaved off during post-translational modification that is not part of the lantibiotic (SEQ ID NO: 1164). SEQ ID NO: 1156 refers to the cleaved, post-translationally modified biologically active MU1140. The post-translationally modified biologically active MU1140 has four rings labeled A, B, C, and D. Two of these rings are formed by lanthionine (Ala-S-Ala) residues, including one in Ring A (Ala₃-S-Ala₇) and one in Ring C (Ala₁₁-S-Ala₂₁); there is a methyl-lanthionine residue (Abu-S-Ala) that forms Ring B comprised of the alpha-aminobutyrate residue in position 8 and the Ala in position 11 (Abu₈-S-Ala₁₁); and the fourth ring, D, is comprised of the Ala in position 19 linked to an aminovinyl group by a thioether linkage (Ala₁₉-S—CH═CH—NH—). The terms “ring” or “rings” may be used interchangeably with “bridge(s)” or “linkage(s).” The term “biologically active” or “biologically functional” refers to polypeptides which can kill or inhibit the growth of gram positive or gram negative bacteria, i.e., they have antimicrobial activity.

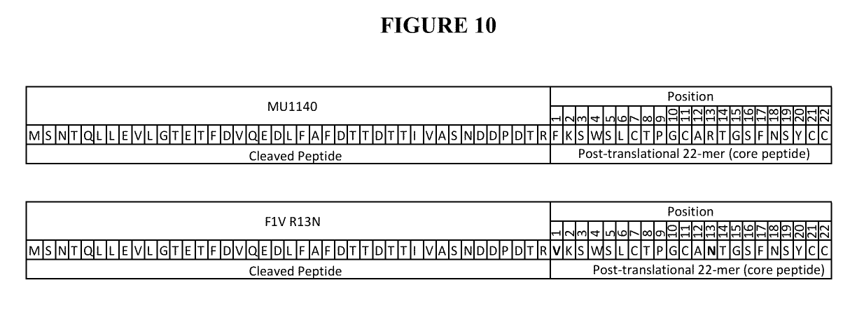

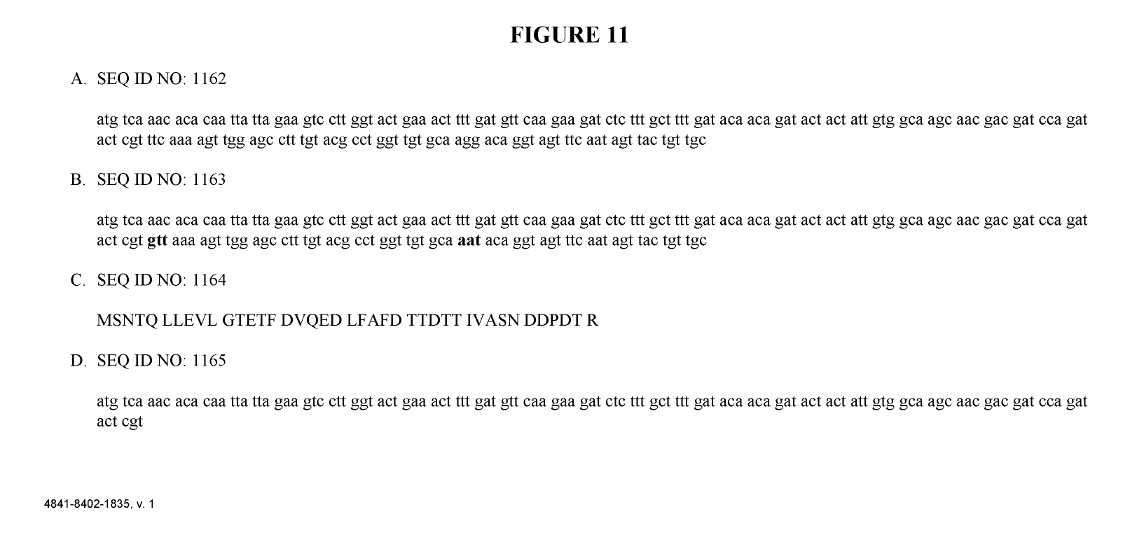

The variants of MU1140 described herein apply to the full-length 63 amino acid pre-protein (SEQ ID NO: 1160), the post-translated, cleaved 22 amino acid polypeptide (SEQ ID NO: 432), and/or the post-translationally modified biologically active MU1140 (SEQ ID NO: 1156). Variants of MU1140 are designated herein by specifying (1) the identity (1 letter designation) of the original amino acid being altered, (2) the location of the amino acid being altered, and (3) the identity of the amino acid in the variant. For clarity, the positional nomenclature used herein refers to the relative position of the 22 amino acid protein. Thus, even when referring to the full-length 63 amino acid pre-protein, “position 1” refers to the first amino acid that would be found in the 22 amino acid post-translated cleaved protein. FIG. 10A, for example, depicts the positional nomenclature of amino acids in MU1140 designated 1 through 22. For example, a variant designated “R13T” refers to a MU1140 polypeptide wherein the 13m amino acid of the 22 amino acid polypeptide (which was originally an arginine, “R”) is substituted with a threonine, “T”). Likewise, if multiple amino acids of wildtype MU1140 are substituted, each of the substituted amino acid is designated in a similar way. For example, a variant designated “F1T R13T” refers to a MU1140 polypeptide wherein the 1^{st} amino acid of the 22 amino acid polypeptide is changed from a phenylalanine, “F,” to a threonine, “T,” and the 13^{th} amino acid of the 22 amino acid polypeptide is changed from an arginine, “R,” to a threonine, “T.” Similar naming conventions apply to variants having three, four, five or more amino acids which differ from wild-type MU1140.

In embodiments, the disclosure provides a lantibiotic comprising the following mutations of the amino acid sequence of the lantibiotic, MU1140 (e.g., SEQ ID NO: 1156):
a. arginine at position 13 changed to asparagine (R13N);
b. phenylalanine at position 17 changed to leucine (F17L) or tyrosine (F17Y);
c. asparagine at position 18 changed to alanine (N18A);
d. tyrosine at position 20 changed to phenylalanine (Y20F); and
e. combinations thereof.

In some embodiments, the non-naturally occurring lantibiotic comprises an amino acid sequence of MU1140 (e.g., SEQ ID NO:1156) and further comprises two mutations, wherein one mutation is:
a. arginine at position 13 changed to asparagine (R13N);
b. phenylalanine at position 17 changed to leucine (F17L) or tyrosine (F17Y);
c. asparagine at position 18 changed to alanine (N18A);
d. tyrosine at position 20 changed to phenylalanine (Y20F); or
e. combinations thereof.

In some embodiments, the non-naturally occurring lantibiotic comprises an amino acid sequence of MU1140 (e.g., SEQ ID NO:1156) and further comprises two mutations, wherein one mutation is arginine at position 13 changed to asparagine (R13N).

In some embodiments, the disclosure provides a non-naturally occurring lantibiotic comprising an amino acid sequence of MU1140 (e.g., SEQ ID NO:1156) and further comprises two mutations, wherein one mutation is:
a. phenylalanine at position 1 changed to valine (F1V);
b. phenylalanine at position 1 changed to alanine (F1A);
c. phenylalanine at position 1 changed to isoleucine (F1I);
d. phenylalanine at position 1 changed to leucine (F1L);
e. phenylalanine at position 1 changed to threonine (F1T); or
f. phenylalanine at position 1 changed to tyrosine (F1Y).

In some embodiments, the disclosure provides a non-naturally occurring lantibiotic comprising an amino acid sequence of MU1140 (e.g., SEQ ID NO:1156) and further comprises two mutations, wherein one mutation is phenylalanine at position 1 changed to valine (F1V).

In some embodiments, the disclosure provides a non-naturally occurring lantibiotic comprising an amino acid sequence of MU1140 (e.g., SEQ ID NO:1156) and further comprises two mutations, wherein one mutation is at position 1 and one mutation is at position 13. In some embodiments, the disclosure provides a non-naturally occurring lantibiotic comprising an amino acid sequence of MU1140 (e.g., SEQ ID NO: 1156) and further comprises two mutations, wherein the phenylalanine at position 1 is changed to valine, and the arginine at position 13 is changed to asparagine (F1V R13N). See, e.g., SEQ ID NO: 550.

In embodiments, the disclosure provides variants of MU1140 (e.g., SEQ ID NO: 1156) having two or more of the above mutations.

In embodiments, the disclosure provides non-naturally occurring lantibiotic comprising the following two mutations of the amino acid sequence of the lantibiotic MU1140 (e.g., SEQ ID NO: 1156):
a. phenylalanine at position 1 changed to alanine in combination with arginine at position 13 changed to:
   i. alanine (F1A R13A),
   ii. valine (F1A R13V),
   iii. asparagine (F1A R13N), or
   iv. serine (F1A R13S),
b. phenylalanine at position 1 changed to glycine in combination with arginine at position 13 changed to:
   i. glycine (F1G R13G);
c. phenylalanine a position 1 changed to histidine in combination with arginine at position 13 changed to:
   i. asparagine (F1H R13N);
d. phenylalanine at position 1 changed to isoleucine in combination with arginine at position 13 changed to:
   i. alanine (F1I R13A);
   ii. glycine (F1I R13G);
   iii. isoleucine (F1I R13I);
   iv. asparagine (F1I R13N);
   v. proline (F1I R13P);
   vi. glutamine (F1I R13Q);
   vii. glutamic acid (F1I R13S);
   viii. serine (F1I R13V); or
   ix. valine (F1I R13E);
e. phenylalanine at position 1 changed to leucine in combination with arginine at position 13 changed to:
   i. alanine (F1L R13A);
   ii. aspartic acid (F1L R13D);
   iii. glycine (F1L R13G);
   iv. asparagine (F1L R13N);
   v. proline (F1L R13P); or
   vi. glutamine (F1L R13Q);
f. phenylalanine at position 1 changed to threonine in combination with arginine at position 13 changed to:
   i. alanine (F1T R13A);
   ii. asparagine (F1T R13N); or
   iii. valine (F1T R13V);
g. phenylalanine at position 1 changed to valine in combination with arginine at position 13 changed to:
   i. alanine (F1V R13A);
   ii. asparagine (F1V R13N);
   iii. glutamine (F1V R13Q);
   iv. aspartic acid (F1V R13D);
   v. valine (F1V R13V); or
   vi. proline (F1V R13P);
h. phenylalanine at position 1 changed to tyrosine in combination with arginine at position 13 changed to:
   i. aspartic acid (F1Y R13D); or
   ii. glycine (F1Y R13G).

In some embodiments, the disclosure provides non-naturally occurring lantibiotic comprising the amino acid sequence of the lantibiotic MU1140 (e.g., SEQ ID NO:1156), wherein the phenylalanine at position 1 is changed to valine and arginine at position 13 changed to:
i. alanine (F1V R13A);
ii. asparagine (F1V R13N);
iii. glutamine (F1V R13Q);
iv. aspartic acid (F1V R13D);
v. valine (F1V R13V); or
vi. proline (F1V R13P).

In embodiments, the disclosure provides a non-naturally occurring lantibiotic having the following combination of three mutations of MU1140 (e.g., SEQ ID NO: 1156):
a. phenylalanine at position 1 changed to isoleucine, in combination with arginine at position 13 changed to alanine, in combination with glycine at position 15 changed to alanine (F1I R13A G15A);
b. phenylalanine at position 1 changed to isoleucine, in combination with arginine at position 13 changed to aspartic acid, in combination with glycine at position 15 changed to alanine (F1R13D G15A);
c. phenylalanine at position 1 changed to isoleucine, in combination with tryptophan at position 4 changed to isoleucine, in combination with arginine at position 13 changed to alanine (F1I W4I R13A);
d. phenylalanine at position 1 changed to isoleucine, in combination with tryptophan at position 4 changed to methionine, in combination with arginine at position 13 changed to aspartic acid (F1I W4M R13D);
e. phenylalanine at position 1 changed to isoleucine, in combination with tryptophan at position 4 changed to methionine, in combination with arginine at position 13 changed to asparagine (F1I W4M R13N);
f. phenylalanine at position 1 changed to isoleucine, in combination with lysine at position 2 changed to alanine, in combination with arginine at position 13 changed to alanine (F1I K2A R13A);
g. phenylalanine at position 1 changed to isoleucine, in combination with leucine at position 6 changed to valine, in combination with arginine at position 13 changed to alanine (F1I L6V R13A);
h. phenylalanine at position 1 changed to isoleucine, in combination with arginine at position 13 changed to alanine, in combination with tyrosine at position 20 changed to phenylalanine (F I R13A Y20F);
i. phenylalanine at position 1 changed to isoleucine, in combination with arginine at position 13 changed to aspartic acid, in combination with tyrosine at position 20 changed to phenylalanine (F1I R13D Y20F);
j. phenylalanine at position 1 changed to isoleucine, in combination with arginine at position 13 changed to asparagine, in combination with tyrosine at position 20 changed to phenylalanine (F1I R13N Y20F);
k. phenylalanine at position 1 changed to leucine, in combination with arginine at position 13 changed to asparagine, in combination with tyrosine at position 20 changed to phenylalanine (F1L R13N Y20F);
l. phenylalanine at position 1 changed to leucine, in combination with arginine at position 13 changed to alanine, in combination with tyrosine at position 20 changed to phenylalanine (F1L R13A Y20F); or
m. phenylalanine at position 1 changed to leucine, in combination with arginine at position 13 changed to aspartic acid, in combination with tyrosine at position 20 changed to phenylalanine (F1L R13D Y20F).

In embodiments, the disclosure provides variants of MU1140 (e.g., SEQ ID NO: 1156) having the following combination of mutations:
a. phenylalanine at position 1 changed to isoleucine, in combination with lysine at position 2 changed to alanine, in combination with tryptophan at position 4 changed to lysine, in combination with arginine at position 13 changed to alanine (F1I K2A W4K R13A); and
b. phenylalanine at position 1 changed to isoleucine, in combination with lysine at position 2 changed to alanine, in combination with tryptophan at position 4 changed to lysine, in combination with arginine at position 13 changed to aspartic acid (F1I K2A W4K R13D).

In embodiments, the disclosure provides variants of MU1140 (e.g., SEQ ID NO: 1156) having the following combination of mutations including phenylalanine at position 1 changed to isoleucine, in combination with lysine at position 2 changed to alanine, in combination with tryptophan at position 4 changed to methionine, in combination with arginine at position 13 changed to alanine, in combination with tyrosine at position 20 changed to phenylalanine (F1I K2A W4K R13A Y20F).

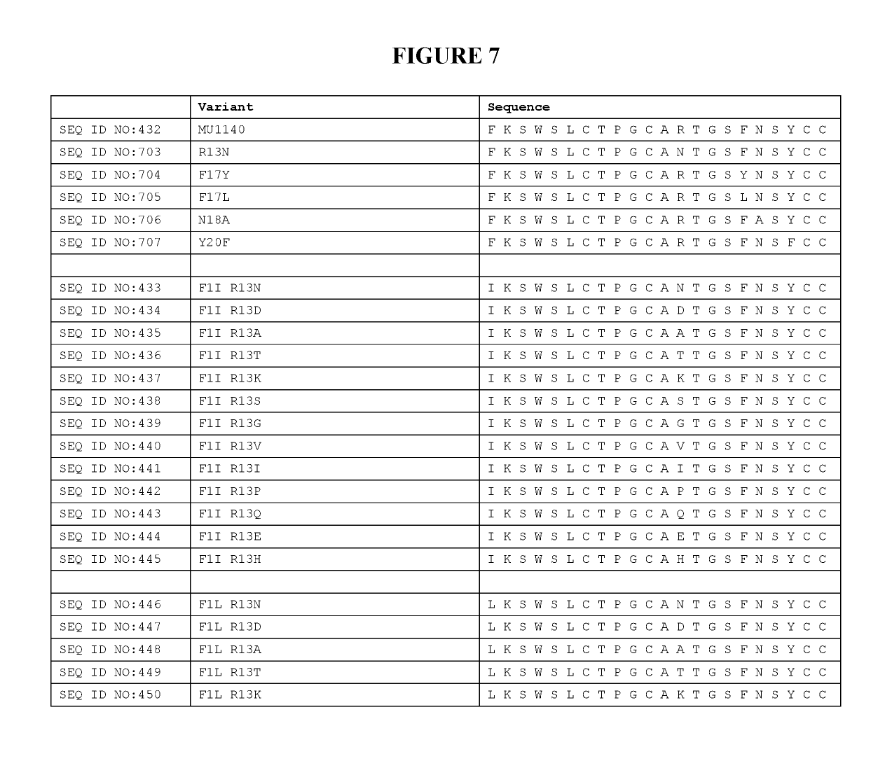

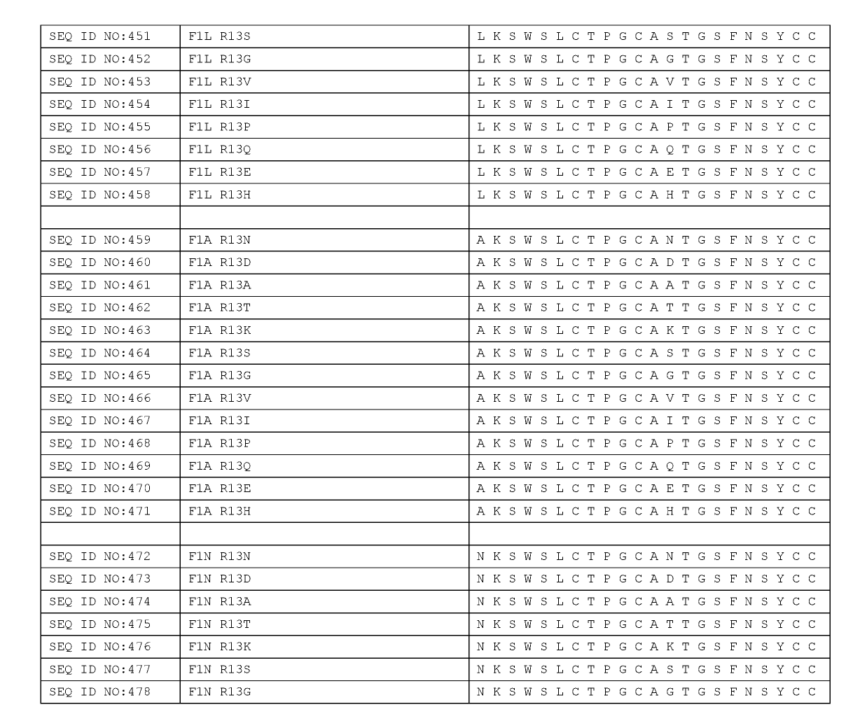

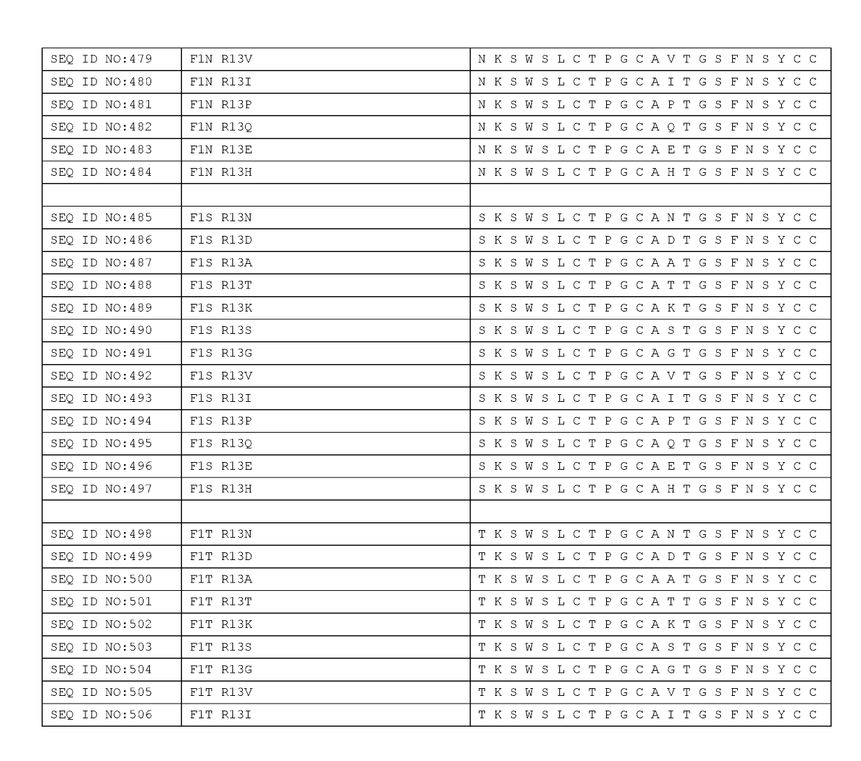

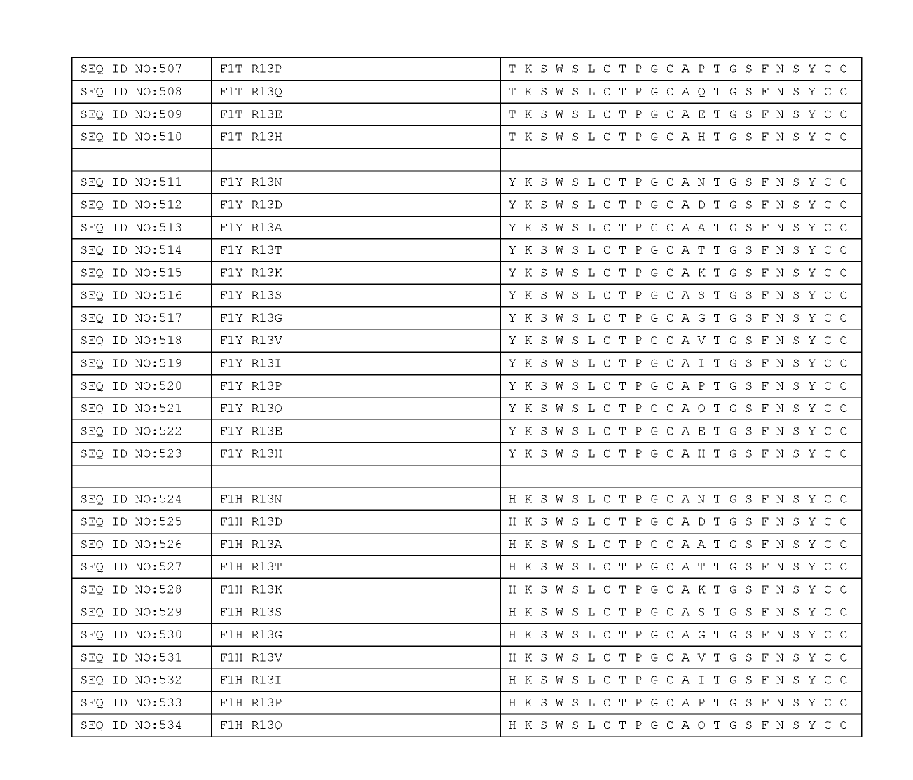

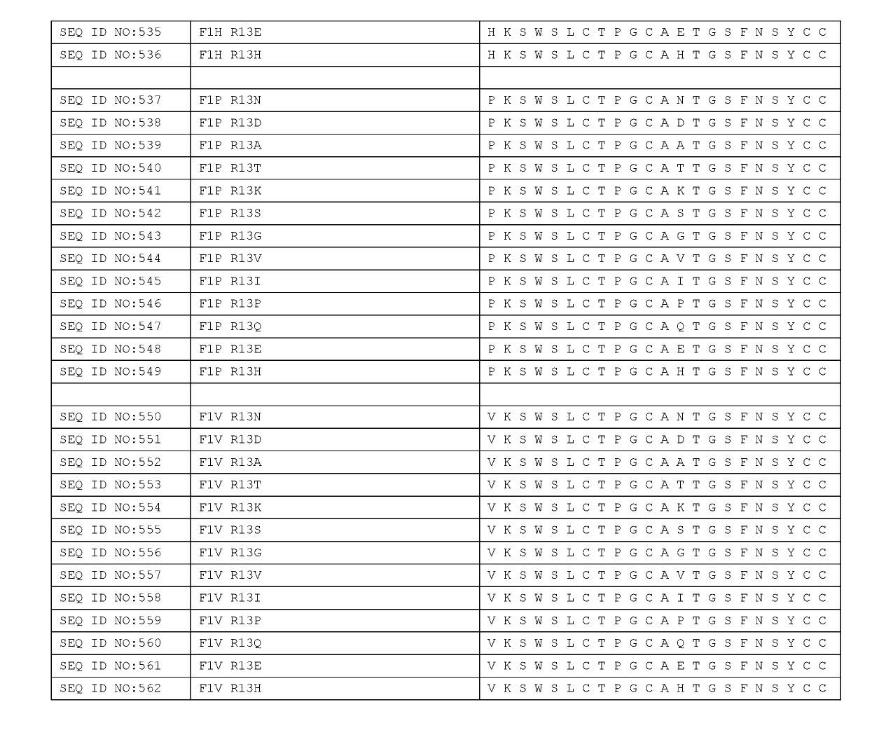

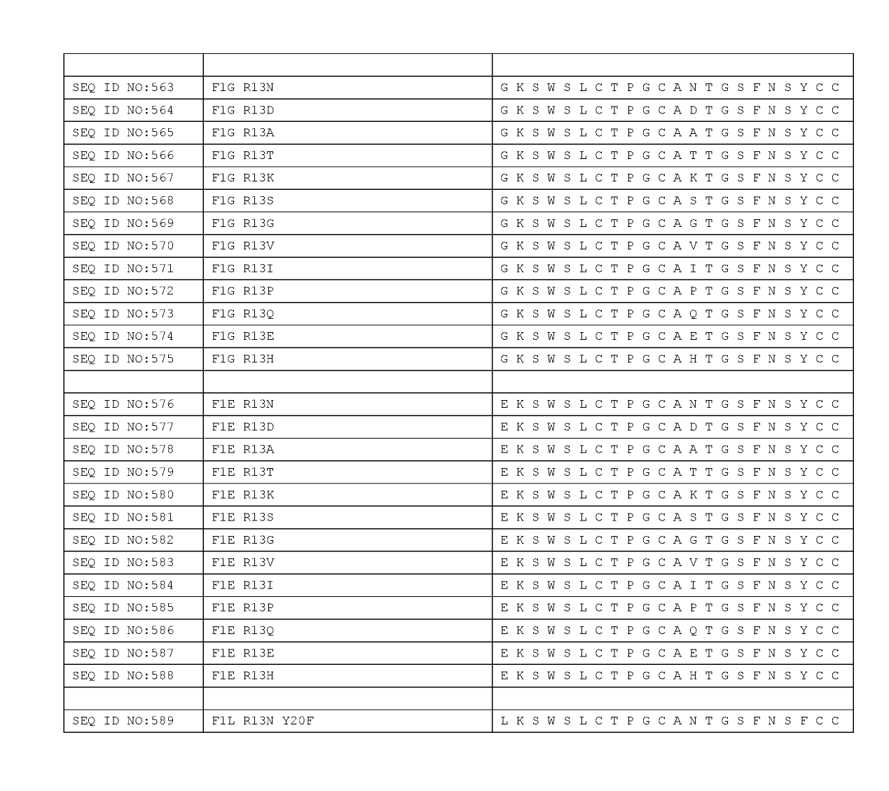

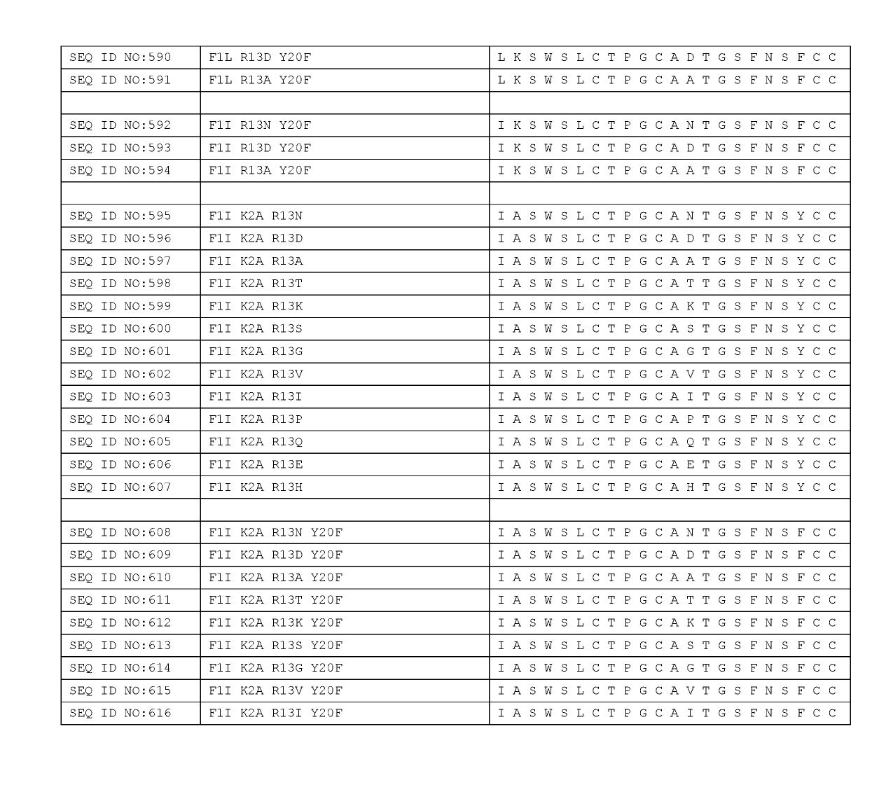

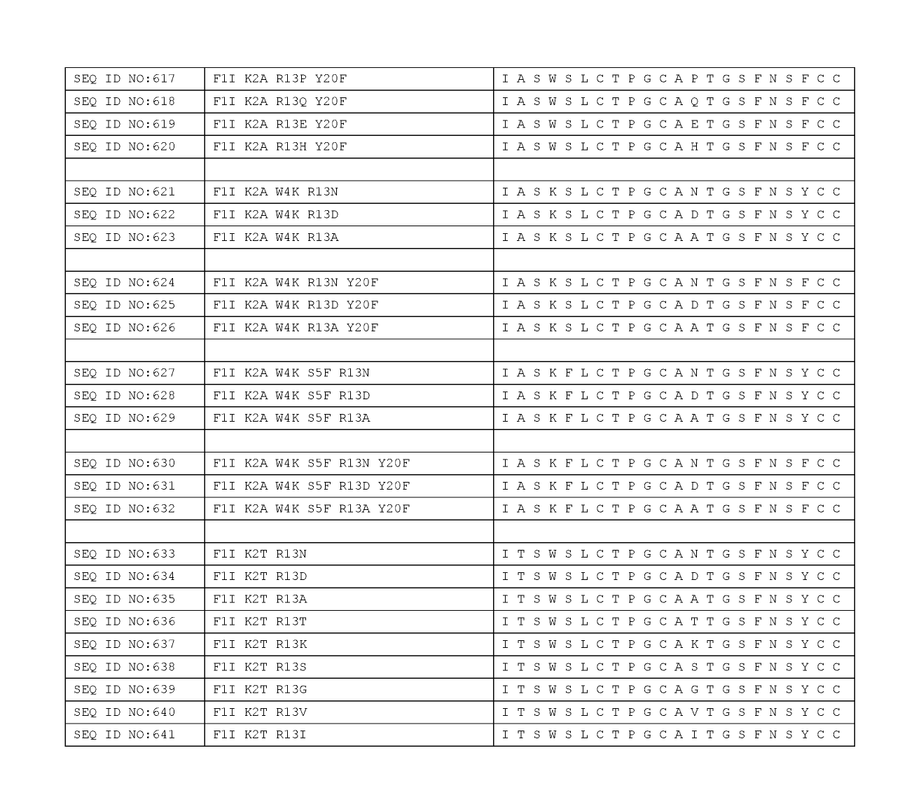

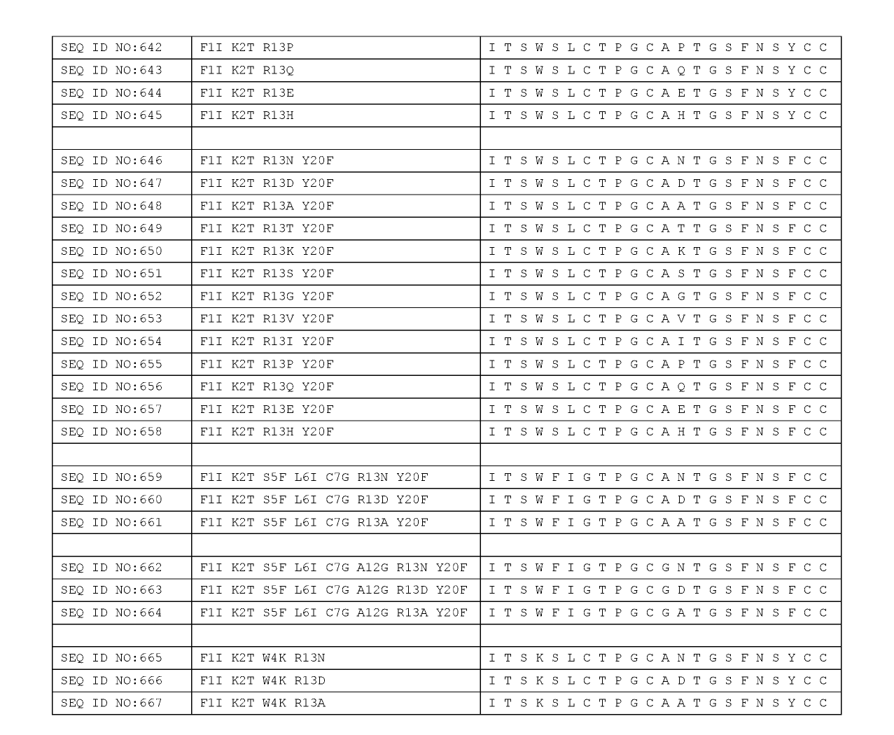

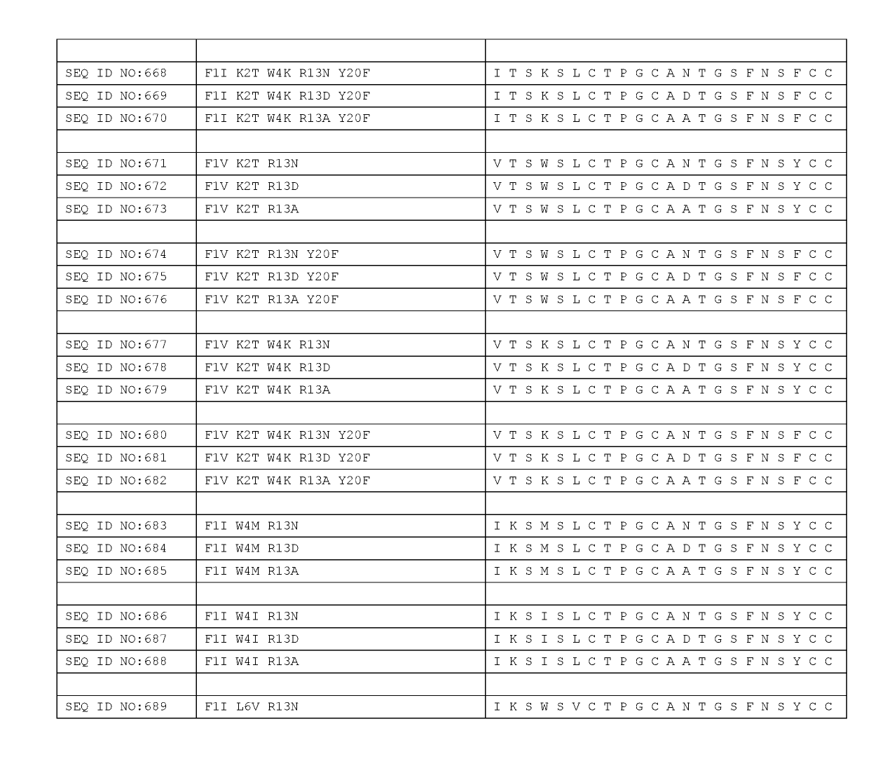

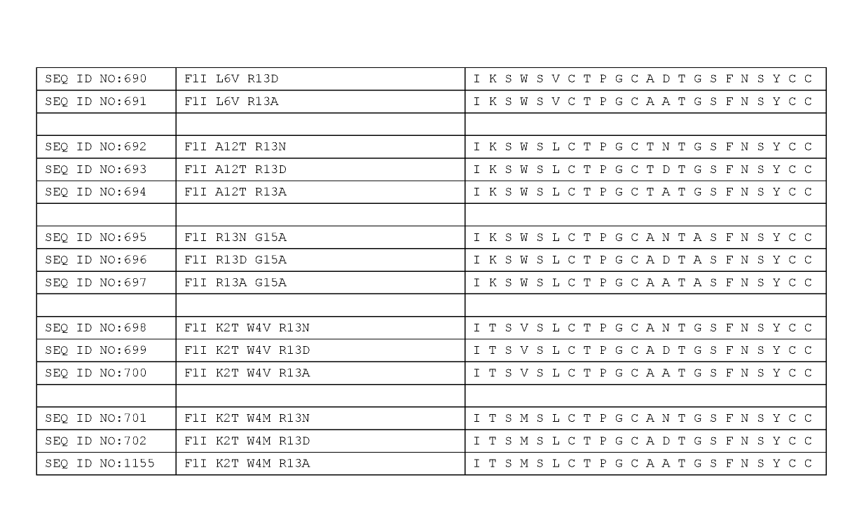

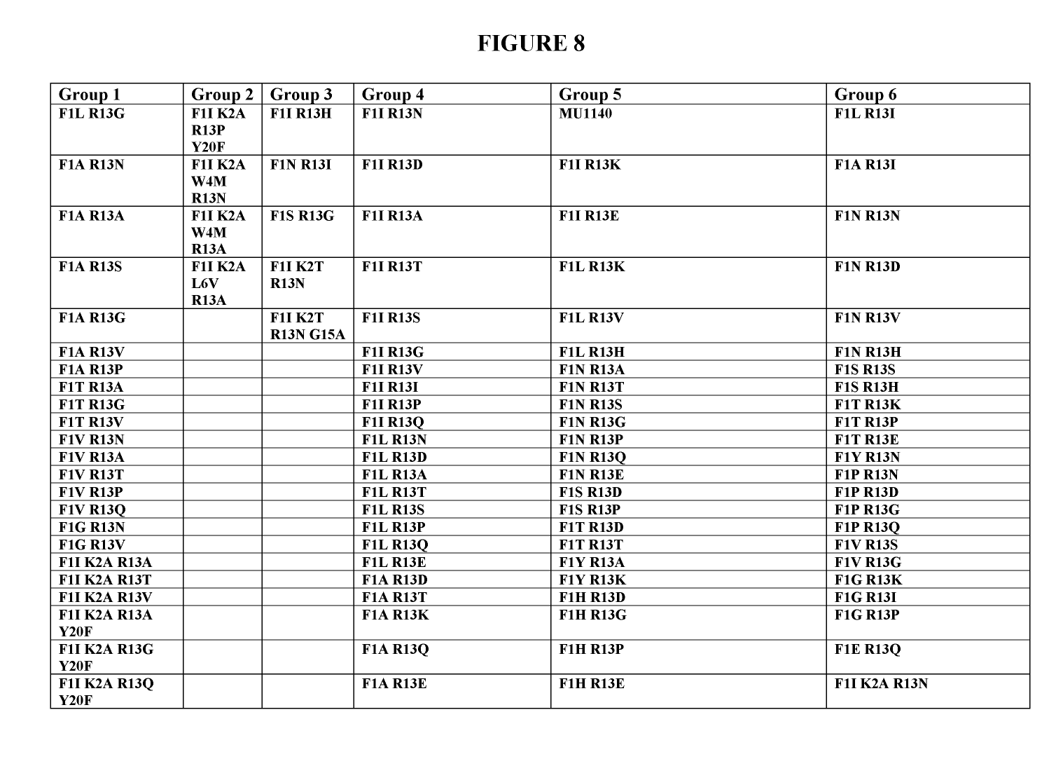

In embodiments, the disclosure provides a non-naturally occurring lantibiotic comprising a single amino acid mutation of MU1140, the lantibiotic having an amino acid sequence encoded by a polynucleotide comprising any one of SEQ ID NOS:2 to 431. The disclosure also provides a polynucleotide sequence encoding a full-length 63 amino acid pre-protein comprising an amino acid sequence encoded by of any one of SEQ ID NOS.: 2 to 431 or 708 to 763. For example, a polynucleotide sequence encoding a pre-protein polypeptide of SEQ ID NO. 764 can comprise SEQ ID NO. 1165 and SEQ ID NO. 2. In embodiments, the disclosure provides a non-naturally occurring lantibiotic comprising one or more amino acid mutations of MU1140, the lantibiotic having an amino acid sequence comprising any one of SEQ ID NOS:433-707. In embodiments, the disclosure provides a non-naturally occurring lantibiotic comprising any variant in FIG. 8: Group 1, Group 2 or Group 3. In embodiments, the disclosure provides a non-naturally occurring lantibiotic comprising any variant in FIG. 8: Group 1. The disclosure also provides a non-naturally occurring full-length 63 amino acid pre-protein comprising any variant in FIG. 8, Group 1, Group 2, or Group 3. The disclosure also provides a non-naturally occurring full-length 63 amino acid pre-protein comprising any variant in FIG. 8, Group 1.

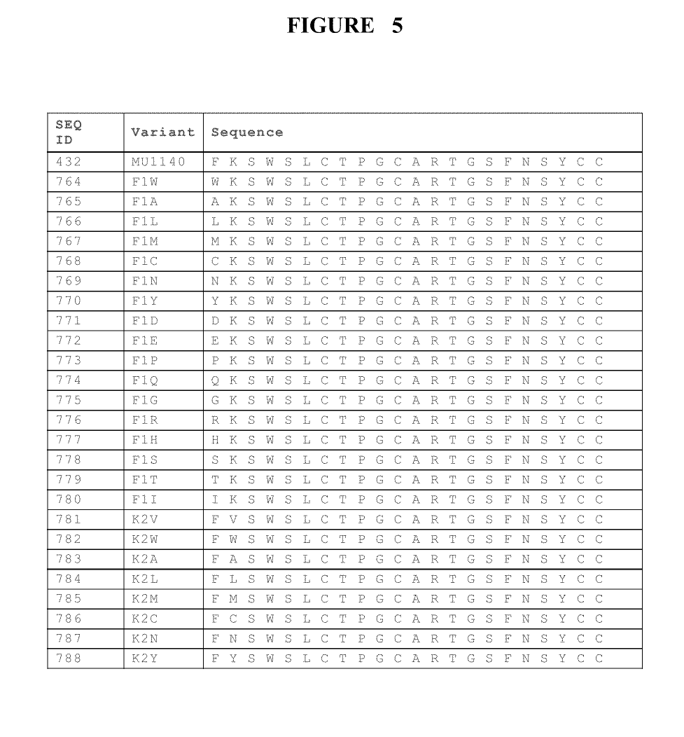

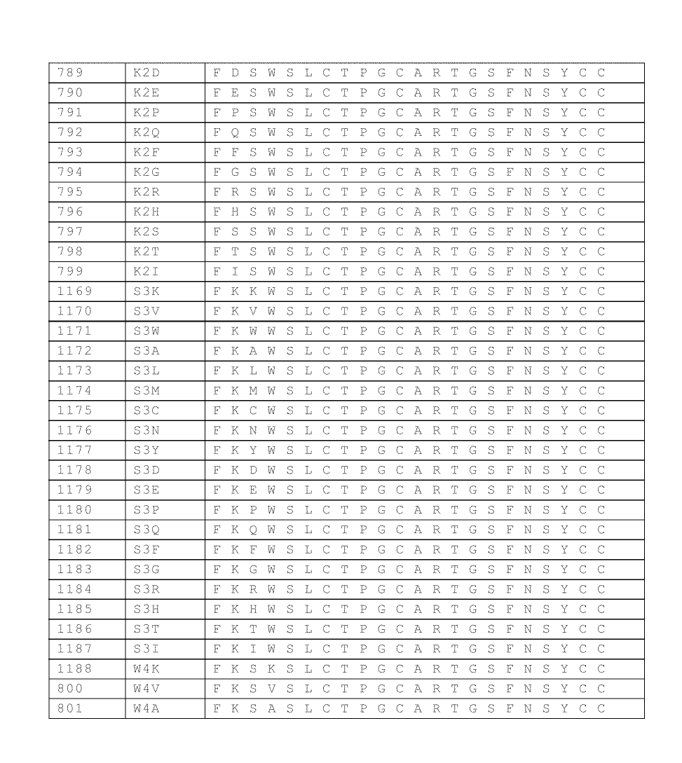

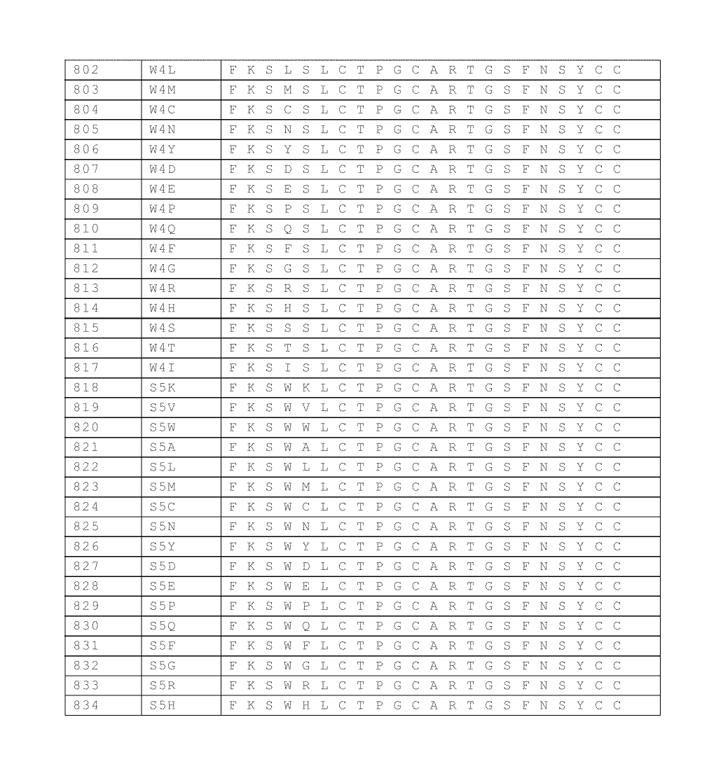

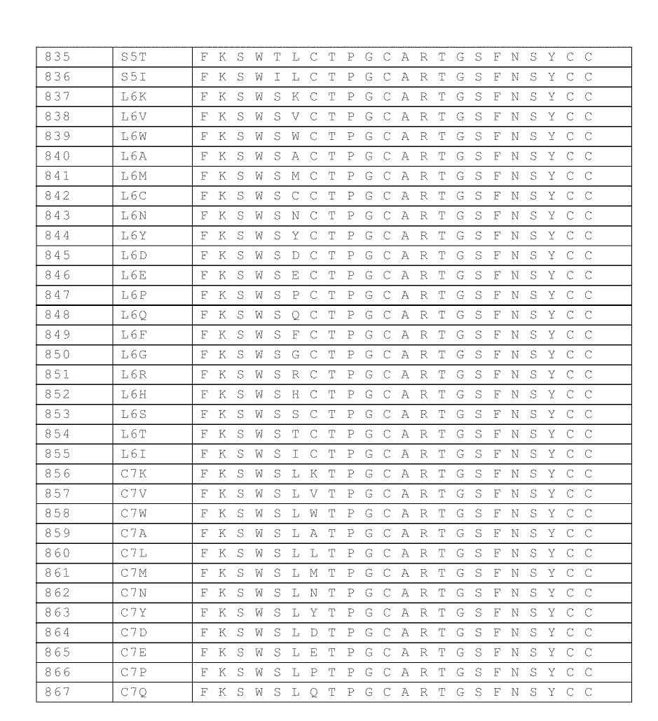

The lantibiotic variants in FIG. 5 comprise single amino acid variants. In some embodiments, the disclosure is directed to a non-naturally occurring lantibiotic comprising a single amino acid mutation of MU1140 as found in FIG. 5. The disclosure also provides for a non-naturally occurring full-length 63 amino acid pre-protein form of the lantibiotic variants described in FIG. 5. For example, a pre-protein form of SEQ ID NO: 766 can comprise SEQ ID NO: 1164 and SEQ ID NO:766, a pre-protein form of SEQ ID NO: 767 can comprise SEQ ID NO: 1164 and SEQ ID NO: 767 and so on.

In some embodiments, the disclosure provides a lantibiotic variant as found in FIG. 7. The lantibiotic variants in FIG. 7 comprise the “core peptide.” The disclosure also provides for a non-naturally occurring full-length 63 amino acid pre-protein form of the “core peptide” variants as described in FIG. 7. For example, a pre-protein form of SEQ ID NO: 703 can comprise SEQ ID NO: 1164 and SEQ ID NO:703.

Lantibiotics of the disclosure comprise lantibiotic variants, which can be non-naturally occurring lantibiotic variants. The terms “lantibiotic variant,” “MU1140 variant,” “non-naturally occurring lantibiotics,” or “variant” are interchangeable and refer to an MU1140 (i.e., wild-type lantibiotic) polypeptide (SEQ ID NO:432), having one or more (e.g., 1, 2, 3, 4, 5, or more amino acid substitutions (including modified amino acid substitutions), deletions, or insertions. The term “modified amino acid substitutions” include a substitution of an amino acid with a modified amino acid. In some embodiments, the term “lantibiotic variant,” “MU1140 variant,” “non-naturally occurring lantibiotics,” or “variant” refers to the translated, full-length 63 amino acid pre-protein of MU1140 (SEQ ID NO:1160) having one or more (e.g., 1, 2, 3, 4, 5, or more amino acid substitutions (including modified amino acid substitutions), deletions, or insertions. In some embodiments, the term “lantibiotic variant,” “MU1140 variant,” “non-naturally occurring lantibiotics,” or “variant” refers to a mature, biologically acitve MU1140 (i.e., wild-type lantibiotic) polypeptide which has been post-translationally modified (SEQ ID NO: 1156) having one or more (e.g., 1, 2, 3, 4, 5, or more amino acid substitutions (including modified amino acid substitutions), deletions, or insertions. Modified amino acids include, for example, 2,3-didehydroalanine (Dha), 2, 3-didehydrobutyrine (Dhb), S-amino vinyi-D-cysteine (AviCys), aminobutyrate (Abu), 2-oxopropionyl, 2-oxobutyryl, and hydroxypropionyl. As used herein, “single-site variant(s)” refers to an MU1140 (i.e., wild-type lantibiotic) polypeptide (e.g., SEQ ID NOs:432 or 1156) having one amino acid substitution (including modified amino acid substitutions), deletion, or insertion and “multi-site variant(s)” refers to an MU1140 (i.e., wild-type lantibiotic) polypeptide (e.g., SEQ ID NOs: 432 or 1156) having more than one (e.g., 2, 3, 4, 5, or more amino acid substitutions (including modified amino acid substitutions), deletions, or insertions. In some embodiments, the “wild-type” lantibiotic MU1140 (i.e., wild-type lantibiotic) polypeptide (SEQ ID NO:432) or any of the variants described herein can be post-translationally modified (e.g., SEQ ID NO: 1156). Post-translational modifications are described in U.S. Pat. No. 6,964,760, incorporated by reference herein in its entirety. Various post-translational modifications can include, e.g., the presence of dehydrated residues and lanthionine rings. In some embodiments, the modified residues 2,3-didehydroalanine (Dha) and 2,3-didehydrobutyrine (Dhb) can be formed from dehydration of serines and threonines, respectively. In some embodiments, the post-translational modification can include the modified residue S-2-aminobutyric acid (Abu) formed from threonine. In some embodiments, the post-translational modification can include the cyclization between a cysteine and either a Dha or a Dhb to form a lanthionine or a methyllanthionine ring, respectively. In some embodiments, the serine at position 3, 5, 16, and/or 19 are modified to Dha residues, position 8 is modified to Abu, and position 14 is modified to Dhb. In some embodiments, the post-translational modification occurs in any of the host cells described herein, e.g., in some embodiments, the lantibiotic variants as provided herein can be modified by Streptococcus mutans.

In some embodiments, the post-translation modification comprises Dha at position 5. In some embodiments, the post-translation modification comprises Abu at position 8. In some embodiments, the post-translation modification comprises Dhb at position 14. In some embodiments, the post-translation modification comprises Dha at position 5, Abu at position 8, and Dhb at position 14. In some embodiments, the post-translation modification comprises a ring formed by lanthionine (Ala-S-Ala) residues between (Ala₃-S-Ala₇) as described in FIG. 1 (Ring A). In some embodiments, the post-translation modification comprises a methyl-lanthionine residue (Abu-S-Ala) forming a ring, Ring B, comprising the alpha-aminobutyrate residue in position 8 and the Ala in position 11 (Abu₈-S-Ala₁₁) as described in FIG. 1. In some embodiments, the post-translation modification comprises a ring formed by lanthionine (Ala-S-Ala) residues between (Ala₁₆-S-Ala₂₁) as described in FIG. 1 (Ring C). In some embodiments, the post-translation modification comprises Ala in position 19 linked to an aminovinyl group by a thioether linkage (Ala₁₉-S—CH═CH—NH—), Ring D, as described in FIG. 1. In some embodiments, the post-translation modification comprises Ring A, B, C and D. In some embodiments, the post-translationally modified MU1140 variant comprises Ring A, Ring B, Ring C and Ring D, wherein: (a) two of these rings are formed by lanthionine (Ala-S-Ala) residues, including one in Ring A (Ala₃-S-Ala₇) and one in Ring C (Ala₁₆-S-Ala₂₁); (b) a methyl-lanthionine residue (Abu-S-Ala) forms Ring B comprising the alpha-aminobutyrate residue in position 8 and the Ala in position 11 (Abu₈-S-Ala₁₁); and (c) the fourth ring, D, is comprised of the Ala in position 19 linked to an aminovinyl group by a thioether linkage (Ala₁₉-S—CH═CH—NH—), and wherein amino acid at position 5 is modified to Dha, the amino acid at position 8 is modified to Abu, and the amino acid at position 14 is modified to Dhb. In some embodiments, the isolated lantibiotic variant is produced in a host cell by expressing a polypeptide from a polynucleotide encoding a lantibiotic variant of FIG. 5 or FIG. 7, and wherein the expressed polypeptide is post-translationally modified in the host cell. In some embodiments, the host cell is S. mutans. In some embodiments, the post-translationally modified lantibiotic variant comprises an amino acid sequence of SEQ ID NO. 1157. In some embodiments, the post-translationally modified lantibiotic variant comprises an amino acid sequence of SEQ ID NO. 1158 (which does not designate secondary structures, e.g., rings). In some embodiments, the post-translationally modified lantibiotic variant comprises an amino acid sequence of SEQ ID NO. 1161.

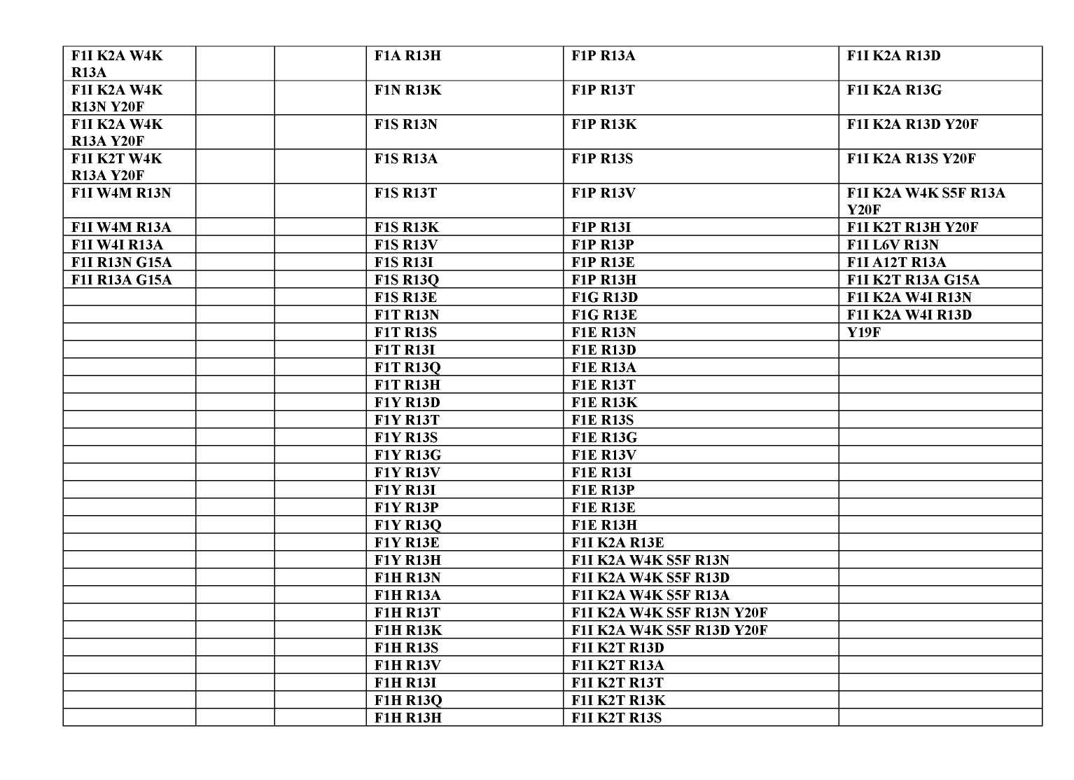

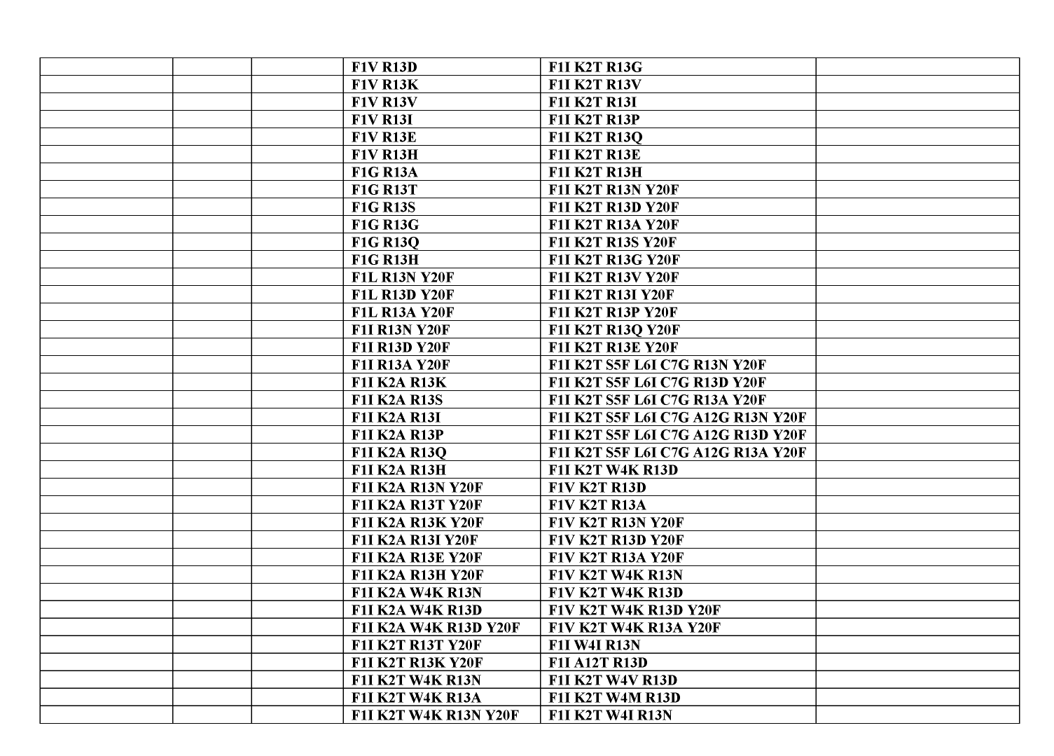

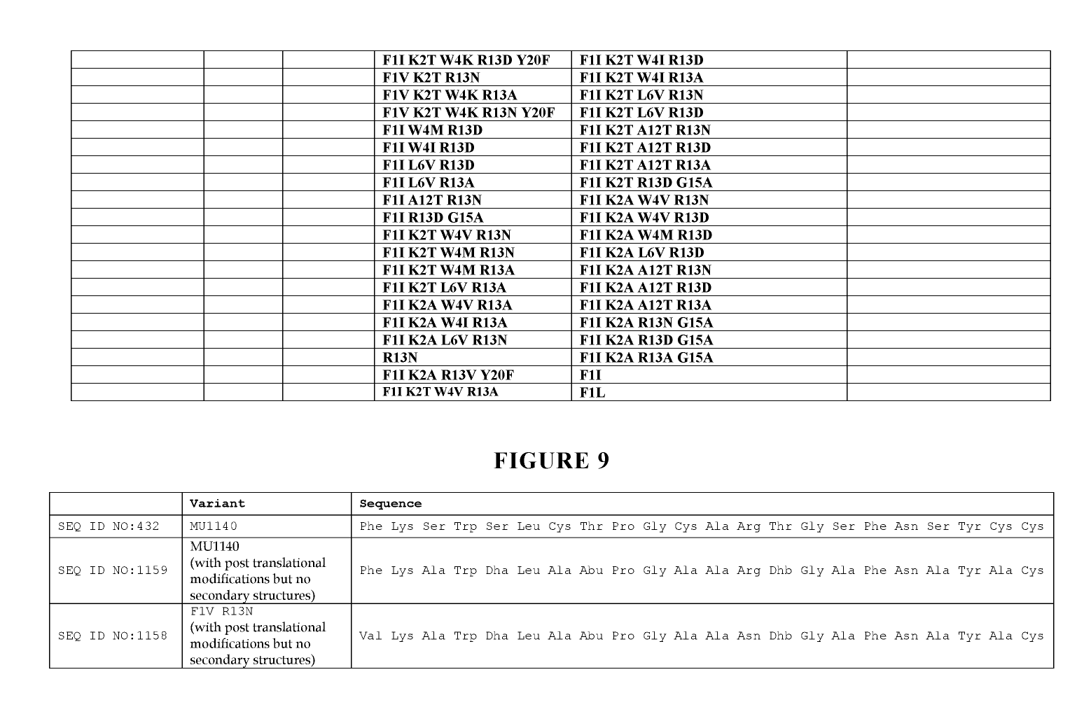

For example, in some embodiments, the lantibiotic variant can include the polypeptide of FIG. 1, SEQ ID NO: 1157. In some embodiments, the lantibiotic variant can include the polypeptide of FIG. 9, SEQ ID NO: 1158. In some embodiments, the lantibiotic variant can include the polypeptide of SEQ ID NO: 1161.

In some embodiments, the disclosure provides for an isolated lantibiotic variant, wherein the isolated lantibiotic variant is produced in a host cell by expressing a polypeptide from a polynucleotide encoding a lantibiotic of FIG. 5 or FIG. 7, wherein the expressed polypeptide can be post-translationally modified in the host cell. Thus, for clarity, the disclosure provides for the lantibiotic variants described herein, as well as lantibiotic variants having post-translational modifications. In some embodiments, the host cell is S. mutans. In some embodiments, the disclosure provides a post-translationally modified, non-naturally occurring lantibiotic comprising an amino acid sequence of SEQ ID NO. 1157.

In embodiments, biologically active equivalents of the lantibiotic variants of the disclosure also have one or more conservative amino acid variations or other minor modifications and retain biological activity in addition to the amino acid changes disclosed above. A biologically active equivalent has substantially equivalent function when compared to the corresponding lantibiotic. In embodiments, a lantibiotic variant of the disclosure has about 1, 2, 3, 4, or 5 conservative amino acid substitutions. A conservative substitution is one in which an amino acid is substituted for another amino acid that has similar properties, such that one skilled in the art of peptide chemistry would expect the secondary structure and general nature of the polypeptide to be substantially unchanged. Examples of conservative substitutions can be found, e.g., in Yampolsky, et al., Genetics 170(4): 1459-1472 (2005). In some embodiments, conservative substitutions can be characterized are according to their class, e.g.,

| Class | Name of Amino Acids |
|---|---|
| Aliphatic | Glycine, Alanine, Valine, Leucine, Isoleucine |
| Hydroxyl or Sulfur/ | Serine, Cysteine, Selenocysteine, Threonine, |
| Selenium-containing | Methionine |
| Cyclic | Proline |
| Aromatic | Phenylalanine, Tyrosine, Tryptophan |
| Basic | Histidine, Lysine, Arginine |
| Acidic and their Amide | Aspartate, Glutamate, Asparagine, Glutamine |

In some embodiments, the conservative substitutions can be made within the following groups:
Group 1: charged positives (R-Arg, H-His and K-Lys)
Group 2: charged negatives (D-Asp and E-Glu)
Group 3: polar uncharged (S-Ser, T-Thr, N-Asn, Q-Gln)
Group 4: hydrophobic (A-Ala, V-Val, I-Ile, L-Leu, M-Met, F-Phe, Y-Tyr, W-Trp)
Group 5: “others” (C-Cys, G-Gly, P-Pro).
   For example, a conservative substitution can be a substitution of H for R because both H and R are in Group 1. By way of example, in some embodiments, the disclosure provides for variants of lantibiotics wherein the phenylalanine of position 1 is substituted with an aliphatic class residue, e.g., glycine, alanine, valine, leucine, isoleucine. The disclosure also provides for variants of lantibiotics wherein the arginine of position 13 is substituted with an acidic and their amide class residue, e.g., aspartate, glutamate, asparagine, glutamine.

In embodiments, the lantibiotics of the disclosure are polypeptides comprising post-translational modifications, i.e., chemical or biochemical modifications after the polypeptide has been translated.

A purified lantibiotic is a lantibiotic preparation that is substantially free of cellular material, other types of polypeptides, chemical precursors, chemicals used in synthesis of the polypeptide, or combinations thereof. A purified lantibiotic preparation that is substantially free of cellular material, culture medium, chemical precursors, chemicals used in synthesis of the polypeptide, etc., has less than about 30%, 20%, 10%, 5%, 1% of other polypeptides, culture medium, chemical precursors, and/or other chemicals used in synthesis. Therefore, a purified lantibiotic is about 70%, 80%, 90%, 95%, 99% or more pure. A purified lantibiotic does not include unpurified or semi-purified cell extracts or mixtures of polypeptides that are less than 70% pure.

A lantibiotic of the disclosure can be covalently or non-covalently linked to an amino acid sequence to which the lantibiotic is not normally associated with in nature, i.e., a heterologous amino acid sequence. For example, a heterologous amino acid sequence is from an organism that does not naturally produce a lantibiotic, a synthetic sequence, or a sequence not usually located at the carboxy or amino terminus of a naturally occurring lantibiotic. Additionally, a lantibiotic of the disclosure can be covalently or non-covalently linked to compounds or molecules other than amino acids such as indicator reagents. A lantibiotic of the disclosure can be covalently or non-covalently linked to an amino acid spacer, an amino acid linker, a signal sequence, a stop transfer sequence, transfer-messenger RNA (TMR) stop transfer sequence, a transmembrane domain, a protein purification ligand, or a combination thereof. A polypeptide can also be linked to a moiety (i.e., a functional group that can be a polypeptide or other compound) that facilitates purification (e.g., affinity tags such as a six-histidine tag (SEQ ID NO: 1166), trpE, glutathione-S-transferase, maltose binding protein, staphylococcal Protein A), or a moiety that facilitates polypeptide stability (e.g., polyethylene glycol; amino terminus protecting groups such as acetyl, propyl, succinyl, benzyl, benzyloxycarbonyl or t-butyloxycarbonyl; carboxyl terminus protecting groups such as amide, methylamide, and ethylamide). In one embodiment of the disclosure a protein purification ligand can be one or more amino acid residues at, for example, the amino terminus or carboxy terminus of a polypeptide of the disclosure. An amino acid spacer is a sequence of amino acids that are not associated with a polypeptide of the disclosure in nature. An amino acid spacer can comprise about 1, 5, 10, 20, 100, or 1,000 amino acids.

In embodiments, a lantibiotic of the disclosure is part of a fusion protein, which can contain heterologous amino acid sequences. Heterologous amino acid sequences can be present at the C or N terminus of a lantibiotic of the disclosure to form a fusion protein. In embodiments, more than one lantibiotic of the disclosure is present in a fusion protein. Fragments of lantibiotics of the disclosure can be present in a fusion protein of the disclosure. A fusion protein of the disclosure can comprise one or more lantibiotic of the disclosure, fragments thereof, or combinations thereof.

Pharmaceutically acceptable salts, esters, amides, and prodrugs are carboxylate salts, amino acid addition salts, esters, amides, and prodrugs of the lantibiotics are part of the present disclosure. These compounds are suitable for use with subjects and do not cause undue toxicity, irritation, or allergic response, are commensurate with a reasonable benefit/risk ratio, and are effective for their intended use. Salts are the substantially non-toxic, inorganic and organic acid addition salts of lantibiotics of the disclosure. Salts include, for example, hydrobromide, hydrochloride, sulfate, bisulfate, nitrate, acetate, trifluoroacetate, formate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as non-toxic ammonium, quaternary ammonium and amine cations including, but not limited to ammonium, tetramethylammonium, tetraethylammonium, methylamine, dimethylamine, trimethylamine, triethylamine, ethylamine, and the like.

Pharmaceutically acceptable, non-toxic esters of lantibiotics of the disclosure include, for example, C1-C6 alkyl esters wherein the alkyl group is a straight or branched chain. Other esters include C5-C7 cycloalkyl esters as well as arylalkyl esters such as, but not limited to benzyl C1-C4 alkyl esters.

Pharmaceutically acceptable, non-toxic amides of lantibiotics of the disclosure include amides derived from ammonia, primary C1-C6 alkyl amines and secondary C1-C6 dialkyl amines wherein the alkyl groups are straight or branched chains. In the case of secondary amines, the amine may be in the form of a 5- or 6-membered heterocycle containing one nitrogen atom. Also included are amides derived from ammonia, C1-C3 alkyl primary amines, and C1-C2 dialkyl secondary amines.

### Systems for Producing Lantibiotics

In some embodiments, a lantibiotic polypeptide of the disclosure can be synthesized using DPOLT methodologies. See e.g., U.S. Pat. No. 7,521,529; U.S. Publ. No. 2009/0215985, each incorporated herein by reference in their entireties. A lantibiotic of the disclosure can be produced recombinantly. In embodiments, a polynucleotide encoding a lantibiotic of the disclosure is introduced into a recombinant expression vector, which is expressed in a suitable expression host cell system using techniques well known in the art. A variety of bacterial, yeast, plant, mammalian, and insect expression systems are available in the art and any such expression system can be used. A lantibiotic of the disclosure can also be purified from S. mutans cell culture.

As noted above, the disclosure provides lantibiotics that are produced recombinantly by expression in a suitable expression host cell system using techniques well known in the art. In embodiments, native lantibiotic-producing bacteria are used to produce the lantibiotics of the disclosure. Such bacteria include, but are not limited to, Streptococcus mutans, Lactococcus lactis, Bacillus subtilis, Streptococcus pyogenes, Staphylococcus epidermis, Staphylococcus gallinarium, Micrococcus varians, Streptococcus salivarius, Lactobacillus sakei, Streptomyces OH-4156, Lactobacillus plantarum, Butyrivibrio fibriosolvens, Streptomyces cinnamoneus, Streptoverticillium hachijoense, Streptoverticillium, Streptomyces griseoluteus, Bacillus sp. strain HIL Y-85, Actinoplanes linguriae, Staphylococcus aureus, Enterococcus faecalis, Ruminococcus gnavus, Carnobacterium piscicola, Streptococcus macedonicus, Streptococcus bovis, Staphylococcus warneri, Streptomyces coelicolor and Streptomyces spp. In some embodiments, the lantibiotic variants described herein can be post-translationally modified by any of the bacteria listed above.

Systems for producing lantibiotics of the disclosure also include known bacteria or yeast that are genetically tractable hosts and are capable of heterologous production of lantibiotics, i.e., yeast or bacteria that do not natively produce lantibiotics. Such organisms include, but are not limited to, E. coli and bacteria of the genus Pseudomonas, a Gram-negative bacteria, and yeasts of the genus Saccharomyces or Pichia. In embodiments, P. fluorescens is used to produce the lantibiotics of the disclosure.

In embodiments, the disclosure provides recombinant bacterial strains that produce a lantibiotic of the disclosure. For example, the disclosure provides recombinant bacterial strains that produce lantibiotics, as listed above, that comprise a polynucleotide that expresses a functional variant MU1140. Biological activity of a lantibiotic variant of MU1140 can be assayed using methods known in the art, e.g., zone of inhibition assays.

In some embodiments, the disclosure is directed to an isolated recombinant Streptococcus mutans strain comprising: (a) a mutation in a polynucleotide involved in lactic acid synthesis such that expression of lactic acid is diminished by about 80% or more as compared to a wildtype S. mutans strain; (b) a recombinant alcohol dehydrogenase polynucleotide; and (c) a recombinant polynucleotide encoding a non-naturally occurring lantibiotic as described herein, e.g., a lantibiotic of FIG. 5 and FIG. 7.

### Polynucleotides

Polynucleotides of the disclosure contain less than an entire microbial genome and can be single- or double-stranded nucleic acids. A polynucleotide can be RNA, DNA, cDNA, genomic DNA, chemically synthesized RNA or DNA or combinations thereof. The polynucleotides can be purified free of other components, such as proteins, lipids and other polynucleotides. For example, the polynucleotide can be 50%, 75%, 90%, 95%, 96%, 97%, 98%, 99%, or 100% purified. A nucleic acid molecule existing among hundreds to millions of other nucleic acid molecules within, for example, cDNA or genomic libraries, or gel slices containing a genomic DNA restriction digest are not to be considered an isolated polynucleotide. The polynucleotides of the disclosure encode the polypeptides described above. SEQ ID NO: 1162 encodes the 63 amino acid pre-protein of MU1140. SEQ ID NO: 1 encodes the core protein of wild-type MU1140. SEQ ID NO: 1165 encodes the cleaved peptide as described herein. In embodiments, the disclosure provides purified polynucleotides shown in SEQ ID NOs: 2-431. In embodiments, the disclosure provides purified polynucleotides shown in SEQ ID NOs: 708-763. In some embodiments, the disclosure provides a purified polynucleotide comprising any one of SEQ ID NOs: 756, 757, 758, 759, 760 or 761. In some embodiments, the disclosure provides a purified polynucleotide comprising any one of SEQ ID NOs: 709, 714, 716, 735, 747, 751, 754, or 758. In some embodiments, the disclosure provides a purified polynucleotide comprising SEQ ID NO: 758 (F1V R13N). In some embodiments, the disclosure provides a purified polynucleotide comprising SEQ ID NO: 1163 (F1V R13N).

The disclosure herein provides for polynucleotides encoding the 63 amino acid pre-protein variants of MU1140. Thus, the disclosure provides for any of the nucleotides described herein encoding the cleaved peptide (SEQ ID NO 1165).

Polynucleotides of the disclosure can consist of less than about 66, 60, 50, 45, 30, 15 (or any range between about 66 and 15) contiguous nucleotides. The purified polynucleotides can comprise additional heterologous polynucleotides. Polynucleotides of the disclosure can comprise other nucleotide sequences, such as sequences coding for linkers, signal sequences, TMR stop transfer sequences, transmembrane domains, or ligands useful in protein purification such as glutathione-S-transferase, histidine tag, and Staphylococcal protein A. One embodiment of the disclosure provides a purified polynucleotide comprising at least about 6, 10, 15, 20, 25, 30, 40, 45, 50, 60, 66, or more contiguous nucleotides of SEQ ID NOs: 2-431. One embodiment of the disclosure provides a purified polynucleotide comprising at least about 6, 10, 15, 20, 25, 30, 40, 45, 50, 60, 66, or more contiguous nucleotides of SEQ ID NO: 1163.

Polynucleotides of the disclosure can be isolated. An isolated polynucleotide is a naturally-occurring polynucleotide that is not immediately contiguous with one or both of the 5′ and 3′ flanking genomic sequences that it is naturally associated with. An isolated polynucleotide can be, for example, a recombinant DNA molecule of any length. Isolated polynucleotides also include non-naturally occurring nucleic acid molecules. Polynucleotides of the disclosure can encode full-length polypeptides, polypeptide fragments, and variant or fusion polypeptides.

Degenerate nucleotide sequences encoding polypeptides of the disclosure, as well as homologous nucleotide sequences that are at least about 80, or about: 90, 95, 96, 97, 98, or 99% identical to the polynucleotide sequences of the disclosure and the complements thereof are also polynucleotides of the disclosure. Degenerate nucleotide sequences are polynucleotides that encode a polypeptide of the disclosure or fragments thereof, but differ in nucleic acid sequence from the given polynucleotide sequence, due to the degeneracy of the genetic code. Percent sequence identity has an art recognized meaning and there are a number of methods to measure identity between two polypeptide or polynucleotide sequences. See, e.g., Lesk, Ed., Computational Molecular Biology, Oxford University Press, New York, (1988); Smith, Ed., Biocomputing: Informatics and Genome Projects, Academic Press, New York, (1993); Griffin & Griffin, Eds. Computer Analysis of Sequence Data, Part I, Humana Press, New Jersey, (1994); von Heinje, Sequence Analysis In Molecular Biology, Academic Press, (1987); and Gribskov & Devereux, Eds., Sequence Analysis Primer, M Stockton Press, New York, (1991). Methods for aligning polynucleotides or polypeptides are codified in computer programs, including the GCG program package (Devereux et al. (1984) Nuc. Acids Res. 12:387), BLASTP, BLASTN, FASTA (Atschul et al. (1990) J. Molec. Biol. 215:403), and Bestfit program (Wisconsin Sequence Analysis Package, Version 8 for Unix, Genetics Computer Group, University Research Park, 575 Science Drive, Madison, Wis. 53711) which uses the local homology algorithm of Smith and Waterman ((1981) Adv. App. Math., 2:482-489). For example, the computer program ALIGN which employs the FAST A algorithm can be used, with an affine gap search with a gap open penalty of −12 and a gap extension penalty of-2.

When using any of the sequence alignment programs to determine whether a particular sequence is, for instance, about 95% identical to a reference sequence, the parameters are set such that the percentage of identity is calculated over the full length of the reference polynucleotide and that gaps in identity of up to 5% of the total number of nucleotides in the reference polynucleotide are allowed.

Polynucleotides of the disclosure can be isolated from nucleic acid sequences present in, for example, a bacterial sample. Polynucleotides can also be synthesized in the laboratory, for example, using an automatic synthesizer. An amplification method such as PCR can be used to amplify polynucleotides from either genomic DNA or cDNA encoding the polypeptides.

Polynucleotides of the disclosure can comprise coding sequences for naturally occurring polypeptides or can encode altered sequences that do not occur in nature. If desired, polynucleotides can be cloned into an expression vector comprising expression control elements, including for example, origins of replication, promoters, enhancers, or other regulatory elements that drive expression of the polynucleotides of the disclosure in host cells. An expression vector can be, for example, a plasmid. Minichromosomes such as MC and MC1, bacteriophages, phagemids, yeast artificial chromosomes, bacterial artificial chromosomes, virus particles, virus-like particles, cosmids (plasmids into which phage lambda cos sites have been inserted) and replicons (genetic elements that are capable of replication under their own control in a cell) can also be used.

Methods for preparing polynucleotides operably linked to an expression control sequence and expressing them in a host cell are well-known in the art. See, e.g., U.S. Pat. No. 4,366,246. A polynucleotide of the disclosure is operably linked when it is positioned adjacent to or close to one or more expression control elements, which direct transcription and/or translation of the polynucleotide.

### Compositions

In embodiments, one or more lantibiotics of the disclosure (e.g., 1, 2, 3, 4, 5, 6, or more) are present in compositions that are antimicrobials, disinfectants, antibiotics, antiseptics, preservatives, antiviral or decontaminating agents. An antimicrobial composition kills microbes or slows the reproduction of microbes, such as bacteria. A disinfectant composition is applied to a non-living object to kill microbes or to slow the reproduction of microbes such as bacteria. An antibiotic kills microbes or slows the reproduction of microbes, such as bacteria, in the body of a subject or in cells or tissues. An antiseptic kills microbes or slows the reproduction of microbes, such as bacteria, on skin, tissue or organs. A preservative composition kills microbes or slows the reproduction of microbes in products such as paints, wood, foods, beverages, biological samples, cell or tissue cultures, or pharmaceutical compositions to prevent decomposition by microbes such as bacteria. A decontaminating agent is a cleaning agent that can be used to kill microbes or to reduce the reproduction of microbes, such as bacteria, in or on a living organism, cells, tissues, or objects.

In embodiments, the disclosure provides compositions comprising one or more lantibiotics of the disclosure that kill bacteria. In embodiments, the compositions comprising the variant MU1140 lantibiotics (i.e. the lantibiotics of the disclosure) kill about: 5, 10, 20, 30, 40, 50, 60, 70, 80, 90, or 100% (or any range between about 5% and 100%) of the bacteria they come in contact with. The difference between whether a lantibiotic acts as bacteriostatic agent or a bacteriocidal agent can be the amount or concentration of lantibiotic delivered to the subject, composition, or object to be treated. In embodiments, the lantibiotics of the disclosure reduce the numbers of bacteria present in a composition, subject, cells, or tissues to be treated. In one embodiment of the disclosure, a composition comprising a lantibiotic of the disclosure reduces the number of bacteria by about 5, 10, 20, 30, 40, 50, 60, 70, 80, 90, or 100% (or any range between about 5% and 100%).

In embodiments, variant lantibiotics of the disclosure are present in antimicrobial compositions comprising one or more isolated lantibiotics of the disclosure and one or more pharmaceutically acceptable carriers, diluents or excipients (solids or liquids). In one embodiment of the disclosure, the variant lantibiotic is present in a composition in an amount effective to substantially reduce bacterial reproduction of at least one type of Gram-positive bacteria by about: 5, 10, 20, 30, 40, 50, 60, 70, 80, 90 or 100% (or any range between about 5 and 100%). In embodiments of the disclosure, the variant MU1140 lantibiotic is present in a composition in an amount effective to substantially reduce the numbers of at least one type of Gram-positive bacteria by about: 5, 10, 20, 30, 40, 50, 60, 70, 80, 90 or 100% (or any range between about 5 and 100%). In embodiments, the at least one type of Gram-positive bacteria is, for example, Staphylococcus aureus, methicillin resistant Staphylococcus aureus, Staphylococcus saprophyticus, Staphylococcus epidermidis, vancomycin resistant Enterococci, vancomycin resistant Enterococcus faecalis, Enterococcus faecalis, Enterococcus faecium, Propionibacterium acnes, Streptococcus salivarius, Streptococcus sanguis, Streptococcus mitis, Streptococcus pyogenes, Lactobacillus salivarius, Listeria monocytogenes, Actinomyces israelii, Actinomyces naeslundii, Actinomyces viscosus, Bacillus anthracis, Streptococcus agalactiae, Streptococcus intermedius, Streptococcus pneumoniae, Corynebacterium diphtheria, Clostridium sporogenes, Clostridium botulinum, Clostridium perfringens, Clostridium tetani, and Clostridium difficile. All Gram-positive species are susceptible to lantibiotic variants of the disclosure.

Furthermore, Gram-negative bacteria can be susceptible to lantibiotic variants of the disclosure. Optionally, the outer membrane of Gram-negative bacteria can be disrupted with, for example, a chelating agent such as Tris, Tris-EDTA, or EDTA. Any membrane disrupting compounds can be added to compositions of the disclosure to increase the sensitivity of Gram negative bacteria to the lantibiotic variants of the disclosure, for example, polymixins, membrane disrupting antibiotics, cecropins (e.g., Musca domestica cecropin, hyalophora cecropins, cecropin B, cecropin P1), G1 OKHc (see Eckert et al., (2006) Antimicrob. Agents Chemother. 50:1480); alpha and beta defensins, ovine derived cathelicidine (see Anderson et al., (2004) Antimicrob. Agents Chemother. 48:673), squalamine derivatives (e.g., SM-7, see Kikuchi et al., (1997) Antimicrob. Agents Chemother. 41:1433), sodium hexametaphosphate, cellular enzymes of granulocytes (van den Broek, (1989) Rev. Infect. Dis. 11:213), EM49 (Rosenthal et al., (1976) Biochemistry, 15:5783), and sodium lauryl sarcosinate. The combination of lantibiotic variants of the disclosure with a membrane disruption agent and/or other antibiotics or drugs that target Gram-negative species can provide a composition effective against both Gram-positive and Gram-negative species. Therefore, the disclosure includes compositions comprising one or more lantibiotics of the disclosure and one or more additional antimicrobial agents or membrane disrupting agents. The one or more additional antimicrobial agents can have Gram-negative bacteriostatic or bacteriocidal activity. The membrane disrupting agent can render Gram-negative bacteria susceptible to a lantibiotic of the disclosure (i.e., the membrane disrupting agent in combination with one or more lantibiotic variants of the disclosure are bacteriostatic or bacteriocidal to Gram-negative bacteria). Gram-negative bacteria include, for example, Acinetobacter baumanii, Bordatella pertussis, Borrelia burgdotieri, Brucella abortus, Brucella canis, Brucella melitensis, Brucella suis, Campylobacter jejuni, Coxiella burnetii, Escherichia coli, Francisella tularensis, Haemophilus influenza, Helicobacter pylori, Klebsiella pneumoniae, Legionella pneumophila, Leptospira interrogans, Neisseria gonorrhoeae, Neisseria meningitides, Pseudomonas aeruginosa, Rickettsia rickettsii, Salmonella enteritidis, Salmonella typhi, Salmonella typhimurium, Serratia marcescens, Shigella sonnei, Treponema pallidum, Vibrio cholera, Yersinia enterocolitica, and Yersinia pestis.

Gram-variable and Gram-indeterminate bacteria can also be susceptible to lantibiotic variants of the disclosure. Optionally, chelating agents such as EDTA can be added to compositions of the disclosure to disrupt the outer membrane of these organisms. Gram-variable and Gram-indeterminate bacteria include, for example, Chlamydia pneumoniae, Chlamydia trachomatis, Chlamydia psittaci, Mycobacterium leprae, Mycobacterium tuberculosis, Mycobacterium ulcerans, and Mycoplasma pneumoniae.

A lantibiotic of the disclosure can be combined in a formulation with one or more pharmaceutically acceptable carriers, other carriers, diluents, adjuvants, excipients or encapsulating substances, which are suitable for administration to an animal, composition, or object. Exemplary pharmaceutically acceptable carriers, other carriers, diluents, adjuvants, excipients or encapsulating substances thereof include sugars, such as lactose, glucose, dextrose, and sucrose; starches, such as cornstarch and potato starch; cellulose and its derivatives, such as sodium carboxymethyl cellulose, ethyl cellulose, hydropropylmethylcellulose, and methyl cellulose; polysaccharides such as latex functionalized SEPHAROSE® and agarose; powdered tragacanth; glycerol; malt; gelatin; talc; solid lubricants, such as stearic acid and magnesium stearate; calcium sulfate; vegetable oils, such as peanut oil, cottonseed oil, sesame oil, olive oil, and corn oil; polyols such as propylene glycol, glycerine, sorbitol, mannitol, propylene glycol, and polyethylene glycol; proteins such as serum albumins, keyhole limpet hemocyanin, immunoglobulin molecules, thyroglobulin, ovalbumin, tetanus toxoid; alginic acid; emulsifiers, such as the TWEEN®s (polysorbate); polylactic acids; polyglycolic acids; polymeric amino acids such as polyglutamic acid, and polylysine; amino acid copolymers; peptoids; lipitoids; inactive avirulent virus particles or bacterial cells; liposomes; hydrogels; cyclodextrins; biodegradable nanocapsules; bioadhesives; wetting agents, such sodium lauryl sulfate; coloring agents; flavoring agents; tableting agents; stabilizers; antioxidants; preservatives; pyrogen-free water; isotonic saline; ethanol; ethyl oleate; pyrrolidone; Ringer's solution, dextrose solution, Hank's solution; sodium alginate; polyvinylpyrrolidone; gum tragacanth; gum acacia; and sterile water and aqueous buffers and solutions such as physiological phosphate-buffered saline. Carriers, such as pharmaceutically acceptable carriers and diluents, for therapeutic use are well known in the art and are described in, for example, Remington's Pharmaceutical Sciences, Mack Publishing Co. (A. R. Gennaro ed. (1985)). Pharmaceutically acceptable salts can also be used in compositions of the disclosure, for example, mineral salts such as hydrochlorides, hydrobromides, phosphates, or sulfates, as well as salts of organic acids such as acetates, proprionates, malonates, or benzoates.

Various dosage forms can comprise the lantibiotic compositions as described herein. In some embodiments, the dosage form can be adapted for oral, rectal, vaginal, urethral topical, (including transmucosal and transdermal), intramuscular, intravenous, intradermal, subcutaneous, intramuscular, or intraperitoneal delivery of the lantibiotic.

The variant lantibiotic compositions can be in a formulation in a form suitable for oral delivery, for example, as tablets, troches, lozenges, mouthwashes, dentifrices, buccal tablets, solutions, aqueous or oily suspensions, dispersible powders or granules, emulsions, hard or soft capsules, or syrups or elixirs. Such compositions can contain one or more agents, such as emulsifying agents, wetting agents, pH buffering agents, sweetening agents, flavoring agents, coloring agents and preserving agents. The lantibiotic compositions can be a dry product for reconstitution with water or other suitable liquid before use.

Lantibiotics of the disclosure can also be administered in the form of suppositories for rectal, vaginal, or urethral administration of the drug. These compositions can be prepared by mixing the variant lantibiotic with a suitable nonirritating carrier that is solid at ordinary temperatures but liquid at the body temperature and will therefore melt in the rectum to release the drug. Such materials are cocoa butter and polyethylene glycols.

A lantibiotic of the disclosure can also be topically administered in the form of, e.g., lotions, gels, or liposome delivery systems, such as small unilamellar vesicles, large unilamellar vesicles, and multilamellar vesicles. Liposomes can be formed from a variety of phospholipids, such as cholesterol, stearylamine or phosphatidylcholines. Other dosage forms include, for example, injectable, sublingual, enema, and nasal dosage forms. Compositions for inhalation typically can be provided in the form of a solution, suspension or emulsion that can be administered as a dry powder or in the form of an aerosol using a conventional propellant (e.g., dichlorodifluoromethane or trichlorofluoromethane).

Formulations can contain between about 0.0001% and about 99.9999% by weight of one or more lantibiotic(s) of the disclosure and usually at least about 5, 10, 15, 20, 25, 30, 40, 50, 60, 70, 80, 90, or 100% (weight %) of one or more lantibiotic variants of the present disclosure. Some embodiments contain from about 25% to about 50% or from 5% to 75% of a lantibiotic of disclosure.

One or more lantibiotics of the disclosure can be combined with one or more antimicrobials, antibiotics, bacteriocins, anti-viral compounds or molecules, virucidal compounds or molecules, or anti-fungal compounds or molecules to form a composition useful in the methods of the disclosure.

Antibiotics include, for example, penicillins, cephalosporins, polymixins, quinolones, sulfonamides, aminoglycosides, macrolides, tetracyclines, cyclic lipopeptides (e.g., daptomycin), glycylcyclines (e.g., tigecycline), and oxazolidinones (e.g., linezoid).

Bacteriocins include, for example, acidocin, actagardine, agrocin, alveicin, aureocin, carnocin, carnocyclin, colicin, curvaticin, divercin, duramycin, enterocin, enterolysin, epidermin, erwiniocin, gallidermin, glycinecin, halocin, haloduracin, lactococin, lacticin, leucoccin, macedocin, mersacidin, mesentericin, microbisporicin, mutacin, nisin, paenibacillin, planosporicin, pediocin, pentocin, plantaricin, reutericin, sakacin, salivaricin, subtilin, sulfolobicin, thuricin, trifolitoxin, variacin, vibriocin, warnericin, and warnerin.

Antifungals include, for example, polyene antifungals (e.g., amphotericin B, natamycin, rimocidin, filipin, nystatin, candicin, hamycin), azole antifungals (e.g., imidazole, triazole, thiazole), imidazoles (e.g., miconazole, ketoconazole, clotrimazole, econazole, omoconazole, bifonazole, butoconazole, fenticonazole, isoconazole, oxiconazole, sertaconazole, sulconazole, tioconazole), triazoles (e.g., fluconazole, itraconazole, isavuconazole, ravuconazole, posaconazole, voriconazole, terconazole, albaconazole), thiazoles (e.g., abagungin), allylamines (e.g., terbinafine, naftifine, butenafine), echinocandins (e.g., anidulafungin, caspofungin, micafungin), polygodial, benzoic acid, ciclopiroxolamine, tolnaftate, undecylenic acid, flucytosine, and griseofulvin.

Antivirals and virucidal agents include, for example, abacavir, aciclovire, acyclovir, adefovir, amantadine, amprenavir, ampligen, arbidol, atazanavir, atripla, boceprevir, cidofovir, combivir, delavirdine, didanosine, docosanol, efavirenz, emtricitabine, enfuvirtide, entecavir, entry inhibitors, famciclovir, fomivirsen, fosamprenavir, foscarnet, fosfonet, ganciclovir, ibacitabine, imunovir, idoxuridine, imiquimod, indinavir, inosine, integrase inhibitor, interferon types i, ii, or iii, interferon, lamivudine, lopinavir, loviride, maraviroc, moroxydine, methisazone, nelfinavir, nevirapine, nexavir, nucleoside analogues, oseltamivir, peginterferon alpha-2a, penciclovir, peramivir, pleconaril, podophyllotoxin, protease inhibitor, raltegravir, reverse transcriptase inhibitor, ribavirin, rimantadine, ritonavir, pyramidine, saquinavir, stavudine, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine.

Compositions Comprising Recombinant S. mutans

Compositions of the disclosure can be expressed by one or more strains of recombinant S. mutans strains as described herein and a pharmaceutically acceptable or nutritionally acceptable carrier. For example, recombinant S. mutans strains can be characterized by: 1) a lactic acid deficiency, and 2) production of a recombinant ADH, 3) variant MU1140 production, 4) optionally, an auxotrophy for a specific organic substance (e.g., a D amino acid such as D-alanine), 5) optionally, a deficiency in ComE expression, or combinations thereof.

The carrier is physiologically compatible with the area of the subject to which it is administered. Carriers can be comprised of solid-based, dry materials for formulation into tablet, capsule, lozenge, or powdered form. A carrier can also be comprised of liquid or gel-based materials.

### Uses of Lantibiotics

In some embodiments, the lantibiotics of the disclosure are used to reduce the growth of bacteria, prevent the growth of bacteria, prevent the reproduction of bacteria, reduce the reproduction of bacteria, or to reduce or eliminate the numbers of bacteria present in or on an object, composition or subject. In one embodiment of the disclosure, the bacteria are at least one type of Gram-positive bacteria, at least one type of Gram-negative bacteria, at least one type of Gram-variable or Gram-indeterminate bacteria, or a combination of at least one type of Gram-positive or at least one type of Gram-negative bacteria or at least one type of Gram-variable or Gram-indeterminate bacteria. In embodiments, the lantibiotics of the disclosure are administered to, added to, or contacted with a composition or subject in need of treatment.

In embodiments of the disclosure, the bacteriostatic action of a lantibiotic of the disclosure reduces reproduction of the bacteria by about: 5, 10, 20, 30, 40, 50, 60, 70, 80, 90, or 100% (or any range between about 5% and 100%). In embodiments, the lantibiotics of the disclosure kill bacteria. In embodiments of the disclosure, the variant MU1140 lantibiotics kill about: 5, 10, 20, 30, 40, 50, 60, 70, 80, 90, or 100% (or any range between about 5% and 100%) of the bacteria they come in contact with. The difference between whether a lantibiotic acts as bacteriostatic agent or a bacteriocidal agent can be the amount or concentration of lantibiotic delivered to the subject, composition, or object to be treated. Lantibiotics of the disclosure can reduce the numbers of bacteria present in a composition, subject, cells, or tissues to be treated. In one embodiment of the disclosure, variant MU1140 lantibiotics reduce the number of bacteria by about 5, 10, 20, 30, 40, 50, 60, 70, 80, 90, or 100% (or any range between about 5% and 100%).

The disclosure therefore provides methods of treating, ameliorating, or preventing a disease, infection, or colonization. A disease is a pathological condition of a part, organ, or system of an organism resulting from infection and characterized by an identifiable group of signs and symptoms. An infection is invasion by and multiplication of pathogenic microorganisms, such as bacteria, in a bodily part or tissue, which may produce a subsequent tissue injury and progress to overt disease through a variety of cellular or toxic mechanisms. Colonization is the act or process of a microorganism, such as bacteria, establishing itself on or within a host or object. Colonization may produce a subsequent biofilm or biofouling condition as described below. In embodiments, lantibiotics of the disclosure are used prophylactically to prevent disease, infection or colonization or to prevent the spread of a disease or infection. Examples of diseases, infections and colonizations that can be treated or prevented by the compositions and methods of the disclosure include, for example, septicemia, bacterial meningitis, cystic fibrosis, bovine mastitis, impetigo, bacterial vaginosis, bacterial pneumonia, urinary tract infections, bacterial gastroenteritis, erysipelas, cellulitis, anthrax, whooping cough, brucellosis, enteritis, opportunistic infections, community acquired respiratory infections, upper and lower respiratory infections, diphtheria, nosocomial infections, diarrhea, ulcer, bronchitis, listeriosis, tuberculosis, gonorrhea, pseudomonas infections, salmonellosis, shigellosis, E. coli infections, staphylococcal infections, streptococcal infections, recurrent or primary C. difficile associated infections, and necrotizing fasciitis.

In embodiments, the disclosure provides methods of treating a subject having a bacterial infection by administering a lantibiotic of the disclosure. In embodiments, the disclosure provides methods of treating a subject that has been diagnosed with a bacterial infection by a health care provider, such as a doctor, nurse or physician's assistant, by administering a lantibiotic of the disclosure. In embodiments, treating comprises reducing or eliminating the number of bacteria in or on the subject. In embodiments, the disclosure further provides methods of preventing a bacterial infection by administering a lantibiotic of the disclosure. In embodiments, the subject is infected with a Gram-positive bacteria. In embodiments, the Gram-positive bacteria is Staphylococcus aureus, methicillin resistant Staphylococcus aureus, Staphylococcus saprophyticus, Staphylococcus epidermidis, vancomycin resistant Enterococci, vancomycin resistant Enterococcus faecalis, Enterococcus faecalis, Enterococcus faecium, Propionibacterium acnes, Streptococcus salivarius, Streptococcus sanguis, Streptococcus mitis, Streptococcus pyogenes, Lactobacillus salivarius, Listeria monocytogenes, Actinomyces israelii, Actinomyces naeslundii, Actinomyces viscosus, Bacillus anthracis, Streptococcus agalactiae, Streptococcus intermedius, Streptococcus pneumoniae, Corynebacterium diphtheria, Clostridium sporogenes Clostridium botulinum, Clostridium petiringens, Clostridium tetani, and Clostridium difficile. In embodiments, the disclosure provides a method of treating a subject having a C. difficile infection, e.g., reducing the number of C. difficile bacteria in a subject, by administering to the subject a lantibiotic of the disclosure. In embodiments, the disclosure provides a method of preventing a C. difficile infection in a subject by administering to the subject a lantibiotic of the disclosure. In embodiments, the subject is a human. Methods of determining whether a subject has a bacterial infection are well-known in the art.

In embodiments, the subject is a mammal, such as a mouse, rabbit, guinea pig, macaque, baboon, chimpanzee, human, cow, sheep, pig, horse, dog, cat, or to a non-mammalian animal such as a chicken, duck, or fish. Lantibiotics of the disclosure can also be administered to plants, seeds, or plant media such as soil.

Administration of the lantibiotics of the disclosure can be by any means known in the art, including injection (e.g., intramuscular, intravenous, intrapulmonary, intramuscular, intradermal, intraperitoneal, intrathecal, or subcutaneous injection), aerosol, intranasal, infusion pump, suppository (rectal, vaginal, urethral), mucosally, topically, buccally, orally, parenterally, infusion techniques, by enemas, by inhalation, enemas, or spray, sublingually, transdermally, as an ophthalmic solution, intraspinal application, or by other means, in dosage unit formulations containing conventional non-toxic pharmaceutically acceptable carriers, diluents, excipients, adjuvants, and vehicles. A combination of administration methods can also be used.

In therapeutic applications, the lantibiotic compositions of the disclosure are administered to subjects to reduce the reproduction of bacteria or reduce the numbers of bacteria, or both. The particular dosages of lantibiotic in a composition will depend on many factors including, but not limited to the species, age, gender, severity of infection, concurrent medication, general condition of the subject to which the composition is administered, and the mode of administration of the composition. An effective amount of the composition of the disclosure can be readily determined using only routine experimentation. A therapeutically effective amount means the administration of that amount to a subject, either in a single dose or as part of a series, which is effective for treatment, amelioration, or prevention of bacterial infection or colonization. A therapeutically effective amount is also an amount effective in alleviating or reducing the symptoms of an infection or in reducing the reproduction of bacteria in or on a subject or reducing the amount of bacteria in or on a subject.

The concentration of lantibiotic in a composition can vary, and will be selected primarily based on activity of the lantibiotic, body weight of the subject, overall health of the subject, etc. as described above, in accordance with the particular mode of administration selected and the subject's needs. Concentrations, however, will typically be selected to provide dosages ranging from about: 0.001, 0.01, 0.1, 1, 5, 10, 20, 30, 40, 50, 75,100,150 mg/kg/day (or any range between about 0.001 and 150 mg/kg/day) and sometimes higher. Typical dosages range from about 0.1 mg/kg/day to about 5 mg/kg/day, from about 0.1 mg/kg/day to about 15 mg/kg/day, from about 0.1 mg/kg/day to about 20 mg/kg/day, and from about 0.1 mg/kg/day to about 50 mg/kg/day.

Lantibiotics of the disclosure are administered over time and several times per day (e.g., 1 day, 3 days, 1 week, 1 month, 2 months, 3 months, 6 months, 1 year or more) or can be administered in maintenance doses for long periods of time to prevent or reduce disease, infection, colonization, biofilms or biofouling conditions.

Lantibiotics of the disclosure can be administered either to an animal that is not infected or colonized with bacteria or can be administered to a bacterially infected or colonized animal.

One embodiment of the disclosure provides a method for decontaminating or reducing bacterial growth on or in an inanimate object comprising contacting the object with a lantibiotic of the disclosure for a period effective to substantially inhibit bacterial growth of at least one type of bacteria. The contacting can be for 1, 15, 30, or 60 minutes, or 2, 3, 10, 12, 24, 36 or 48 hours (or any range between about 1 minute and 48 hours). An object can be, for example, a food preparation surface, food preparation equipment, industrial equipment, pipes, or a medical device such as catheter, scalpel, knife, scissors, spatula, expander, clip, tweezers, speculum, retractor, suture, surgical mesh, chisel, drill, level, rasp, saw, splint, caliper, clamp, forceps, hook, lancet, needle, cannula, curette, depressor, dilator, elevator, articulator, extractor, probe, staple, artificial joint, wound dressing, catheter, stent, tubing, bowl, tray, sponge, snare, spoon, syringe, pacemaker, screw, plate, pin, wire, guide wire, pacemaker lead, implant, sensor, glucose sensor, blood bypass tubing, i.v. bag, ventricular assist device components, ophthalmic lens, and balloon.

Other objects that can be decontaminated include textiles such as a woven (woven from natural or non-natural materials or a blend of natural and synthetic materials) or nonwoven material (e.g., elastic or non-elastic thermoplastic polymers). The textiles can be used for, e.g., a protective article worn by patients, healthcare workers, or other persons who may come in contact with potentially infectious agents or microbes, such as a gown, robe, face mask, head cover, shoe cover, or glove. Other protective textiles can include surgical drapes, surgical covers, drapes, sheets, bedclothes or linens, padding, gauze dressing, wipe, sponge, and other antimicrobial articles for household, institutional, health care, and industrial applications.

In embodiments of the disclosure, a lantibiotic is coated onto, immobilized, linked, or bound to a solid surface such as a food preparation surface, food preparation equipment, industrial equipment, pipes, or a medical device such as catheter, scalpel, knife, scissors, spatula, expander, clip, tweezers, speculum, retractor, suture, surgical mesh, chisel, drill, level, rasp, saw, splint, caliper, clamp, forceps, hook, lancet, needle, cannula, curette, depressor, dilator, elevator, articulator, extractor, probe, staple, artificial joint, wound dressing, catheter, stent, tubing, bowl, tray, sponge, snare, spoon, syringe, pacemaker, screw, plate, pin, wire, guide wire, pacemaker lead, implant, sensor, glucose sensor, blood bypass tubing, i.v. bag, ventricular assist device components, ophthalmic lens, balloon, and textiles as described above.

In another embodiment of the disclosure, lantibiotic compositions of the disclosure are present in a transdermal formulation. A transdermal formulation can be designed so the lantibiotic composition acts locally at the point of administration or systemically by entering an animal or human's blood circulation. Therefore, delivery can occur by direct topical application of the lantibiotic composition in the form of an ointment or lotion, or by adhesion of a patch embedded with the lantibiotic composition or with a reservoir that holds the lantibiotic composition and releases it to the skin all at once or in a time-controlled fashion.

Optionally, lantibiotic compositions can be contained within vesicles such as microparticles, microspheres, liposomes, lipid vesicles, or transfersomes for transdermal or topical delivery. Ultrasound devices to generate shock waves to enlarge pores, use of electric current to drive substances across skin, and the use of microneedles to pierce skin and deliver lantibiotic compositions into the bloodstream can also be used with transdermal or topical administration. Methods of coating, binding, or immobilizing peptides, such as the lantibiotics of the disclosure onto surfaces are well-known in the art. See e.g., Modern Methods of Protein Immobilization, William H. Scouten, First Ed. (2001) CRC Press; Protein Immobilization (Biotechnology and Bioprocessing), Richard F. Taylor (1991) CRC Press.

Methods of the disclosure can also be used to ameliorate, reduce, remove, or prevent biofouling or biofilms. Biofouling is the undesirable accumulation of microorganisms, such as bacteria on structures exposed to solvent. Biofouling can occur, for example on the hulls of ships, in membrane systems, such as membrane bioreactors and reverse osmosis spiral wound membranes, water cooling systems of large industrial equipment and power stations, and oil pipelines carrying, e.g., used oils, cutting oils, soluble oils or hydraulic oils.

A biofilm can cause biofouling and is an aggregate of organisms wherein the organisms are adhered to each other, to a surface, or a combination thereof. A biofilm can comprise one or more species of bacteria, fungi, filamentous fungi, yeasts, algae, cyanobacteria, viruses, and protozoa and combinations thereof. Microorganisms present in a biofilm can be embedded within a self-produced matrix of extracellular polymeric substances. When a microorganism switches to a biofilm mode of growth, it can undergo a phenotypic shift in behavior wherein large suites of genes are differentially regulated. Nearly every species of microorganism can form biofilms. Biofilms can be found on or in living organisms or in or on non-living structures. Biofilms can be present on structures contained in naturally occurring bodies of water or man-made bodies of water, on the surface of water, surfaces exposed to moisture, interiors of pipes, cooling water systems, marine systems, boat hulls, on teeth, on plant surfaces, inside plants, on human and animal body surfaces, inside humans and animals, on contact lenses, on catheters, prosthetic cardiac valves, other prosthesis, intrauterine devices, and other structures/devices.

Biofilms can cause corrosion of metal surfaces, inhibit vessel speed, cause plant diseases, and can cause human and animal diseases. Biofilms are involved in human and animal infections, including, for example, urinary tract infections, catheter infections, middle-ear infections, dental plaque, gingivitis, dental caries, periodontal diseases, endocarditis, infections in cystic fibrosis, chronic sinusitis, and infections of permanent indwelling devices such as joint prostheses and heart valves. Biofilms can also impair cutaneous wound healing and reduce topical antibacterial efficiency in healing or treating infected skin wounds.

Some microorganisms that can form biofilms, cause biofouling and/or cause disease in humans and animals include, for example, bacteria, fungi, yeast, algae, protozoa, and viruses as described above. Biofilms can be treated in living organisms as described above. Biofilms and biofouling conditions on non-living surfaces can be treated by applying the lantibiotics of the disclosure onto the nonliving surface or to the area surrounding the surface. Lantibiotic of the disclosure can also be added to the water, oil, or other fluid surrounding and in contact with the nonliving surface.

The disclosure provides methods of ameliorating or preventing a biofouling condition or a biofilm condition, caused by one or more microorganisms, such as bacteria. The methods comprise administering one or more of the variant lantibiotics to the biofouling condition or biofilm condition, wherein the biofouling condition or biofilm condition is ameliorated.

The one or more lantibiotics can be administered to a surface that has a biofilm or biofouling condition or can be administered to a surface as a prophylactic measure. The lantibiotics can be in a dried form (e.g., lyophilized or tablet form) or a liquid solution or suspension form. The dried or liquid forms can be swabbed, poured, sprayed, flushed through the surface (e.g., pipes or membranes) or otherwise applied to the surface. Lantibiotics of the disclosure can be present in a composition with a carrier or diluent in an amount from about 0.001, 0.01, 0.1, 1, 5, 10, 20, 30, 40, 50, 75, 100, 150 mg/m² (or any range between about 0.001 and about 30 150 mg/m²) and sometimes higher.

Where the biofilm is present or potentially present on an artificial surface within a human or animal (e.g., a catheter or medical device), the artificial surface can be contacted with the one or more lantibiotics prior to insertion into the human or animal. Optionally, the lantibiotics can be delivered to the surface after the artificial surface is inserted into the human or animal.

In one embodiment of the disclosure, a variant lantibiotic can be used for decontaminating or reducing bacterial reproduction or bacterial numbers in a biological tissue or cell culture. The lantibiotic can be present in a pharmaceutically acceptable carrier, diluent or excipient at the dosage rates as for pharmaceutical compositions described above. The lantibiotic or lantibiotic composition can be contacted with the tissue or cell culture for a period effective to substantially inhibit bacterial growth of at least one type of bacteria. The lantibiotic can be provided in an amount effective to maintain the physiological characteristics of the biological tissue or cells and/or in an amount effective to substantially maintain the viability of the biological tissue or cells.

One embodiment of the disclosure provides a method for preparing isograft organs, tissues or cells, autograft tissues or cells, allograft organs, tissues or cells, xenograft organs, tissues or cells, or other cells or tissue for transplantation. The method comprises contacting the organs, cells or tissues with a lantibiotic composition of the disclosure for a period effective to inhibit or reduce bacterial growth or bacterial numbers of at least one type of Gram-positive bacteria. The cells, organs or tissues can be, for example, a heart valve, a blood vessel, pericardium or musculoskeletal tissue, ligaments such as anterior cruciate ligaments, knee joints, hip joints, ankle joints, meniscal tissue, skin, cornea, heart, lung, small bowel, intestine, liver, kidney, bone marrow, bone, and tendons.

The contacting step can be performed at a temperature from about 2° C. to about 42° C. for about: 0.5, 1, 2, 3, 5, 10, 24, 36, or 48 hours. The lantibiotic composition can further comprise a physiological solution further comprising one or more broad spectrum antimicrobials and/or one or more antifungal agents, such as, for example vancomycin, imipenem, amikacin, and amphotericin B.

Lantibiotic compositions of the disclosure can also be used as a preservative for allograft and xenograft process solutions, and cell culture and tissue solutions. The solutions can comprise an effective amount of one or more lantibiotics in a physiological solution at a pH of between 3 and 8.

One or more lantibiotics of the disclosure can be added to foods or beverages as a preservative. Examples of foods include, processed cheese products, pasteurized dairy products, canned vegetables, high moisture, hot baked flour products, pasteurized liquid egg, and natural cheese products. Lantibiotics of the disclosure can also be used to control Listeria in foods, to control spoilage by lactic acid bacteria in, e.g., beer, wine, alcohol production and low pH foods such as salad dressings. Lantibiotics of the disclosure can be used as an adjunct in food processing technologies such as higher pressure sterilization and electroporation. Lantibiotics can be present in a food or beverages in an amount from about: 0.001, 0.01, 0.1, 1, 5, 10, 20, 30, 40, 50, 75, 100, 150, 250, 300, 400, 500, 600, 700, 800, 900, 1,000 or more mg/kg or mg/L (or any range between about 0.001 and about 1,000 mg/kg or 10 mg/l and sometimes higher.

Lantibiotics of the disclosure can also be used as molecular wires, molecular switches, or molecular based memory systems. Therefore, variant lantibiotics and wild-type lantibiotics have potential use for building nano-circuitry, as well as other nano-based applications. Molecular wires (also known as molecular nanowires) are molecular-scale substances that conduct electrical current, which are the fundamental building blocks for molecular electronic devices. The typical diameter of molecular wires is less than three nanometers, while the length can extend to centimeters or more. A molecular wire allows the flow of electrons from one end of the wire to the other end of the wire. Molecular wires can comprise at least two terminals for contacting additional components of a nano-electronic device.

A molecular switch (also known as a controllable wire) is a molecular structure where the electron flow can be turned on and off on demand. A molecular based memory system is one or more molecule wires or switches that have the ability to alter its conductivity by storing electrons. A molecular wire, switch, or molecular based memory system can be present on or anchored to substrates such as silicon wafers, synthetic polymer supports, glass, agarose, nitrocellulose, nylon, Au, Cu, Pd, Pt, Ni, Al, Al₂O₃, nickel grids or disks, carbon supports, aminosilane-treated silica, polylysine coated glass, mica, and semiconductors.

In embodiments, the disclosure provides recombinant S. mutans strains that produce one or more lantibiotic variants of the disclosure. Examples of such strains include JH1000 or JH1140. Recombinant S. mutans strains can further contain an erythromycin gene in the mutA′-mutB intergenic region. Examples of such strains include SM152. Recombinant S. mutans strains can also be strains that produce for example, 2×, 3×, 4×, 5×, 6×, 7×, 8×, 9×, 10×, 100×, 1000× more lantibiotic than wild type recombinant S. mutans strains.

In embodiments, the disclosure provides recombinant S. mutans strains that produce one or more lantibiotic variants of the disclosure to outcompete and substantially eliminate wild-type, cariogenic S. mutans from the oral cavity of a host (e.g., reduce the number of wild-type S. mutans by about: 5, 10, 25, 50, 75, 90, 95, 99, or 100% (or any range between about 5% and about 100%)). Production of lantibiotic variants of the invention with enhanced biological activity as compared to a wild-type MU1140 lantibiotic can therefore provide an S. mutans with a selective advantage over MU1140 producing, non-MU1140 lantibiotic producing, or non-lantibiotic producing S. mutans strains present in the oral cavity of a host. The lantibiotic variants of the disclosure, when expressed by a recombinant S. mutans strain of the disclosure, eliminates the resident, lantibiotic-susceptible S. mutans strains, thus interfering with colonization of lantibiotic-susceptible strains and promoting recombinant S. mutans colonization of the oral cavity. Since the wild-type, native S. mutans is displaced from the oral cavity, the incidence and/or severity of dental caries is reduced.

In one embodiment of the disclosure, the recombinant strain can additionally express lanB, lanC, lanD, lanE, lanF, lanG, lanK, lanM, lanP, lanR, lanT, lanI, mutR, mutAA′, mutBCDPT, mutFEG or combinations of two or more of these S. mutans polypeptides.

In embodiments, the recombinant S. mutans strains of the disclosure are lactic acid deficient, meaning that a recombinant S. mutans strain produces substantially decreased amounts of lactic acid relative to wild-type S. mutans. Substantially decreased amounts of lactic acid are about 40, 50, 60, 70, 80, 90, 95, or 100% (or any range between about 40% and about 100%) less lactic acid than is produced by a wild-type S. mutans strain (e.g. S. mutans strain UA159 (ATCC 700610)) or other species belonging to the Streptococcus genus including Streptococcus sobrinus (e.g. S. sobrinus strain SL1 (ATCC 33478)), Streptococcus rattus (e.g., S. rattus strain FA1 (ATCC 19645)), Streptococcus cricetus (S. crecitus strain HS6 (ATCC 19642)), and Streptococcus ferus (S. ferus strain 8S1)). In one embodiment of the disclosure, a lactic acid deficient S. mutans effector strain produces no detectable lactic acid. Lactic acid expression can be detected as described in, e.g., Hillman et al., Infect. Immun. 62:60 (1994); Hillman et al., Infect. Immun. 64:4319 (1996); Hillman et al., 1990, Infect. Immun., 58:1290-1295.

Recombinant S. mutans strains of the disclosure can be lactic acid deficient as a result of a non-functional, inactivated, partially functional, or partially inactivated regulatory region, translational signal, transcriptional signal, or structural sequence in the lactic acid synthesis pathway as disclosed in International Application No. PCT/US2013/027340 or U.S. Pat. No. 5,607,672, each of which is incorporated by reference herein in its entirety.

Because defects in lactic acid synthesis are lethal for S. mutans, the defect in the recombinant, lactic acid-deficient S. mutans strains must be complemented by the production of a recombinant alcohol dehydrogenase (ADH). See e.g., Hillman et al., Infect. Immun. 64:4319 (1996). Production of the recombinant ADH prevents accumulation of metabolites, e.g., pyruvate, that otherwise causes the death of lactic acid-deficient S. mutans.

A S. mutans strain can be genetically engineered to express a recombinant alcohol dehydrogenase as disclosed in International Application No. PCT/US2013/027340, incorporated herein by reference in its entirety. Recombinant S. mutans strains of the disclosure can optionally be genetically engineered to be auxotrophic for an organic substance not normally present in the oral cavity or diet of a host so that the oral cavity colonization by the recombinant S. mutans strains can be controlled. That is, the recombinant S. mutans strains can optionally be genetically engineered so that they are unable to synthesize a particular organic compound required for growth, as disclosed in International Application No. PCT/US2013/027340.

Optionally, a recombinant S. mutans strain of the disclosure can comprise an inactivated or non-functional comE gene. “ComE deficient” or “deficiency in ComE production” means that a recombinant S. mutans strain produces substantially decreased amounts of ComE protein relative to wild-type S. mutans. Substantially decreased amounts of ComE are about 40, 50, 60, 70, 80, 90, 95, or 100% (or any range between about 40% and about 100%) less ComE protein than is produced by a wild-type S. mutans strain. In one embodiment of the disclosure, a ComE deficient recombinant S. mutans strain produces no detectable ComE protein. ComE expression can be assayed as described in, e.g., Chen & Gotschlich, J. Bact. 183:3160 (2001). Recombinant S. mutans strains of the disclosure can be ComE deficient as a result of a non-functional, inactivated, partially functional, or partially inactivated regulatory region, translational signal, transcriptional signal, or structural sequence in ComE synthesis, as disclosed in International Application No. PCT/US2013/027340.

In embodiments, the recombinant S. mutans of the disclosure are present in a composition of the disclosure in a therapeutically effective amount. Therapeutically effective means effective to prevent or reduce the number or incidence (e.g., 5, 10, 20, 30, 40, 50, 60, 70, 80, 90 or 100% fewer cavities than controls that did not receive the composition) and/or reduce the severity (e.g., 5, 10, 20, 30, 40, 50, 60, 70, 80, 90 or 100% less severe cavities than controls that did not receive the composition) of cavities.

A therapeutically effective amount or dosage is an amount or dosage of a composition of the disclosure at high enough levels to prevent caries and/or reduce caries number and/or caries severity, but low enough to avoid serious side effects (at a reasonable benefit/risk ratio), within the scope of sound medical/dental judgment. The compositions of the disclosure can be applied in a therapeutically effective amount to the oral cavity of a host for the treatment and/or prevention of cavities using methods known in the art.

In one embodiment of the disclosure a composition can comprise one or more isolated recombinant strains of the disclosure along with one or more isolated Streptococcus oralis strains, one or more isolated S. rattus strains, and/or one or more isolated Streptococcus uberis strains using methods known in the art.

One embodiment of the disclosure provides a method for treating dental caries comprising administering a composition comprising one or more recombinant S. mutans strains of the disclosure to the oral cavity of a subject in need thereof using methods known in the art.

The disclosure also provides a method of reducing the amount of bacteria that can cause dental caries in a subject. Another embodiment of the disclosure provides a method of preventing dental caries in a subject, for example, compositions comprising the recombinant S. mutans strains of the disclosure are administered to the oral cavity of a host or subject such as an animal, including a mammal, for example, a human, a non-human primate, a dog, a cat, a horse, a bovine, a goat, or a rabbit using methods known in the art. The compositions of the disclosure can be orally administered using methods known in the art.

### Kits

Compositions of the disclosure can be present in a kit comprising a container of one or more lantibiotics of the disclosure. The lantibiotics can be lyophilized and in the form of a lyophilized powder or tablet or can be in a solution or suspension optionally with buffers, excipients, diluents, adjuvants, or pharmaceutically acceptable carriers. A kit can also comprise one or more applicators for the one or more lantibiotics to a body part or tissue or surface. The applicator can be, for example, a swab, a syringe (with or without a needle), a dropper, a sprayer, a surgical dressing, wound packing, or a bandage. Optionally, the kit can comprise one or more buffers, diluents, adjuvants, therapeutically acceptable carriers, or pharmaceutically acceptable carriers for reconstituting, diluting, or preparing the one or more variant MU1140 lantibiotics.

A kit of the disclosure can contain a single dose, a one week, one month, two month, three month, four month, five month, six month, or 12 month supply of a composition of the disclosure. A composition of the disclosure can be packaged and, in turn, a plurality of the packaged compositions can be provided in a storage container or outer package or carton. Where the one or more strains of S. mutans are auxotrophic, the kit can include a bacterial auxotroph-maintaining amount of an organic substance, e.g., a composition comprising a D-amino acid such as D-alanine.

All patents, patent applications, and other scientific or technical writings referred to anywhere herein are incorporated by reference herein in their entirety. The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically disclosed by embodiments, optional features, modification and variation of the concepts herein disclosed may be resorted to by those skilled in the art, and that such modifications and variations are considered to be within the scope of this disclosure as defined by the description and the appended claims.

The following are provided for exemplification purposes only and are not intended to limit the scope of the disclosure described in broad terms above.

### EXAMPLES

The present disclosure is further defined in the following Examples. The Examples set forth herein, represent certain features, elements and embodiments of the disclosure, and is not intended, nor should be construed, to limit the scope of the disclosure.

MU1140 variants (i.e., MU1140 polypeptide variants including non-naturally occurring variants) with improved antimicrobial/antibiotic activity and/or stability were produced by mutating select single and multiple combinations of amino acids within the (mature) 22 amino acid native (wild-type) MU1140 polypeptide sequence (SEQ ID NO:1156, FIG. 1). The mutA variants were constructed using pLAN042, from which a linear amplicon was used to substitute the native mutA sequence with a mutA sequence containing codon substitutions. Following transformation of the mutA variant library into SM152 (JH1140 lanA′B::erm) and verification of codon substitution in mutA, S. mutans variant clones were picked, expressed and analyzed for antimicrobial activity (see, Table 1 and Table 2, below) and stability. Data was interpreted by qualitative comparison of the size of the zone of clearing for each lantibiotic variant producing strain with the mutA invariant control SM152 and the wild-type parent strain JH1140.

### Example 1

### MU1140 Single and Multi-Site Variants

Construction of mutA Variant Strains

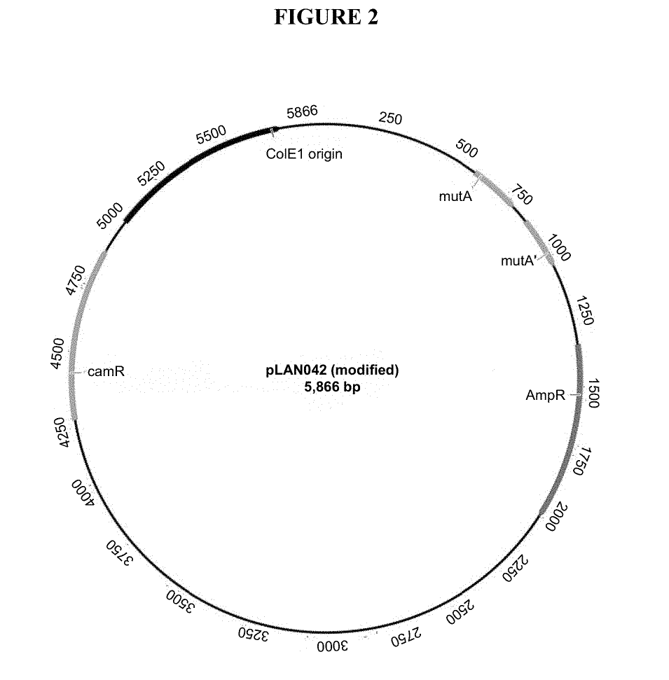

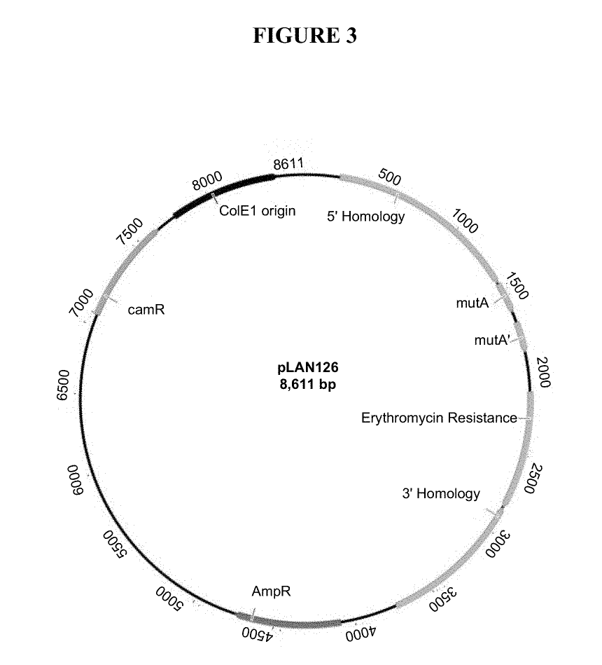

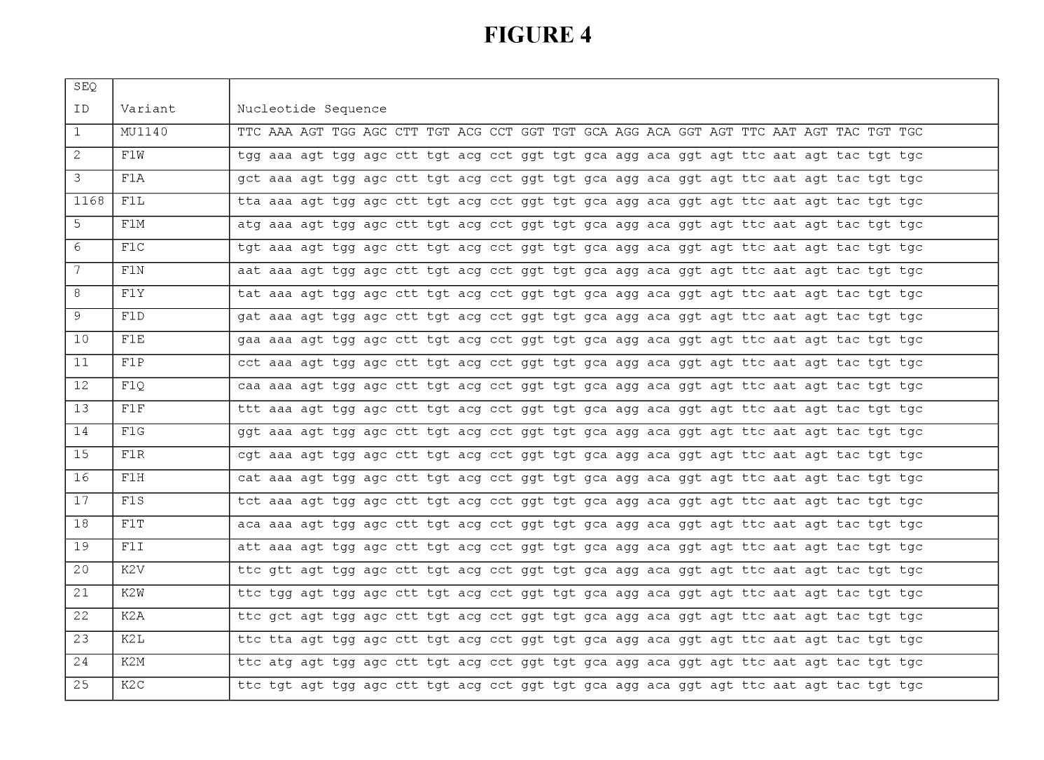

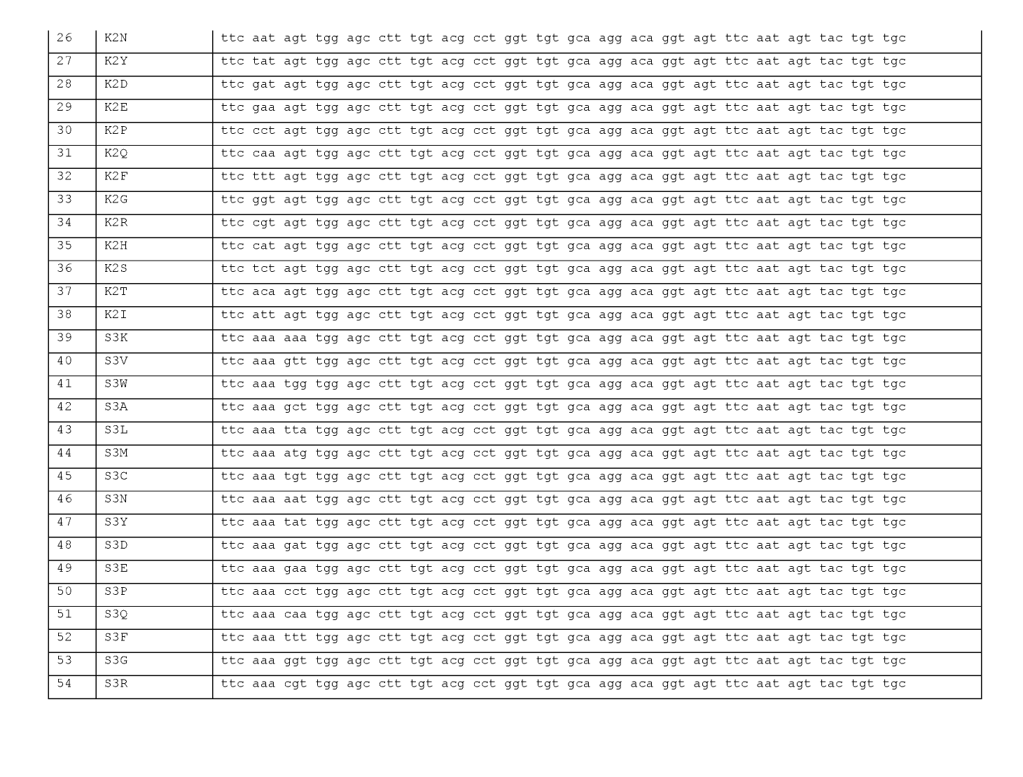

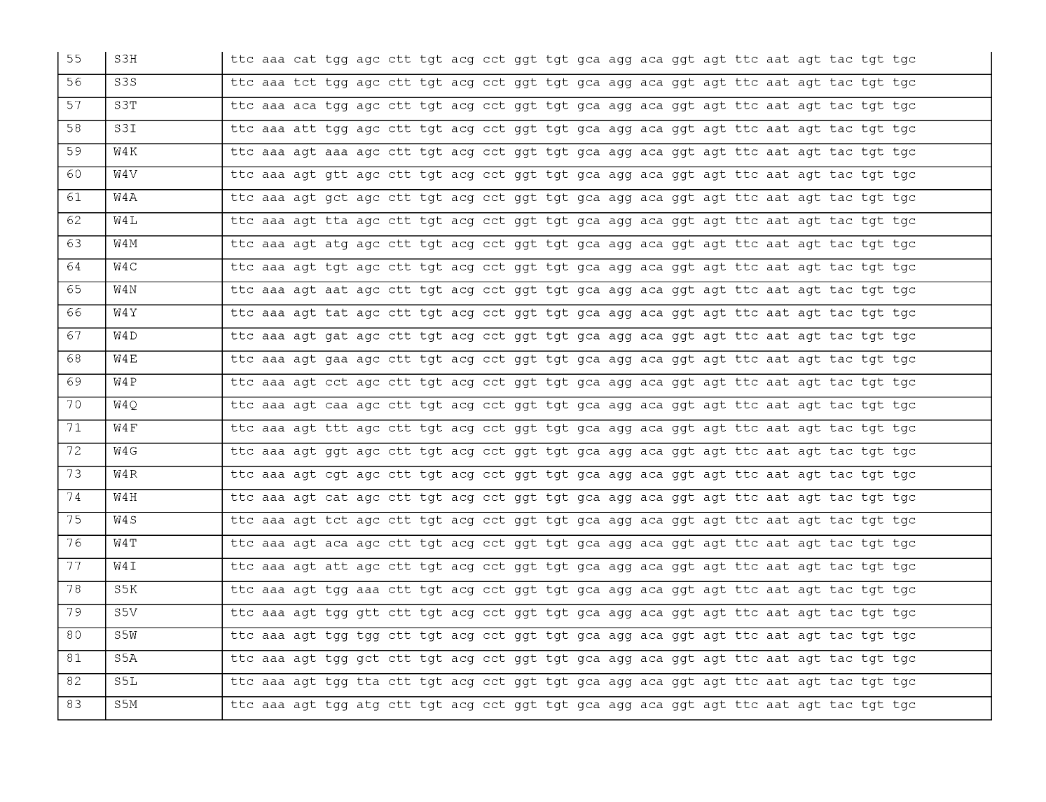

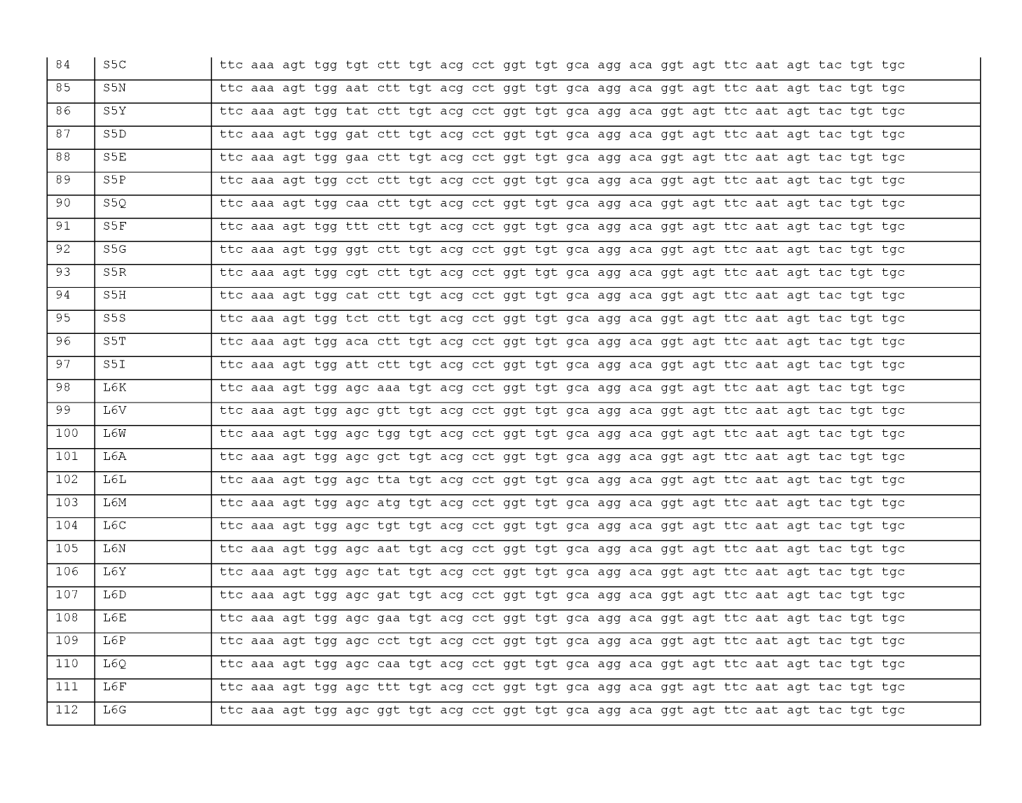

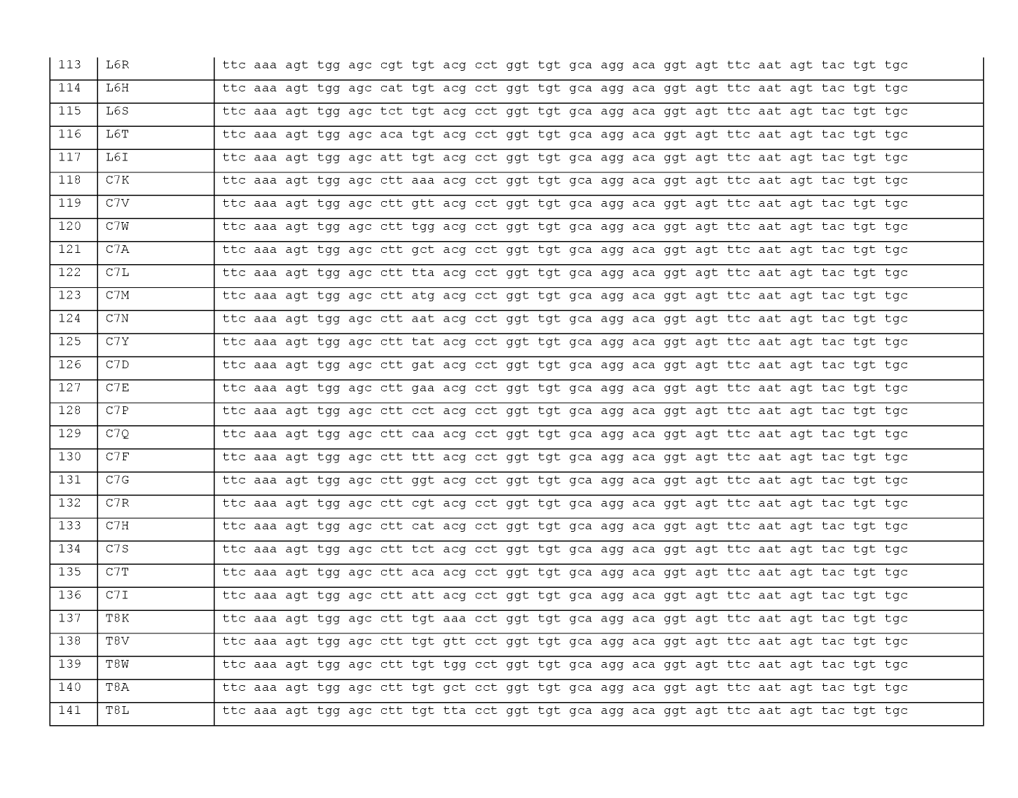

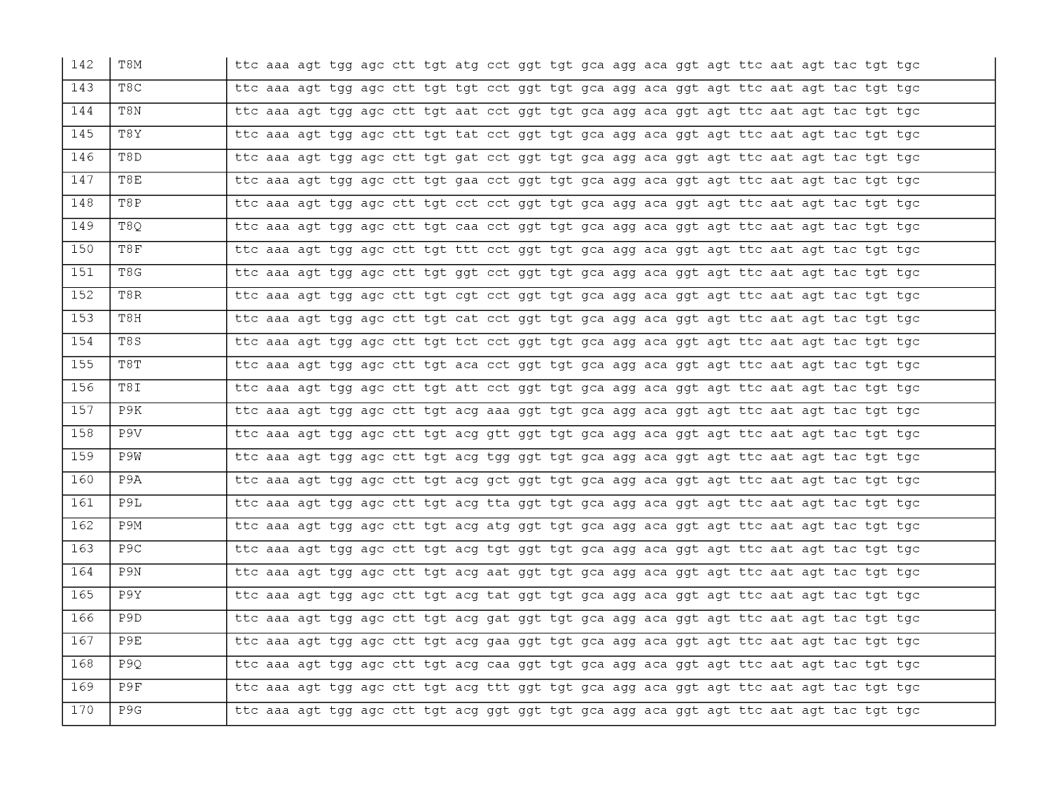

A plasmid for construction of MU1140 variants was constructed using mutA, the primary structural gene encoding the MU1140 propeptide in S. mutans. The episomal plasmid was constructed using plasmid pMK4 (GenBank Accession No.: EU549778.1), which is a plasmid with a ColE1 origin of replication, ampicillin resistance in E. coli, and chloramphenicol resistance in S. mutans. A DNA fragment of S. mutans JH1140 (ATCC Deposit: 55676) containing the mutAA′ locus was ligated into pMK4 and designated pLAN042 (FIG. 2). Plasmid pLAN042 was used as a template to build mutA variants using PCR mutagenesis (Raman and Martin, Nature Methods, 11, 2014), using the codon substitutions listed in Table 1A.

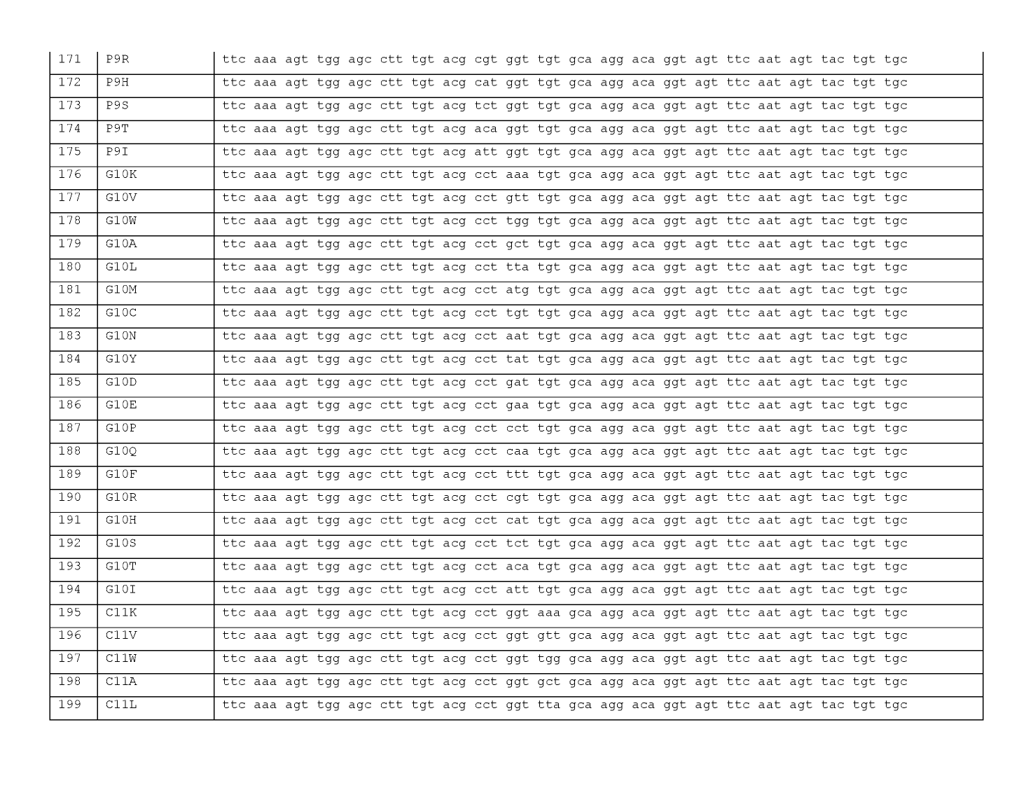

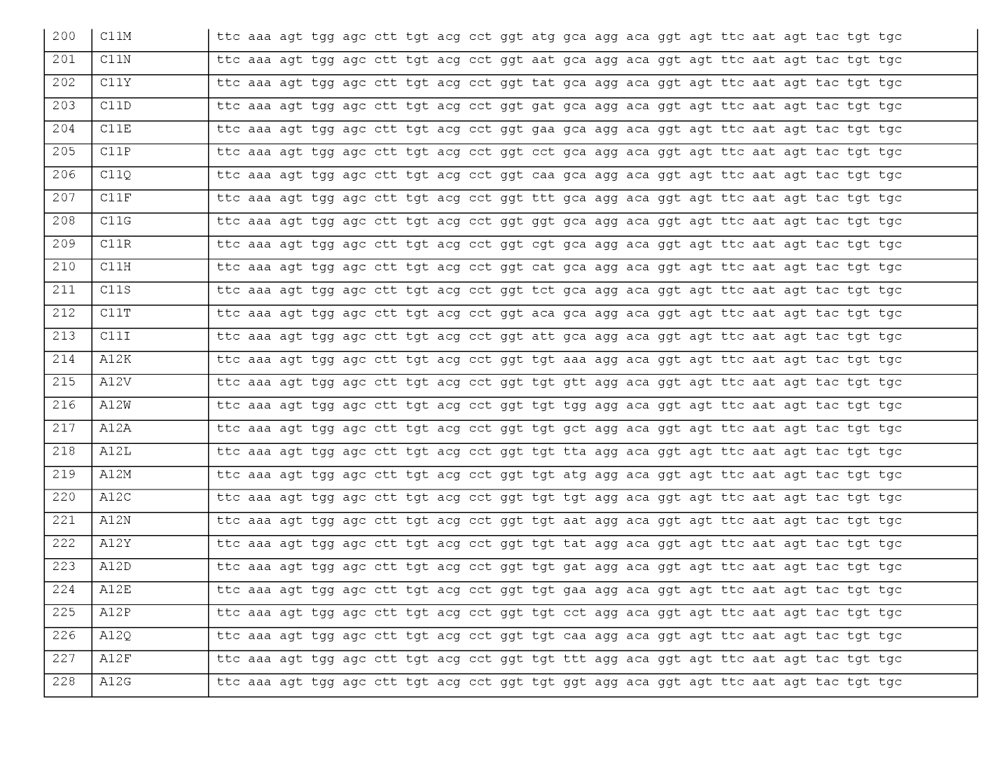

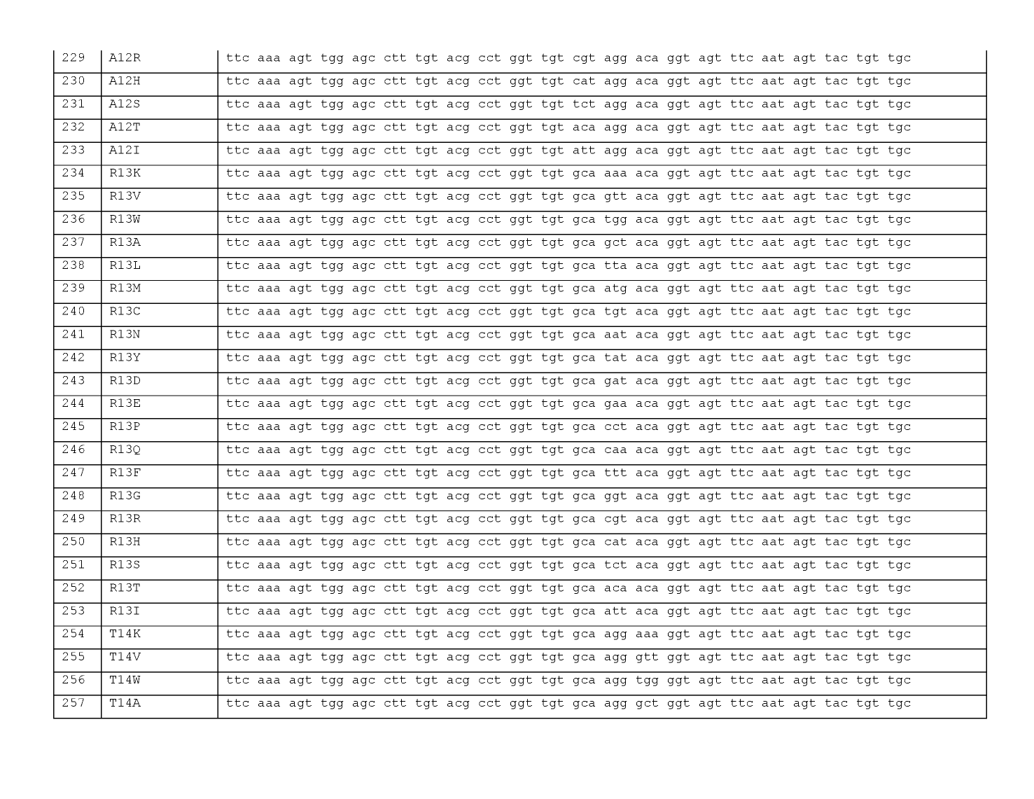

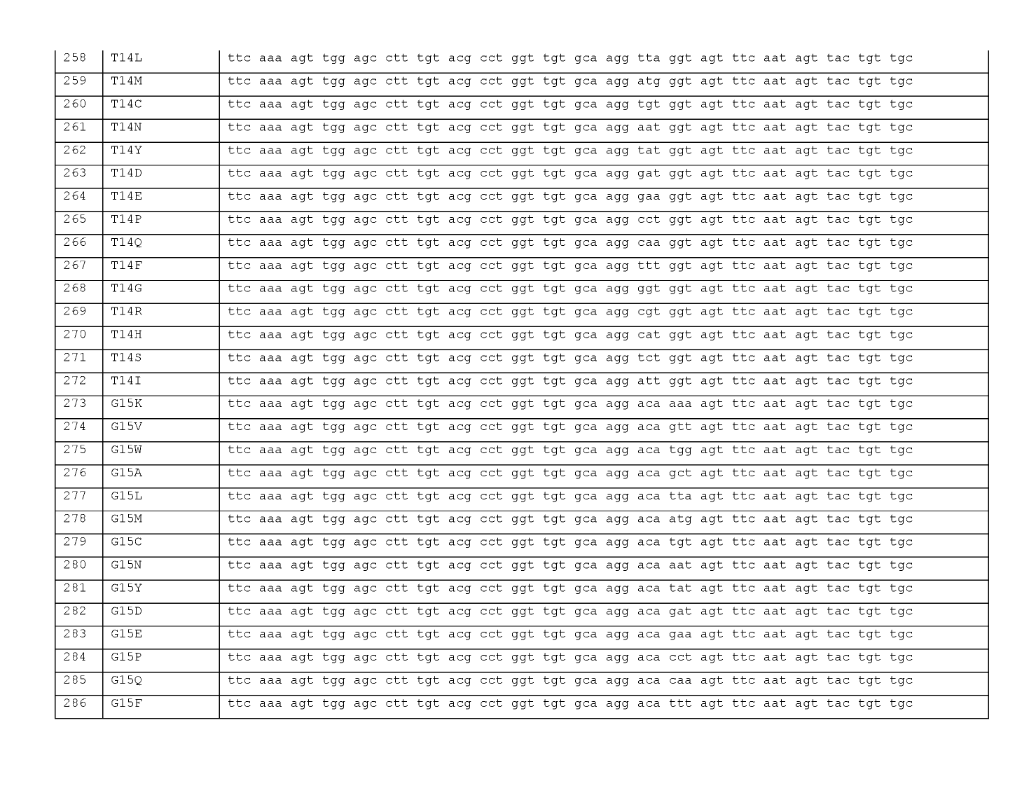

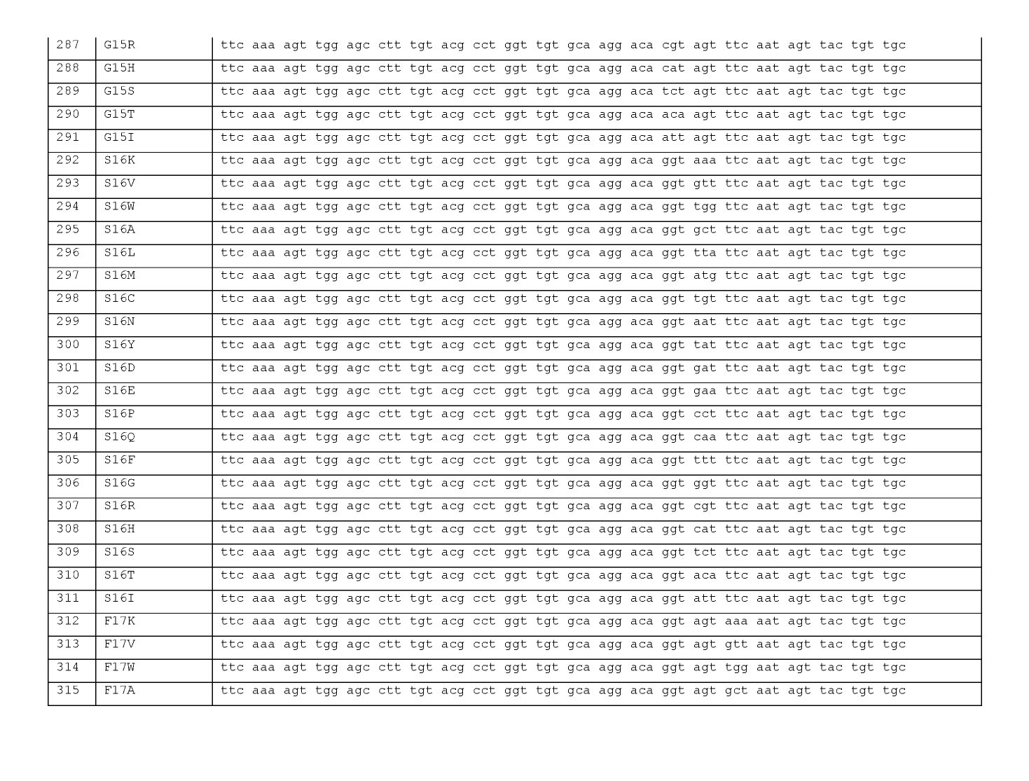

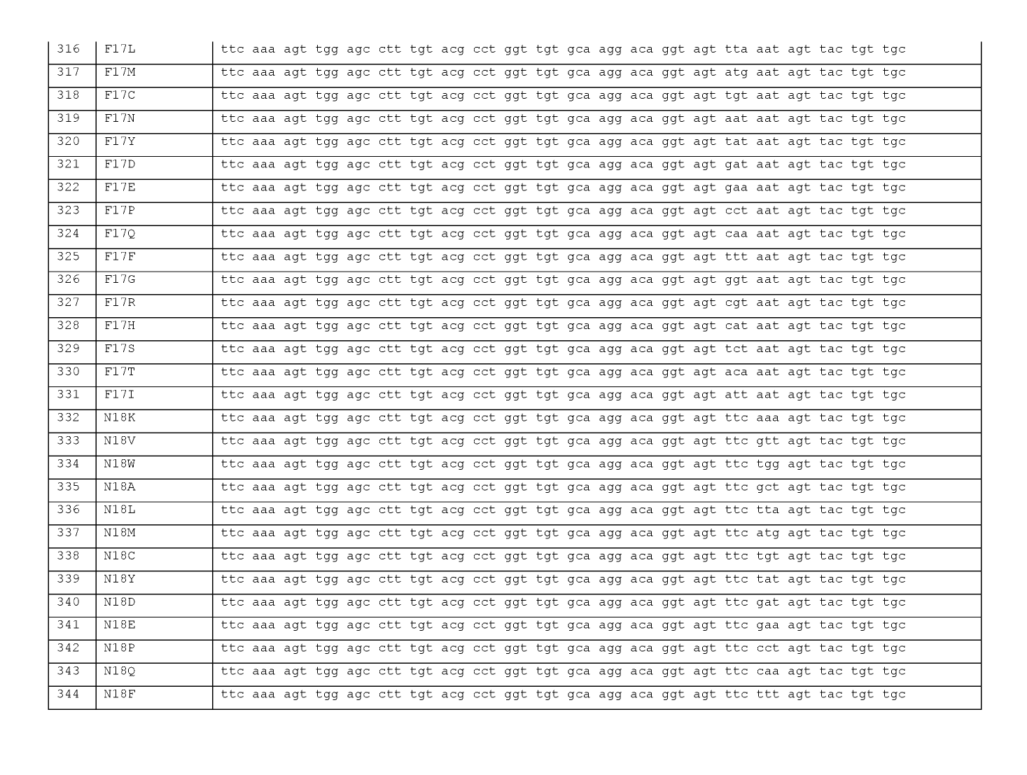

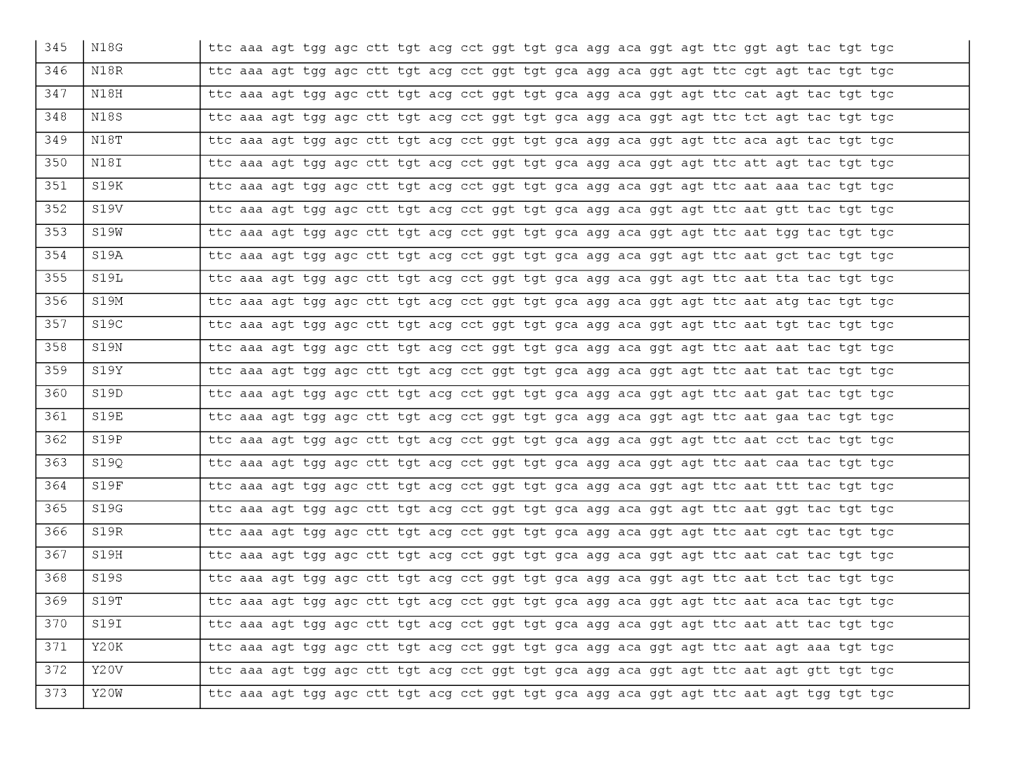

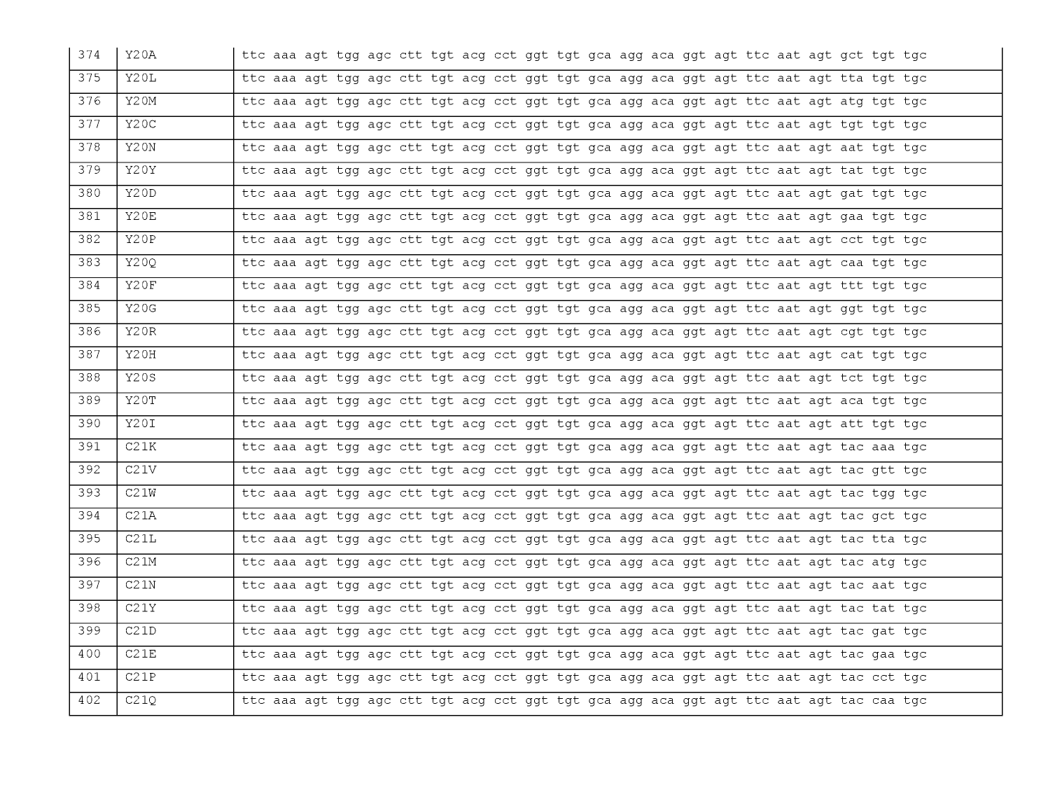

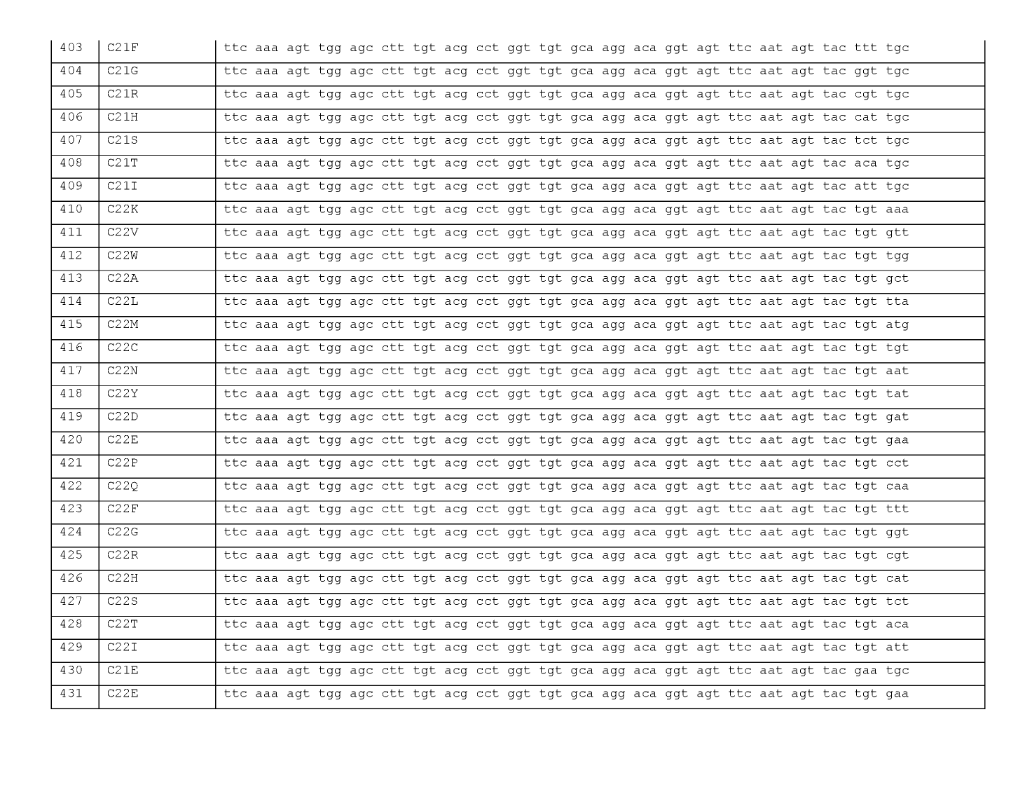

A chromosomal integration strategy using homologous recombination was employed to produce MU1140 variants in S. mutans JH1140 by replacement of the native chromosomal mutA gene with mutA variants encoding codon substitutions. An integration arm upstream of mutAA′, an integration arm downstream of mutAA′, and an erythromycin resistance gene were amplified by PCR and assembled using splicing overlap extension PCR (SOE PCR). The SOE product was ligated to pMK4, transformed into E. coli, verified by DNA sequencing, and designated pLAN126 (FIG. 3).

MutA variant libraries were constructed by PCR amplification of upstream and downstream recombination arms from the pLAN126 template and subsequent assembly with PCR amplified mutA variants from the pLAN042 mutA variant library (Table 1A) or an oligonucleotide library encoding mutA codon variants using SOE PCR (Table IB). A final PCR reaction was used to amplify the single SOE product containing 750 bp recombination arms.

DNA vectors were transformed into S. mutans JH1140 using standard techniques (Biswas et al., Biotechniques 42.4:487 (2007)). Transformants were plated onto TSYEX agar (Ghobrial 2008, Pharmacokinetics and pharmacodynamics of the lantibiotic MU1140. Ph.D. dissertation, University of Florida) containing 3 μg/mL erythromycin, and incubated in a candle jar for 3 days at 37° C. PCR and Sanger DNA sequencing was utilized to confirm replacement of the chromosomal copy of mutA with the mutA variant encoded on the integration vector.

TABLE 1A
Nucleotide identities of mutA variants.

| Mutation | Core peptide sequence | | | | | | | | | | | |
|---|---|---|---|---|---|---|---|---|---|---|---|---|
| MU1140 | TTC | AAA | AGT | TGG | AGC | CTT | TGT | ACG | CCT | GGT | TGT | GCA |
| (Wild-type) | | | | | | | | | | | | |
| SEQ ID NO: 1 | | | | | | | | | | | | |
| F1I | ATT | AAA | AGT | TGG | AGC | CTT | TGT | ACG | CCT | GGT | TGT | GCA |
| SEQ ID NO: 19 | | | | | | | | | | | | |
| F1L | CTT | AAA | AGT | TGG | AGC | CTT | TGT | ACG | CCT | GGT | TGT | GCA |
| SEQ ID NO: 4 | | | | | | | | | | | | |
| R13N | TTC | AAA | AGT | TGG | AGC | CTT | TGT | ACG | CCT | GGT | TGT | GCA |
| SEQ ID NO: 241 | | | | | | | | | | | | |
| F17L | TTC | AAA | AGT | TGG | AGC | CTT | TGT | ACG | CCT | GGT | TGT | GCA |
| SEQ ID NO: 316 | | | | | | | | | | | | |
| F17Y | TTC | AAA | AGT | TGG | AGC | CTT | TGT | ACG | CCT | GGT | TGT | GCA |
| SEQ ID NO: 320 | | | | | | | | | | | | |
| N18A | TTC | AAA | AGT | TGG | AGC | CTT | TGT | ACG | CCT | GGT | TGT | GCA |
| SEQ ID NO: 335 | | | | | | | | | | | | |
| Y20F | TTC | AAA | AGT | TGG | AGC | CTT | TGT | ACG | CCT | GGT | TGT | GCA |
| SEQ ID NO: 384 | | | | | | | | | | | | |

| Mutation | Core peptide sequence | | | | | | | | | |
|---|---|---|---|---|---|---|---|---|---|---|
| MU1140 | AGG | ACA | GGT | AGT | TTC | AAT | AGT | TAC | TGT | TGC |
| (Wild-type) | | | | | | | | | | |
| SEQ ID NO: 1 | | | | | | | | | | |
| F1I | AGG | ACA | GGT | AGT | TTC | AAT | AGT | TAC | TGT | TGC |
| SEQ ID NO: 19 | | | | | | | | | | |
| F1L | AGG | ACA | GGT | AGT | TTC | AAT | AGT | TAC | TGT | TGC |
| SEQ ID NO: 4 | | | | | | | | | | |
| R13N | AAT | ACA | GGT | AGT | TTC | AAT | AGT | TAC | TGT | TGC |
| SEQ ID NO: 241 | | | | | | | | | | |
| F17L | AGG | ACA | GGT | AGT | TTA | AAT | AGT | TAC | TGT | TGC |
| SEQ ID NO: 316 | | | | | | | | | | |
| F17Y | AGG | ACA | GGT | AGT | TAT | AAT | AGT | TAC | TGT | TGC |
| SEQ ID NO: 320 | | | | | | | | | | |
| N18A | AGG | ACA | GGT | AGT | TTC | GCT | AGT | TAC | TGT | TGC |
| SEQ ID NO: 335 | | | | | | | | | | |
| Y20F | AGG | ACA | GGT | AGT | TTC | AAT | AGT | TTT | TGT | TGC |
| SEQ ID NO: 384 | | | | | | | | | | |

TABLE 1B
Nucleotide identities of multisite mutA variants and control strains (single site variants).

| Mutation | Core peptide sequence | | | | | | | | | | | |
|---|---|---|---|---|---|---|---|---|---|---|---|---|
| MU1140 | TTC | AAA | AGT | TGG | AGC | CTT | TGT | ACG | CCT | GGT | TGT | GCA |
| (Wild-type) | | | | | | | | | | | | |
| SEQ ID NO: 1 | | | | | | | | | | | | |
| F1I | ATT | AAA | AGT | TGG | AGC | CTT | TGT | ACG | CCT | GGT | TGT | GCA |
| SEQ ID NO: 19 | | | | | | | | | | | | |
| F1L | CTT | AAA | AGT | TGG | AGC | CTT | TGT | ACG | CCT | GGT | TGT | GCA |
| SEQ ID NO: 4 | | | | | | | | | | | | |
| R13N | TTC | AAA | AGT | TGG | AGC | CTT | TGT | ACG | CCT | GGT | TGT | GCA |
| SEQ ID NO: 241 | | | | | | | | | | | | |
| F17L | TTC | AAA | AGT | TGG | AGC | CTT | TGT | ACG | CCT | GGT | TGT | GCA |
| SEQ ID NO: 316 | | | | | | | | | | | | |
| F17Y | TTC | AAA | AGT | TGG | AGC | CTT | TGT | ACG | CCT | GGT | TGT | GCA |
| SEQ ID NO: 320 | | | | | | | | | | | | |
| N18A | TTC | AAA | AGT | TGG | AGC | CTT | TGT | ACG | CCT | GGT | TGT | GCA |
| SEQ ID NO: 335 | | | | | | | | | | | | |
| Y20F | TTC | AAA | AGT | TGG | AGC | CTT | TGT | ACG | CCT | GGT | TGT | GCA |
| SEQ ID NO: 384 | | | | | | | | | | | | |
| F1A R13A | GCT | AAA | AGT | TGG | AGC | CTT | TGT | ACG | CCT | GGT | TGT | GCA |
| SEQ ID NO: 1167 | | | | | | | | | | | | |
| F1A R13G | GCT | AAA | AGT | TGG | AGC | CTT | TGT | ACG | CCT | GGT | TGT | GCA |
| SEQ ID NO: 708 | | | | | | | | | | | | |
| F1A R13N | GCT | AAA | AGT | TGG | AGC | CTT | TGT | ACG | CCT | GGT | TGT | GCA |
| SEQ ID NO: 709 | | | | | | | | | | | | |
| F1A R13S | GCT | AAA | AGT | TGG | AGC | CTT | TGT | ACG | CCT | GGT | TGT | GCA |
| SEQ ID NO: 710 | | | | | | | | | | | | |
| F1A R13V | GCT | AAA | AGT | TGG | AGC | CTT | TGT | ACG | CCT | GGT | TGT | GCA |
| SEQ ID NO: 711 | | | | | | | | | | | | |
| F1G R13A | GGT | AAA | AGT | TGG | AGC | CTT | TGT | ACG | CCT | GGT | TGT | GCA |
| SEQ ID NO: 712 | | | | | | | | | | | | |
| F1G R13G | GGT | AAA | AGT | TGG | AGC | CTT | TGT | ACG | CCT | GGT | TGT | GCA |
| SEQ ID NO: 713 | | | | | | | | | | | | |
| F1G R13N | GGT | AAA | AGT | TGG | AGC | CTT | TGT | ACG | CCT | GGT | TGT | GCA |
| SEQ ID NO: 714 | | | | | | | | | | | | |
| F1G R13V | GGT | AAA | AGT | TGG | AGC | CTT | TGT | ACG | CCT | GGT | TGT | GCA |
| SEQ ID NO: 715 | | | | | | | | | | | | |
| F1H R13N | CAT | AAA | AGT | TGG | AGC | CTT | TGT | ACG | CCT | GGT | TGT | GCA |
| SEQ ID NO: 716 | | | | | | | | | | | | |
| F1I R13A G15A | ATT | AAA | AGT | TGG | AGC | CTT | TGT | ACG | CCT | GGT | TGT | GCA |
| SEQ ID NO: 717 | | | | | | | | | | | | |
| F1I R13D G15A | ATT | AAA | AGT | TGG | AGC | CTT | TGT | ACG | CCT | GGT | TGT | GCA |
| SEQ ID NO: 718 | | | | | | | | | | | | |
| F1I R13N G15A | ATT | AAA | AGT | TGG | AGC | CTT | TGT | ACG | CCT | GGT | TGT | GCA |
| SEQ ID NO: 719 | | | | | | | | | | | | |
| F1I W4I R13A | ATT | AAA | AGT | ATT | AGC | CTT | TGT | ACG | CCT | GGT | TGT | GCA |
| SEQ ID NO: 720 | | | | | | | | | | | | |
| F1I W4I R13D | ATT | AAA | AGT | ATT | AGC | CTT | TGT | ACG | CCT | GGT | TGT | GCA |
| SEQ ID NO: 721 | | | | | | | | | | | | |
| F1I W4M R13A | ATT | AAA | AGT | ATG | AGC | CTT | TGT | ACG | CCT | GGT | TGT | GCA |
| SEQ ID NO: 722 | | | | | | | | | | | | |
| F1I W4M R13N | ATT | AAA | AGT | ATG | AGC | CTT | TGT | ACG | CCT | GGT | TGT | GCA |
| SEQ ID NO: 723 | | | | | | | | | | | | |
| F1I K2A R13A | ATT | GCT | AGT | TGG | AGC | CTT | TGT | ACG | CCT | GGT | TGT | GCA |
| SEQ ID NO: 724 | | | | | | | | | | | | |
| F1I K2A W4K | ATT | GCT | AGT | AAA | AGC | CTT | TGT | ACG | CCT | GGT | TGT | GCA |
| R13A | | | | | | | | | | | | |
| SEQ ID NO: 725 | | | | | | | | | | | | |
| F1I K2A W4K | ATT | GCT | AGT | AAA | AGC | CTT | TGT | ACG | CCT | GGT | TGT | GCA |
| R13D | | | | | | | | | | | | |
| SEQ ID NO: 726 | | | | | | | | | | | | |
| F1I K2A W4K | ATT | GCT | AGT | AAA | AGC | CTT | TGT | ACG | CCT | GGT | TGT | GCA |
| R13A Y20F | | | | | | | | | | | | |
| SEQ ID NO: 727 | | | | | | | | | | | | |
| F1I L6V R13A | ATT | AAA | AGT | TGG | AGC | GTT | TGT | ACG | CCT | GGT | TGT | GCA |
| SEQ ID NO: 728 | | | | | | | | | | | | |
| F1I R13A | ATT | AAA | AGT | TGG | AGC | CTT | TGT | ACG | CCT | GGT | TGT | GCA |
| SEQ ID NO: 729 | | | | | | | | | | | | |
| F1I R13A Y20F | ATT | AAA | AGT | TGG | AGC | CTT | TGT | ACG | CCT | GGT | TGT | GCA |
| SEQ ID NO: 730 | | | | | | | | | | | | |
| F1I R13D | ATT | AAA | AGT | TGG | AGC | CTT | TGT | ACG | CCT | GGT | TGT | GCA |
| SEQ ID NO: 731 | | | | | | | | | | | | |
| F1I R13D Y20F | ATT | AAA | AGT | TGG | AGC | CTT | TGT | ACG | CCT | GGT | TGT | GCA |
| SEQ ID NO: 732 | | | | | | | | | | | | |
| F1I R13G | ATT | AAA | AGT | TGG | AGC | CTT | TGT | ACG | CCT | GGT | TGT | GCA |
| SEQ ID NO: 733 | | | | | | | | | | | | |
| F1I R13I | ATT | AAA | AGT | TGG | AGC | CTT | TGT | ACG | CCT | GGT | TGT | GCA |
| SEQ ID NO: 734 | | | | | | | | | | | | |
| F1I R13N | ATT | AAA | AGT | TGG | AGC | CTT | TGT | ACG | CCT | GGT | TGT | GCA |
| SEQ ID NO: 735 | | | | | | | | | | | | |
| F1I R13N Y20F | ATT | AAA | AGT | TGG | AGC | CTT | TGT | ACG | CCT | GGT | TGT | GCA |
| SEQ ID NO: 736 | | | | | | | | | | | | |
| F1I R13P | ATT | AAA | AGT | TGG | AGC | CTT | TGT | ACG | CCT | GGT | TGT | GCA |
| SEQ ID NO: 737 | | | | | | | | | | | | |
| F1I R13Q | ATT | AAA | AGT | TGG | AGC | CTT | TGT | ACG | CCT | GGT | TGT | GCA |
| SEQ ID NO: 738 | | | | | | | | | | | | |
| F1I R13S | ATT | AAA | AGT | TGG | AGC | CTT | TGT | ACG | CCT | GGT | TGT | GCA |
| SEQ ID NO: 739 | | | | | | | | | | | | |
| F1I R13V | ATT | AAA | AGT | TGG | AGC | CTT | TGT | ACG | CCT | GGT | TGT | GCA |
| SEQ ID NO: 740 | | | | | | | | | | | | |
| F1I R13E | ATT | AAA | AGT | TGG | AGC | CTT | TGT | ACG | CCT | GGT | TGT | GCA |
| SEQ ID NO: 741 | | | | | | | | | | | | |
| F1L R13A | CTT | AAA | AGT | TGG | AGC | CTT | TGT | ACG | CCT | GGT | TGT | GCA |
| SEQ ID NO: 742 | | | | | | | | | | | | |
| F1L R13A Y20F | CTT | AAA | AGT | TGG | AGC | CTT | TGT | ACG | CCT | GGT | TGT | GCA |
| SEQ ID NO: 743 | | | | | | | | | | | | |
| F1L R13D | CTT | AAA | AGT | TGG | AGC | CTT | TGT | ACG | CCT | GGT | TGT | GCA |
| SEQ ID NO: 744 | | | | | | | | | | | | |
| F1L R13D Y20F | CTT | AAA | AGT | TGG | AGC | CTT | TGT | ACG | CCT | GGT | TGT | GCA |
| SEQ ID NO: 745 | | | | | | | | | | | | |
| F1L R13G | CTT | AAA | AGT | TGG | AGC | CTT | TGT | ACG | CCT | GGT | TGT | GCA |
| SEQ ID NO: 746 | | | | | | | | | | | | |
| F1L R13N | CTT | AAA | AGT | TGG | AGC | CTT | TGT | ACG | CCT | GGT | TGT | GCA |
| SEQ ID NO: 747 | | | | | | | | | | | | |
| F1L R13P | CTT | AAA | AGT | TGG | AGC | CTT | TGT | ACG | CCT | GGT | TGT | GCA |
| SEQ ID NO: 748 | | | | | | | | | | | | |
| FIL R13Q | CTT | AAA | AGT | TGG | AGC | CTT | TGT | ACG | CCT | GGT | TGT | GCA |
| SEQ ID NO: 749 | | | | | | | | | | | | |
| F1L R13N Y20F | CTT | AAA | AGT | TGG | AGC | CTT | TGT | ACG | CCT | GGT | TGT | GCA |
| SEQ ID NO: 750 | | | | | | | | | | | | |
| F1S R13N | AGT | AAA | AGT | TGG | AGC | CTT | TGT | ACG | CCT | GGT | TGT | GCA |
| SEQ ID NO: 751 | | | | | | | | | | | | |
| F1T R13A | ACA | AAA | AGT | TGG | AGC | CTT | TGT | ACG | CCT | GGT | TGT | GCA |
| SEQ ID NO: 752 | | | | | | | | | | | | |
| F1T R13G | ACA | AAA | AGT | TGG | AGC | CTT | TGT | ACG | CCT | GGT | TGT | GCA |
| SEQ ID NO: 753 | | | | | | | | | | | | |
| F1T R13N | ACA | AAA | AGT | TGG | AGC | CTT | TGT | ACG | CCT | GGT | TGT | GCA |
| SEQ ID NO: 754 | | | | | | | | | | | | |
| F1T R13V | ACA | AAA | AGT | TGG | AGC | CTT | TGT | ACG | CCT | GGT | TGT | GCA |
| SEQ ID NO: 755 | | | | | | | | | | | | |
| F1V R13A | GTT | AAA | AGT | TGG | AGC | CTT | TGT | ACG | CCT | GGT | TGT | GCA |
| SEQ ID NO: 756 | | | | | | | | | | | | |
| F1V R13D | GTT | AAA | AGT | TGG | AGC | CTT | TGT | ACG | CCT | GGT | TGT | GCA |
| SEQ ID NO: 757 | | | | | | | | | | | | |
| F1V R13N | GTT | AAA | AGT | TGG | AGC | CTT | TGT | ACG | CCT | GGT | TGT | GCA |
| SEQ ID NO: 758 | | | | | | | | | | | | |
| F1V R13Q | GTT | AAA | AGT | TGG | AGC | CTT | TGT | ACG | CCT | GGT | TGT | GCA |
| SEQ ID NO: 759 | | | | | | | | | | | | |
| F1V R13V | GTT | AAA | AGT | TGG | AGC | CTT | TGT | ACG | CCT | GGT | TGT | GCA |
| SEQ ID NO: 760 | | | | | | | | | | | | |
| F1V R13P | GTT | AAA | AGT | TGG | AGC | CTT | TGT | ACG | CCT | GGT | TGT | GCA |
| SEQ ID NO: 761 | | | | | | | | | | | | |
| F1Y R13D | TAT | AAA | AGT | TGG | AGC | CTT | TGT | ACG | CCT | GGT | TGT | GCA |
| SEQ ID NO: 762 | | | | | | | | | | | | |
| F1Y R13G | TAT | AAA | AGT | TGG | AGC | CTT | TGT | ACG | CCT | GGT | TGT | GCA |
| SEQ ID NO: 763 | | | | | | | | | | | | |

| Mutation | Core peptide sequence | | | | | | | | | |
|---|---|---|---|---|---|---|---|---|---|---|
| MU1140 | AGG | ACA | GGT | AGT | TTC | AAT | AGT | TAC | TGT | TGC |
| (Wild-type) | | | | | | | | | | |
| SEQ ID NO: 1 | | | | | | | | | | |
| F1I | AGG | ACA | GGT | AGT | TTC | AAT | AGT | TAC | TGT | TGC |
| SEQ ID NO: 19 | | | | | | | | | | |
| F1L | AGG | ACA | GGT | AGT | TTC | AAT | AGT | TAC | TGT | TGC |
| SEQ ID NO: 4 | | | | | | | | | | |
| R13N | AAT | ACA | GGT | AGT | TTC | AAT | AGT | TAC | TGT | TGC |
| SEQ ID NO: 241 | | | | | | | | | | |
| F17L | AGG | ACA | GGT | AGT | TTA | AAT | AGT | TAC | TGT | TGC |
| SEQ ID NO: 316 | | | | | | | | | | |
| F17Y | AGG | ACA | GGT | AGT | TAT | AAT | AGT | TAC | TGT | TGC |
| SEQ ID NO: 320 | | | | | | | | | | |
| N18A | AGG | ACA | GGT | AGT | TTC | GCT | AGT | TAC | TGT | TGC |
| SEQ ID NO: 335 | | | | | | | | | | |
| Y20F | AGG | ACA | GGT | AGT | TTC | AAT | AGT | TTT | TGT | TGC |
| SEQ ID NO: 384 | | | | | | | | | | |
| F1A R13A | GCT | ACA | GGT | AGT | TTC | AAT | AGT | TAC | TGT | TGC |
| SEQ ID NO: 1167 | | | | | | | | | | |
| F1A R13G | GGT | ACA | GGT | AGT | TTC | AAT | AGT | TAC | TGT | TGC |
| SEQ ID NO: 708 | | | | | | | | | | |
| F1A R13N | AAT | ACA | GGT | AGT | TTC | AAT | AGT | TAC | TGT | TGC |
| SEQ ID NO: 709 | | | | | | | | | | |
| F1A R13S | AGT | ACA | GGT | AGT | TTC | AAT | AGT | TAC | TGT | TGC |
| SEQ ID NO: 710 | | | | | | | | | | |
| F1A R13V | GTT | ACA | GGT | AGT | TTC | AAT | AGT | TAC | TGT | TGC |
| SEQ ID NO: 711 | | | | | | | | | | |
| F1G R13A | GCT | ACA | GGT | AGT | TTC | AAT | AGT | TAC | TGT | TGC |
| SEQ ID NO: 712 | | | | | | | | | | |
| F1G R13G | GGT | ACA | GGT | AGT | TTC | AAT | AGT | TAC | TGT | TGC |
| SEQ ID NO: 713 | | | | | | | | | | |
| F1G R13N | AAT | ACA | GGT | AGT | TTC | AAT | AGT | TAC | TGT | TGC |
| SEQ ID NO: 714 | | | | | | | | | | |
| F1G R13V | GTT | ACA | GGT | AGT | TTC | AAT | AGT | TAC | TGT | TGC |
| SEQ ID NO: 715 | | | | | | | | | | |
| F1H R13N | AAT | ACA | GGT | AGT | TTC | AAT | AGT | TAC | TGT | TGC |
| SEQ ID NO: 716 | | | | | | | | | | |
| F1I R13A G15A | GCT | ACA | GCT | AGT | TTC | AAT | AGT | TAC | TGT | TGC |
| SEQ ID NO: 717 | | | | | | | | | | |
| F1I R13D G15A | GAT | ACA | GCT | AGT | TTC | AAT | AGT | TAC | TGT | TGC |
| SEQ ID NO: 718 | | | | | | | | | | |
| F1I R13N G15A | AAT | ACA | GCT | AGT | TTC | AAT | AGT | TAC | TGT | TGC |
| SEQ ID NO: 719 | | | | | | | | | | |
| F1I W4I R13A | GCT | ACA | GGT | AGT | TTC | AAT | AGT | TAC | TGT | TGC |
| SEQ ID NO: 720 | | | | | | | | | | |
| F1I W4I R13D | GAT | ACA | GGT | AGT | TTC | AAT | AGT | TAC | TGT | TGC |
| SEQ ID NO: 721 | | | | | | | | | | |
| F1I W4M R13A | GCT | ACA | GGT | AGT | TTC | AAT | AGT | TAC | TGT | TGC |
| SEQ ID NO: 722 | | | | | | | | | | |
| F1I W4M R13N | AAT | ACA | GGT | AGT | TTC | AAT | AGT | TAC | TGT | TGC |
| SEQ ID NO: 723 | | | | | | | | | | |
| F1I K2A R13A | GCT | ACA | GGT | AGT | TTC | AAT | AGT | TAC | TGT | TGC |
| SEQ ID NO: 724 | | | | | | | | | | |
| F1I K2A W4K | GCT | ACA | GGT | AGT | TTC | AAT | AGT | TAC | TGT | TGC |
| R13A | | | | | | | | | | |
| SEQ ID NO: 725 | | | | | | | | | | |
| F1I K2A W4K | GAT | ACA | GGT | AGT | TTC | AAT | AGT | TAC | TGT | TGC |
| R13D | | | | | | | | | | |
| SEQ ID NO: 726 | | | | | | | | | | |
| F1I K2A W4K | GCT | ACA | GGT | AGT | TTC | AAT | AGT | TTT | TGT | TGC |
| R13A Y20F | | | | | | | | | | |
| SEQ ID NO: 727 | | | | | | | | | | |
| F1I L6V R13A | GCT | ACA | GGT | AGT | TTC | AAT | AGT | TAC | TGT | TGC |
| SEQ ID NO: 728 | | | | | | | | | | |
| F1I R13A | GCT | ACA | GGT | AGT | TTC | AAT | AGT | TAC | TGT | TGC |
| SEQ ID NO: 729 | | | | | | | | | | |
| F1I R13A Y20F | GCT | ACA | GGT | AGT | TTC | AAT | AGT | TTT | TGT | TGC |
| SEQ ID NO: 730 | | | | | | | | | | |
| F1I R13D | GAT | ACA | GGT | AGT | TTC | AAT | AGT | TAC | TGT | TGC |
| SEQ ID NO: 731 | | | | | | | | | | |
| F1I R13D Y20F | GAT | ACA | GGT | AGT | TTC | AAT | AGT | TTT | TGT | TGC |
| SEQ ID NO: 732 | | | | | | | | | | |
| F1I R13G | GGT | ACA | GGT | AGT | TTC | AAT | AGT | TAC | TGT | TGC |
| SEQ ID NO: 733 | | | | | | | | | | |
| F1I R13I | ATT | ACA | GGT | AGT | TTC | AAT | AGT | TAC | TGT | TGC |
| SEQ ID NO: 734 | | | | | | | | | | |
| F1I R13N | AAT | ACA | GGT | AGT | TTC | AAT | AGT | TAC | TGT | TGC |
| SEQ ID NO: 735 | | | | | | | | | | |
| F1I R13N Y20F | AAT | ACA | GGT | AGT | TTC | AAT | AGT | TTT | TGT | TGC |
| SEQ ID NO: 736 | | | | | | | | | | |
| F1I R13P | CCT | ACA | GGT | AGT | TTC | AAT | AGT | TAC | TGT | TGC |
| SEQ ID NO: 737 | | | | | | | | | | |
| F1I R13Q | CAA | ACA | GGT | AGT | TTC | AAT | AGT | TAC | TGT | TGC |
| SEQ ID NO: 738 | | | | | | | | | | |
| F1I R13S | AGT | ACA | GGT | AGT | TTC | AAT | AGT | TAC | TGT | TGC |
| SEQ ID NO: 739 | | | | | | | | | | |
| F1I R13V | GTT | ACA | GGT | AGT | TTC | AAT | AGT | TAC | TGT | TGC |
| SEQ ID NO: 740 | | | | | | | | | | |
| F1I R13E | GAA | ACA | GGT | AGT | TTC | AAT | AGT | TAC | TGT | TGC |
| SEQ ID NO: 741 | | | | | | | | | | |
| F1L R13A | GCT | ACA | GGT | AGT | TTC | AAT | AGT | TAC | TGT | TGC |
| SEQ ID NO: 742 | | | | | | | | | | |
| F1L R13A Y20F | GCT | ACA | GGT | AGT | TTC | AAT | AGT | TTT | TGT | TGC |
| SEQ ID NO: 743 | | | | | | | | | | |
| F1L R13D | GAT | ACA | GGT | AGT | TTC | AAT | AGT | TAC | TGT | TGC |
| SEQ ID NO: 744 | | | | | | | | | | |
| F1L R13D Y20F | GAT | ACA | GGT | AGT | TTC | AAT | AGT | TTT | TGT | TGC |
| SEQ ID NO: 745 | | | | | | | | | | |
| F1L R13G | GGT | ACA | GGT | AGT | TTC | AAT | AGT | TAC | TGT | TGC |
| SEQ ID NO: 746 | | | | | | | | | | |
| F1L R13N | AAT | ACA | GGT | AGT | TTC | AAT | AGT | TAC | TGT | TGC |
| SEQ ID NO: 747 | | | | | | | | | | |
| F1L R13P | CCT | ACA | GGT | AGT | TTC | AAT | AGT | TAC | TGT | TGC |
| SEQ ID NO: 748 | | | | | | | | | | |
| FIL R13Q | CAA | ACA | GGT | AGT | TTC | AAT | AGT | TAC | TGT | TGC |
| SEQ ID NO: 749 | | | | | | | | | | |
| F1L R13N Y20F | AAT | ACA | GGT | AGT | TTC | AAT | AGT | TTT | TGT | TGC |
| SEQ ID NO: 750 | | | | | | | | | | |
| F1S R13N | AAT | ACA | GGT | AGT | TTC | AAT | AGT | TAC | TGT | TGC |
| SEQ ID NO: 751 | | | | | | | | | | |
| F1T R13A | GCT | ACA | GGT | AGT | TTC | AAT | AGT | TAC | TGT | TGC |
| SEQ ID NO: 752 | | | | | | | | | | |
| F1T R13G | GGT | ACA | GGT | AGT | TTC | AAT | AGT | TAC | TGT | TGC |
| SEQ ID NO: 753 | | | | | | | | | | |
| F1T R13N | AAT | ACA | GGT | AGT | TTC | AAT | AGT | TAC | TGT | TGC |
| SEQ ID NO: 754 | | | | | | | | | | |
| F1T R13V | GTT | ACA | GGT | AGT | TTC | AAT | AGT | TAC | TGT | TGC |
| SEQ ID NO: 755 | | | | | | | | | | |
| F1V R13A | GCT | ACA | GGT | AGT | TTC | AAT | AGT | TAC | TGT | TGC |
| SEQ ID NO: 756 | | | | | | | | | | |
| F1V R13D | GAT | ACA | GGT | AGT | TTC | AAT | AGT | TAC | TGT | TGC |
| SEQ ID NO: 757 | | | | | | | | | | |
| F1V R13N | AAT | ACA | GGT | AGT | TTC | AAT | AGT | TAC | TGT | TGC |
| SEQ ID NO: 758 | | | | | | | | | | |
| F1V R13Q | CAA | ACA | GGT | AGT | TTC | AAT | AGT | TAC | TGT | TGC |
| SEQ ID NO: 759 | | | | | | | | | | |
| F1V R13V | GTT | ACA | GGT | AGT | TTC | AAT | AGT | TAC | TGT | TGC |
| SEQ ID NO: 760 | | | | | | | | | | |
| F1V R13P | CCT | ACA | GGT | AGT | TTC | AAT | AGT | TAC | TGT | TGC |
| SEQ ID NO: 761 | | | | | | | | | | |
| F1Y R13D | GAT | ACA | GGT | AGT | TTC | AAT | AGT | TAC | TGT | TGC |
| SEQ ID NO: 762 | | | | | | | | | | |
| F1Y R13G | GGT | ACA | GGT | AGT | TTC | AAT | AGT | TAC | TGT | TGC |
| SEQ ID NO: 763 | | | | | | | | | | |

In this manner, a plasmid library of 418 mutA variants was thus constructed using pLAN042 as the base vector. The mutA variant library was constructed in an addressable fashion, with each of the 418 vector designs containing a single codon mutation to confer a designated amino acid change. Confirmation of the codon change for each of the 418 variants was confirmed by DNA sequencing.

Multi-site mutA variants were also constructed using a similar genomic integration strategy to improve levels and consistency of MU1140 variant production.

### Example 2

### Non-Normalized Testing of MU1140 Single and Multi-Site Variants

In order to determine if engineered S. mutans mutA variant strains produce MU1140 variants with antimicrobial activity, mutA variant strain culture supernatants were initially tested for their ability to induce formation of zones of clearing against Micrococcus luteus 272, an MU1140 sensitive antibiotic indicator strain.

### High-Throughput (HTP) Growth

S. mutans colonies were picked into 2.2 mL 96 deep well plates containing 1 mL of TSYEX (3% tryptic soy broth and 0.3% yeast extract) and 3 μg/mL erythromycin. The plates were grown micro-aerobically overnight at 37° C. without shaking. One hundred (100) L of overnight culture was transferred to 2.2 mL 96 well deep well plates containing 900 gAL Ghobrial expression media (5% Yeast Extract, 4% Glucose, 0.1% CaCl₂), 100 mM bis-tris Buffer pH 6.7; Ghobrial, 2008). The expression plates were sealed with porous seals and shaken for 20 hours at 37° C. and 900 rpm. The cell cultures were centrifuged for 10 minutes at 3220×g and the supernatant was removed for testing in the Micrococcus luteus bioassay. Freezer stocks were constructed by diluting overnight TSYEX cultures of S. mutans strains with 25% glycerol (final volume) and incubation at −80° C.

Micrococcus luteus HTP Bioassay

M. luteus (ATCC Deposit No.: 272) was streaked onto an LB agar plate and grown at 30° C. for 48 hours, then 5 mL of LB broth was transferred to a 14 mL culture tube. M. luteus cells were inoculated from plate cultures and grown overnight at 30° C. in a shaker at 250 rpm. Cells were diluted 1:100 using in a volume of 50 mL LB broth in a 250 mL Erlenmeyer flask and grown for 2 hours at 30° C. Subsequently, M. luteus cells were centrifuged for 5 minutes at 3220×g, re-suspended in 5 mL of fresh LB, inoculated into 45° C. molten 125 mL soft nutrient agar (5 g/L Bacto agar, 8 g/L Difco nutrient broth) at OD600 0.1 and poured into a QTray (Genetix). The QTray was dried with the lid removed in a laminar flow hood for 30 minutes. A QPIX™ 450 gridding robot equipped with a 96-metal pin head was used to replicate spot each variant 10 times from a 96-well source plate (shallow Greiner U-bottom) containing 150 μL medium to the QTray. The QTrays were incubated overnight at 30° C. and imaged using an EPSON® Expression 10,000 XL optical scanner. Zones of clearing were scored as less than or equal activity to SM152 (indicated as “1”), and greater than SM152 activity (indicated as “2”).

### Results of MU1140 Single Site Variants (Expressed on Episomal Plasmid)

MU1140 variant producing strains were assessed for improvements in antimicrobial activity using the M. luteus zone of clearing assay using non-normalized culture supernatants obtained from overnight growth of S. mutans strains (Table 2). Consistent (well-to-well and plate-to-plate) zones of clearing were observed for both S. mutans strains JH1140 and SM152. Strain S. mutans SM152 (mutA invariant) and variant producing mutA variant strains contain an erythromycin resistance gene in the mutA′ mutB intergenic region from the pLAN126-based integration vector (FIG. 3). During validation of the SM152 mutA invariant strain, it was found that insertion of the erythromycin resistance cassette caused a consistent gain of function improvement in the size of the zone of activity observed compared to the size of the zone of activity observed in the parent strain JH1140 (Table 2). The increase in antimicrobial activity observed may be due to deregulation of the regulatory region leading to higher levels of expression of the upstream mutAA′ or downstream mutBCDPT operons. Changes in the size of the zone of clearing in this assay could be due to increases in specific antimicrobial activity, stability, titer, and/or diffusibility, or a combination of such factors.

Multi-site MU1140 variant producing strains were subsequently tested to determine if mutation of more than one amino acid position would confer improved performance in the M. luteus zone of clearing assay (Table 3). Multi-site variants tested exhibited larger zones of clearing than SM152 and the single amino acid variant strains F1I and F1L, indicating possible increases in specific antimicrobial activity, stability, titer, and/or diffusibility, or a combination of such factors.

TABLE 2
Non-normalized size of zones of clearing detected in M. luteus zone
of clearing assay. A score of one (1) indicates size of zone of clearing
produced by parental strain JH1140. A score of two (2) indicates zone
of clearing greater in size than JH1140. Strains were replicated
in a minimum of two independent experiments.

| Variant | Zone of clearing 1 | Zone of clearing 2 |
|---|---|---|
| JH1140 | 1 | 1 |
| (wt) | | |
| SM152 | 2 | 2 |
| F1I | 2 | 2 |
| F1L | 2 | 2 |
| R13N | 2 | 2 |
| F17I | 2 | 2 |
| F17L | 2 | 2 |
| N18A | 2 | 2 |
| Y20F | 2 | 2 |

TABLE 3
Non-normalized size of zones of clearing detected in M. luteus zone
of clearing assay. A score of one (1) indicates size of zone of clearing
produced by control strain SM152. A score of two (2) indicates zone
of clearing greater in size than SM152. Strains were replicated in
a minimum of two independent replicate experiments.

| | zone of | zone of |
|---|---|---|
| Variant | clearing 1 | clearing 2 |
| SM152 | 1 | 1 |
| F1I | 1 | 1 |
| F1L | 1 | 1 |
| F1A R13A | 2 | 2 |
| F1A R13V | 2 | 2 |
| F1A R13G | 2 | 2 |
| F1A R13N | 2 | 2 |
| F1A R13S | 2 | 2 |
| F1G R13A | 2 | 2 |
| F1G R13G | 2 | 2 |
| F1G R13N | 2 | 2 |
| F1G R13V | 2 | 2 |
| F1H R13N | 2 | 2 |
| F1I R13A G15A | 2 | 2 |
| F1I R13D G15A | 2 | 2 |
| F1I R13N G15A | 2 | 2 |
| F1I W4I R13A | 2 | 2 |
| F1I W4I R13D | 2 | 2 |
| F1I W4M R13A | 2 | 2 |
| F1I W4M R13N | 2 | 2 |
| F1I K2A R13A | 2 | 2 |
| F1I K2A W4K R13A | 2 | 2 |
| F1I K2A W4K R13D | 2 | 2 |
| F1I K2A W4K R13A Y20F | 2 | 2 |
| F1I L6V R13A | 2 | 2 |
| F1I R13A | 2 | 2 |
| F1I R13A Y20F | 2 | 2 |
| F1I R13D | 2 | 2 |
| F1I R13D Y20F | 2 | 2 |
| F1I R13G | 2 | 2 |
| F1I R13I | 2 | 2 |
| F1I R13N | 2 | 2 |
| F1I R13N Y20F | 2 | 2 |
| F1I R13P | 2 | 2 |
| F1I R13Q | 2 | 2 |
| F1I R13S | 2 | 2 |
| F1I R13V | 2 | 2 |
| F1I R13E | 1 | 1 |
| F1L R13A | 2 | 2 |
| F1L R13A Y20F | 2 | 2 |
| F1L R13D | 2 | 2 |
| F1L R13G | 2 | 2 |
| F1L R13N | 2 | 2 |
| F1L R13P | 2 | 2 |
| F1L R13Q | 2 | 2 |
| F1L R13N Y20F | 2 | 2 |
| F1L R13D Y20F | 2 | 2 |
| F1S R13N | 2 | 2 |
| F1T R13A | 2 | 2 |
| F1T R13G | 2 | 2 |
| F1T R13N | 2 | 2 |
| F1T R13V | 2 | 2 |
| F1V R13A | 2 | 2 |
| F1V R13N | 2 | 2 |
| F1V R13Q | 2 | 2 |
| F1V R13D | 2 | 2 |
| F1V R13P | 2 | 2 |
| F1V R13V | 2 | 2 |
| F1Y R13D | 2 | 2 |
| F1Y R13G | 2 | 2 |

Following transformation of the addressable 418 mutA variant library, two S. mutans SM126 ΔmutAA′ pLAN042 variant clones per mutA variant were picked, expressed and analyzed for antimicrobial activity (see, Table 4, plates 1-6 below). Data was interpreted by qualitative comparison of the size of the zone of clearing for each variant producing strain with the positive control SM126 ΔmutAA′ pLAN042 and the negative control SM126 ΔmutAA′ pMK4 (empty vector). Forty one (41) variants exhibited greater antimicrobial activity than the positive control for at least one of the two clones (Table 4). Seventy three (73) variants exhibited antimicrobial activity equal to the control for at least one of the two clones. Eighty three (83) variants exhibited less activity than the positive control, but still generated a detectable zone of clearing. Two hundred twenty one (221) variants did not generate detectable zones of clearing for either clone tested.

Variation was observed from clone to clone and experiment to experiment using this plasmid-borne complementation system, as controls included in each 96-well plate experiment demonstrated significant variation, from no detectable antimicrobial activity to greater antimicrobial activity than control (Table 5). Individual clones were spot-sequenced to exclude DNA mutation of the pLAN042 plasmid as a source of experimental error.

TABLE 4
ZONES OF CLEARING GENERATED BY MU1140 VARIANTS

| | | | |
|---|---|---|---|
| | | M luteus Zone | M luteus Zone |
| | | of Clearing | of Clearing |
| Well | Mutation | Colony 1 | Colony 2 |

Plate 1

| | | | |
|---|---|---|---|
| A01 | F1K | 1 | 1 |
| A02 | F1N | 2 | 3 |
| A03 | F1R | 2 | 2 |
| A04 | K2A | 2 | 2 |
| A05 | K2P | 0 | 0 |
| A06 | K2T | 0 | 0 |
| A07 | S3M | 0 | 0 |
| A08 | S3G | 0 | 0 |
| A10 | W4Q | 1 | 1 |
| A11 | S5V | 0 | 0 |
| A12 | S5D | 0 | 0 |
| B01 | F1V | 3 | 3 |
| B02 | F1Y | 2 | 1 |
| B03 | F1H | 3 | 2 |
| B04 | K2L | 2 | 1 |
| B05 | K2Q | 2 | 1 |
| B06 | K2I | 2 | 2 |
| B07 | S3C | 0 | 0 |
| B08 | S3R | 0 | 0 |
| B09 | W4M | 3 | 3 |
| B10 | W4F | 3 | 3 |
| B11 | S5W | 0 | 2 |
| B12 | S5P | 0 | 0 |
| C01 | F1W | 0 | 1 |
| C02 | F1D | 0 | 0 |
| C03 | F1S | 3 | 3 |
| C04 | K2M | 2 | 2 |
| C05 | K2F | 2 | 2 |
| C06 | S3K | 0 | 0 |
| C07 | S3N | 0 | 0 |
| C08 | S3H | 0 | 0 |
| C09 | W4C | 0 | 0 |
| C10 | W4R | 0 | 1 |
| C11 | S5A | 0 | 2 |
| C12 | S5Q | 1 | 2 |
| D01 | F1A | 3 | 1 |
| D02 | F1P | 3 | 2 |
| D03 | F1T | 2 | 3 |
| D04 | K2C | 1 | 2 |
| D05 | K2G | 1 | 0 |
| D06 | S3V | 0 | 0 |
| D07 | S3Y | 0 | 0 |
| D09 | W4N | 1 | 0 |
| D10 | W4H | 3 | 2 |
| D11 | S5L | 1 | 2 |
| D12 | S5F | 1 | 0 |
| E01 | F1L | 3 | 2 |
| E02 | F1Q | 1 | 1 |
| E04 | K2N | 2 | 2 |
| E05 | K2R | 0 | 0 |
| E06 | S3W | 0 | 0 |
| E07 | S3D | 0 | 0 |
| E08 | S3T | 1 | 0 |
| E09 | W4Y | 2 | 1 |
| E10 | W4I | 0 | 3 |
| E11 | S5C | 0 | 0 |
| E12 | S5G | 1 | 0 |
| F01 | WT | 2 | 2 |
| F02 | WT | 2 | 2 |
| F03 | WT | 2 | 2 |
| F04 | WT | 2 | 2 |
| F05 | WT | 2 | 2 |
| F06 | WT | 2 | 2 |
| F07 | pMK4 | 0 | 0 |
| F08 | pMK4 | 0 | 0 |
| F09 | pMK4 | 0 | 0 |
| F10 | pMK4 | 0 | 0 |
| F11 | pMK4 | 0 | 0 |
| F12 | pMK4 | 0 | 0 |
| G01 | F1M | 2 | 3 |
| G03 | K2V | 0 | 2 |
| G04 | K2Y | 0 | 1 |
| G05 | K2H | 1 | 2 |
| G06 | S3A | 0 | 0 |
| G07 | S3P | 0 | 0 |
| G08 | W4K | 1 | 1 |
| G09 | W4P | 0 | 0 |
| G10 | S5K | 0 | 0 |
| G11 | S5N | 1 | 0 |
| G12 | S5R | 0 | 0 |
| H01 | F1C | 1 | 0 |
| H02 | F1G | 2 | 3 |
| H03 | K2W | 1 | 0 |
| H04 | K2D | 1 | 0 |
| H05 | K2S | 1 | 2 |
| H06 | S3L | 1 | 0 |
| H07 | S3F | 0 | 0 |
| H08 | W4V | 0 | 2 |
| H09 | EMPTY | | |
| H10 | EMPTY | | |
| H11 | EMPTY | | |
| H12 | EMPTY | | |

Plate 2

| A01 | S5H | 3 | 2 |
|---|---|---|---|
| A02 | L6C | 0 | 1 |
| A03 | L6G | 0 | 1 |
| A04 | C7W | 0 | 1 |
| A05 | C7F | 0 | 0 |
| A06 | T8K | 0 | 0 |
| A07 | T8N | 0 | 0 |
| A08 | T8R | 0 | 0 |
| A09 | P9A | 0 | 0 |
| A10 | P9F | 0 | 0 |
| A11 | G10K | 0 | 0 |
| A12 | G10Y | 0 | 0 |
| B01 | S5T | 2 | 0 |
| B02 | L6N | 2 | 2 |
| B03 | L6R | 2 | 0 |
| B04 | C7A | 1 | 0 |
| B05 | C7G | 0 | 0 |
| B06 | T8V | 0 | 0 |
| B07 | T8Y | 0 | 1 |
| B08 | T8H | 0 | 1 |
| B09 | P9L | 0 | 1 |
| B10 | P9R | 0 | 1 |
| B11 | G10V | 0 | 0 |
| B12 | G10D | 0 | 1 |
| C01 | S5I | 2 | 0 |
| C02 | L6Y | 2 | 0 |
| C03 | L6H | 2 | 2 |
| C04 | C7L | 0 | 1 |
| C05 | C7R | 0 | 2 |
| C06 | T8W | 0 | 1 |
| C07 | T8D | 0 | 1 |
| C08 | T8S | 2 | 2 |
| C09 | P9M | 1 | 2 |
| C10 | P9H | 2 | 2 |
| C11 | G10L | 1 | 1 |
| C12 | G10P | 1 | 1 |
| D01 | L6V | 2 | 0 |
| D02 | L6D | 1 | 0 |
| D03 | L6T | 2 | 3 |
| D04 | C7M | 0 | 2 |
| D05 | C7H | 0 | 1 |
| D06 | T8A | 0 | 2 |
| D07 | T8P | 0 | 1 |
| D09 | P9N | 1 | 2 |
| D10 | P9S | 0 | 1 |
| D11 | G10M | 0 | 1 |
| D12 | G10Q | 0 | 1 |
| E01 | L6W | 3 | 1 |
| E02 | L6P | 0 | 1 |
| E03 | L6I | 0 | 2 |
| E04 | C7D | 0 | 0 |
| E05 | C7S | 0 | 0 |
| E06 | T8L | 0 | 0 |
| E07 | T8Q | 0 | 0 |
| E08 | T8I | 0 | 0 |
| E09 | P9D | 1 | 1 |
| E10 | P9T | 2 | 0 |
| E11 | G10C | 1 | 1 |
| E12 | G10F | 1 | 1 |
| F01 | WT | 2 | 2 |
| F02 | WT | 2 | 2 |
| F03 | WT | 2 | 2 |
| F04 | WT | 2 | 2 |
| F05 | WT | 2 | 2 |
| F06 | WT | 2 | 2 |
| F07 | pMK4 | 0 | 0 |
| F08 | pMK4 | 0 | 0 |
| F09 | pMK4 | 0 | 0 |
| F10 | pMK4 | 0 | 0 |
| F11 | pMK4 | 0 | 0 |
| F12 | pMK4 | 0 | 0 |
| G01 | L6A | 2 | 2 |
| G02 | L6Q | 3 | 0 |
| G03 | C7K | 1 | 0 |
| G04 | C7P | 2 | 0 |
| G05 | C7T | 0 | 0 |
| G06 | T8M | 0 | 2 |
| G07 | T8F | 0 | 0 |
| G08 | P9V | 1 | 0 |
| G09 | P9Q | 0 | 1 |
| G10 | P9I | 0 | 1 |
| G11 | G10N | 0 | 1 |
| G12 | G10R | 0 | 1 |
| H02 | L6F | 1 | 1 |
| H03 | C7V | 1 | 0 |
| H04 | C7Q | 1 | 0 |
| H05 | C7I | 0 | 0 |
| H06 | T8C | 0 | 0 |
| H07 | T8G | 0 | 0 |
| H08 | P9W | 0 | 0 |
| H09 | EMPTY | | |
| H10 | EMPTY | | |
| H11 | EMPTY | | |
| H12 | EMPTY | | |
| | | M Luteus Zone | M Luteus Zone |
| | | of Clearing: | of Clearing: |
| Well | Mutation | Colony 1 | Colony 2 |

Plate 3

| | | | |
|---|---|---|---|
| A01 | C21N | 0 | 0 |
| A02 | C21R | 1 | 0 |
| A03 | C22W | 0 | 0 |
| A04 | C22F | 0 | 0 |
| A05 | S3E | 0 | 0 |
| A06 | G10E | 0 | 0 |
| A07 | N18E | 0 | 0 |
| A08 | W4D | 0 | 0 |
| A09 | L6M | 2 | 2 |
| A10 | P9G | 0 | 0 |
| A11 | A12C | 0 | 0 |
| B01 | C21Y | 0 | 0 |
| B02 | C21H | 0 | 0 |
| B03 | C22A | 0 | 0 |
| B04 | C22R | 0 | 0 |
| B05 | W4E | 0 | 0 |
| B06 | C11E | 2 | 3 |
| B07 | S19E | 0 | 0 |
| B08 | W4G | 0 | 0 |
| B09 | L6S | 1 | 1 |
| B10 | G10W | 1 | 0 |
| B11 | A12P | 1 | 1 |
| B12 | R13T | 1 | 2 |
| C01 | C21D | 0 | 1 |
| C02 | C21S | 0 | 1 |
| C03 | C22L | 1 | 0 |
| C04 | C22S | 0 | 0 |
| C05 | S5E | 0 | 0 |
| C06 | R13E | 0 | 3 |
| C07 | Y20E | 1 | 1 |
| C08 | W4S | 0 | 1 |
| C09 | C7N | 0 | 0 |
| C10 | G10A | 1 | 1 |
| C11 | A12G | 2 | 1 |
| C12 | G15K | 1 | 0 |
| D01 | C21P | 1 | 0 |
| D02 | C21T | 0 | 2 |
| D03 | C22M | 2 | 0 |
| D04 | C22T | 0 | 0 |
| D05 | L6E | 0 | 1 |
| D06 | T14E | 0 | 2 |
| D07 | C21E | 0 | 1 |
| D08 | S5M | 2 | 2 |
| D09 | P9K | 1 | 2 |
| D10 | GIOS | 2 | 1 |
| D11 | A12T | 1 | 3 |
| D12 | G15W | 1 | 0 |
| E01 | C21Q | 0 | 1 |
| E02 | C21I | 2 | 0 |
| E04 | C22I | 0 | 0 |
| E05 | C7E | 0 | 0 |
| E06 | G15E | 0 | 1 |
| E07 | C22E | 0 | 1 |
| E08 | S5Y | 2 | 0 |
| E09 | P9C | 2 | 1 |
| E10 | G10I | 2 | 1 |
| Ell | R13V | 1 | 1 |
| E12 | G15A | 1 | 3 |
| F01 | WT | 2 | 2 |
| F02 | WT | 2 | 2 |
| F03 | WT | 2 | 2 |
| F04 | WT | 2 | 2 |
| F05 | WT | 2 | 2 |
| F06 | WT | 2 | 2 |
| F07 | pMK4 | 0 | 0 |
| F08 | pMK4 | 0 | 0 |
| F09 | pMK4 | 0 | 0 |
| F10 | pMK4 | 0 | 0 |
| F11 | pMK4 | 0 | 0 |
| F12 | pMK4 | 0 | 0 |
| G01 | C21F | 0 | 0 |
| G02 | C22K | 0 | 0 |
| G03 | C22D | 0 | 1 |
| G04 | F1E | 0 | 1 |
| G05 | T8E | 0 | 1 |
| G06 | S16E | 0 | 0 |
| G07 | S3Q | 0 | 0 |
| G09 | P9Y | 1 | 0 |
| G10 | C11A | 2 | 3 |
| G11 | R13N | 2 | 1 |
| G12 | G15D | 1 | 0 |
| H01 | C21G | 0 | 2 |
| H02 | C22V | 0 | 0 |
| H03 | C22P | 0 | 0 |
| H04 | K2E | 2 | 3 |
| H05 | P9E | 1 | 2 |
| H06 | F17E | 1 | 0 |
| H07 | W4L | 1 | 3 |
| H08 | L6K | 0 | 0 |
| H09 | EMPTY | | |
| H10 | EMPTY | | |
| H11 | EMPTY | | |
| H12 | EMPTY | | |

Plate 4

| A01 | G10H | 0 | 0 |
|---|---|---|---|
| A02 | C11N | 0 | 0 |
| A03 | C11R | 0 | 0 |
| A04 | A12W | 0 | 0 |
| A05 | A12Q | 0 | 0 |
| A06 | R13W | 1 | 0 |
| A07 | R13Q | 0 | 0 |
| A08 | T14A | 0 | 0 |
| A09 | T14P | 0 | 0 |
| A10 | T14S | 0 | 0 |
| A11 | G15Y | 0 | 0 |
| A12 | G15T | 0 | 0 |
| B01 | G10T | 0 | 0 |
| B02 | C11Y | 0 | 0 |
| B03 | C11H | 0 | 0 |
| B05 | A12F | 0 | 0 |
| B06 | R13A | 2 | 0 |
| B07 | R13F | 0 | 3 |
| B08 | T14L | 0 | 0 |
| B09 | T14Q | 2 | 0 |
| B10 | G15V | 0 | 0 |
| B11 | G15P | 0 | 0 |
| B12 | S16K | 0 | 0 |
| C01 | C11K | 0 | 0 |
| C02 | C11D | 0 | 0 |
| C03 | C11S | 0 | 0 |
| C04 | A12L | 0 | 0 |
| C05 | A12R | 0 | 0 |
| C06 | R13L | 0 | 0 |
| C08 | T14M | 0 | 0 |
| C09 | T14F | 0 | 0 |
| C10 | G15L | 0 | 0 |
| C11 | G15Q | 0 | 0 |
| C12 | S16V | 0 | 0 |
| D01 | C11V | 0 | 0 |
| D02 | C11P | 0 | 0 |
| D03 | C11T | 0 | 0 |
| D04 | A12M | 0 | 0 |
| D05 | A12H | 0 | 0 |
| D06 | R13M | 0 | 0 |
| D07 | R13H | 0 | 0 |
| D08 | T14C | 0 | 0 |
| D09 | T14G | 0 | 0 |
| D10 | G15M | 0 | 0 |
| D11 | G15F | 0 | 0 |
| D12 | S16W | 0 | 0 |
| E01 | C11W | 0 | 0 |
| E02 | C11Q | 2 | 0 |
| E03 | c11I | 0 | 0 |
| E04 | A12N | 0 | 0 |
| E05 | A12S | 0 | 0 |
| E06 | R13C | 0 | 0 |
| E07 | R13S | 2 | 3 |
| E08 | T14N | 0 | 0 |
| E09 | T14R | 0 | 0 |
| E10 | G15C | 0 | 0 |
| E11 | G15H | 0 | 0 |
| E12 | S16A | 0 | 0 |
| F01 | WT | 2 | 2 |
| F02 | WT | 2 | 2 |
| F03 | WT | 2 | 2 |
| F04 | WT | 2 | 2 |
| F05 | WT | 2 | 2 |
| F06 | WT | 2 | 2 |
| F07 | pMK4 | 0 | 0 |
| F08 | pMK4 | 0 | 0 |
| F09 | pMK4 | 0 | 0 |
| F10 | pMK4 | 0 | 0 |
| F11 | pMK4 | 0 | 0 |
| F12 | pMK4 | 0 | 0 |
| G01 | C11L | 0 | 0 |
| G02 | C11F | 0 | 0 |
| G03 | A12K | 0 | 0 |
| G04 | A12Y | 0 | 0 |
| G05 | A12I | 0 | 0 |
| G06 | R13Y | 0 | 0 |
| G07 | R13I | 0 | 0 |
| G08 | T14Y | 0 | 0 |
| G09 | T14H | 0 | 0 |
| G10 | G15N | 2 | 3 |
| G11 | G15S | 2 | 3 |
| G12 | S16L | 0 | 0 |
| H01 | C11M | 0 | 0 |
| H02 | C11G | 2 | 0 |
| H03 | A12V | 2 | 0 |
| H04 | A12D | 2 | 0 |
| H05 | R13K | 0 | 0 |
| H06 | R13P | 0 | 0 |
| H07 | T14K | 0 | 0 |
| H08 | T14D | 0 | 0 |
| H09 | EMPTY | | |
| H10 | EMPTY | | |
| H11 | EMPTY | | |
| H12 | EMPTY | | |
| | | M luteus Zone | M luteus Zone |
| | | of Clearing | of Clearing |
| Well | Mutation | Colony 1 | Colony 2 |

Plate 5

| | | | |
|---|---|---|---|
| A01 | S16M | 0 | 0 |
| A02 | S16G | 0 | 0 |
| A03 | F17W | 0 | 0 |
| A04 | F17Q | 2 | 0 |
| A05 | N18V | 0 | 0 |
| A06 | N18D | 0 | 0 |
| A07 | N18T | 0 | 0 |
| A08 | S19D | 0 | 0 |
| A09 | S19S | 1 | 2 |
| A10 | Y20M | 1 | 2 |
| A11 | Y20F | 3 | 3 |
| A12 | Y20I | 1 | 0 |
| B01 | S16C | 0 | 0 |
| B02 | S16R | 0 | 0 |
| B03 | F17L | 2 | 0 |
| B05 | N18W | 0 | 0 |
| B06 | N18P | 0 | 0 |
| B07 | S19V | 0 | 0 |
| B08 | S19P | 0 | 0 |
| B09 | S19T | 0 | 0 |
| B10 | Y20C | 0 | 0 |
| B11 | Y20G | 0 | 0 |
| B12 | C21V | 2 | 0 |
| C01 | S16N | 0 | 0 |
| C02 | S16H | 0 | 0 |
| C03 | F17C | 0 | 0 |
| C04 | F17G | 0 | 0 |
| C05 | N18A | 2 | 0 |
| C06 | N18Q | 0 | 0 |
| C07 | S19A | 0 | 0 |
| C08 | S19Q | 0 | 0 |
| C09 | Y20K | 0 | 0 |
| C10 | Y20N | 0 | 0 |
| C11 | Y20R | 0 | 0 |
| C12 | C21W | 0 | 0 |
| D01 | S16Y | 2 | 0 |
| D02 | S16T | 0 | 0 |
| D03 | F17N | 0 | 0 |
| D04 | F17H | 2 | 3 |
| D05 | N18L | 0 | 0 |
| D06 | N18F | 0 | 0 |
| D07 | S19M | 0 | 0 |
| D08 | S19F | 0 | 0 |
| D09 | Y20V | 0 | 0 |
| D11 | Y20H | 0 | 0 |
| D12 | C21A | 0 | 0 |
| E01 | S16D | 0 | 0 |
| E02 | S16I | 0 | 0 |
| E03 | F17Y | 2 | 3 |
| E04 | F17S | 0 | 0 |
| E05 | N18M | 0 | 0 |
| E06 | N18G | 0 | 0 |
| E07 | S19C | 0 | 0 |
| E08 | S19G | 0 | 0 |
| E09 | Y20W | 0 | 0 |
| E10 | Y20D | 0 | 0 |
| E11 | Y20S | 0 | 0 |
| E12 | C21L | 1 | 0 |
| F01 | WT | 2 | 2 |
| F02 | WT | 2 | 2 |
| F03 | WT | 2 | 2 |
| F04 | WT | 2 | 2 |
| F05 | WT | 2 | 2 |
| F06 | WT | 2 | 2 |
| F07 | pMK4 | 0 | 0 |
| F08 | pMK4 | 0 | 0 |
| F09 | pMK4 | 0 | 0 |
| F10 | pMK4 | 0 | 0 |
| F11 | pMK4 | 0 | 0 |
| F12 | pMK4 | 0 | 0 |
| G01 | S16P | 0 | 0 |
| G02 | F17K | 0 | 0 |
| G03 | F17D | 0 | 0 |
| G04 | F17T | 2 | 0 |
| G05 | N18C | 0 | 0 |
| G06 | N18H | 0 | 0 |
| G07 | S19N | 0 | 0 |
| G08 | S19R | 0 | 0 |
| G09 | Y20L | 2 | 0 |
| G10 | Y20Q | 0 | 0 |
| G11 | Y20T | 0 | 0 |
| G12 | C21M | 0 | 0 |
| H01 | S16Q | 0 | 0 |
| H02 | F17V | 0 | 0 |
| H03 | F17P | 0 | 0 |
| H04 | F17I | 2 | 0 |
| H05 | N18Y | 0 | 0 |
| H06 | N18S | 0 | 0 |
| H07 | S19Y | 0 | 0 |
| H08 | S19H | 0 | 0 |
| H09 | EMPTY | | |
| H10 | EMPTY | | |
| H11 | EMPTY | | |
| H12 | EMPTY | | |

Plate 6

| | | | |
|---|---|---|---|
| A01 | G15R | 0 | 0 |
| A02 | N18K | 0 | 0 |
| A03 | Y20A | 1 | 0 |
| A04 | A12E | 0 | 2 |
| A05 | T14I | 0 | 0 |
| A06 | R13Q | 1 | 3 |
| A07 | T14P | 0 | 3 |
| A08 | T14S | 2 | 0 |
| A09 | G15Y | 0 | 0 |
| A10 | G15P | 0 | 0 |
| A11 | S16K | 0 | 0 |
| A12 | R13L | 3 | 3 |
| B01 | G15I | 2 | 2 |
| B02 | N18R | 0 | 0 |
| B03 | C21K | 0 | 0 |
| B04 | S3I | 0 | 0 |
| B05 | Y20P | 0 | 0 |
| B06 | C11V | 0 | 0 |
| B07 | R13M | 0 | 0 |
| B08 | A12I | 0 | 0 |
| B09 | T14H | 2 | 1 |
| B11 | C21M | 0 | 1 |
| B12 | EMPTY | 0 | 0 |
| C01 | S16F | 0 | 0 |
| C02 | N18I | 0 | 1 |
| C03 | C22N | 0 | 1 |
| C04 | W4T | 1 | 0 |
| C05 | EMPTY | 0 | 0 |
| C06 | EMPTY | 0 | 0 |
| C07 | EMPTY | 0 | 0 |
| C08 | EMPTY | 0 | 0 |
| C09 | EMPTY | 0 | 0 |
| C10 | EMPTY | 0 | 0 |
| C11 | EMPTY | 0 | 0 |
| C12 | EMPTY | 0 | 0 |
| D02 | S19K | 0 | 0 |
| D03 | C22Y | 0 | 0 |
| D04 | C7Y | 0 | 0 |
| D05 | EMPTY | 0 | 0 |
| D06 | EMPTY | 0 | 0 |
| D07 | EMPTY | 0 | 0 |
| D08 | EMPTY | 0 | 0 |
| D09 | EMPTY | 0 | 0 |
| D10 | EMPTY | 0 | 0 |
| D11 | EMPTY | 0 | 0 |
| D12 | EMPTY | 0 | 0 |
| E01 | F17A | 3 | 0 |
| E02 | S19W | 0 | 0 |
| E03 | C22Q | 0 | 0 |
| E04 | R13G | 0 | 0 |
| E05 | EMPTY | 0 | 0 |
| E06 | EMPTY | 0 | 0 |
| E07 | EMPTY | 0 | 0 |
| E08 | EMPTY | 0 | 0 |
| E09 | EMPTY | 0 | 0 |
| E10 | EMPTY | 0 | 0 |
| E11 | EMPTY | 0 | 0 |
| E12 | EMPTY | 0 | 0 |
| F01 | WT | 2 | 2 |
| F02 | WT | 2 | 2 |
| F03 | WT | 2 | 2 |
| F04 | WT | 2 | 2 |
| F05 | WT | 2 | 2 |
| F06 | WT | 2 | 2 |
| F07 | pMK4 | 0 | 0 |
| F08 | pMK4 | 0 | 0 |
| F09 | pMK4 | 0 | 0 |
| F10 | pMK4 | 0 | 0 |
| F11 | pMK4 | 0 | 0 |
| F12 | pMK4 | 0 | 0 |
| G01 | F17M | 3 | 0 |
| G02 | S19L | 0 | 0 |
| G03 | C22G | 0 | 0 |
| G04 | T14V | 0 | 0 |
| G05 | EMPTY | 0 | 0 |
| G06 | EMPTY | 0 | 0 |
| G07 | EMPTY | 0 | 0 |
| G08 | EMPTY | 0 | 0 |
| G09 | EMPTY | 0 | 0 |
| G10 | EMPTY | 0 | 0 |
| G11 | EMPTY | 0 | 0 |
| G12 | EMPTY | 0 | 0 |
| H01 | F17R | 3 | 2 |
| H02 | S19I | 0 | 0 |
| H03 | C22H | 0 | 0 |
| H04 | T14W | 0 | 0 |
| H05 | EMPTY | 0 | 0 |
| H06 | EMPTY | 0 | 0 |
| H07 | EMPTY | 0 | 0 |
| H08 | EMPTY | 0 | 0 |
| H09 | EMPTY | 0 | 0 |
| H10 | EMPTY | 0 | 0 |
| H11 | EMPTY | 0 | 0 |
| H12 | EMPTY | 0 | 0 |

Zones were scored as 0 (no zone of clearing), 1 (zone of clearing smaller than positive control), 2 (zone of clearing same as positive control), or 3 (zone of clearing larger than positive control).

TABLE 5
INCONSISTENCY OBSERVED IN M. LUTEUS ZONE OF
CLEARING SIZES BY IDENTICAL GENOTYPES GROWN IN
DIFFERENT 96-WELL PLATES AND WELLS

| | | | | |
|---|---|---|---|---|
| plate 1 | G02 | F1F | 1 | 2 |
| plate 1 | D08 | S3S | 2 | 0 |
| plate 3 | G08 | S5S | 3 | 2 |
| plate 2 | H01 | L6L | 1 | 1 |
| plate 2 | D08 | T8T | 0 | 1 |
| plate 4 | B04 | A12A | 0 | 0 |
| plate 4 | C07 | R13R | 0 | 0 |
| Plate 6 | D01 | S16S | 1 | 0 |
| plate 5 | B04 | F17F | 0 | 3 |
| plate 5 | A09 | S19S | 1 | 2 |
| plate 5 | D10 | Y20Y | 0 | 0 |
| plate 3 | E03 | C22C | 1 | 0 |

Construction and testing of the saturated scanning mutagenesis library containing 418 mutA variants enables unbiased sampling of the MU1140 amino acid sequence for screening of variants that confer improved antimicrobial activity against a sensitive indicator microbe (i.e., M. luteus). The mutA variant plasmid expression system used in this study may not be sufficiently robust for the determination of differential changes in antimicrobial activity, as experimental variation was observed using this plasmid-based system. Antimicrobial activity levels from plasmid-based mutA expression were low, inconsistent and not normalized against levels of protein production (e.g., see Table 5). Without wishing to be bound by any particular theory, Applicants contemplate that certain MU1140 variants set forth in Table 1 may have such high levels of antibiotic/antimicrobial activity that these variants may severely inhibit or kill the S. mutans SM126 host cell.

In total, 41 mutA variants generated larger zones of clearing relative to the positive control strain (i.e., SM126 ΔmutAA′) for at least one of the two clones tested, representing 12 of the 22 possible amino acid positions of the MU1140 polypeptide. As set forth in Example 2 below, genomic integration of mutA variants was selected as an alternative expression strategy to improve levels and consistency of MU1140 variant production.

Results of MU1140 Single Site Variants (Stably Integrated into Chromosome)

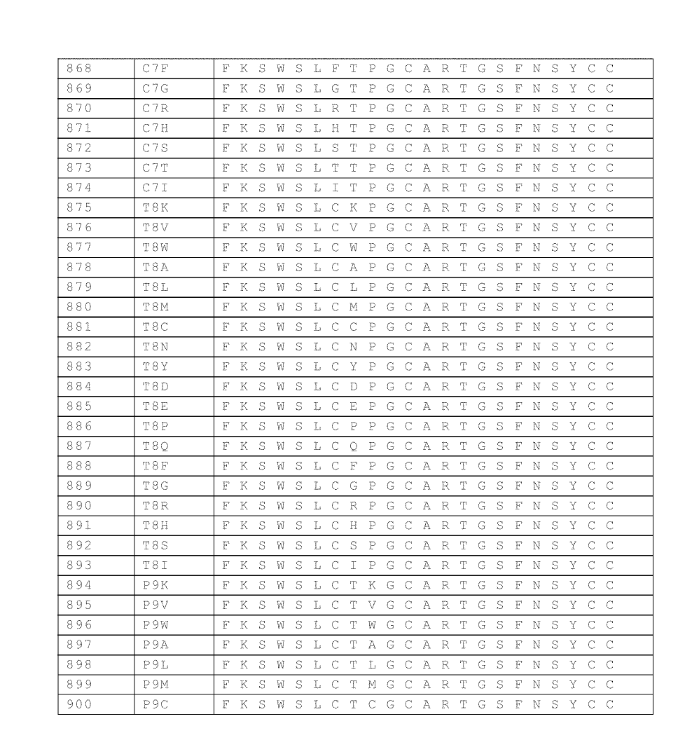

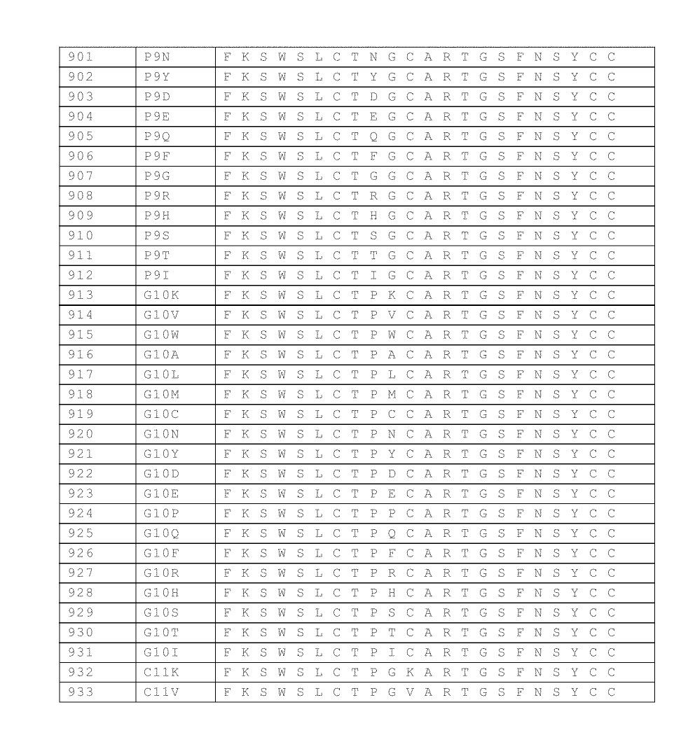

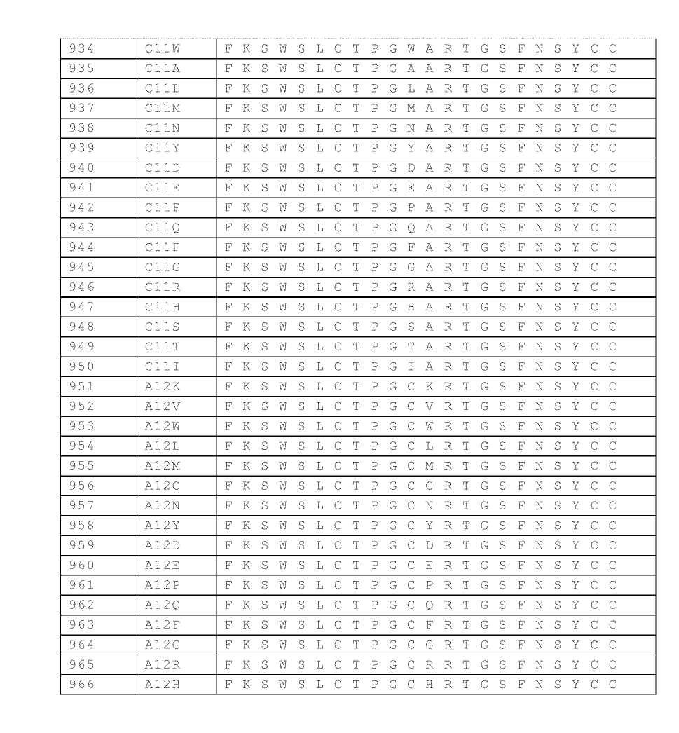

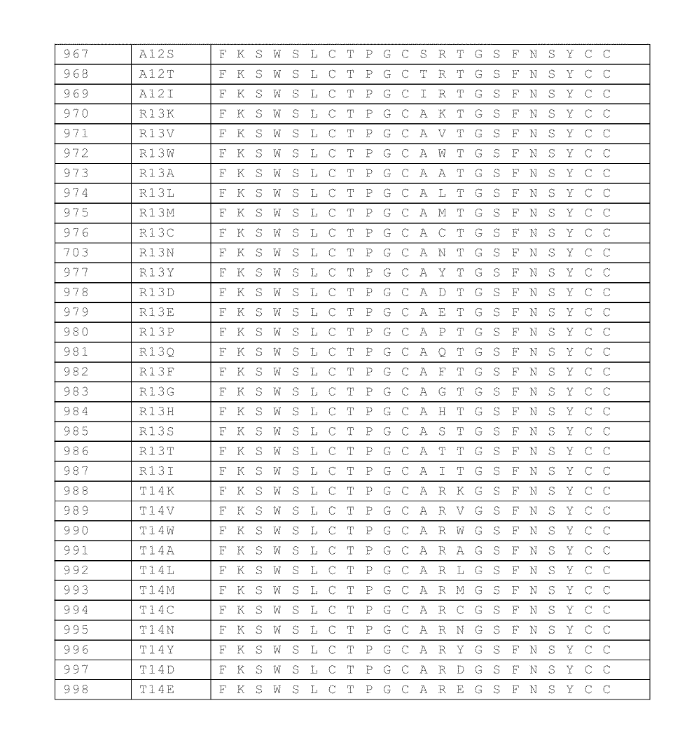

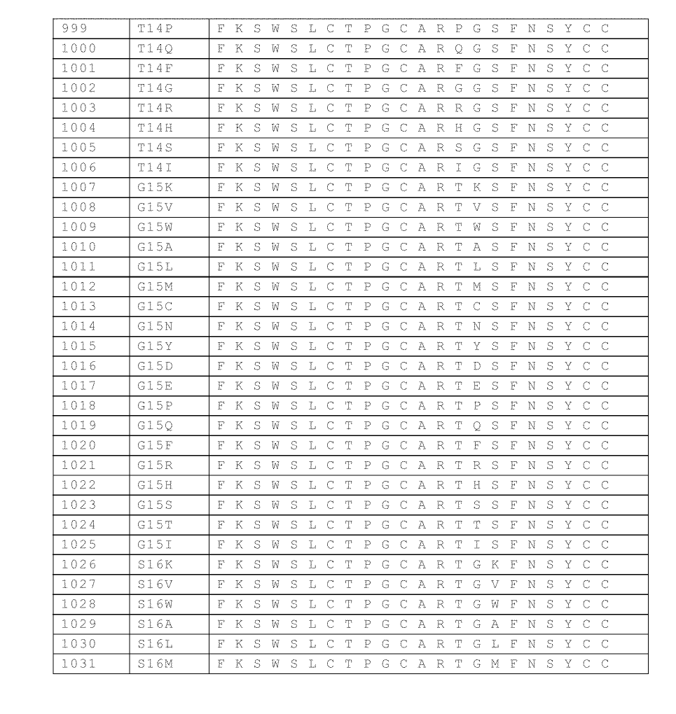

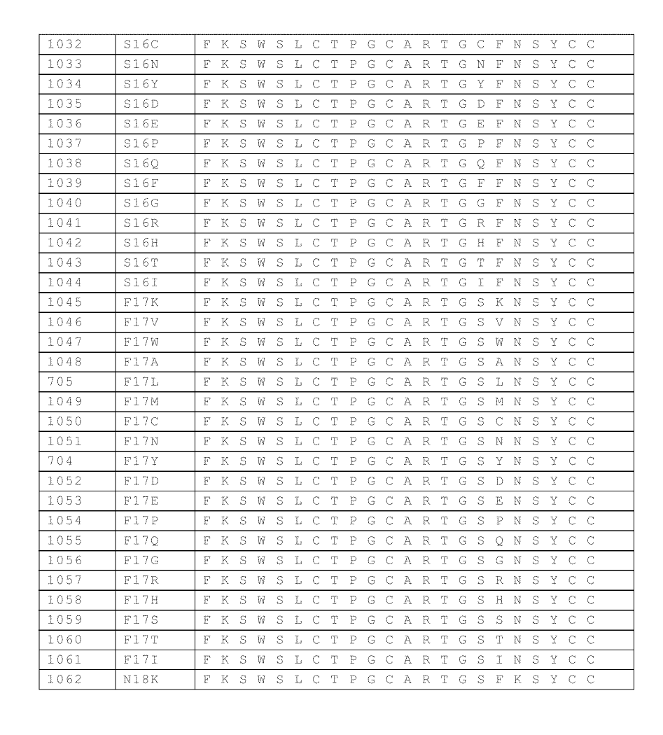

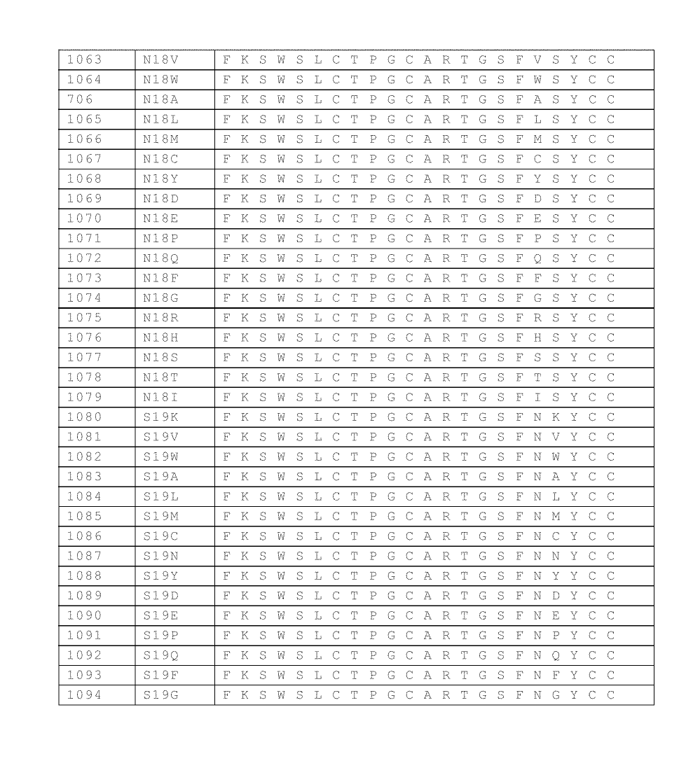

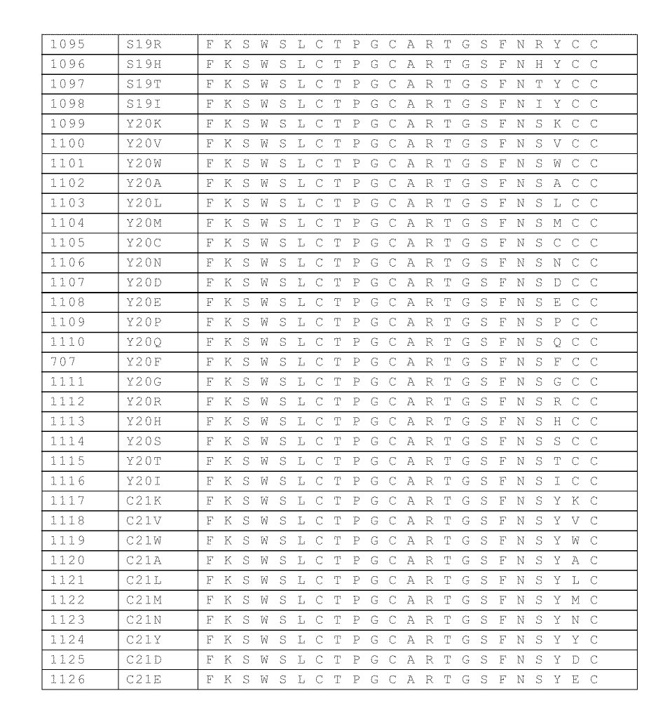

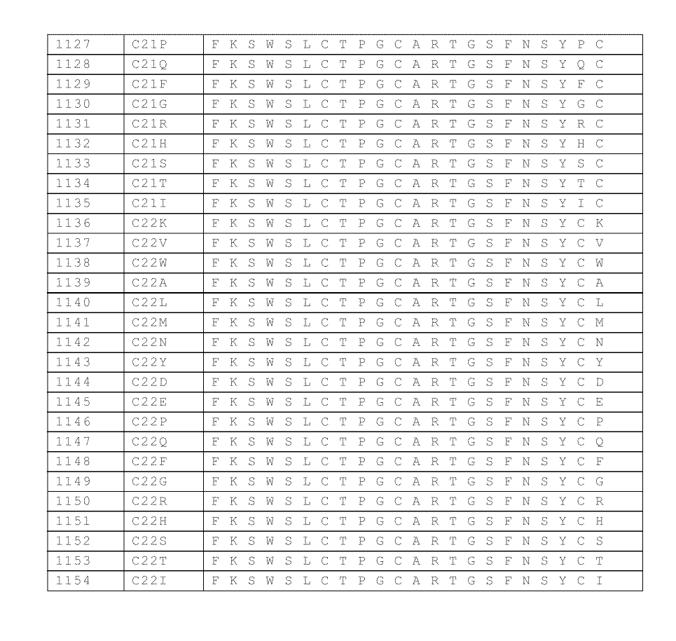

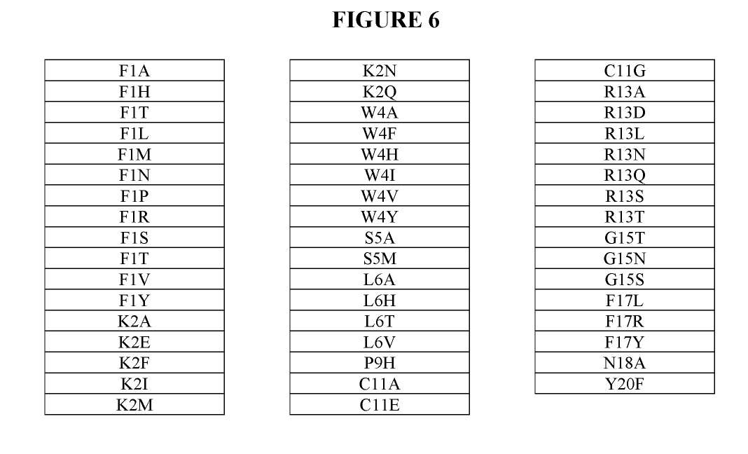

A first generation library of 418 MU1140 variants was constructed and tested in S. mutans (see, Example 1), resulting in detection of 114 variants that demonstrated equal or improved antimicrobial activity against M. luteus (i.e., relative to native (wild-type) MU1140). A 58 member subset of the 114 MU1140 variant producing strains were reconstructed using vectors designed to integrate site specifically at the mutA locus on the S. mutans chromosome, replacing the native copy of mutA. The 58 MU1140 variants were selected based on improved antimicrobial activity against M. luteus. See FIG. 6.

The 58 MU1140 variant producing strains were assessed for improvements in antimicrobial activity using the M. luteus zone of clearing assay using non-normalized culture supernatants obtained from overnight growth of S. mutans strains. Consistent (well-to-well and plate-to-plate) zones of clearing were observed for both S. mutans strains JH1140 and SM152. Strains expressing twenty six (26) different variants that generated zones of clearing larger than the positive control (strain SM152) were identified for further analysis. Strains expressing an additional 15 variants (with zones of clearing of similar or smaller than the control strain SM152) were identified representing novel and/or underrepresented amino acid positions. In total, 41 variants were identified for further analysis based on antimicrobial activity and positional diversity criteria (Table 6).

TABLE 6
STRAINS SELECTED FOR FURTHER ANALYSIS
BASED ON THE SIZE OF THE NON-NORMALIZED ZONE
OF CLEARING AND/OR POSITIONAL DIVERSITY
Zone of clearing

| Larger than | Similar to | Smaller than |
|---|---|---|
| SM152 | SM152 | SM152 |
| F1A | F1N | W4V |
| F1I | F1L | S5M |
| F1S | K2I | P9H |
| F1T | K2M | C11E |
| F1Y | S5A | |
| K2A | L6H | |
| K2E | L6N | |
| K2N | L6V | |
| K2Q | C11A | |
| W4A | R13S | |
| W4F | Y20F | |
| W4Y | | |
| L6A | | |
| L6T | | |
| C11G | | |
| R13A | | |
| R13D | | |
| R13L | | |
| R13N | | |
| R13Q | | |
| R13T | | |
| G15N | | |
| G15S | | |
| F17L | | |
| F17Y | | |
| N18A | | |

### Example 3

### Mass Spectra of Native MU1140 and MU1140 Variants

Mass Spectrometry was used to confirm identity of the MU1140 variants that were produced using codon substitutions of the mutA gene in S. mutans JH1140. The theoretical molecular weight of each variant was calculated based on the published 2,266 Dalton (Da) molecular weight of MU1140 (Chen et al., Microbiology, 79(13), 4015-23, 2013), by subtraction of the molecular weight of the specific amino acid residue, addition of the molecular weight of the replacement amino acid residue, and adjustment for ring hydrolysis (18 Da) or 2, 3-didehydroalanine (DHA) substitution (2 Da) as appropriate.

### Results

The multi-site codon MU1140 variants were independently resolved by LC-MS using standard methods. The results for the single site variants and the multi-site variants are provided in Table 7 and Table 8, respectively.

TABLE 7
Mass spectral analysis of single site MU1140 variants

| | Retention | | |
|---|---|---|---|
| | time for LC- | Calculated | MW by MS |
| Mutation | MS | MW | analysis |
| F1I | 2.26 | 2232 | 2231.1 |
| F1L | 2.29 | 2232 | 2230.5 |
| R13N | 2.63 | 2223.9 | 2223 |
| F17L | 2.35 | 2232 | 2230.8 |
| F17Y | 2.16 | 2282 | 2280.6 |
| N18A | 2.42 | 2223 | 2220 |
| Y20F | 2.53 | 2268 | 2248.2 |

TABLE 8
Mass spectral analysis of multi-site MU1140 variants

| | | Measured | |
|---|---|---|---|
| | Calculated | masses | Retention time |
| Analogue | MW | (monoisotopic) | LC-MS [min]¹ |
| F1I | 2232 | 2231 | 16.06 |
| F1A R13A | 2104.8 | 2102.8 | 16.94 |
| F1I R13D | 2190.9 | 2189.9 | 17.58 |
| F1T R13G | 2120.8 | 2119.8 | 16.78 |
| F1L R13D | 2190.9 | 2188.9 | 17.73 |
| F1I R13N | 2189.9 | 2187.9 | 17.23 |
| F1I R13N G15A | 2203.9 | 2201.9 | 17.51 |
| F1I W4M R13A | 2091.9 | 2089.9 | 16.51 |
| F1I R13A | 2146.9 | 2144.9 | 17.58 |
| F1L R13N | 2189.9 | 2187.9 | 17.35 |
| F1L R13A | 2146.9 | 2144.9 | 17.68 |
| F1S R13N | 2163.8 | 2161.9 | 16.45 |
| F1V R13N | 2175.8 | 2173.9 | 16.93 |
| F1G R13N | 2133.8 | 2131.8 | 16.42 |
| F1I R13N Y20F | 2173.9 | 2171.9 | 17.91 |

Variant molecular weights were found to agree with the calculated molecular weight within 2.0 daltons. The agreement between the experimental and calculated molecular weights confirms production of the variants as engineered in S. mutans.

### Example 4

### Therapeutic Properties of MU1140 Variants

### Scale-Up and Purification

S. mutans strains were grown in 1 L shake flasks or 1 L fermenters. Clarified culture supernatants were recovered using chloroform extraction (Chen et al. Microbiology, 79(13), 4015-23, 2013), and purified using flash chromatography to greater than 90% purity based on absorbance at 280 nm using standard techniques. Variants were lyophilized to dryness, quantified by dry weight and UPLC, and then assayed for activity using C. difficile MIC and simulated intestinal fluid stability assays.

Follow-up testing of MU1140 variant performance in an animal model required more than 200 mg pure variant per treatment. Select variants were scaled to 5 L fermenters and purified using two step chromatographic purification to greater than 90% purity based on absorbance at 280 nm.

C. difficile MIC Assay

A minimum inhibitory concentration (MIC) assessment was performed using the anaerobic human gastrointestinal pathogen Clostridium difficile. A modification of the CLSI M11-A8 broth dilution method for Bacteroides fragilis susceptibility testing was used for C. difficile. (Methods for Dilution In Antimicrobial Susceptibility Tests for Bacteria That Grow Aerobically; Approved Standard-Eighth Edition. CLSI document M07-A8 (ISBN 1-56238-689-1). Vol. 29 No. 2. Clinical and Laboratory Standards Institute, USA, 2009). The testing determined the MIC of vancomycin and MU1140 variants, defined as the lowest concentration of an antimicrobial agent that completely prevents visible growth of the bacteria in broth medium. C. difficile was streaked and incubated on blood agar plates under anaerobic conditions. Vancomycin and MU1140 variants were dissolved and diluted with 100% DMSO in 2-fold dilutions. C. difficile strains (ATCC 9689, BAA-1805, ATCC 70057, ATCC 43596, ATCC 43255, BI1 and BAA-1875 for selected single and/or multi-site variants and UNT103-1, UNT107-1, ATCC BAA 1874, ATCC 43597 & ATCC BAA-1808 for selected muti-site variants) were each tested at 11 concentrations by 2-fold serial dilutions from 64 to 0.0625 μg/mL of vanomycin or MU1140 variant. A 4 μL aliquot of each dilution was added to 196 μL of Reinforced Clostridial Medium (RCM), with test strains in wells of a 96 well plate. The final volume was 200 μL in each well. The assay plates were loosely wrapped in a plastic bag and incubated 46-48 hours at 36° C. for C. difficile under anaerobic conditions and then visually examined and scored positive (+) for inhibition of growth or turbidity or negative (−) for no effect upon growth or turbidity.

### MIC Assay Against Other Gram Positive and Acid-Fast Micro-Organisms

A minimum inhibitory concentration (MIC) assessment against a number of other Gram positive microorganisms and Mycobacterium phlei (an acid-fast organism) was done using the broth dilution assay as described by the CLSI (CLSI document M07-A8). Single site variants were tested against three Vancomycin resistant Enterococci (VRE; two Enterococcus faecalis strains (ATCC 51575 and ATCC 51299), one Enterococcus faecium strain (ATCC 700221)), five Staphylococcus aureus strains (ATCC 29213, 19636, 33591, 700699 and BAA-1717), Streptococcus pneumoniae (ATCC 700675), and Mycobacterium phlei (ATCC 11758). Multi-site variants were tested against three VRE strains (two Enterococcus faecalis strains (CCUG 47775 and ATCC 700221), one Enterococcus faecium strain (ATCC 700221)), Clostridium scindens (ATCC 35704), Bifidobacterium breve (ATCC 15700), Lactobacillus acidophilus (CCUG 4356), Peptostreptococcus anaerobius (ATCC 27337) and Eggerthella lenta (ATCC 27337). Samples and a standard reference agent for each strain (Linezolid, Ampicillin, Gentamicin and Vancomycin, as applicable) were dissolved and diluted in 100% DMSO and 4 μL was added per well to 96-well plate with 196 μL of broth seeded with the test organism. Samples were tested at 11 concentrations from 64 to 0.0625 μg/mL. The assay plates were loosely wrapped in a plastic bag and incubated 20-24 hours at 36° C. under aerobic conditions for Enterococcus Spp., S. aureus, M. phlei and P. aeruginosa and under 5% CO2 for S. pneumoniae, C. scindens, B. breve, L. acidophilus, P. anaerobius and E. lenta, and then visually examined and scored positive (+) for inhibition of growth or turbidity or negative (−) for no effect upon growth or turbidity.

MIC Against Gram Negatives (Including H. pylori)

A minimum inhibitory concentration (MIC) assessment against Pseudomonas aerigunosa (a Gram negative bacterium) was performed for single site variants in the broth dilution assay as described by the CLSI (CLSI document M07-A8). Samples and Ofloxacin control were dissolved and serially diluted in 100% DMSO and 4 μL was added per well to 96-well plate with 196 μL of Cation Adjusted Mueller Hinton II Broth seeded with P. aeruginosa. The compounds were tested at 11 concentrations from 64 to 0.0625 μg/mL. The assay plates were loosely wrapped in a plastic bag and incubated 20-24 hours at 36° C. (aerobic conditions) and then visually examined and scored positive (+) for inhibition of growth or turbidity or negative (−) for no effect upon growth or turbidity.

A minimum inhibitory concentration (MIC) assessment against Heliobacter pylori (a Gram negative) was performed for multi-site variants in the broth dilution assay as described by the CLSI (CLSI document M07-A8) with and without the addition of 0.05M EDTA (Ethylenediaminetetraacetic acid, a cell membrane sensitizing agent). Samples and Tetracycline control were dissolved and serially diluted in 100% DMSO and 4 μL was added per well to 96-well plate with 196 μL of Brucella Broth—7% FBS seeded with H. pylori ±0.05 M EDTA. The compounds were tested at 11 concentrations from 64 to 0.0625 μg/mL. The assay plates were loosely wrapped in a plastic bag and incubated 20-24 hours at 36° C. under 5% CO₂ (anaerobic conditions) and then visually examined and scored positive (+) for inhibition of growth or turbidity or negative (−) for no effect upon growth or turbidity.

### Simulated Intestinal Fluid Assay

An in vitro simulated intestinal fluid stability assay was performed to simulate the stability of MU1140 variants in the gastrointestinal tract in the presence of simulated intestinal fluid and proteases. Assay incubation conditions included 475 μL simulated intestinal fluid (FaSSIF-v2 (Ekarat et al., Pharm Res. Vol. 25, No. 7, July 2008) containing 1% porcine pancreatin, 3 μg/ml bovine trypsin, and 3 μg/ml bovine chymotrypsin mixed with 25 μl of 1 mg/mL variant for a final concentration of 50 μg/ml variant in a 96 well format. Samples were incubated at 37° C. and analyzed using LC-MS/MS for loss of substrate. The percent remaining, elimination rate constant (Ke), and in vitro protein half-lives were calculated.

### Simulated Gastric Fluid Assay

An in vitro simulated gastric fluid stability assay was performed to simulate the stability of nine MU1140 variants in the gastric chamber in the presence of gastric fluid and proteases. Assay incubation conditions included 242.5 L simulated gastric fluid (SGF, USP 39 Test Solution; 2 g/L NaCl, 7 mL/L glacial HCl, pH 1.4) containing 1.75 mg/mL Pepsin (3200-4500 U/mg) mixed with 7.5 μL of 10 mg/mL variant (or positive control, NH₂-Met-Arg-Phe-Ala-OH, MRFA, CAS#67368-29-0) for a final concentration of 300 μg/ml variant in a microfuge tube. Samples were incubated at 37° C. and analyzed using HPLC or LC-MS for loss of substrate. The percent remaining, elimination rate constant (Ke), and in vitro protein half-lives were calculated. A control of SGF without enzyme was also run and analyzed in the same manner for each variant.

### Animal Study

A cannulated Syrian hamster C. difficile model was used to assess efficacy of MU1140 variants in the treatment of C. difficile infection. Following ileal cannulation surgery and recovery, male Golden Syrian hamsters were inoculated with Clostridium difficile UNT103-1 (VA11, non epidemic (cdtB-, REA group J) Curtis Donskey, Cleveland VA Hospital, Cleveland, Ohio). On Day 1, at 24 hrs after infection, all animals received a single subcutaneous injection of clindamycin (10 mg/kg). Test article formulations were administered three times per day (TID), starting on Day 2 at 18 hours after Clindamycin injection, for 5 consecutive days (Days 2 through 6). Vancomycin (positive control) was administered once a day at 20 mg/kg on Days 2 through 6, and followed until Day 22 to assess recurrence. The cecal contents from all hamsters that died on study or euthanized at the end (Day 21) were tested for C. difficile Toxin A and Toxin B (tgc BIOMICS ELISA Kit, cat# TGC-E002-1) and for C. difficile Spore counts. Spore titer was determined by incubating the cecal contents in 50% final concentration of ethanol for 60 minutes at room temperature, centrifuging and resuspending the pellet in nano-pure water. The resuspension is incubated at 65° C. for 15 minutes, centrifuged again and the pellet resuspended again in nano-pure water. The sample is diluted and spotted onto Colombia agar plates with 7% laked house blood, 0.5 mg/mL cylcoserine, 15 μg/mL cefoxitin and 1 mg/mL taurocholate. After 48 hours of anaerobic incubation, spore counts are determined as colony forming units (CFUs).

A follow up non-cannulated Syrian hamster C. difficile infection model was used to assess efficacy of seven MU1140 variants in the treatment of C. difficile infection following an oral gavage regimen. Following the recovery phase, male Golden Syrian hamsters were inoculated with Clostridium difficile UNT103-1 (VA11, non epidemic (cdtB-, REA group J) Curtis Donskey, Cleveland VA Hospital, Cleveland, Ohio). On Day 1, at 24 hrs after infection, all animals received a single subcutaneous injection of clindamycin (10 mg/kg). Test article formulations were administered three times per day (TID), starting on Day 2 at 18 hours after Clindamycin injection, for 5 consecutive days (Days 2 through 6), and followed until Day 22 to assess recurrence. Vancomycin (positive control) was administered once a day at 20 mg/kg on Days 2 through 6. Cecal spore counts and Toxins A and B were determined as described above

A second non-cannulated Syrian hamster C. difficile infection model was used to better define the optimal therapeutic regimen of two MU1140 variants in the treatment of C. difficile infection following an oral gavage regimen. Following the recovery phase, male Golden Syrian hamsters were inoculated with Clostridium difficile UNT103-1 (VA11, non epidemic (cdtB-, REA group J) Curtis Donskey, Cleveland VA Hospital, Cleveland, Ohio). On Day 1, at 24 hrs after infection, all animals received a single subcutaneous injection of clindamycin (10 mg/kg). Test article formulated at 3 to 5 different doses were administered three times per day (TID), starting on Day 2 at 18 hours after Clindamycin injection, for 5 consecutive days (Days 2 through 6), and followed until Day 22 to assess recurrence. Vancomycin (positive control) was administered once a day at 20 mg/kg on Days 2 through 6. Cecal spore counts and Toxins A and B were determined as described above.

### Results

Variants were tested for specific antimicrobial activity against the anaerobic Gram-positive pathogen C. difficile (Table 9) on a weight/volume basis. Three individual C. difficile strains were evaluated to assess the spectrum of activity of MU1140 variants against C. difficile. Although improvements in antimicrobial activity were observed in non-normalized culture supernatants against M. luteus, in a normalized assay the variants did not demonstrate improvements in antimicrobial activity against C. difficile relative to MU1140. Nevertheless, variants tested demonstrated specific antimicrobial activity that was better than vancomycin, a current standard of care for C. difficile infection.

Multisite variants were subsequently tested for specific antimicrobial activity in a C. difficile MIC assay (Table 10) (Methods for Antimicrobial Susceptibility Testing of Anaerobic Bacteria Approved Standard-Seventh Edition. CLSI document M11-A8 (ISBN 1-56238-790-1). Vol. 32 No. 5. Clinical and Laboratory Standards Institute, USA, 2012).

TABLE 9
MIC assay single site variants to C. difficile

| | C. difficile MIC (μg/ml) | | | | | | |
|---|---|---|---|---|---|---|---|
| Variant/ | ATCC | BAA- | BAA- | ATCC | ATCC | ATCC | |
| Vancomycin | 9689 | 1805 | 1875 | 43596 | 43255 | 70057 | BI1 |
| MU1140 | 1, 1 | 0.25 | 0.5 | 0.5 | 0.25 | 0.06, 0.1 | 0.11 |
| F1I | 4 | N/A | N/A | N/A | N/A | 0.06 | 0.49 |
| F1L | 4, 4 | 0.5 | 0.5 | 0.5 | 0.5 | 0.07, 0.5 | 0.07 |
| R13N | 4, 4 | 0.5 | 0.5 | 0.5 | 0.5 | 1.6, 0.5 | 0.81 |
| F17L | 4 | N/A | N/A | N/A | N/A | 0.07 | 0.07 |
| F17Y | 2 | N/A | N/A | N/A | N/A | 0.07 | 2.4 |
| N18A | 2, 4 | 0.5 | 2 | 0.5 | 0.5 | 0.2, 2 | 0.19 |
| Y20F | 2, 0.5 | 0.25 | 0.25 | 0.25 | 0.25 | N/A, 0.25 | N/A |
| Vancomycin | 4, 8 | 1 | 1 | 1 | 1 | 0.5, 1 | 8 |

Note,
two values provided (x, y) for variants that were tested at two independent times

TABLE 10
MIC assay multi-site variants to C. difficile

| | C. difficile MIC (ug/ml) | | | | | | | |
|---|---|---|---|---|---|---|---|---|
| | | | | | | ATCC | | ATCC |
| | ATCC | ATCC | ATCC BAA- | | | BAA- | ATCC | BAA- |
| Variant | 9689 | BAA-1805 | 1875 | 103-1 | 107-1 | 1874 | 43597 | 1808 |
| Vancomycin | 0.5 | 1 | 0.5 | 1 | 4 | 0.5 | 0.5 | 0.5 |
| F11 standard | 0.5 | 0.25 | 0.25 | 0.5 | 0.5 | 0.25 | 0.25 | 0.125 |
| F11 | 0.5 | 0.5 | 0.25 | 0.06249 | 0.25 | 0.25 | 0.25 | 0.25 |
| F1A R13A | 2 | 2 | 2 | 0.5 | 4 | 4 | 2 | 1 |
| F1I R13D | 2 | 1 | 1 | 0.5 | 2 | 0.5 | 1 | 1 |
| F1T R13G | 8 | 8 | 8 | 4 | 16 | 8 | 8 | 8 |
| F1L R13D | 1 | 1 | 1 | 0.5 | 2 | 1 | 1 | 1 |
| F1I R13N | 0.5 | 0.25 | 0.5 | 0.125 | 1 | 0.5 | 0.25 | 0.5 |
| F1I R13N | 8 | 8 | 8 | 2 | 8 | 8 | 8 | 8 |
| G15A | | | | | | | | |
| F1I W4M | 8 | 4 | 4 | 1 | 4 | 4 | 4 | 8 |
| R13A | | | | | | | | |
| F1I R13A | 1 | 0.25 | 0.5 | 0.25 | 1 | 0.5 | 0.5 | 0.25 |
| F1L R13N | 0.5 | 0.25 | 0.5 | 0.125 | 0.5 | 0.25 | 0.25 | 0.25 |
| F1L R13A | 0.5 | 0.25 | 0.5 | 0.25 | 0.5 | 0.5 | 0.5 | 0.5 |
| F1S R13N | 16 | 8 | 16 | 2 | 16 | 16 | 8 | 8 |
| F1V R13N | 0.5 | 0.25 | 0.5 | 0.25 | 0.5 | 0.5 | 0.25 | 0.25 |
| F1G R13N | 8 | 8 | 8 | 2 | 16 | 16 | 8 | 8 |
| F1I R13N | 0.5 | 0.5 | 1 | 0.25 | 0.5 | 0.5 | 0.5 | 0.25 |
| Y20F | | | | | | | | |
| F1A R13G | 8 | 8 | 8 | 2 | 8 | 8 | 4 | 8 |

Single site variants were tested for specific antimicrobial activity against a host of gram positive and an acid fast microorganisms (Table 11) on a weight/volume basis. Three individual Enterococci strains were evaluated to assess the spectrum of activity of MU1140 variants against VRE, five strains of S. aureus, S. pneumonia and the acid-fast strain M. phlei. While variants tested did not show significant differences than MU1140, all demonstrated specific antimicrobial activity that were in the tested range (≤64 μg/mL) against all the organisms tested, though many were less susceptible than to the control.

Multisite variants were subsequently tested for specific antimicrobial activity against a host of gram positive microorganisms (Table 12) and a weight/volume basis. Individual Enterococci strains were evaluated to assess the spectrum of activity of MU1140 variants against VRE, and strains of C. scindens, B. breve, L. acidophilus, P. anaerobius, E. lenta were also assessed. Multi-site variants demonstrated MICs against most of the organisms tested, though many were less susceptible than the control. A few of the multisite variants did not demonstrate any activity against some of the VRE strains tested.

TABLE 11
Other Gram Positives - MIC assay single site variants

| | Gram Positive/Acid-Fast Microorganism MIC (μg/mL) | | | | | | | | | |
|---|---|---|---|---|---|---|---|---|---|---|
| | | | | S. | S. | SS. | S. | S. | | |
| | VRE | VRE | VRE | aureus | aureus | aureus | aureus | aureus | | |
| Variant/ | ATCC | ATCC | ATCC | ATCC | ATCC | ATCC | BAA- | ATCC | S. | pM. |
| Control | 51575 | 51299 | 700221 | 29213 | 19636 | 33591 | 1717 | 700699 | pneumonia | phlei |
| MU1140 | 4 | 4 | 2 | 2 | 1 | 1 | 2 | 2 | 8 | 2 |
| F1I | 8 | 8 | 4 | 4 | 4 | 0.5 | 2 | 4 | 16 | 2 |
| F1L | 8 | 4 | 2 | 4 | 2 | 0.5 | 2 | 2 | 16 | 2 |
| R13N | 8 | 16 | 4 | 2 | 2 | 0.5 | 2 | 4 | 32 | 8 |
| F17L | 8 | 16 | 2 | 4 | 2 | 1 | 2 | 2 | 64 | 2 |
| F17Y | 8 | 8 | 4 | 2 | 1 | 1 | 2 | 2 | 32 | 2 |
| N18A | 16 | 8 | 2 | 4 | 4 | 2 | 2 | 2 | 32 | 2 |
| Y20F | 64 | 4 | 1 | 2 | 1 | 1 | 2 | 2 | 8 | 2 |
| Control | L | L | L | G | G | G | G | V | A | V |
| MIC | 2 | 2 | 2 | 0.25 | 0.125 | 1 | 0.25 | 2 | 1 | 0.25 |

Controls:
L = Linezoid,
G = Gentamicin,
V = Vancomycin,
A = Ampicillin

TABLE 12
Other Gram Positives - MIC assay multi-site variants

| | Gram Positive Microorganism MIC (μg/mL) | | | | | | | |
|---|---|---|---|---|---|---|---|---|
| | VRE | VRE | VRE | | | | | |
| Variant / | CCUG | ATCC | ATCC | C. | | L. | P. | |
| Control | 47775 | 51575 | 700221 | scindens | B. breve | acidophilus | anaerobius | E. lenta |
| F11 standard | 8 | 4 | 4 | 2 | 0.5 | 0.25 | 0.25 | 0.25 |
| F1I | 8 | 4 | 4 | 2 | 0.5 | 0.5 | 0.25 | 0.5 |
| F1A R13A | 32 | 32 | 16 | 16 | 4 | 8 | 2 | 4 |
| F1I R13D | 32 | 32 | 32 | 8 | 2 | 2 | 1 | 2 |
| F1T R13G | 64 | 64 | 32 | 64 | 16 | 8 | 8 | 8 |
| F1L R13D | 32 | 64 | 32 | 8 | 2 | 8 | 2 | 1 |
| F1I R13N | 16 | 16 | 32 | 2 | 1 | 0.5 | 0.25 | 0.5 |
| F1I R13N G15A | >64 | 32 | 32 | 16 | 8 | 8 | 4 | 8 |
| F1I W4M R13A | >64 | >64 | >64 | 32 | 8 | 32 | 8 | 16 |
| F1I R13A | 16 | 16 | 8 | 4 | 1 | 0.5 | 1 | 2 |
| F1L R13N | 16 | 16 | 16 | 4 | 1 | 0.5 | 0.25 | 0.25 |
| F1L R13A | 16 | 32 | 16 | 4 | 1 | 0.5 | 0.5 | 0.5 |
| F1S R13N | >64 | >64 | 64 | 32 | 16 | 16 | 8 | 16 |
| F1V R13N | 32 | 32 | 16 | 4 | 1 | 0.5 | 0.25 | 0.5 |
| F1G R13N | >64 | >64 | 32 | 16 | 16 | 8 | 4 | 16 |
| F1I R13N Y20F | 16 | 8 | 8 | 2 | 0.5 | 0.5 | 0.125 | 0.125 |
| F1A R13G | >64 | >64 | 32 | 32 | 8 | 16 | 4 | 16 |
| Control | L | L | L | V | V | V | V | L |
| MIC | 2 | 2 | 4 | 1 | 0.5 | 1 | 1 | 1 |

Controls:
L = Linezoid,
V = Vancomycin

Single site variants were tested for specific antimicrobial activity against P. aeruginosa (a Gram negative microorganism) on a weigh/volume basis (Table 13). All single-site variants demonstrated no inhibition of growth at all the dilutions tested (MIC>64 μg/mL), consistent with the specificity of MU1140 for activity against Gram positive organisms.

The multi-site variants were tested for specific antimicrobial activity against H. pylori (a Gram negative microorganism) with and without 0.05 M EDTA (a membrane sensitizing agent) on a weigh/volume basis (Table 14). Inhibition of growth was observed for some of the variants, but at levels significantly higher than the Tetracyline control. The addition of 0.05M EDTA had a notable positive effect on the MIC values of several variants but not all.

TABLE 13
MIC Assay of Single Site Variants to a Gram Negative

| Variant/Control | MIC to P. aeruginosa |
|---|---|
| MU1140 | >64 μg/mL |
| F1I | >64 μg/mL |
| F1L | >64 μg/mL |
| R13N | >64 μg/mL |
| F17L | >64 μg/mL |
| F17Y | >64 μg/mL |
| N18A | >64 μg/mL |
| Y20F | >64 μg/mL |
| Ofloxacin Control | 1 μg/mL |

TABLE 14
MIC Assay Multi-Site Variants to H. pylori

| | MIC to H. Pylori without | MIC to H. Pylori with |
|---|---|---|
| Variant/Control | EDTA (μg/mL) | 0.05M EDTA (μg/mL) |
| F11 standard | 16 | 8 |
| F1I | 16 | 8 |
| F1A R13A | >64 | 64 |
| F1I R13D | 8 | 8 |
| F1T R13G | >64 | >64 |
| F1L R13D | 8 | 4 |
| F1I R13N | 8 | 8 |
| F1I R13N G15A | >64 | 64 |
| F1I W4M R13A | 64 | 64 |
| F1I R13A | 8 | 8 |
| F1L R13N | 8 | 8 |
| F1L R13A | 8 | 8 |
| F1S R13N | >64 | >64 |
| F1V R13N | 8 | 8 |
| F1G R13N | >64 | >64 |
| F1I R13N Y20F | 8 | 4 |
| F1A R13G | >64 | >64 |
| Tetracycline Control | 0.5 | 0.25 |

MU1140 variants were tested in simulated intestinal fluid assay to determine stability of variants in the presence of pancreatin, trypsin, and chymotrypsin (Table 15). A half-life of 53.2 to 72.4 min was observed for MU1140, while substitution at position 1 (a putative pepsin cleavage site) prolonged half-lives of variants F1I and F1L to 95 and 77.3 minutes, respectively. Mutation at position 13, a putative trypsin cleavage site, prolonged the half-life of variant R13N to >240 min. Mutation at position 20, a putative chymotrypsin site, prolonged the half-life of variant Y20F to 148 min. Mutation of position 18 to N18A however, rendered the molecule susceptible to proteolytic degradation and shortened the half-life to 24 min.

TABLE 15
Simulated Intestinal Fluid Assay

| | Half-life in FaSSIF+ |
|---|---|
| Variant | (min) |
| MU1140 | 53.2 |
| SM152 | 72.4 |
| F1I | 95 |
| F1L | 77.3 |
| R13N | >240 |
| N18A | 24 |
| Y20F | 148 |

Next, the hypothesis that independent mutations increase variant stability in an additive manner was tested. Multi-site variants were tested in simulated intestinal fluid containing pancreatin, trypsin, and chymotrypsin to determine if the half-lives of multi-site variants demonstrate improved survival (Table 16). Ten of the fifteen multi-site variants tested exhibited a half-life of more than 720 minutes, compared to a half-life of 48 and 58 minutes observed for the SM253 reference standard and the positive control F1I that was purified using the same conditions as the multi-site variants. The improved stability of multi-site variants relative to the single-site variant SM253 indicates that the putative protease cleavage sites in native MU1140 are susceptible to protease-mediated cleavage under physiological conditions despite the extensive post-translational modifications of the lantibiotic.

TABLE 16
Simulated Intestinal Fluid Assay

| | FaSSIF | FaSSIF + 1% |
|---|---|---|
| | only | pancreatin + 3 μg/ml |
| | half-life | trypsin + 3 μg/ml chymotrypsin |
| Variant | (min) | half-life (min) |
| F1I reference | >720 | 48 & 58 (2 tests) |
| standard | | |
| F1A R13A | >720 | 130 |
| F1I R13D | >720 | >720 |
| F1T R13G | >720 | 114 |
| F1L R13D | >720 | >720 |
| F1I R13N | >720 | >720 |
| F1I R13N G15A | >720 | >720 |
| F1I W4M R13A | >720 | 349 |
| F1I R13A | >720 | >720 |
| F1L R13N | >720 | >720 |
| F1L R13A | >720 | >720 |
| F1S R13N | >720 | >720 |
| F1V R13N | >720 | >720 |
| F1G R13N | >720 | 225 |
| F1I R13N Y20F | >720 | >720 |
| F1A R13G | 299 | 124 |

Next, the hypothesis that lantibiotics are not degraded in gastric fluid with pepsin (due to the lack of a c-terminal Amino Acid) and that key independent mutations did not effect that stability was tested. Multi-site variants that demonstrated improved stability in FaSSIF and that had equal or better MICs than F1I (SM253) were tested in simulated gastric fluid (pH 1.5) containing pepsin to determine if the half-lives of multi-site variants demonstrate differences in degradation (Table 17). All eight of the variants tested demonstrated a half-life of more than 1440 minutes (24 hours) which was comparable to F1I. The prolonged stability of the multi-site variants indicates that the mutations within the peptide that lead to improved performance do not lead to reduced stability during upper gastric transit.

TABLE 17
Simulated Gastric Fluid Assay

| | SGF only | SGF + |
|---|---|---|
| | half-life | 1.75 mg/ml Pepsin |
| Variant | (min) | half-life (min) |
| F1I reference | >1440 | >1440 |
| standard | | |
| F1I R13D | >1440 | >1440 |
| F1L R13D | >1440 | >1440 |
| F1I R13N | >1440 | >1440 |
| F1I R13A | >1440 | >1440 |
| F1L R13N | >1440 | >1440 |
| F1L R13A | >1440 | >1440 |
| F1V R13N | >1440 | >1440 |
| F1I R13N Y20F | >1440 | >1440 |
| Positive Control | >1440 | 906 |

Six variants were evaluated in vivo using a C. difficile cannulated Syrian Hamster Model to determine if improvements in the efficacy of C. difficile treatment could be observed relative to a current clinical standard of care, vancomycin (Surawicz et al. Am J Gastroenterol., 108(4):478-98, 2013). The survival results for C. difficile infected hamsters treated with vehicle, vancomycin, or MU1140 variants are presented in Table 18. By the end of the study at Day 21, variants F1I, R13N, and Y20F exhibited superiority to vancomycin, as up to 100% survival was observed in variant treated hamsters compared to the 33% survival observed for the vancomycin control. The spore counts and toxin levels in animals at the end of the study were consistent with the clinical survival picture.

TABLE 18
Efficacy study in Ileal Cannulated Hamster C. difficile Infection Model

| | | Mean LOG₁₀ | |
|---|---|---|---|
| | | CFU/mL of feces | |
| | Percent | homogenates (cecal | |
| | Survival at | spore counts) at | Toxins (A/B) levels |
| Treatment | Day-22 | Day-22 | at Day-22 (ng/mL) |
| Vehicle | 0 | 4.09 | 1955.0/1368.7 |
| Vancomycin | 33 | 3.24 | 2435.9/1209.1 |
| F17L | 33 | 3.33 | 1490.4/1149.9 |
| F1I | 100 | <1.62* | <1.6/<1.6** |
| Y20F | 67 | 2.21 | 369.5/331.3 |
| F1L | 17 | 4.31 | 269.5/328.3 |
| N18A | 33 | 3.52 | 684.2/614.6 |
| R13N | 67 | 2.24 | 1944.0/956.3 |

*Limit of Detection (LOD) = 1.62 LOG10 CFU/gram of sample
**LOD = 1.6 ng/mL toxin

Eight variants were evaluated in vivo using a C. difficile Syrian Hamster Model where the peptides were delivered by oral gavage, to determine if improvements in the efficacy of C. difficile treatment could be observed relative to a current clinical standard of care, vancomycin. The survival results for C. difficile infected hamsters treated with vehicle, vancomycin, or MU1140 variants are presented in Table 19. By the end of the study at Day 22, variants F1V R13N and F1I R13N Y20F exhibited equal or superior efficacy, as compared to Vancomycin-treated animals. The spore counts and toxin levels in animals at the end of the study were consistent with the clinical survival picture.

TABLE 19
Efficacy study in Hamster C. difficile Infection Model by Oral Gavage

| | | Mean LOG₁₀ | |
|---|---|---|---|
| | | CFU/mL of feces | |
| | Percent | homogenates | |
| | Survival | (cecal spore | Toxins (A/B) levels |
| Treatment | at Day-22 | counts) at Day-22 | at Day-22 (ng/mL) |
| Vehicle | 0 | 7.23 | 6630.9/2351.1 |
| Vancomycin | 83 | 2.96 | 84.2/8 |
| F1I | 0 | 7.21 | 4313.7/1488.3 |
| F1I R13D | 50 | 4.8 | 648.5/216.6 |
| F1I R13N | 66 | 3.64 | 138.1/120.2 |
| F1I R13A | 50 | 4.67 | 147/205.5 |
| F1L R13N | 0 | 7.36 | 5477.8/2189.1 |
| F1L R13A | 0 | 7.28 | 4532.5/1795.5 |
| F1V R13N | 100 | 2.4* | 0.8/0.8** |
| F1I R13N Y20F | 83 | 2.65 | 5.1/6 |

*Limit of detection = 2.40
**Limit of detection = 0.8

Two selected variants were evaluated in vivo using a C. difficile Syrian Hamster Model where the peptides were delivered by oral gavage, and following an ascending dose regimen, to determine if improvements in the efficacy of C. difficile treatment could be observed relative to a current clinical standard of care, vancomycin. The survival results for C. difficile infected hamsters treated with vehicle, vancomycin, or MU1140 variants are presented in Table 20. By the end of the study at Day 21, variants F1V R13N exhibited superior efficacy, as compared to Vancomycin-treated animals at the same dose, and followed a dose-dependent response. The spore counts and toxin levels in animals at the end of the study were consistent with the clinical survival picture.

TABLE 20
Efficacy study and Dose Range-Finding in Hamster C. difficile
Infection Model by Oral Gavage.

| | | Mean LOG₁₀ | |
|---|---|---|---|
| | Percent | CFU/mL of feces | |
| | Survival | homogenates | Toxins (A/B) |
| | at | (cecal spore | levels at Day-22 |
| Treatment (dose) | Day-22 | counts) at Day-22 | (ng/mL) |
| Vehicle | 0 | 5.15 | 2857.1/1852.8 |
| Vancomycin | 50 | 6.66 | 292.6/71.1 |
| F1V R13N (2 mg/Kg) | 0 | 6.13 | 578.5/462.7 |
| F1V R13N (6 mg/Kg) | 50 | 4.27 | 266.2/238.1 |
| F1V R13N (20 mg/Kg) | 66 | 3.88 | 74.2/57.8 |
| F1V R13N (35 mg/Kg) | 100 | 2.40* | 0.8/0.8** |
| F1V R13N (60 mg/Kg) | 83 | 2.51 | 7.3/5.5 |
| F1I R13N Y20F | 0 | 5.47 | 215.5/242.2 |
| (6 mg/Kg) | | | |
| F1I R13N Y20F | 33 | 5.37 | 165.5/272.3 |
| (20 mg/Kg) | | | |
| F1I R13N Y20F | 83 | 3.18 | 47.6/108.7 |
| (60 mg/Kg) | | | |

*Limit of detection = 2.40
**Limit of detection = 0.8

### Example 5

### Multiple Amino Acid Substitutions

As a follow-up to production of the variants comprising single amino acid substitutions, 305 variants were designed to combine up to eight amino acid substitutions per variant, at positions 1, 2, 4, 5, 6, 7, 12, 13, 15, and 20 of the 22 amino acid mature peptide sequence. 270 variant strains were successfully built, as verified by colony PCR and DNA sequence verification of the mutA locus. Although several attempts at vector construction and transformation were attempted, we cannot determine whether strain construction failed due to technical reasons or biological reasons (the variant was toxic to the host).

Variant producing strains were assessed for improvements in antimicrobial activity using the M. luteus zone of clearing assay using non-normalized culture supernatants obtained from overnight growth of S. mutans strains. It is not known if the larger zones of clearing are due to production of variants with higher specific antimicrobial activity, or if higher titers are responsible for the larger zones of clearing. Consistent production of zones of clearing was observed for both S. mutans strains JH1140 and SM253 from well-to-well and plate-to-plate. Strains encoding F1N, F1P, F1E, S5F, L6I, and A12T mutations resulted in either low levels of antimicrobial activity or activity below the level of detection. The majority of the variant library demonstrated equal or greater antimicrobial activity than the current lead candidate and control strain F1I.

Variant stability is a pharmacological property that is essential for successful development of MU1140 as a successful therapeutic for gastrointestinal applications. Native MU1140 contains a trypsin cleavage site at position R13, although this site may be protected from efficient protease cleavage (Chen et al., Microbiology, 79(13), 4015-23, 2013). Amino acid substitutions to F1 and R13 have each resulted in extended half-lives of purified MU1140 variants in simulated intestinal fluid.

Mutation of F1 and K2 residues may impact maturation of the MU1140 propeptide by the protease mutP as well as cleavage by pepsin and chymotrypsin. Strains containing F1E and F1P substitutions each underperformed the SM253 control strain (F1I). However, F1A, F1G, F1L, and F1V substitutions generally yielded higher activity than SM253.

Thirteen different amino acid substitutions were made at position 13. All were functional and performed ≥SM253 in at least one variant. R13A mutations were over-represented in the high performing group of variants that generated zones of clearing >0.319 cm².

A summary of the amino acid substitutions is found in FIG. 8, separated into 6 different groups:
Group 1: >activity than SM253 control strain (>0.319 cm² zone of clearing)
Group 2: >activity than SM253 in 2 replicate experiments (1 replicate experiment
Group 3: >activity than SM253 in 1 replicate and ≤activity in 1 replicate experiment
Group 4: >activity than SM253 control strain (>0.167 cm² zone of clearing but <0.319 cm² zone of clearing)
Group 5: ≤activity than SM253 control (≤0.167 cm² zone of clearing)
Group 6: Undetermined

What is claimed is:

1. A non-naturally occurring lantibiotic comprising an amino acid sequence of MU1140 and further comprising a mutation including:
a. arginine at position 13 changed to asparagine (R13N);
b. phenylalanine at position 17 changed to leucine (F17L) or tyrosine (F17Y);
c. asparagine at position 18 changed to alanine (N18A);
d. tyrosine at position 20 changed to phenylalanine (Y20F); or
e. combinations thereof.

2. A non-naturally occurring lantibiotic comprising an amino acid sequence of MU1140 and further comprising two mutations, wherein one mutation includes:
a. arginine at position 13 changed to asparagine (R13N);
b. phenylalanine at position 17 changed to leucine (F17L) or tyrosine (F17Y);
c. asparagine at position 18 changed to alanine (N18A);
d. tyrosine at position 20 changed to phenylalanine (Y20F); or
e. combinations thereof.

3. The non-naturally occurring lantibiotic of claim 2, wherein one mutation is arginine at position 13 changed to asparagine (R13N).

4. The non-naturally occurring lantibiotic of claim 2 or 3, wherein one mutation is phenylalanine at position 1 changed to valine (F1V).

5. A non-naturally occurring lantibiotic comprising an amino acid sequence of MU1140 and further comprising two mutations, wherein one mutation includes:
a. phenylalanine at position 1 changed to valine (F1V);
b. phenylalanine at position 1 changed to alanine (F1A);
c. phenylalanine at position 1 changed to isoleucine (F1I);
d. phenylalanine at position 1 changed to leucine (F1L);
e. phenylalanine at position 1 changed to threonine (F1T); or
f. phenylalanine at position 1 changed to tyrosine (F1Y).

6. The non-naturally occurring lantibiotic of claim 5, wherein one mutation is phenylalanine at position 1 changed to valine (F1V).

7. The non-naturally occurring lantibiotic of claim 5 or 6, wherein one mutation is arginine at position 13 changed to asparagine (R13N).

8. A non-naturally occurring lantibiotic comprising an amino acid sequence of MU1140 and further comprising two mutations including:
a. phenylalanine at position 1 changed to alanine in combination with arginine at position 13 changed to:
i. alanine (F1A R13A),
ii. valine (F1A R13V),
iii. asparagine (F1A R13N), or
iv. serine(F1A R13S);
b. phenylalanine at position 1 changed to glycine in combination with arginine at position 13 changed to:
i. glycine (F1G R13G);
c. phenylalanine a position 1 changed to histidine in combination with arginine at position 13 changed to:
i. asparagine (F1H R13N);
d. phenylalanine at position 1 changed to isoleucine in combination with arginine at position 13 changed to:
i. alanine (F1I R13A),
ii. glycine (F1I R13G),
iii. isoleucine (F1I R13I),
iv. asparagine (F1I R13N),
v. proline (F1I R13P),
vi. glutamine (F1I R13Q),
vii. glutamic acid (F1I R13S),
viii. serine (F1R13V), or
ix. valine (F1I R13E);
e. phenylalanine at position 1 changed to leucine in combination with arginine at position 13 changed to:
i. alanine (F1L R13A),
ii. aspartic acid (F1L R13D),
iii. glycine (F1L R13G),
iv. asparagine (F1L R13N),
v. proline (F1L R13P), or
vi. glutamine (F1L R13Q);
f. phenylalanine at position 1 changed to threonine in combination with arginine at position 13 changed to:
i. alanine (F1T R13A),
ii. asparagine (F1T R13N), or
iii. valine (F1T R13V);
g. phenylalanine at position 1 changed to valine in combination with arginine at position 13 changed to:
i. alanine (F1V R13A),
ii. asparagine (F1V R13N),
iii. glutamine (F1V R13Q),
iv. aspartic acid (F1V R13D),
v. valine (F1V R13V), or
vi. proline (F1V R13P); or
h. phenylalanine at position 1 changed to tyrosine in combination with arginine at position 13 changed to:
i. aspartic acid (F1Y R13D), or
ii. glycine (F1Y R13G).

9. The non-naturally occurring lantibiotic of claim 8, comprising two mutations including phenylalanine at position 1 changed to valine in combination with arginine at position 13 changed to:
i. alanine (F1V R13A),
ii. asparagine (F1V R13N),
iii. glutamine (F1V R13Q),
iv. aspartic acid (F1V R13D),
v. valine (F1V R13V), or
vi. proline (F1V R13P).

10. The non-naturally occurring lantibiotic of claim 9, wherein the two mutations consist of phenylalanine at position 1 changed to valine in combination with arginine at position 13 changed to asparagine (F1V R13N).

11. A non-naturally occurring lantibiotic comprising an amino acid sequence of MU1140 and further comprising three mutations including:
a. phenylalanine at position 1 changed to isoleucine, in combination with arginine at position 13 changed to alanine, in combination with glycine at position 15 changed to alanine (F1I R13A G15A);
b. phenylalanine at position 1 changed to isoleucine, in combination with arginine at position 13 changed to aspartic acid, in combination with glycine at position 15 changed to alanine (F1I R13D G15A);
c. phenylalanine at position 1 changed to isoleucine, in combination with tryptophan at position 4 changed to isoleucine, in combination with arginine at position 13 changed to alanine (F1I W4I R13A);
d. phenylalanine at position 1 changed to isoleucine, in combination with tryptophan at position 4 changed to methionine, in combination with arginine at position 13 changed to aspartic acid (F1I W4M R13D);
e. phenylalanine at position 1 changed to isoleucine, in combination with tryptophan at position 4 changed to methionine, in combination with arginine at position 13 changed to asparagine (F1I W4M R13N);
f. phenylalanine at position 1 changed to isoleucine, in combination with lysine at position 2 changed to alanine, in combination with arginine at position 13 changed to alanine (F1I K2A R13A);
g. phenylalanine at position 1 changed to isoleucine, in combination with leucine at position 6 changed to valine, in combination with arginine at position 13 changed to alanine (F1I L6V R13A);
h. phenylalanine at position 1 changed to isoleucine, in combination with arginine at position 13 changed to alanine, in combination with tyrosine at position 20 changed to phenylalanine (F1I R13A Y20F);
i. phenylalanine at position 1 changed to isoleucine, in combination with arginine at position 13 changed to aspartic acid, in combination with tyrosine at position 20 changed to phenylalanine (F1I R13D Y20F);
j. phenylalanine at position 1 changed to isoleucine, in combination with arginine at position 13 changed to asparagine, in combination with tyrosine at position 20 changed to phenylalanine (F1I R13N Y20F);
k. phenylalanine at position 1 changed to leucine, in combination with arginine at position 13 changed to asparagine, in combination with tyrosine at position 20 changed to phenylalanine (F1L R13N Y20F);
l. phenylalanine at position 1 changed to leucine, in combination with arginine at position 13 changed to alanine, in combination with tyrosine at position 20 changed to phenylalanine (F1L R13A Y20F); or
m. phenylalanine at position 1 changed to leucine, in combination with arginine at position 13 changed to aspartic acid, in combination with tyrosine at position 20 changed to phenylalanine (F1L R13D Y20F).

12. A non-naturally occurring lantibiotic comprising an amino acid sequence of MU1140 and further comprising mutations wherein:
a. phenylalanine at position 1 changed to isoleucine, in combination with lysine at position 2 changed to alanine, in combination with tryptophan at position 4 changed to lysine, in combination with arginine at position 13 changed to alanine (F1I K2A W4K R13A); or
b. phenylalanine at position 1 changed to isoleucine, in combination with lysine at position 2 changed to alanine, in combination with tryptophan at position 4 changed to lysine, in combination with arginine at position 13 changed to aspartic acid (F1I K2A W4K R13D).

13. A non-naturally occurring lantibiotic comprising an amino acid sequence of MU1140 and further comprising mutations wherein phenylalanine at position 1 changed to isoleucine, in combination with lysine at position 2 changed to alanine, in combination with tryptophan at position 4 changed to methionine, in combination with arginine at position 13 changed to alanine, in combination with tyrosine at position 20 changed to phenylalanine (F1I K2A W4K R13A Y20F).

14. A non-naturally occurring, post-translationally modified lantibiotic comprising an amino acid sequence of MU1140 (SEQ ID NO: 1156) and further comprising a mutation including:
a. arginine at position 13 changed to asparagine (R13N);
b. phenylalanine at position 17 changed to leucine (F17L) or tyrosine (F17Y);
c. asparagine at position 18 changed to alanine (N18A);
d. tyrosine at position 20 changed to phenylalanine (Y20F); or
e. combinations thereof.

15. A non-naturally occurring, post-translationally modified lantibiotic comprising an amino acid sequence of MU1140 (SEQ ID NO: 1156) and further comprising two mutations, wherein one mutation includes:
a. arginine at position 13 changed to asparagine (R13N);
b. phenylalanine at position 17 changed to leucine (F17L) or tyrosine (F17Y);
c. asparagine at position 18 changed to alanine (N18A);
d. tyrosine at position 20 changed to phenylalanine (Y20F); or
e. combinations thereof.

16. The non-naturally occurring, post-translationally modified lantibiotic of claim 15, wherein one mutation is arginine at position 13 changed to asparagine (R13N).

17. The non-naturally occurring, post-translationally modified lantibiotic of claim 15 or 16, wherein one mutation is phenylalanine at position 1 changed to valine (F1V).

18. A non-naturally occurring, post-translationally modified lantibiotic comprising an amino acid sequence of MU1140 (SEQ ID NO: 1156) and further comprising two mutations, wherein one mutation includes:
a. phenylalanine at position 1 changed to valine (F V);
b. phenylalanine at position 1 changed to alanine (F1A);
c. phenylalanine at position 1 changed to isoleucine (F1I);
d. phenylalanine at position 1 changed to leucine (F1L);
e. phenylalanine at position 1 changed to threonine (F1T); or
f. phenylalanine at position 1 changed to tyrosine (F1Y).

19. The non-naturally occurring, post-translationally modified lantibiotic of claim 18, wherein one mutation is phenylalanine at position 1 changed to valine (F1V).

20. The non-naturally occurring, post-translationally modified lantibiotic of claim 18 or 19, wherein one mutation is arginine at position 13 changed to asparagine (R13N).

21. A non-naturally occurring, post-translationally modified lantibiotic comprising an amino acid sequence of MU1140 (SEQ ID NO: 1156) and further comprising two mutations including:
a. phenylalanine at position 1 changed to alanine in combination with arginine at position 13 changed to:
i. alanine (F1A R13A),
ii. valine (F1A R13V),
iii. asparagine (F1A R13N), or
iv. serine(F1A R13S);
b. phenylalanine at position 1 changed to glycine in combination with arginine at position 13 changed to:
i. glycine (F1G R13G);
c. phenylalanine a position 1 changed to histidine in combination with arginine at position 13 changed to:
i. asparagine (F1H R13N);
d. phenylalanine at position 1 changed to isoleucine in combination with arginine at position 13 changed to:
i. alanine (F1I R13A),
ii. glycine (F1I R13G),
iii. isoleucine (F1I R13I),
iv. asparagine (F1I R13N),
v. proline (F1I R13P),
vi. glutamine (F1I R13Q),
vii. glutamic acid (F1I R13S),
viii. serine (F1I R13V), or
ix. valine (F1I R13E);
e. phenylalanine at position 1 changed to leucine in combination with arginine at position 13 changed to:
i. alanine (F1L R13A),
ii. aspartic acid (F1L R13D),
iii. glycine (F1L R13G),
iv. asparagine (F1L R13N),
v. proline (F1L R13P), or
vi. glutamine (F1L R13Q);
f. phenylalanine at position 1 changed to threonine in combination with arginine at position 13 changed to:
i. alanine (F1T R13A),
ii. asparagine (F1T R13N), or
iii. valine (F1T R13V);
g. phenylalanine at position 1 changed to valine in combination with arginine at position 13 changed to:
i. alanine (F1V R13A),
ii. asparagine (F1V R13N),
iii. glutamine (F1V R13Q),
iv. aspartic acid (F1V R13D),
v. valine (F1V R13V), or
vi. proline (F1V R13P); or
h. phenylalanine at position 1 changed to tyrosine in combination with arginine at position 13 changed to:
i. aspartic acid (F1Y R13D), or
ii. glycine (F1Y R13G).

22. The non-naturally occurring, post-translationally modified lantibiotic of claim 21, comprising two mutations including phenylalanine at position 1 changed to valine in combination with arginine at position 13 changed to:
i. alanine (F1V R13A),
ii. asparagine (F1V R13N),
iii. glutamine (F1V R13Q),
iv. aspartic acid (F1V R13D),
v. valine (F1V R13V), or
vi. proline (F1V R13P).

23. The non-naturally occurring, post-translationally modified lantibiotic of claim 22, wherein the two mutations consist of phenylalanine at position 1 changed to valine in combination with arginine at position 13 changed to asparagine (F1V R13N) (SEQ ID NO: 550).

24. A non-naturally occurring, post-translationally modified lantibiotic comprising an amino acid sequence of MU1140 (SEQ ID NO: 1156) and further comprising three mutations including:
a. phenylalanine at position 1 changed to isoleucine, in combination with arginine at position 13 changed to alanine, in combination with glycine at position 15 changed to alanine (F1I R13A G15A);
b. phenylalanine at position 1 changed to isoleucine, in combination with arginine at position 13 changed to aspartic acid, in combination with glycine at position 15 changed to alanine (F1I R13D G15A);
c. phenylalanine at position 1 changed to isoleucine, in combination with tryptophan at position 4 changed to isoleucine, in combination with arginine at position 13 changed to alanine (F1I W4I R13A);
d. phenylalanine at position 1 changed to isoleucine, in combination with tryptophan at position 4 changed to methionine, in combination with arginine at position 13 changed to aspartic acid (F1I W4M R13D);
e. phenylalanine at position 1 changed to isoleucine, in combination with tryptophan at position 4 changed to methionine, in combination with arginine at position 13 changed to asparagine (F1I W4M R13N);
f. phenylalanine at position 1 changed to isoleucine, in combination with lysine at position 2 changed to alanine, in combination with arginine at position 13 changed to alanine (F1I K2A R13A);
g. phenylalanine at position 1 changed to isoleucine, in combination with leucine at position 6 changed to valine, in combination with arginine at position 13 changed to alanine (F1I L6V R13A);
h. phenylalanine at position 1 changed to isoleucine, in combination with arginine at position 13 changed to alanine, in combination with tyrosine at position 20 changed to phenylalanine (F1I R13A Y20F);
i. phenylalanine at position 1 changed to isoleucine, in combination with arginine at position 13 changed to aspartic acid, in combination with tyrosine at position 20 changed to phenylalanine (F1I R13D Y20F);
j. phenylalanine at position 1 changed to isoleucine, in combination with arginine at position 13 changed to asparagine, in combination with tyrosine at position 20 changed to phenylalanine (F1I R13N Y20F);
k. phenylalanine at position 1 changed to leucine, in combination with arginine at position 13 changed to asparagine, in combination with tyrosine at position 20 changed to phenylalanine (F1L R13N Y20F);
l. phenylalanine at position 1 changed to leucine, in combination with arginine at position 13 changed to alanine, in combination with tyrosine at position 20 changed to phenylalanine (F1L R13A Y20F); or
m. phenylalanine at position 1 changed to leucine, in combination with arginine at position 13 changed to aspartic acid, in combination with tyrosine at position 20 changed to phenylalanine (F1L R13D Y20F).

25. A non-naturally occurring, post-translationally modified lantibiotic comprising an amino acid sequence of MU1140 (SEQ ID NO: 1156) and further comprising mutations wherein:
a. phenylalanine at position 1 changed to isoleucine, in combination with lysine at position 2 changed to alanine, in combination with tryptophan at position 4 changed to lysine, in combination with arginine at position 13 changed to alanine (F1I K2A W4K R13A); or
b. phenylalanine at position 1 changed to isoleucine, in combination with lysine at position 2 changed to alanine, in combination with tryptophan at position 4 changed to lysine, in combination with arginine at position 13 changed to aspartic acid (F1I K2A W4K R13D).

26. A non-naturally occurring, post-translationally modified lantibiotic comprising an amino acid sequence of MU1140 (SEQ ID NO: 1156) and further comprising mutations wherein phenylalanine at position 1 changed to isoleucine, in combination with lysine at position 2 changed to alanine, in combination with tryptophan at position 4 changed to methionine, in combination with arginine at position 13 changed to alanine, in combination with tyrosine at position 20 changed to phenylalanine (F1I K2A W4K R13A Y20F).

27. A non-naturally occurring lantibiotic comprising a single amino acid mutation of MU1140, the lantibiotic having an amino acid sequence encoded by of any one of SEQ ID NOS:2 to 431.

28. A non-naturally occurring lantibiotic comprising multisite amino acid mutations of MU1140, the lantibiotic being a variant as described in FIG. 7.

29. A non-naturally occurring lantibiotic comprising any variant as described in FIG. 8: Group 1, Group 2 or Group 3.

30. A non-naturally occurring lantibiotic comprising any variant as described in FIG. 8: Group 1.

31. A non-naturally occurring lantibiotic comprising a single amino acid mutation of MU1140 as described in FIG. 6.

32. A non-naturally occurring lantibiotic comprising an amino acid sequence of SEQ ID NO. 550.

33. The non-naturally occurring lantibiotic of any one of claim 27 to 32, wherein the lantibiotic is post-translationally modified.

34. The non-naturally occurring lantibiotic of claim 33, wherein the post-translation modification comprises Dha at position 5.

35. The non-naturally occurring lantibiotic of claim 33 or claim 34, wherein the post-translation modification comprises Abu at position 8.

36. The non-naturally occurring lantibiotic of any one of claim 33 to claim 35, wherein the post-translation modification comprises Dhb at position 14.

37. The non-naturally occurring lantibiotic of any one of claim 33 to claim 36, wherein the post-translation modification comprises Dha at position 5, Abu at position 8, and Dhb at position 14.

38. The non-naturally occurring lantibiotic of any one of claim 33 to claim 37, wherein the post-translation modification comprises a ring formed by lanthionine (Ala-S-Ala) residues between (Ala₃-S-Ala₇) as described in FIG. 1 (Ring A).

39. The non-naturally occurring lantibiotic of any one of claim 33 to claim 38, wherein the post-translation modification comprises a methyl-lanthionine residue (Abu-S-Ala) forming a ring, Ring B, comprising the alpha-aminobutyrate residue in position 8 and the Ala in position 11 (Abu₈-S-Ala₁₁) as described in FIG. 1.

40. The non-naturally occurring lantibiotic of any one of claim 33 to claim 39, wherein the post-translation modification comprises a ring formed by lanthionine (Ala-S-Ala) residues between (Ala₁₆-S-Ala₂₁) as described in FIG. 1 (Ring C).

41. The non-naturally occurring lantibiotic of any one of claim 33 to claim 40, wherein the post-translation modification comprises Ala in position 19 linked to an aminovinyl group by a thioether linkage (Ala₁₉-S—CH═CH—NH—), Ring D, as described in FIG. 1.

42. The non-naturally occurring lantibiotic of any one of claim 33 to claim 41, wherein the post-translation modification comprises Ring A, B, C and D.

43. The non-naturally occurring lantiobiotic of any one of claims 33 to 42, wherein the post-translationally modified MU1140 variant comprises Ring A, Ring B, Ring C and Ring D, wherein:
a. two of these rings are formed by lanthionine (Ala-S-Ala) residues, including one in Ring A (Ala₃-S-Ala₇) and one in Ring C (Ala₁₆-S-Ala₂₁);
b. a methyl-lanthionine residue (Abu-S-Ala) forms Ring B comprising the alpha-aminobutyrate residue in position 8 and the Ala in position 11 (Abu₈-S-Ala₁₁); and
c. the fourth ring, D, is comprised of the Ala in position 19 linked to an aminovinyl group by a thioether linkage (Ala₁₉-S—CH═CH—NH—).
and wherein amino acid at position 5 is modified to Dha, the amino acid at position 8 is modified to Abu, and the amino acid at position 14 is modified to Dhb.

44. An isolated lantibiotic variant, wherein the isolated lantibiotic variant is produced in a host cell by expressing a polypeptide from a polynucleotide encoding a lantibiotic variant of FIG. 5 or FIG. 7, and wherein the expressed polypeptide is post-translationally modified in the host cell.

45. The isolated lantibiotic variant of claim 44, wherein the host cell is S. mutans.

46. A post-translationally modified, non-naturally occurring lantibiotic comprising an amino acid sequence of SEQ ID NO. 1157.

47. An antimicrobial composition comprising a non-naturally occurring lantibiotic of any one of claims 1 to 26 or 33 to 46 and a pharmaceutically acceptable carrier, pharmaceutically acceptable diluent, other diluent or excipient.

48. The antimicrobial composition of claim 47, further comprising an antifungal agent, an additional antimicrobial agent, a membrane disrupting agent, or a combination thereof.

49. The antimicrobial composition of claim 48, wherein the additional antimicrobial agent has Gram negative bacteriostatic or bacteriocidal activity and the membrane disrupting agent renders Gram negative bacteria susceptible to the variant lantibiotic.

50. The antimicrobial composition of any one of claims 47 to 49, wherein the non-naturally occurring lantibiotics is present in the composition at about 0.001, 0.01, 0.1, 1, 5, 10, 20, 30, 40, 50, 75, 100, 150, 200, 300, 400, 500, 600, 700, 800, 900, or 1,000 mg/kg or mg/L.

51. A method of reducing reproduction of bacteria or reducing numbers of bacteria present in or on a subject, comprising administering to the subject a therapeutically effective amount of the antimicrobial composition of any one of claims 47 to 50.

52. The method of claim 51, wherein the subject is a human.

53. The method of claim 47 or 52, wherein the composition is administered orally, topically, nasally, buccally, sublingually, transmucosally, rectally, transdermally, by inhalation, by injection or intrathecally.

54. The method of claim 53, wherein the injection is intramuscular, intravenous, intrapulmonary, intramuscular, intradermal, intraperitoneal, intrathecal, or subcutaneous injection.

55. A preservative comprising an effective amount of the non-naturally occurring lantibiotic of any one of claims 1 to 26 or 33 to 46 in a physiological solution at a pH of between 3 and 8.

56. A food, beverage, gum, or dentifrice composition comprising an amount of the non-naturally occurring lantibiotic of any one of claims 1 to 26 or 33 to 46 sufficient to reduce the reproduction of bacteria or numbers of bacteria in the composition.

57. A method of reducing reproduction of bacteria or reducing numbers of bacteria present in or on a composition or object, comprising contacting the antimicrobial composition of any one of claims 1 to 26 or 33 to 46 with the composition or object for a period effective to reduce reproduction of bacteria or reduce numbers of bacteria in or on the composition or object.

58. The method of claim 57, wherein the composition is a food, beverage, gum, or dentifrice.

59. A composition comprising a solid surface or a textile with the lantibiotic composition of any one of claims 1 to 26 or 33 to 46 or coated onto, immobilized, linked, or bound to the solid surface or textile.

60. A method of reducing a biofilm or biofouling condition comprising contacting the antimicrobial composition of any one of claims 47 to 50 with the biofilm or biofouling condition for a period effective to reduce reproduction of bacteria or reduce numbers of bacteria in or on the biofilm or biofouling condition.

61. A kit comprising the lantibiotic of any one of claims 1 to 26 or 33 to 46 and one or more applicators.

62. A method of preventing or treating a subject diagnosed with a bacterial infection, comprising administering the non-naturally occurring lantibiotic of any one of claims 1 to 26 or 33 to 46.

63. The method of claim 62, wherein the subject is a human.

64. The method of claim 62 or 63, wherein the subject is infected with a Gram-positive bacteria.

65. The method of claim 64, wherein the Gram-positive bacteria is one or more of Staphylococcus epidermidis, vancomycin resistant Enterococci, vancomycin resistant Enterococcus faecalis, Enterococcus faecalis, Enterococcus faecium, Propionibacterium acnes, Streptococcus salivarius, Streptococcus sanguis, Streptococcus mitis, Streptococcus pyogenes, Lactobacillus salivarius, Listeria monocytogenes, Actinomyces israelii, Actinomyces naeslundii, Actinomyces viscosus, Bacillus anthracis, Streptococcus agalactiae, Streptococcus intermedius, Streptococcus pneumoniae, Corynebacterium diphtheria, Clostridium sporogenes Clostridium botulinum, Clostridium perfringens, Clostridium tetani, or Clostridium difficile.

66. The method of claim 65, wherein the Gram-positive bacteria is Clostridium difficile.

67. The method of claim 62 or 63, wherein the subject is infected with a Gram-negative bacteria.

68. The method of claim 67, wherein the Gram-negative bacteria is one or more of Acinetobacter baumanii, Bordatella pertussis, Borrelia burgdotieri, Brucella abortus, Brucella canis, Brucella melitensis, Brucella suis, Campylobacter jejuni, Coxiella burnetii, Escherichia coli, Francisella tularensis, Haemophilus influenza, Helicobacter pylori, Klebsiella pneumoniae, Legionella pneumophila, Leptospira interrogans, Neisseria gonorrhoeae, Neisseria meningitides, Pseudomonas aeruginosa, Rickettsia rickettsii, Salmonella enteritidis, Salmonella typhi, Salmonella typhimurium, Serratia marcescens, Shigella sonnei, Treponema pallidum, Vibrio cholera, Yersinia enterocolitica, or Yersinia pestis.

69. The method of any one of claims 62 to 68, wherein the non-naturally occurring lantibiotic further comprises one or more additional antimicrobial agents, membrane disrupting agents, or combinations thereof.

70. An antimicrobial composition, comprising the non-naturally occurring lantibiotic of claim 46, and a pharmaceutically acceptable carrier, pharmaceutically acceptable diluent, other diluent, excipient, or combinations thereof.

71. The antimicrobial composition of claim 47, further comprising an antifungal agent, an additional antimicrobial agent, a membrane disrupting agent, or a combination thereof.

72. The antimicrobial composition of claim 48, wherein the additional antimicrobial agent has Gram negative bacteriostatic or bacteriocidal activity and the membrane disrupting agent renders Gram negative bacteria susceptible to the variant lantibiotic.

73. The antimicrobial composition of any one of claims 70 to 72, wherein the non-naturally occurring lantibiotic is present in the composition at about 0.001, 0.01, 0.1, 1, 5, 10, 20, 30, 40, 50, 75, 100, 150, 200, 300, 400, 500, 600, 700, 800, 900, or 1,000 mg/kg or mg/L.

74. A method of reducing reproduction of bacteria or reducing numbers of bacteria present in or on a subject, comprising administering to the subject a therapeutically effective amount of the antimicrobial composition of any one of claims 70 to 73.

75. The method of claim 74, wherein the subject is a human.

76. The method of claim 73 or 74, wherein the composition is administered orally, topically, nasally, buccally, sublingually, transmucosally, rectally, transdermally, by inhalation, by injection, or intrathecally.

77. The method of claim 75, wherein the injection is intramuscular, intravenous, intrapulmonary, intramuscular, intradermal, intraperitoneal, intrathecal, or subcutaneous injection.

78. A preservative comprising an effective amount of the non-naturally occurring lantibiotic of claim 46 in a physiological solution at a pH of between 3 and 8.

79. A food, beverage, gum, or dentifrice composition comprising an amount of the non-naturally occurring lantibiotic of claim 46 sufficient to reduce the reproduction of bacteria or numbers of bacteria in the composition.

80. A method of reducing reproduction of bacteria or reducing numbers of bacteria present in or on a composition or object, comprising contacting the non-naturally occurring lantibiotic of claim 46 with the composition or object for a period effective to reduce reproduction of bacteria or reduce numbers of bacteria in or on the composition or object.

81. The method of claim 80, wherein the composition is a food, beverage, gum, or dentifrice.

82. A composition comprising a solid surface or a textile with the non-natrually occurring lantibiotic of claim 46 or coated onto, immobilized, linked, or bound to the solid surface or textile.

83. A method of reducing a biofilm or biofouling condition comprising contacting the non-naturally occurring lantibiotic of claim 46 with the biofilm or biofouling condition for a period effective to reduce reproduction of bacteria or reduce numbers of bacteria in or on the biofilm or biofouling condition.

84. A kit comprising the lantibiotic of claim 46 and one or more applicators.

85. A method of preventing or treating a subject diagnosed with a bacterial infection, comprising administering the non-naturally occurring lantibiotic of claim 46.

86. The method of claim 85, wherein the subject is a human.

87. The method of claim 85 or 86, wherein the subject is infected with a Gram-positive bacteria.

88. The method of claim 87, wherein the Gram-positive bacteria is one or more of Staphylococcus epidermidis, vancomycin resistant Enterococci, vancomycin resistant Enterococcus faecalis, Enterococcus faecalis, Enterococcus faecium, Propionibacterium acnes, Streptococcus salivarius, Streptococcus sanguis, Streptococcus mitis, Streptococcus pyogenes, Lactobacillus salivarius, Listeria monocytogenes, Actinomyces israelii, Actinomyces naeslundii, Actinomyces viscosus, Bacillus anthracis, Streptococcus agalactiae, Streptococcus intermedius, Streptococcus pneumoniae, Corynebacterium diphtheria, Clostridium sporogenes Clostridium botulinum, Clostridium perfringens, Clostridium tetani, or Clostridium difficile.

89. The method of claim 88, wherein the Gram-positive bacteria is Clostridium difficile.

90. The method of claim 85 or 86, wherein the subject is infected with a Gram-negative bacteria.

91. The method of claim 90, wherein the Gram-negative bacteria is one or more of Acinetobacter baumanii, Bordatella pertussis, Borrelia burgdotieri, Brucella abortus, Brucella canis, Brucella melitensis, Brucella suis, Campylobacter jejuni, Coxiella burnetii, Escherichia coli, Francisella tularensis, Haemophilus influenza, Helicobacter pylori, Klebsiella pneumoniae, Legionella pneumophila, Leptospira interrogans, Neisseria gonorrhoeae, Neisseria meningitides, Pseudomonas aeruginosa, Rickettsia rickettsii, Salmonella enteritidis, Salmonella typhi, Salmonella typhimurium, Serratia marcescens, Shigella sonnei, Treponema pallidum, Vibrio cholera, Yersinia enterocolitica, or Yersinia pestis.

92. The method of any one of claims 85 to 91, wherein the non-naturally occurring lantibiotic further comprises one or more additional antimicrobial agents, membrane disrupting agents, or combinations thereof.

93. A purified polynucleotide comprising any one of SEQ ID NOs: 2-431 or encoding a variant as described in FIG. 7 or combinations thereof.

94. A purified polynucleotide comprising any one of SEQ ID NOs: 756, 757, 758, 759, or 760.

95. A purified polynucleotide comprising any one of SEQ ID NOs: 709, 714, 716, 735, 747, 751, 754, or 758.

96. A purified polynucleotide comprising SEQ ID NO: 758.

97. A purified polynucleotide comprising SEQ ID NO: 1163.

98. A host cell comprising a polynucleotide of SEQ ID NO: 758.

99. A host cell comprising a polynucleotide of SEQ ID NO: 1163.

100. An isolated recombinant Streptococcus mutans strain comprising a polynucleotide of SEQ ID NO: 758.

101. An isolated recombinant Streptococcus mutans strain comprising a polynucleotide of SEQ ID NO: 1163.

102. A non-naturally occurring polypeptide comprising an amino acid sequence of SEQ ID NO. 1161.

103. An isolated recombinant Streptococcus mutans strain comprising: (a) a mutation in a polynucleotide involved in lactic acid synthesis such that expression of lactic acid is diminished by about 80% or more as compared to a wildtype S. mutans strain; (b) a recombinant alcohol dehydrogenase polynucleotide; and (c) a recombinant polynucleotide encoding the non-naturally occurring lantibiotic of any one of claims 1 to 26 or 33 to 46
